SEQUENCE LISTING (80 sequences)

SEQ ID NO: 1 - DNA, 1047 bp, Artificial sequence
  C. pomonella CPO_CPRQ desaturase (AHW98354) codon optimized for Y. lipolytica; mRNA-coding sequence
  misc_feature (1)..(1047): C. pomonella CPO_CPRQ desaturase (AHW98354) codon optimized for Y. lipolytica; mRNA-coding sequence
atgcctccccgagagtctaagaaggtggccctgcgatcttacgagacccctgtcgcttctctccctcctcgaaagtacgagattatctacctcaacctcttcctgcacatcgctggacatatctccgccgtctacggcctgtatctgtgcttcaccgccgcccagtggaagaccatcttctttgcctacctgtggctgttgatgggcgagctcggcgtggtgtgtggcgctcacagattgtggtctcaccgttctttcaaggtgaagcctcctctcgagatcatgctgatgctgttcaactgtattggattccagaacaccgccactgactgggtccgaaaccaccggctccatcacaagcactctgacactgacgccgacccccataactctaaccgaggaatgctgttctcccacattggctggctgtgtgtgcgaaagcacccagatgttaaagaacgaggcaagaccaccgacatgtctgacatctactctaaccccgtgctccgattccagaagaagcacaaggtaccccttttcggcgccatgtgtttcggcctgcccacccttattcccaccctgtggggagaggacatcgtcaccgcttggcacgtcaacctgctgcgattcgttcttaatctgaactctatcctgctggtcaactccattgctcataagtacggcacccgaccctacgatcgaaccatctgccctcgacaaaacaccacctgtaacatgatgactcttggagagggcttccacaactaccaccacacctttccttgggactaccgatctgccgagctgggaaagaactacctgaacttcaccaagtggttcatcgacttcttcgccctgattggatgggcctacgacctgaagaccgttcctgacgatatgatccagcgacgaatgaaaagaaccggagacggatccaactcgtggggatggggagacaaggacatgactaaggaggagcgagactctgctactatcatttatcccgagaagaaggatgatattaagatgatctccaaaaagaactaa

SEQ ID NO: 2 - protein, 348 aa, Cydia pomonella
  MISC_FEATURE (1)..(348): C. pomonella CPO_CPRQ desaturase (AHW98354)
MPPRESKKVALRSYETPVASLPPRKYEIIYLNLFLHIAGHISAVYGLYLCFTAAQWKTIFFAYLWLLMGELGVVCGAHRLWSHRSFKVKPPLEIMLMLFNCIGFQNTATDWVRNHRLHHKHSDTDADPHNSNRGMLFSHIGWLCVRKHPDVKERGKTTDMSDIYSNPVLRFQKKHKVPLFGAMCFGLPTLIPTLWGEDIVTAWHVNLLRFVLNLNSILLVNSIAHKYGTRPYDRTICPRQNTTCNMMTLGEGFHNYHHTFPWDYRSAELGKNYLNFTKWFIDFFALIGWAYDLKTVPDDMIQRRMKRTGDGSNSWGWGDKDMTKEERDSATIIYPEKKDDIKMISKKN

SEQ ID NO: 3 - DNA, 384 bp, Artificial sequence
  H. armigera cytochrome b5 (AAC33731) codon optimized for Y. lipolytica; mRNA-coding sequence
  misc_feature (1)..(384): H. armigera cytochrome b5 (AAC33731) codon optimized for Y. lipolytica; mRNA-coding sequence
atgaccgtgcgacagttcacccgagtcgaggtgtctaagtggaccactcgagaggaagccgtgttcatcatcgacaacgtggtgtacaacgtgaccaagttcctggacgagcaccccggtggacacgaggtgctggtgaacgtggccggcaaggacgcctctgaggacttcgacgacgtgggccactctctggacgccaaggaactgatgaagaagtacgtcgtcggcgaggtggtcgaggccgagcgacgacacatccagaagcgacagatctcttgggaagattctaaggtggactctgactcttctttcacctcttcgtggaagttccccgtgctgctgggcatcgtggtgaccctgctgtacacctacctgttcggctaa

SEQ ID NO: 4 - protein, 127 aa, Helicoverpa armigera
  MISC_FEATURE (1)..(127): H. armigera cytochrome b5 (AAC33731)
MTVRQFTRVEVSKWTTREEAVFIIDNVVYNVTKFLDEHPGGHEVLVNVAGKDASEDFDDVGHSLDAKELMKKYVVGEVVEAERRHIQKRQISWEDSKVDSDSSFTSSWKFPVLLGIVVTLLYTYLFG

SEQ ID NO: 5 - DNA, 441 bp, Artificial sequence
  V. stercoraria hemoglobin (AAT01097) codon optimized for Y. lipolytica; mRNA-coding sequence
  misc_feature (1)..(441): V. stercoraria hemoglobin (AAT01097) codon optimized for Y. lipolytica; mRNA-coding sequence
atgctggaccagcagaccgtggacacctctaaggccaccgtgcctgtgctgaaggaacacggcgtgaccatcaccaccaccttctaccagaacctgttcgctaagcaccccgaggtgcgacccctgttcgatatgggccgacaggcctctctcgagcagcccaaggctctggccatgaccgtgggagccgccgctcagaacatcgagaacctgcctgccattctgcccgccgtgcagaagatcgccgtcaagcactgccaggccggcgtggccgctcgacactaccccatcgtgggccaagagctgctgggcgccatcaaggaactgctgggtgacgccgccaccgacgacatcctggacgcctggggcaaggcctacggcgtgatcgccgacgtgttcatccaggtcgaggccgacctgtacgcccaggacgccgagtaa

SEQ ID NO: 6 - protein, 146 aa, Vitreoscilla stercoraria
  MISC_FEATURE (1)..(146): V. stercoraria hemoglobin (AAT01097)
MLDQQTVDTSKATVPVLKEHGVTITTTFYQNLFAKHPEVRPLFDMGRQASLEQPKALAMTVGAAAQNIENLPAILPAVQKIAVKHCQAGVAARHYPIVGQELLGAIKELLGDAATDDILDAWGKAYGVIADVFIQVEADLYAQDAE

SEQ ID NO: 7 - DNA, 1557 bp, Artificial sequence
  T. alba fatty acyl reductase (NP_001289627) codon optimized for Y. lipolytica; mRNA-coding sequence
  misc_feature (1)..(1557): T. alba fatty acyl reductase (NP_001289627) codon optimized for Y. lipolytica; mRNA-coding sequence
atggtgtctatccccgagtactacgagggcaagaacatcctgctgaccggcgccaccggcttcatgggcaaggtgctgctcgagaagctgctgcgatcttgccccaaggtgaaggccgtgtacgtgctggtgcgacacaaggccggacagacccctgaggctcgaatcgaggaaatcaccaactgcaagctgttcgaccgactgcgagatgagcagcccgacttcaaggccaagatcatcgtgatcacctctgagctgacccagcctgagctggacctgtctgagcccatcaaggaaaagctgatcgagcgaatcaacatcatcttccactgcgccgccaccgtgcgattcaacgagactctccgagatgccgtgcagctgaacgtgaccgctactcagcagctcctgttcctggctcagcgaatgaagaacctggaagtgttcatgcacgtgtctaccgcctacgcctactgcaaccgaaagcagatcgaagagatcgtgtaccctcctccagtggaccccaagaagctgattgactctctcgagtggatggacgacggcctggtgaacgacatcacccctaagctcatcggcgaccgacctaacacctacacttacaccaaggctctggccgagtacgtggtgcagcaagagggcgccaagctgaacaccgccatcattcgaccctctatcgtgggcgcctcttggaaggaaccctttcctggctggatcgacaacttcaacggcccctctggcctgttcattgccgccggaaagggcatcctgcgaaccatgcgagcctctaactctgccgtggccgacctggtgcctgtggacgtggtggtgaacaccactctggccgctgcctggtactctggcgtgaaccgacctcgaaacgtgatgatctacaactgcaccaccggcggcactaaccccttccactggggcgaagtgggctaccacatcaacctgaacttcaagatcaaccctctcgagaacgccgtgcgacaccccaactgttctctgcagtctaaccctctgctccatcagtactggaccgccgtgtctcacaccatgcctgcctttctgctggacctcctgctgcgactgaccggacacaagccctggatgatgaagaccatcactcgactgcacaaggccatgatgctcctcgagtacttcacctccaactcttggatctggaacaccgagaacatgaccatgctgatgaaccagctgaaccccgaggacaagaagaccttcaacttcgacgtgcgacagctgcactgggctgagtacatggaaaactactgcatgggcaccaagaagtacgtcctgaacgaggaaatgtctggactgcccgctgccagaaagcacctgaacaagctgcgaaacatccgatacggcttcaacaccgtgctggtcatcctgatctggcgaatcttcattgcccgatctcagatggcccgaaacatctggtacttcgtggtgtctctgtgctacaagttcctgtcttacttccgagcctcttctaccatgcgatactctaagctgtag

SEQ ID NO: 8 - protein, 515 aa, Tyto alba
  MISC_FEATURE (1)..(515): T. alba fatty acyl reductase (NP_001289627)
MVSIPEYYEGKNILLTGATGFMGKVLLEKLLRSCPKVKAVYVLVRHKAGQTPEARIEEITNCKLFDRLRDEQPDFKAKIIVITSELTQPELDLSEPIKEKLIERINIIFHCAATVRFNETLRDAVQLNVTATQQLLFLAQRMKNLEVFMHVSTAYAYCNRKQIEEIVYPPPVDPKKLIDSLEWMDDGLVNDITPKLIGDRPNTYTYTKALAEYVVQQEGAKLNTAIIRPSIVGASWKEPFPGWIDNFNGPSGLFIAAGKGILRTMRASNSAVADLVPVDVVVNTTLAAAWYSGVNRPRNVMIYNCTTGGTNPFHWGEVGYHINLNFKINPLENAVRHPNCSLQSNPLLHQYWTAVSHTMPAFLLDLLLRLTGHKPWMMKTITRLHKAMMLLEYFTSNSWIWNTENMTMLMNQLNPEDKKTFNFDVRQLHWAEYMENYCMGTKKYVLNEEMSGLPAARKHLNKLRNIRYGFNTVLVILIWRIFIARSQMARNIWYFVVSLCYKFLSYFRASSTMRY

SEQ ID NO: 9 - DNA, 1377 bp, Artificial sequence
  A. segetum fatty acyl reductase (AGP26039) codon optimized for Y. lipolytica; mRNA-coding sequence
  misc_feature (1)..(1377): A. segetum fatty acyl reductase (AGP26039) codon optimized for Y. lipolytica; mRNA-coding sequence
atgcccgtgctgacctcgcgagaggacgagaagctgtctgtgcccgagttctacgccggcaagtctatcttcgtgaccggcggcaccggattcctcggcaaggtgttcattgagaagctgctctactgctgccccgacatcgacaagatctacatgctgatccgagagaagaagaacctgtctatcgacgagcgaatgtctaagttcctggacgaccctctgttctctcgactgaaggaagaacgacccggcgacctcgagaagatcgtgctgatccccggcgacatcaccgctcctaacctgggcctgtctgccgagaacgaacgaatcctgctcgagaaggtgtccgtgatcatcaactctgccgccaccgtgaagttcaacgagcccctgcctatcgcctggaagatcaacgtcgagggcacccgaatgctgctggccctgtctcgacgaatgaagcgaatcgaggtgtttatccacatctctaccgcctactctaacgcctcttctgaccgaatcgtggtggacgagattctgtaccccgctcctgccgacatggaccaggtgtaccagctcgtgaaggacggcgtgaccgaggaagagactgagcgactgctgaacggactgcccaacacctacaccttcaccaaggctctgaccgagcacctggtggccgagcaccagacctacgtgcccaccatcatcattcgaccctccgtggtggcctctatcaaggacgagcccatccgaggctggctgtgcaactggttcggcgccaccggcatctctgtgttcaccgccaagggcctgaaccgagtgctgctcggaaaggcctctaacatcgtggacgtgatccccgtggactacgtggccaacctggtgatcgtggctggcgccaagtctggcggccagaagtctgacgagctgaagatctataactgctgttcttctgactgcaaccccgtgactctgaagaagatcatcaaggaattcaccgaggacaccatcaagaacaagtctcacatcatgcctctgcctggctggttcgtgttcaccaagtacaagtggctgctgaccctcctgaccatcatcttccagatgctgcccatgtacctggccgacgtgtaccgagtcctgaccggcaagattccccggtacatgaagctgcaccacctggtcattcagacccgactgggaatcgacttcttcacctctcactcttgggtgatgaagaccgaccgagtgcgagagctgttcggctctctgtctctggccgagaagcacatgttcccttgcgacccctcttccatcgactggaccgactacctgcagtcttactgctacggcgtgcgacgattcctggaaaagaagaagtag

SEQ ID NO: 10 - protein, 458 aa, Agrotis segetum
  MISC_FEATURE (1)..(458): A. segetum fatty acyl reductase (AGP26039)
MPVLTSREDEKLSVPEFYAGKSIFVTGGTGFLGKVFIEKLLYCCPDIDKIYMLIREKKNLSIDERMSKFLDDPLFSRLKEERPGDLEKIVLIPGDITAPNLGLSAENERILLEKVSVIINSAATVKFNEPLPIAWKINVEGTRMLLALSRRMKRIEVFIHISTAYSNASSDRIVVDEILYPAPADMDQVYQLVKDGVTEEETERLLNGLPNTYTFTKALTEHLVAEHQTYVPTIIIRPSVVASIKDEPIRGWLCNWFGATGISVFTAKGLNRVLLGKASNIVDVIPVDYVANLVIVAGAKSGGQKSDELKIYNCCSSDCNPVTLKKIIKEFTEDTIKNKSHIMPLPGWFVFTKYKWLLTLLTIIFQMLPMYLADVYRVLTGKIPRYMKLHHLVIQTRLGIDFFTSHSWVMKTDRVRELFGSLSLAEKHMFPCDPSSIDWTDYLQSYCYGVRRFLEKKK

SEQ ID NO: 11 - DNA, 1368 bp, Artificial sequence
  H. armigera fatty acyl reductase (ATJ44471) codon optimized for Y. lipolytica; mRNA-coding sequence
  misc_feature (1)..(1368): H. armigera fatty acyl reductase (ATJ44471) codon optimized for Y. lipolytica; mRNA-coding sequence
atggtggtcctgacctctaaggagactaagccctccgtggccgagttctacgctggcaagtctgtcttcatcaccggcggaaccggtttcctgggcaaggtcttcattgagaagctgctgtactcctgtcccgacatcggcaacatctacatgctgatccgagagaagaagggactgtctgtgtccgagcgaattaagcacttcctggacgaccccctgttcacccgactgaaggagaagcgacccgccgacctggagaagatcgtgctgattcccggagacatcaccgctcccgacctgggtattacctctgagaacgagaagatgctgatcgagaaggtgtctgtcatcattcactccgccgctaccgtcaagttcaacgagcccctgcccaccgcctggaagatcaacgtggagggaacccgaatgatgctggctctgtctcgacgaatgaagcgaattgaggtcttcatccacatttccaccgcctacaccaacaccaaccgagaggtggtggacgagatcctgtaccctgctcctgctgacattgaccaggtgcaccgatacgtcaaggacggtatctctgaggaagagactgagaagattctgaacggccgacccaacacctacaccttcaccaaggccctgaccgagcacctggtggctgagaaccaggcttacgtgcccaccatcattgtccgaccctccgtggtcgccgctatcaaggacgagcccattaagggatggctgggtaactggtacggagctaccggactgaccgtgttcaccgctaagggtctgaaccgagtcatctacggccactcttccaacatcgtggacctgattcccgtggactacgtcgccaacctggtcattgccgctggcgctaagtcttccaagtccaccgagctgaaggtgtacaactgttgctcttccgcctgcaaccccatcaccattggaaagctgatgtctatgttcgccgaggacgctatcaagcagaagtcctacgctatgcccctgcccggttggtacatcttcaccaagtacaagtggctggtcctgctgctgaccattctgttccaggtcatccccgcctacattaccgacctgtaccgacacctgatcggcaagaacccccgatacattaagctgcagtctctggtcaaccagacccgatcttccattgacttcttcacctctcactcctgggtcatgaaggctgaccgagtccgagagctgttcgcctctctgtcccccgctgacaagtacctgttcccctgtgaccccaccgacatcaactggacccactacattcaggactactgctggggagtgcgacacttcctggagaagaagtcctacgagtag

SEQ ID NO: 12 - protein, 456 aa, Helicoverpa armigera
  MISC_FEATURE (1)..(456): H. armigera fatty acyl reductase (ATJ44471)
MVVLTSKETKPSVAEFYAGKSVFITGGTGFLGKVFIEKLLYSCPDIGNIYMLIREKKGLSVSERIKQFLDDPLFTRLKEKRPADLEKIVLIPGDITAPDLGITSENEKILIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTDLKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKTTNK

SEQ ID NO: 13 - DNA, 915 bp, Yarrowia lipolytica
  misc_feature (1)..(915): Y. lipolytica fatty acid elongase 1 (YALI0_F06754g)
atgctctcgtcaatctcgcccgacctatactcgtccttctcgttcaaaaactcgctcgccgaggccatgccctccgtgccacacgaactcatcaactcaaaaacactctcatggatgtacaatgcctctctggacattcgggttcctctgactatcggaaccatctacgccgtctccgtgcacctgaccaactcatctgaacgaatcaagaaacgccagcccattgcctttgccaagaccgcactcttcaagtggctctgtgtcctccacaatgcaggtctgtgtctctactcagcatggacctttgtcggtatcctcaacgccgtcaaacacgcctaccaaatcacaggagacagctccgcccccttctccttcaacaccctctggggatcgttttgttcacgtgactccctctgggtcaccggcctcaactactacggatactggttctatctgtccaaattctacgaagtggtggacaccatgatcatcctcgcaaagggaaaaccgtcctcaatgctccagacataccaccacaccggcgccatgttctccatgtgggccggcatccgattcgcctctccccccatctggatctttgtggttttcaactccctcatccacacaatcatgtacttttactacaccctcaccaccctcaagatcaaggttcccaagatcctcaaggcatctctgaccaccgcccagatcacccagattgtcggaggtggcatcctggctgcctcccacgcctttatttattacaaggaccaccagactgagaccgtctgttcttgtctcactacccagggtcagtttttcgctctcgccgtcaatgtcatctatctgagtcctctggcctatctctttattgccttctggattcgatcttacttgaaggccaagtccaactag

SEQ ID NO: 14 - protein, 304 aa, Yarrowia lipolytica
  MISC_FEATURE (1)..(304): Y. lipolytica fatty acid elongase 1 (XP_505094)
MLSSISPDLYSSFSFKNSLAEAMPSVPHELINSKTLSWMYNASLDIRVPLTIGTIYAVSVHLTNSSERIKKRQPIAFAKTALFKWLCVLHNAGLCLYSAWTFVGILNAVKHAYQITGDSSAPFSFNTLWGSFCSRDSLWVTGLNYYGYWFYLSKFYEVVDTMIILAKGKPSSMLQTYHHTGAMFSMWAGIRFASPPIWIFVVFNSLIHTIMYFYYTLTTLKIKVPKILKASLTTAQITQIVGGGILAASHAFIYYKDHQTETVCSCLTTQGQFFALAVNVIYLSPLAYLFIAFWIRSYLKAKSN

SEQ ID NO: 15 - DNA, 6337 bp, Yarrowia lipolytica
  misc_feature (1)..(6337): Y. lipolytica fatty acid synthase 1 (YALI0_B15059g)
atggtgagtatcgaccgaagcaggatgatctctacatgagatatgcaacgcgtacgtgattcaattgatcctaacacagtaccctaccacaggtgtcaacaccccccagagcgccgcctcattaagaccactggtgctatcgcacggccaaactgagcactcgctgctggtgcccacctctctgtacatcaactgcaccacgctccgagaccagttctacgcctctctacctccagccactgaagacaaggccgacgatgatgagccctcctcctccacagagcttctagctgccttcctgggatttactgccaagaccgtcgaggaagagcccggaccatacgacgacgttctctctctcgtgcttaacgagtttgagacccggtacttgcgaggtaacgacatccacgctgtggcctcctccttgttacaagacgaggacgtgcctaccaccgttggtaagatcaagagggtgattcgagcctactacgccgcacgaattgcctgcaaccggcccatcaaggcccactcgtcggctctgttccgagccgcatctgaagactcggacaacgtctctctgtacgccatcttcggtggccagggaaacaccgaggactactttgaggaactgcgggagatttacgacatctaccaggggctggtcggcgacttcattcgggaatgtggagcccagcttctggcgctgtctcgagatcacattgctgctgagaaaatttataccaagggctttgatatcgtcaagtggctggaacaccccgagaccatccccgactttgagtacctaatttctgctcccatctctgtacccatcatcggtgttatccagctggcacactacgctgtcacctgtcgagttttgggtcttaatcctggccaggtccgagacaacctcaagggtgccactggccattctcagggtctgatcaccgcaattgccatctctgcctccgactcgtgggacgagttctataactctgcctctcgaattctcaagatcttcttcttcatcggtgtccgtgtccaacaggcttacccctccactttcctgcctccctccactctggaagacagtgtcaagcagggtgagggcaagcccactcccatgctgtccatccgagacctgtctctcaaccaggttcaggagttcgtcgatgccaccaacttgcatttgcccgaagataagcagatcgtcgtgtctctgatcaatggtcctcgaaacgttgtcgttactggccccccccagtctctgtatggtctgtgtcttgtgcttcgaaaacagaaggccgagaccggtctggaccaaagccgagtgccccacagtcagcgaaagctcaaattcacacatcgtttcctgcccatcacctctcctttccactcgtacctgctggagaagagcacggatctgatcatcaacgacctggagtcttccggtgtggagtttgtgtcctccgagctcaaggtgcctgtttacgacacctttgatggctccgtgctgtctcagctacccaagggtatcgtcagccgtctggtcaacctcatcactcatctgcccgtcaagtgggagaaggccactcagtttcaggcctcccacattgtggactttggtcccggtggcgcttctggtcttggtctgttgacccacaagaacaaggatggaactggagtgcgaactattcttgctggtgtcattgaccagcccctcgagttcggcttcaagcaggagctgtttgaccgacaggagtcgtccattgtttttgctcaaaactgggccaaggagttttctcccaagctcgtcaagatctcctccaccaacgaggtctatgtcgacaccaaattctctcgtctgactggccgagcccccatcatggtcgctggtatgacccctaccactgtcaaccccaaatttgtggctgccactatgaactccggctaccacatcgagcttggtggtggaggctactttgcccccggtatgatgaccaaggcccttgaacacattgagaagaacactcctcccggatccggtatcaccatcaacctgatctacgtcaaccctcgactgattcaatggggtattcctctgattcaggagcttcgacagaagggtttccccattgaaggtctcaccattggtgccggtgtgccctctctggaggttgctaacgagtggattcaggatctgggcgtcaagcacatcgccttcaagcctggatccatcgaggccatctcctcggtgattcgaatcgccaaggccaacccagactttcctatcatccttcagtggaccggaggtcgaggaggaggacatcattcgtttgaggacttccacgctcccattctgcagatgtactccaagatccgacgatgcagcaacattgtgctgattgccggatctggtttcggtgcttctaccgactcctacccatacctcaccggttcatggtcccgagactttgactaccctcccatgccctttgacggtatcctggttggttctcgagtcatggttgccaaggaggctttcacttctctgggagccaagcagctcattgttgactctccgggtgttgaggattctgagtgggagaaaacctacgacaagcccactggtggcgtcatcaccgttctctccgagatgggtgagcctatccacaagctcgccactcgaggtgtgctcttctggcacgagatggacaagaccgtgttctccctgcccaagaagaagcgtctggaagtgctcaagtccaagcgagcctacatcatcaagcgtctcaacgacgacttccagaagacttggtttgccaagaacgcccagggacaggtgtgtgatctcgaagacctcacctacgcggaggtcatccagcgacttgttgacctcatgtacgtgaagaaggaaagccgatggatcgatgtcactctccgaaatcttgccggcactttcattcgacgagttgaggagcgattctccaccgagacaggtgcctcttctgtgttgcagagcttttccgagctggattccgagcccgagaaggttgtcgagcgggtgtttgagctcttccctgcctctactacccagatcatcaacgctcaagacaaggaccacttcctcatgctgtgtctcaaccccatgcagaagcccgtgcccttcatccctgttctggatgacaactttgagttcttcttcaagaaggactctctgtggcagtgcgaggacctcgcagctgttgtggacgaagacgttggacgaatctgtattcttcagggtcccgttgctgtcaagcactccaagattgtcaacgagcccgtcaaggagattctcgactccatgcacgaaggtcacatcaagcagctgcttgaggatggcgagtacgctggcaacatggccaacatcccccaggtcgaatgctttggtggaaagcctgctcagaacttcggtgacgttgctctcgactctgtcatggttcttgatgacctcaacaagaccgtgttcaagattgagaccggcacctctgctctgccttctgctgcagattggttctctctgctggccggtgacaagaactcttggcgacaggtcttcctgtccactgacaccattgtgcagaccaccaagatgatctccaaccctctgcatcgacttctggagcccatcgcaggtttgcaggttgagattgagcaccctgatgagcccgagaacaccgtcatctctgctttcgagcccatcaacggcaaggtcaccaaggtgctggagctgcgaaagggtgccggagacgtcatttcgctgcagctgatcgaagcgcgtggcgttgaccgagtccccgttgctcttcctctggaattcaagtaccagccccagattggctacgctcccattgttgaggttatgaccgacaggaacacccgaatcaaggagttctactggaagctgtggtttggccaggactccaagtttgagattgacaccgacatcaccgaggaaatcattggcgatgacgttaccatctctggcaaggccattgccgactttgtccacgctgttggcaacaagggcgaggcctttgttggtcgatctacctctgctggtactgtcttcgctcccatggactttgccattgttttgggctggaaggccattatcaaggcaatctttccccgagcaattgatgctgacattctgcgtctggtacatctgtccaacggcttcaagatgatgcctggcgccgaccctctgcagatgggtgatgttgtttccgccactgccaagatcgacactgtcaagaactccgctaccggcaagactgttgctgttcgaggtcttctcacccgagacggcaagcctgtcatggaggttgtttccgaattcttctaccgaggcgaattctccgacttccagaacacttttgagcgacgagaggaggtacccatgcaactgaccctcaaggacgccaaggccgtggccattctctgctccaaggagtggtttgagtacaatggcgacgataccaaggacctcgagggcaagaccattgtgttccgaaactcgtcattcatcaagtacaagaatgagaccgtcttctcttctgtgcacaccaccggtaaggtattgatggagctgccctccaaggaggtcattgagattgccactgttaactaccaggctggcgagtctcatggcaatcccgtcattgattacctggagcgaaatggaaccaccattgagcagcctgttgagtttgagaagcccatccctctgtccaaggcagatgatcttctctccttcaaggctccttcttccaacgagccctacgctggtgtgtccggtgactacaatcccatccacgtgtctcgagcctttgcttcctatgcatcccttcctggaaccatcacccacggtatgtactcttctgctgctgttcgatctctgattgaggtctgggctgccgagaacaatgtgtctcgagttcgagccttctcctgtcagttccagggcatggttttgcccaacgacgagattgtgactcgactggagcacgttggcatgatcaacggtcgaaagatcatcaaggttacctccaccaaccgggagaccgaggctgttgttctgtctggcgaggctgaggtcgagcagcccatctccacctttgtctttactggccagggctctcaggagcagggcatgggtatggacctgtacgcctcttccgaggtggccaagaaggtctgggacaaggctgacgagcacttcttgcagaactacggtttctccatcatcaagatcgttgtggagaaccccaaggagctggatattcattttggaggccccaagggtaagaagatccgagacaactatatctctatgatgttcgagaccattgatgagaagaccggcaacctcatttccgagaagatcttcaaggagattgacgagaccaccgactctttcaccttcaagtcccccaccggtctgctttctgctacccagttcactcagcccgctctgaccctcatggagaaggcgtcctttgaggacatgaaggctaagggtcttgtccccgtggatgcaacctttgctggtcactcccttggtgagtactccgctcttgcttctcttggtgatgtcatgcccatcgagtctcttgttgatgtcgtcttctaccgaggtatgactatgcaggttgctgttccccgagatgcccagggtcggtccaattacggtatgtgcgctgtcaacccctctcgaatctctaccaccttcaacgacgctgctcttcggtttgtcgttgaccacatctccgagcagaccaagtggctgcttgagattgtcaactacaacgttgagaactctcagtacgtgactgccggtgacctgcgagctctcgacaccctcaccaatgtgctcaacgtgctcaaactcgagaagatcaacattgacaagctgctcgagtctctgcctctggagaaggtcaaggagcacctttctgagatcgtcaccgaggtggccaagaagtccgttgctaagcctcagcccattgagctggaacgaggctttgccgtgatccctctcaagggcatctctgtgcctttccactcttcgtacctgcgaaatggtgtcaagcccttccaaaacttcctggtgaagaaggtgcccaagaacgctgtcaaacctgccaacctcattggcaagtacatccccaacctcactgccaagccctttgagatcaccaaggagtactttgaagaggtttacaagctcaccggttccgagaaggtcaagagcatcatcaacaactgggagtcttatgagtccaagcagtaa

SEQ ID NO: 16 - protein, 2086 aa, Yarrowia lipolytica
  MISC_FEATURE (1)..(2086): Y. lipolytica fatty acid synthase 1 (XP_500912)
MYPTTGVNTPQSAASLRPLVLSHGQTEHSLLVPTSLYINCTTLRDQFYASLPPATEDKADDDEPSSSTELLAAFLGFTAKTVEEEPGPYDDVLSLVLNEFETRYLRGNDIHAVASSLLQDEDVPTTVGKIKRVIRAYYAARIACNRPIKAHSSALFRAASEDSDNVSLYAIFGGQGNTEDYFEELREIYDIYQGLVGDFIRECGAQLLALSRDHIAAEKIYTKGFDIVKWLEHPETIPDFEYLISAPISVPIIGVIQLAHYAVTCRVLGLNPGQVRDNLKGATGHSQGLITAIAISASDSWDEFYNSASRILKIFFFIGVRVQQAYPSTFLPPSTLEDSVKQGEGKPTPMLSIRDLSLNQVQEFVDATNLHLPEDKQIVVSLINGPRNVVVTGPPQSLYGLCLVLRKQKAETGLDQSRVPHSQRKLKFTHRFLPITSPFHSYLLEKSTDLIINDLESSGVEFVSSELKVPVYDTFDGSVLSQLPKGIVSRLVNLITHLPVKWEKATQFQASHIVDFGPGGASGLGLLTHKNKDGTGVRTILAGVIDQPLEFGFKQELFDRQESSIVFAQNWAKEFSPKLVKISSTNEVYVDTKFSRLTGRAPIMVAGMTPTTVNPKFVAATMNSGYHIELGGGGYFAPGMMTKALEHIEKNTPPGSGITINLIYVNPRLIQWGIPLIQELRQKGFPIEGLTIGAGVPSLEVANEWIQDLGVKHIAFKPGSIEAISSVIRIAKANPDFPIILQWTGGRGGGHHSFEDFHAPILQMYSKIRRCSNIVLIAGSGFGASTDSYPYLTGSWSRDFDYPPMPFDGILVGSRVMVAKEAFTSLGAKQLIVDSPGVEDSEWEKTYDKPTGGVITVLSEMGEPIHKLATRGVLFWHEMDKTVFSLPKKKRLEVLKSKRAYIIKRLNDDFQKTWFAKNAQGQVCDLEDLTYAEVIQRLVDLMYVKKESRWIDVTLRNLAGTFIRRVEERFSTETGASSVLQSFSELDSEPEKVVERVFELFPASTTQIINAQDKDHFLMLCLNPMQKPVPFIPVLDDNFEFFFKKDSLWQCEDLAAVVDEDVGRICILQGPVAVKHSKIVNEPVKEILDSMHEGHIKQLLEDGEYAGNMANIPQVECFGGKPAQNFGDVALDSVMVLDDLNKTVFKIETGTSALPSAADWFSLLAGDKNSWRQVFLSTDTIVQTTKMISNPLHRLLEPIAGLQVEIEHPDEPENTVISAFEPINGKVTKVLELRKGAGDVISLQLIEARGVDRVPVALPLEFKYQPQIGYAPIVEVMTDRNTRIKEFYWKLWFGQDSKFEIDTDITEEIIGDDVTISGKAIADFVHAVGNKGEAFVGRSTSAGTVFAPMDFAIVLGWKAIIKAIFPRAIDADILRLVHLSNGFKMMPGADPLQMGDVVSATAKIDTVKNSATGKTVAVRGLLTRDGKPVMEVVSEFFYRGEFSDFQNTFERREEVPMQLTLKDAKAVAILCSKEWFEYNGDDTKDLEGKTIVFRNSSFIKYKNETVFSSVHTTGKVLMELPSKEVIEIATVNYQAGESHGNPVIDYLERNGTTIEQPVEFEKPIPLSKADDLLSFKAPSSNEPYAGVSGDYNPIHVSRAFASYASLPGTITHGMYSSAAVRSLIEVWAAENNVSRVRAFSCQFQGMVLPNDEIVTRLEHVGMINGRKIIKVTSTNRETEAVVLSGEAEVEQPISTFVFTGQGSQEQGMGMDLYASSEVAKKVWDKADEHFLQNYGFSIIKIVVENPKELDIHFGGPKGKKIRDNYISMMFETIDEKTGNLISEKIFKEIDETTDSFTFKSPTGLLSATQFTQPALTLMEKASFEDMKAKGLVPVDATFAGHSLGEYSALASLGDVMPIESLVDVVFYRGMTMQVAVPRDAQGRSNYGMCAVNPSRISTTFNDAALRFVVDHISEQTKWLLEIVNYNVENSQYVTAGDLRALDTLTNVLNVLKLEKINIDKLLESLPLEKVKEHLSEIVTEVAKKSVAKPQPIELERGFAVIPLKGISVPFHSSYLRNGVKPFQNFLVKKVPKNAVKPANLIGKYIPNLTAKPFEITKEYFEEVYKLTGSEKVKSIINNWESYESKQ

SEQ ID NO: 17 - DNA, 5947 bp, Yarrowia lipolytica
  misc_feature (1)..(5947): Y. lipolytica fatty acid synthase 2 (YALI0_B19382g)
agtgagtcttgaaatatgggatatgaggaggggtttgaagaggttgcaatcgataactcacgacacggacgaaaaagaataaggaccaacacgatctccagacaaccacagatcagcagtcgaacccccctcaacagcagacaaatgatgttgtggaattgcagtagatgatttcttctgcgacgctagtattggctgtcggcgacactattctctgacagtgcccaatggtctttttattgtgcaccaaccgctgatttgtggctcaggttttgtgacggcgagagtcattctcgtgatgcatgggatgattggtctctttgaagccgacagatcgacatatttccacacacagcaacgacaatgttatcttatccattgccattctaacccagtgcaccccgaagtcgaacaagaactcgcccacgtgctcctgacggagctgctggcctaccaatttgcctcgcccgtgcgatggatcgagacccaggacgtgctgttcaagcagttcaatgtcgagcgagtcgtcgaagtcggcccatccccaactctcgccggcatggcccagcgaacccttaagtccaagtacgagtcatacgacgctgctctgtctctgcagcgagagatcctgtgttactccaaggaccagaaggacatctactaccttgccgatgaggccgatgaagcccctgcccccgctgctggtggtgatgcccccgctgctcctgccgctgccgctcctgccgccgctgccgctcctgctgccgctgccgccccctctggccccgttgccaaggttgaggacgcccccgtcaaggcccaggagattctccacgccctggtcgcccataagctcaagaagacccccgagcaggtgcccctgtccaaggccatcaaagaccttgttggtggtaagtctaccatccagaacgagattctcggtgatctcggaaaggaatttggtgccacccctgagaagcccgaggatactccccttggcgagctggctgagtccttccaggcctcctttgacggcaagctcggtaagcagtcttcttctctcattgcccgactcatgtcctccaagatgcccggagggttctctctcacctctgctcgatcctacctcgacagcagatggggcctggctgctggccgacaggactccgttctgcttgttgctctgatgaacgaacccaagaaccgacttggctctgaagccgaggccaaggcctacctcgacgagcagacccagaagtatgctgcttctgccggtcttaacctgtctgcccccgctggtggtgccgagggtggcaatggcggtggcgccgtcattgactccgctgcctttgacgctctcaccaaggaccagcgatacctggtccagcagcaactcgagttgtttgccaactacctgaagcaggatctgcgacagggctccaaggtggctgctgcccagaaggaggccatggatattctgcaagctgaactggatctttggaactccgagcacggcgaggtctacgctgagggcatcaagcccgccttctctgccctgaaggcccgtgtctacgactcgtactggaactgggctcgacaggactcgctctccatgtactttgacattgttttcggtcgtctctccaccgttgaccgagagattatggctaagtgtatccacctgatgaaccgaaccaaccacaacctgatcgactacatgcagtaccacatggaccacgtccccgttcacaagggagccacctacgagcttgccaagcagctcggtctgcagctcctcgagaactgtaaggagactctcaccgaggcccccgtctacaaggatgtctcttaccccactggaccccagaccaccattgatgtcaagggtaacattgtttacaacgaggtgccccgacccaatgtccgaaagctcgagcagtatgtccacgagatggcctgtggtggtgagctgaccaaggacccctcttttgttggagaaggtgtccagggcgagctcaagaagctgtactctcagatctctgctcttgccaagacccagaccggctctaccctcgacatcgaggctctgtactccgacctggtcgctaagatctcccaggccgaggacgcgtccaagcctgtcgttgagaacaaggctgtttctgcctccatcactcccggcactctcccttttctccacatcaagaagaagaccgaacttggtgcctggaattacgacagcgagaccaccgccacctacctcgatggtcttgaggttgctgcccgtgatggtctcactttccagggcaagactgctctgatcaccggtgctggtgctggctccattggtgcctcaatcctccagggtctcatttccggaggctgcaaagtcattgtcacaacctctcgatactcccgaaaggtgaccgagtactaccagtccctctacaccaagttcggtgctaagggttccactctgattgttgtccccttcaaccaaggctccaagaaggacgtggacgagctggtgtcgttcatctacaacgaccccaagaacggcggtcttggctgggatctggactttgttgttccctttgctgctctgcccgagaacggtattgagctggagcacattgactcaaagtccgagcttgcccatcgaatcatgctcaccaacctcctgcgtctgcttggtaacgtcaagaagcagaaagtggcccattcctacgagactcgacccgcccaggtcatgctgcccctgtcgcccaaccatggcaacttcggctccgatggtctgtactccgagtccaagatctctctcgagactctgttcaaccggtggcacaccgagtcctggggctcttatctcaccattgttggtgtggtgattggctggacccgaggtaccggtctgatgagcgccaacaacatcaccgccgagggtctggagcagctcggcgtccgaaccttctcccagactgagatggccttttccatcatgggtctcatgaccaaggacattgtgcgactggcccagaactcccccgtgtgggccgatctcaacggtggcttccagtacattcccgacctcaagggagttgttggaaagatccgacgagacattgtggagacctccgagatccgacgggctgtggctcaggagactgccattgaacagaaggtggtcaacggcccccacgccgatcttccttaccagaaggtcgaggtcaagccccgagccaacctcaagtttgacttccccaccctcaaatcctacgccgaggtcaaggagctgtctcctgctggtgatgctctggagggtcttctggatctctcttccgtcattgttgtcactggtttcgccgaggtcggtccttggggtaacgcccgaacccgatgggacatggaggccaacggtgtcttctcccttgagggtgccattgagatggcctggatcatgggtctgatcaagcaccacaatggtcccctgcccggcatgcctcagtactctggctggatcgataccaagaccaagcagcccgtcgatgaccgagatatcaagaccaagtacgaggactacctgcttgagcacgccggtatccgactcattgagcctgagctgttccacggctacaaccccaagaagaagaccttcctccaggaggttattgtggagcacgatctcgagccctttgaggcctccaaggagtctgctgagcaatttgctctcgagcagggcgcgaacgttgagatcttcgccgtccccgagtccgaccagtggactgtgcgacttctcaagggcgccaagctcctcattcccaaggccctcaagtttgaccgacttgtggccggccagattcccactggatgggatgcccgacgatacggtattcccgaggacatttgtgaccaggttgaccccatcactctgtacgctcttgtctccactgttgaggctctgttggcctccggtattaccgacccctacgagttctacaagtacgtccacgtgtccgaggtcggtaactgttccggttccggtatgggtggtatcaccgccctgcgaggcatgttcaaggaccggttcatggacaagcctgttcagaacgatattctccaggagtccttcatcaacaccatgtctgcctgggtcaacatgttgctgctctcctcttccggtcccatcaagacccccgttggagcttgtgccactgctgtcgagtctgtggacattggttgcgaaaccattctgtccggcaaggccagaatctgtctggtcggtggttacgatgatttccaggaggagtcttctcaggagtttgcaaacatgaacgcaacatccaacgctgagaccgagatcactcacggccgaactccggccgagatgtctcgacccatcacttccacacgagccggtttcatggaggctcagggtgctggaacccaggtgctgatggccgccgacctcgccatcgccatgggtgtgcccatctactgtatcgttggttacgtcaacactgccaccgacaagattggccgatctgtgcctgctcccggtaagggtatcctgaccactgctcgagagcaccagactctcaaacacgccaaccctctcctcaacatcaagtaccgaaagcgacagctcgattctcgactccgagacattaagcgatgggctgagggcgaaatggaggctattgacattgagcttgacgacgtgtctgacgccgacaaggagtccttcatccaggagcgatctgcccacatccagtctcagtccgatcgaatgatccgagaggctaagaactcttggggtaacgcctttttcaagcaggacgcccgaatctcccccatccgaggagcgctggcaacctacggtctcaccattgatgacatctccgtcgcttctttccatggtacatccaccaaggccaacgagaagaacgagaccaccaccgtcaacgccatgctggagcatctcggcagaacccggggtaaccctgtctacggtatcttccagaagtaccttactggtcaccccaagggagctgctggtgcctggatgctcaacggagccatccaatgcctcaactctggtatcatccctggtaaccgaaacgccgataacgtggatgcctactttgagcagtgccagcacgtggtgttcccctcgcgatctctgcagaccgatggcctcaaggctgcttccgtgacctcctttggtttcggtcagaagggtgcccaggccattgtcatccaccccgactacctgtacgctgccctgacaccctccgagtactccgagtacaccacccgagtcgcccagcgatacaagaaggcttaccgatactaccacaacgccattgccgaggagtccatgttccaggccaaggacaaggctccctactctgctgagctggagcaggaggtctacctggatcctcttgtgcgagtccaccagaacgaggacaccgagcagtactccttcaacgccaaggacctcgctgcctccgcctttgtcaagaactcccacaaggacaccgccaaggtgcttgccaacctcacctcccaggtgtccggttctggtaagaacgttggtgtcgacgttgaggccatctccgccatcaacattgataacgacaccttccttgaccgaaacttcaccgccaacgagcaggcctactgcttcaaggccccctccccccagtcttctttcgctggcacttggtctgccaaggaggctgttttcaagtctctgggcgtcaagtcccagggcggaggagctgagctcaagtccattgagatcactcgagatggcaacggagctcccgtcgtggttcttcacggagctgccaaggacgctgctgcttctaagggtatctccaccgtcaaggtgtccatttcccatgacgactctcaggccgtggctgttgctgttgccgagtag

SEQ ID NO: 18 - protein, 1850 aa, Yarrowia lipolytica
  MISC_FEATURE (1)..(1850): Y. lipolytica fatty acid synthase 2 (XP_501096)
MHPEVEQELAHVLLTELLAYQFASPVRWIETQDVLFKQFNVERVVEVGPSPTLAGMAQRTLKSKYESYDAALSLQREILCYSKDQKDIYYLADEADEAPAPAAGGDAPAAPAAAAPAAAAAPAAAAAPSGPVAKVEDAPVKAQEILHALVAHKLKKTPEQVPLSKAIKDLVGGKSTIQNEILGDLGKEFGATPEKPEDTPLGELAESFQASFDGKLGKQSSSLIARLMSSKMPGGFSLTSARSYLDSRWGLAAGRQDSVLLVALMNEPKNRLGSEAEAKAYLDEQTQKYAASAGLNLSAPAGGAEGGNGGGAVIDSAAFDALTKDQRYLVQQQLELFANYLKQDLRQGSKVAAAQKEAMDILQAELDLWNSEHGEVYAEGIKPAFSALKARVYDSYWNWARQDSLSMYFDIVFGRLSTVDREIMAKCIHLMNRTNHNLIDYMQYHMDHVPVHKGATYELAKQLGLQLLENCKETLTEAPVYKDVSYPTGPQTTIDVKGNIVYNEVPRPNVRKLEQYVHEMACGGELTKDPSFVGEGVQGELKKLYSQISALAKTQTGSTLDIEALYSDLVAKISQAEDASKPVVENKAVSASITPGTLPFLHIKKKTELGAWNYDSETTATYLDGLEVAARDGLTFQGKTALITGAGAGSIGASILQGLISGGCKVIVTTSRYSRKVTEYYQSLYTKFGAKGSTLIVVPFNQGSKKDVDELVSFIYNDPKNGGLGWDLDFVVPFAALPENGIELEHIDSKSELAHRIMLTNLLRLLGNVKKQKVAHSYETRPAQVMLPLSPNHGNFGSDGLYSESKISLETLFNRWHTESWGSYLTIVGVVIGWTRGTGLMSANNITAEGLEQLGVRTFSQTEMAFSIMGLMTKDIVRLAQNSPVWADLNGGFQYIPDLKGVVGKIRRDIVETSEIRRAVAQETAIEQKVVNGPHADLPYQKVEVKPRANLKFDFPTLKSYAEVKELSPAGDALEGLLDLSSVIVVTGFAEVGPWGNARTRWDMEANGVFSLEGAIEMAWIMGLIKHHNGPLPGMPQYSGWIDTKTKQPVDDRDIKTKYEDYLLEHAGIRLIEPELFHGYNPKKKTFLQEVIVEHDLEPFEASKESAEQFALEQGANVEIFAVPESDQWTVRLLKGAKLLIPKALKFDRLVAGQIPTGWDARRYGIPEDICDQVDPITLYALVSTVEALLASGITDPYEFYKYVHVSEVGNCSGSGMGGITALRGMFKDRFMDKPVQNDILQESFINTMSAWVNMLLLSSSGPIKTPVGACATAVESVDIGCETILSGKARICLVGGYDDFQEESSQEFANMNATSNAETEITHGRTPAEMSRPITSTRAGFMEAQGAGTQVLMAADLAIAMGVPIYCIVGYVNTATDKIGRSVPAPGKGILTTAREHQTLKHANPLLNIKYRKRQLDSRLRDIKRWAEGEMEAIDIELDDVSDADKESFIQERSAHIQSQSDRMIREAKNSWGNAFFKQDARISPIRGALATYGLTIDDISVASFHGTSTKANEKNETTTVNAMLEHLGRTRGNPVYGIFQKYLTGHPKGAAGAWMLNGAIQCLNSGIIPGNRNADNVDAYFEQCQHVVFPSRSLQTDGLKAASVTSFGFGQKGAQAIVIHPDYLYAALTPSEYSEYTTRVAQRYKKAYRYYHNAIAEESMFQAKDKAPYSAELEQEVYLDPLVRVHQNEDTEQYSFNAKDLAASAFVKNSHKDTAKVLANLTSQVSGSGKNVGVDVEAISAINIDNDTFLDRNFTANEQAYCFKAPSPQSSFAGTWSAKEAVFKSLGVKSQGGGAELKSIEITRDGNGAPVVVLHGAAKDAAASKGISTVKVSISHDDSQAVAVAVAE

SEQ ID NO: 19 - DNA, 1062 bp, Yarrowia lipolytica
  misc_feature (1)..(1062): Y. lipolytica YALI0_F14729g
atgtctcttcttgaacgagagcttcaaattgaggagattgatatcaatctctaccggtctgccaaggagctttggcgacctatcggtcagcgaggtatctttggcggctctgtcattgctcaggccctgatggctgctaccaaaactgtgcccccagagttcattatccattccatgcactgctactttgtgttatctggaaaccccgaccaccccgtgctctaccacgttgagcgggtccgagatggcagaagcttcgctacccgaacagtccaggccaaacagcggggacgtgtgatcttcaccactacatgctctttccaggttgacaagggcaacggaaacatgcatcatcagagccgaatgtacgagcgagaggtcaagagcagtggaaaggcttttgatggcgaacacgaggccaccaacggaattcctgctcccgagaattgcgtctcctcgctggaggtgtccaagtacctcaacaagcagggcgtgatcagtgacgatattctcaagaagatggtggatcgatcagttgaggatcccattgaaattagactagtgaccggtcttctgaacaaggacgatggtctgcttcctcatgaacgaagaatcaagttctgggttcgatgcaaacctgttattgagcgagacgacgttcagtcggtcggtattgcttacctcagtgactctttcctgctgggaacagctatccgagtccagcccctcaatcccggtgctgcctctatggttgtttccctggaccacacaatctactttcatggcaagttccgagctgatgaatggctgctgcacgtgattgattccaactggagtggaaacgagcgggcactggtccgaggacgactctacaaccaacagggagtcttggtcgccacagtgttccaggagggtgtcattcgattgaaggagaaatacaaaggcaaggctgtagagaccacagatgactatcttagcagcggaacgagaactgatgctgagaaggaggagtctaagaagaagggagccatggctgctaagagtattgacagtaagctgtaa

SEQ ID NO: 20 - protein, 353 aa, Yarrowia lipolytica
  MISC_FEATURE (1)..(353): Y. lipolytica XP_505426
MSLLERELQIEEIDINLYRSAKELWRPIGQRGIFGGSVIAQALMAATKTVPPEFIIHSMHCYFVLSGNPDHPVLYHVERVRDGRSFATRTVQAKQRGRVIFTTTCSFQVDKGNGNMHHQSRMYEREVKSSGKAFDGEHEATNGIPAPENCVSSLEVSKYLNKQGVISDDILKKMVDRSVEDPIEIRLVTGLLNKDDGLLPHERRIKFWVRCKPVIERDDVQSVGIAYLSDSFLLGTAIRVQPLNPGAASMVVSLDHTIYFHGKFRADEWLLHVIDSNWSGNERALVRGRLYNQQGVLVATVFQEGVIRLKEKYKGKAVETTDDYLSSGTRTDAEKEESKKKGAMAAKSIDSKL

SEQ ID NO: 21 - DNA, 2076 bp, Yarrowia lipolytica
  misc_feature (1)..(2076): Y. lipolytica fatty acyl-CoA synthase (YALI0_D17864g)
atggtcggatacacaatttcctcaaagcccgtgtcggtggaggtcggccccgccaagcctggcgagactgccccccgacgaaacgtcattgccaaggacgcccctgtcgtcttccccgacaacgactcgtccctgaccaccgtctacaagctgttcaaaaagtacgccgagatcaacagcgagcgaaaggccatgggatggcgagacaccatcgacatccacgtggagaccaaacaggtgaccaaggtcgtggacggagtggagaagaaggtgcccaaggaatggaagtactttgagatgggcccttacaagtggctctcatacaaggaggcccttaagctggtccatgattatggagctggtcttcgacacctcggaatcaagcccaaggagaagatgcacatttacgcccagacctcccaccgatggatgctctctggcctggcttctctgtctcagggtattcccattgtcactgcctacgacactcttggagaggagggtctcactcgatctctccaggagaccaactcggtcatcatgtttaccgacaaggctctgctgagctctctcaaggtctctctcaagaagggcaccgatctgcgaatcatcatctacggaggtgatctgacccccgacgacaagaaggccggaaacacggagattgacgccatcaaggagattgttccagatatgaagatctacaccatggacgaggttgtcgctctcggccgagaacacccccaccccgtggaggaggtcgactatgaggacctggccttcatcatgtacacctctggttctaccggtgtccccaagggtgtggttctgcagcacaagcagatcctcgcctctgtggccggtgtcaccaagatcattgaccgatctatcatcggcaacacagaccggcttctcaacttcctgcccctcgcacacattttcgagtttgtgttcgagatggtcaccttctggtggggtgcttctctgggttacggaaccgtcaagaccatttccgatctgtccatgaagaactgtaagggagacattcgagagctcaagcccaccatcatggtcggcgttcccgctgtctgggaacctatgcgaaagggtattcttggcaagatcaaggagctgtctcctctgatgcagcgggtcttctgggcctcatttgccgccaagcagcgtctcgacgagaacggactccctggtggatctatcctcgactcgctcattttcaagaaggtcaaggacgccactggaggctgtctccgatacgtgtgtaacggaggtgctccagtatctgtcgacacccagaagttcatcaccactctcatctgtcccatgctgattggatgcggtctgaccgagactacagccaacaccaccatcatgtcgcctaaatcgtacgcctttggcaccattggtgagcccaccgccgccgtgaccctcaagctcattgacgtgcctgaagccggctacttcgccgagaacaaccagggagagctgtgcatcaagggcaacgtcgtgatgaaggagtactacaagaacgaggaggagaccaagaaggcgttctccgacgatggctatttcctcaccggtgatattgccgagtggaccgccaatggccagctcagaatcattgaccgacgaaagaacctcgtcaagacccagaacggagagtacattgctctggagaagctcgagacacagtaccgatcgtcgtcgtacgtggccaacctgtgtgtgtacgccgaccagaaccgagtcaagcccattgctctggtcattcctaacgagggccccaccaagaagcttgcccagagcttgggcgtcgattctgacgactgggacgccgtctgttccaacaaaaaggtggtcaaggctgtgctcaaggacatgctcgataccggccgatctctgggtctgtccggcattgagctgctgcaaggcattgtgttgctgcctggcgagtggactcctcagaacagctacctgactgctgcccagaagctcaaccgaaagaagattgtggatgataacaagaaggaaattgatgagtgctacgagcagtcttag

SEQ ID NO: 22 - protein, 691 aa, Yarrowia lipolytica
  MISC_FEATURE (1)..(691): Y. lipolytica fatty acyl-CoA synthase (XP_502959)
MVGYTISSKPVSVEVGPAKPGETAPRRNVIAKDAPVVFPDNDSSLTTVYKLFKKYAEINSERKAMGWRDTIDIHVETKQVTKVVDGVEKKVPKEWKYFEMGPYKWLSYKEALKLVHDYGAGLRHLGIKPKEKMHIYAQTSHRWMLSGLASLSQGIPIVTAYDTLGEEGLTRSLQETNSVIMFTDKALLSSLKVSLKKGTDLRIIIYGGDLTPDDKKAGNTEIDAIKEIVPDMKIYTMDEVVALGREHPHPVEEVDYEDLAFIMYTSGSTGVPKGVVLQHKQILASVAGVTKIIDRSIIGNTDRLLNFLPLAHIFEFVFEMVTFWWGASLGYGTVKTISDLSMKNCKGDIRELKPTIMVGVPAVWEPMRKGILGKIKELSPLMQRVFWASFAAKQRLDENGLPGGSILDSLIFKKVKDATGGCLRYVCNGGAPVSVDTQKFITTLICPMLIGCGLTETTANTTIMSPKSYAFGTIGEPTAAVTLKLIDVPEAGYFAENNQGELCIKGNVVMKEYYKNEEETKKAFSDDGYFLTGDIAEWTANGQLRIIDRRKNLVKTQNGEYIALEKLETQYRSSSYVANLCVYADQNRVKPIALVIPNEGPTKKLAQSLGVDSDDWDAVCSNKKVVKAVLKDMLDTGRSLGLSGIELLQGIVLLPGEWTPQNSYLTAAQKLNRKKIVDDNKKEIDECYEQS

SEQ ID NO: 23 - DNA, 968 bp, Artificial sequence
  H. armigera cytochrome b5 reductase (XP_021183830) codon optimized for Y. lipolytica; mRNA-coding sequence
  misc_feature (1)..(968): H. armigera cytochrome b5 reductase (XP_021183830) codon optimized for Y. lipolytica; mRNA-coding sequence
tgtctaacgtcgaggtggccgtggacgacgccttcggcatcctgaccgtgctgcccatcgtggtgggcgtgtctgccgccgtggtgctggtgtctgtgatcgccaactgcttctggggcaagaaggacaagaaggctgctcccaagaagtcctctcagctgatcaccctggtggaccccaacgtgaagtacgctctgcccctgatcgagcgagaggaaatctctcacgacacccgacgattccgattcggactgccctcttcggagcacgtcctgggactgcccattggccagcacatccacctgtctgccaagatcgacgacgacctggtgatccgatcttacacccctgtgtcctctgacgaagagaagggctacgtcgagctggtgatcaaggtgtacttcaagaacgtgcaccctaagttccccgacggcggcaagatgtctcagcacctgaactccctgaagatcaacgacaccatcgacgtgcgaggcccctctggccgactgcagtacgccggcaacggcctgttcctgatcaagaagatgcgaaaggaccctcctgtcgagctgcgagccaagaagctgaacatgattgccggcggaaccggaatcgctcccatgctgcagctgatccgacacatctgcaaggacgcctctgatcccaccgagatgcgactgctgttcgccaaccagaccgaagaggacatcctgctgcgaaacgagctggaaaagtaccaggctgagcaccccgagcagttcaagctgtggtacaccctggaccgacctaacgaaggctggaagtactctgtgggcttcatcaacgacgagatgatcaaggaacacctgttcgctcccgccgacgacgtgctggtgctgatgtgcggccctcctcctatgatcaacttcgcttgcaaccccgctctcgagaagctgggctaccccgagtctcagcgattcgcctactaa

SEQ ID NO: 24 - protein, 322 aa, Helicoverpa armigera
  MISC_FEATURE (1)..(322): H. armigera cytochrome b5 reductase (XP_021183830)
MSNVEVAVDDAFGILTVLPIVVGVSAAVVLVSVIANCFWGKKDKKAAPKKSSQLITLVDPNVKYALPLIEREEISHDTRRFRFGLPSSEHVLGLPIGQHIHLSAKIDDDLVIRSYTPVSSDEEKGYVELVIKVYFKNVHPKFPDGGKMSQHLNSLKINDTIDVRGPSGRLQYAGNGLFLIKKMRKDPPVELRAKKLNMIAGGTGIAPMLQLIRHICKDASDPTEMRLLFANQTEEDILLRNELEKYQAEHPEQFKLWYTLDRPNEGWKYSVGFINDEMIKEHLFAPADDVLVLMCGPPPMINFACNPALEKLGYPESQRFAY

SEQ ID NO: 25 - DNA, 627 bp, Artificial sequence
  E. coli thioesterase (AAB40248) codon optimized for Y. lipolytica; mRNA-coding sequence
  misc_feature (1)..(627): E. coli thioesterase (AAB40248) codon optimized for Y. lipolytica; mRNA-coding sequence
atgatgaacttcaacaacgtgttccgatggcatctgccctttctgtttctggtgctgctgaccttccgagccgccgctgctgacaccctgctgatcctgggcgactctctgtctgccggctaccgaatgtctgcctctgccgcttggcccgctctgctgaacgacaagtggcagtctaagacctctgtggtgaacgcctctatctctggcgacacctctcagcagggcctcgctcgactgcctgctctgctcaagcagcatcagccccgatgggtgctcgtcgagcttggcggcaacgacggcctgcgaggcttccagcctcagcagaccgagcagaccctgcgacagattctgcaggacgtgaaggccgccaacgctgagcctctgctgatgcagattcgactgcccgccaactacggccgacgatacaacgaggccttctctgctatctaccccaagctggccaaggaattcgacgtgcccctgctgccattcttcatggaagaggtgtacctgaagcctcagtggatgcaggacgacggcattcaccccaaccgagatgctcagcccttcattgccgactggatggccaagcagctgcagcctctggtgaaccacgactcttaa

SEQ ID NO: 26 - protein, 218 aa, Escherichia coli
  MISC_FEATURE (1)..(218): E. coli thioesterase (AAB40248)
MLPLTDGLLKMMNFNNVFRWHLPFLFLVLLTFRAAAADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 27 - DNA, 531 bp, Yarrowia lipolytica
  misc_feature (1)..(531): Y. lipolytica TEFintron promoter
agagaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtgagtttcagaggcagcagcaattgccacgggctttgagcacacggccgggtgtggtcccattcccatcgacacaagacgccacgtcatccgaccagcactttttgcagtactaaccgcag

SEQ ID NO: 28 - DNA, 1002 bp, Yarrowia lipolytica
  misc_feature (1)..(1002): Y. lipolytica EXP promoter
aaggagtttggcgcccgttttttcgagccccacacgtttcggtgagtatgagcggcggcagattcgagcgtttccggtttccgcggctggacgagagcccatgatgggggctcccaccaccagcaatcagggccctgattacacacccacctgtaatgtcatgctgttcatcgtggttaatgctgctgtgtgctgtgtgtgtgtgttgtttggcgctcattgttgcgttatgcagcgtacaccacaatattggaagcttattagcctttctattttttcgtttgcaaggcttaacaacattgctgtggagagggatggggatatggaggccgctggagggagtcggagaggcgttttggagcggcttggcctggcgcccagctcgcgaaacgcacctaggaccctttggcacgccgaaatgtgccacttttcagtctagtaacgccttacctacgtcattccatgcatgcatgtttgcgccttttttcccttgcccttgatcgccacacagtacagtgcactgtacagtggaggttttgggggggtcttagatgggagctaaaagcggcctagcggtacactagtgggattgtatggagtggcatggagcctaggtggagcctgacaggacgcacgaccggctagcccgtgacagacgatgggtggctcctgttgtccaccgcgtacaaatgtttgggccaaagtcttgtcagccttgcttgcgaacctaattcccaattttgtcacttcgcacccccattgatcgagccctaacccctgcccatcaggcaatccaattaagctcgcattgtctgccttgtttagtttggctcctgcccgtttcggcgtccacttgcacaaacacaaacaagcattatatataaggctcgtctctccctcccaaccacactcacttttttgcccgtcttcccttgctaacacaaaagtcaagaacacaaacaaccaccccaacccccttacacacaagacatatctacagca

SEQ ID NO: 29 - DNA, 1029 bp, Yarrowia lipolytica
  misc_feature (1)..(1029): Y. lipolytica YEF3 promoter
gatagctgagggatatggtgtagataaggggtgtccgagggccgtgttagcactccagagagagtgcacatggagctcgggctggggtctcgaagcgttgtccggcagcttttttgccacccccaaaactgaacatcaccctttgaccctgtcccacaatcagccgtatacttgggttcagcaactctggatactacaggaaaactatgcagagatccaaccacacacggccaatgtcctctatggagctgcgtgggagatattgggtaagtcctaagtggctggaaaagggggattgagcccgcgtcctaggccatggtccatcccgttgctctcaatgccggcctatagaacgggttccaacactacacacacccactaatgcacccctccccctcgtgttagccgaggagagatggtatgagtgagtagacaagaagagatggtgatgaccgagaacgccgatagtatcagcgagatacacgccaacaaccaaacaacttggttgccctcaaatcaagcccctcttcgccattcggttccttccagaccattccagatcaatccacctcttcttatctcaggtgggtgtgctgacatcagaccccgtagcccttctcccagtggcgaacagcaggcataaaacagggccattgagcagagcaaacaaggtcggtgaaatcgtcgaaaaagtcggaaaacggttgcaagaaattggagcgtcacctgccaccctccaggctctatataaagcattgccccaattgctaacgcttcatatttacacctttggcaccccagtccatccctccaataaaatgtactacatgggacacaacaagagaggatgcgcgcccaaaccctaacctagcacatgcacgatgattctctttgtctgtgaaaaaatttttccaccaaaatttccccattgggatgaaaccctaaccgcaaccaaaagtttttaactatcatcttgtacgtcacggtttccgattcttctcttctctttcatcatcatcacttgtgacc

SEQ ID NO: 30 - DNA, 2247 bp, Saccharomyces cerevisiae
atgacgagacgtactactattaatcccgattcggtggttctgaatcctcaaaaatttatccagaaagaaagggcggattcgaaaatcaaagttgaccaagttaacacatttttagagtcatccccggagaggagaactctgacgcacgccttaatagaccaaatagtgaatgatcctatattgaaaactgatacggactattacgatgctaaaaaaatgcaagagagagaaattactgccaaaaaaatagctaggcttgctagttatatggagcacgatatcaaaacagtgcgcaaacactttcgcgacactgacctgatgaaagagttgcaagcaaatgatccagacaaagcttcgcctttaacaaacaaagacctttttatattcgataagagattgtcacttgtagcaaatattgatcctcaattgggtacgcgcgtgggtgtacacttggggctatttggtaattgtatcaagggcaatggtactgatgagcaaatccggtattggttgcaggagagaggtgccactttgatgaaaggtatatatggctgttttgcaatgactgagttaggacatggttccaatgttgcccagctgcagactagggctgtgtacgataagcaaaatgatacttttgtaattgatacacctgatctaactgccaccaaatggtggattggtggggctgcccattctgccacgcacgctgccgtgtacgccagattgatcgttgaaggtaaagactacggtgtaaaaacattcgttgttcctctgagagacccttcgactttccaactgttagctggtgtttccataggggatattggagcgaagatgggtcgtgacggtattgataatggctggatccagttcagaaacgtagttatccctagagaatttatgctaagtagatttaccaaagttgtccgttctccagatggttcagtcaccgtcaaaactgagccacaattggatcaaatttctggttatagtgcattgttaagtggtagagttaacatggtcatggattcatttaggtttggctccaaatttgctactattgctgtacgttacgcggttggtcgtcagcaattcgcacctagaaagggattgtctgaaacacaattaatcgactatccccttcaccaatatcgtgttttaccacaattgtgtgttccatatttggtgtcacctgtagcttttaagttaatggacaactattattccactttggacgagttatacaacgcttcctcatctgcatacaaagctgctctggttaccgtgagtaaaaagttgaagaatttatttattgatagcgccagcttgaaagccaccaatacttggttaattgctacactgattgatgagttgagacagacttgcggaggacatgggtattcacagtataacggatttggtaaaggctatgacgactgggtggttcagtgcacatgggagggtgataataatgttttatctttaacttcagcaaaatcaatattgaaaaaatttatcgattcagccacaaagggtagatttgacaacacactggatgtggactcattctcttacttaaaacctcagtacataggatctgtggtttctggagaaataaagagtggtttaaaggagttgggtgattatactgaaatttggtctatcaccttaatcaaattactggcacatattggtactttagttgaaaaatcaagaagtattgatagcgtttctaagcttttagtcttagtatccaaatttcatgccttgcgctgcatgttgaaaacctattacgacaagttaaactctcgtgattcacatatttccgatgaaattacaaaggaatctatgtggaatgtttataagttattttccttgtattttattgacaagcattccggagaattccaacaattcaagatcttcactcctgatcagatctctaaagttgtgcagccacaactattggctcttttgccaattgtgaggaaagactgtataggtctgacagactcctttgaattacctgacgcgatgttaaattctcctataggttactttgatggcgatatctatcacaattacttcaatgaagtttgccgcaataatccagtggaggcagatggggcagggaagccttcttatcatgcgctgttgagcagcatgctcggtagaggtttcgaatttgaccaaaagttaggtggtgcagctaatgcggaaattttatcgaaaataaacaagtga

SEQ ID NO: 31 - protein, 748 aa, Saccharomyces cerevisiae
MTRRTTINPDSVVLNPQKFIQKERADSKIKVDQVNTFLESSPERRTLTHALIDQIVNDPILKTDTDYYDAKKMQEREITAKKIARLASYMEHDIKTVRKHFRDTDLMKELQANDPDKASPLTNKDLFIFDKRLSLVANIDPQLGTRVGVHLGLFGNCIKGNGTDEQIRYWLQERGATLMKGIYGCFAMTELGHGSNVAQLQTRAVYDKQNDTFVIDTPDLTATKWWIGGAAHSATHAAVYARLIVEGKDYGVKTFVVPLRDPSTFQLLAGVSIGDIGAKMGRDGIDNGWIQFRNVVIPREFMLSRFTKVVRSPDGSVTVKTEPQLDQISGYSALLSGRVNMVMDSFRFGSKFATIAVRYAVGRQQFAPRKGLSETQLIDYPLHQYRVLPQLCVPYLVSPVAFKLMDNYYSTLDELYNASSSAYKAALVTVSKKLKNLFIDSASLKATNTWLIATLIDELRQTCGGHGYSQYNGFGKGYDDWVVQCTWEGDNNVLSLTSAKSILKKFIDSATKGRFDNTLDVDSFSYLKPQYIGSVVSGEIKSGLKELGDYTEIWSITLIKLLAHIGTLVEKSRSIDSVSKLLVLVSKFHALRCMLKTYYDKLNSRDSHISDEITKESMWNVYKLFSLYFIDKHSGEFQQFKIFTPDQISKVVQPQLLALLPIVRKDCIGLTDSFELPDAMLNSPIGYFDGDIYHNYFNEVCRNNPVEADGAGKPSYHALLSSMLGRGFEFDQKLGGAANAEILSKINK

SEQ ID NO: 32 - DNA, 1236 bp, Artificial Sequence
  codon-optimized nucleotide sequence
  misc_feature (1)..(1236): Y. lipolytica codon-optimized nucleotide sequence of thioesterase from Cuphea palustris CpFATB2
atggtggccgctgccgcctccgccgccttcttctctgtggccacccctcgaactaacatctctccctcgtctctgtctgtgcccttcaagcccaagtctaaccacaacggcggcttccaggtgaaggccaacgcctctgcccaccccaaggccaacggatctgccgtgtctctgaagtctggctctctcgagactcaggaggacaagacctcttcttcgtcgcctcctccccgaaccttcatcaaccagctgcccgtgtggtctatgctgctgtctgccgtgaccaccgtgttcggcgtggccgagaagcagtggcccatgctggaccgaaagtctaagcgacccgacatgctggtcgagcccctgggcgtggaccgaatcgtgtacgacggcgtgtctttccgacagtctttctctatccgatcttacgagatcggcgctgaccgaaccgcctctatcgagactctgatgaacatgttccaggagacttctctgaaccactgcaagatcatcggcctgctgaacgacggcttcggccgaacccctgagatgtgcaagcgagatctgatttgggtggtgaccaagatgcagatcgaggtgaaccgataccccacctggggcgacaccattgaggtgaacacctgggtgtctgcctctggcaagcacggcatgggccgagactggctgatctctgactgccacaccggcgagatcctgatccgagccacctctgtgtgggccatgatgaaccagaagacccgacgactgtctaagatcccctacgaggtgcgacaggagatcgagccccagttcgtggactctgctcccgtgatcgtggacgaccgaaagttccacaagctggacctcaagaccggcgactctatctgcaacggcctgacccctcgatggaccgacctggacgtgaaccagcacgtgaacaacgtgaagtacatcggctggatcctgcagtctgtgcccaccgaggtgtttgagactcaggagctgtgcggcctgaccctcgagtaccgacgagagtgcggccgagactctgtgctcgagtctgtgaccgccatggacccctctaaggagggcgaccgatctctgtaccagcacctcctgcgactcgaggacggcgccgacatcgtgaagggccgaaccgagtggcgacccaagaacgctggcgccaagggcgccatcctgaccggcaagacctctaacggcaactctatctcttaa

SEQ ID NO: 33 - protein, 411 aa, Cuphea palustris
MVAAAASAAFFSVATPRTNISPSSLSVPFKPKSNHNGGFQVKANASAHPKANGSAVSLKSGSLETQEDKTSSSSPPPRTFINQLPVWSMLLSAVTTVFGVAEKQWPMLDRKSKRPDMLVEPLGVDRIVYDGVSFRQSFSIRSYEIGADRTASIETLMNMFQETSLNHCKIIGLLNDGFGRTPEMCKRDLIWVVTKMQIEVNRYPTWGDTIEVNTWVSASGKHGMGRDWLISDCHTGEILIRATSVWAMMNQKTRRLSKIPYEVRQEIEPQFVDSAPVIVDDRKFHKLDLKTGDSICNGLTPRWTDLDVNQHVNNVKYIGWILQSVPTEVFETQELCGLTLEYRRECGRDSVLESVTAMDPSKEGDRSLYQHLLRLEDGADIVKGRTEWRPKNAGAKGAILTGKTSNGNSIS

SEQ ID NO: 34 - DNA, 1149 bp, Artificial Sequence
  codon-optimized nucleotide sequence
  misc_feature (1)..(1149): Y. lipolytica codon-optimised nucleotide sequence of thioesterase from Cinnamomum camphora CcFatB1
atggctaccacctctctggcctctgccttctgctctatgaaggccgtgatgctggcccgagatggccgaggaatgaagccccgatcttctgacctgcagctgcgagccggcaacgcccagacctctctgaagatgatcaacggcaccaagttctcttacaccgagtcgctgaagaagctgcccgactggtctatgctgttcgccgtgatcaccaccatcttctctgccgccgagaagcagtggaccaacctcgagtggaagcccaagcctaaccctcctcagctgctggacgaccacttcggaccccacggcctggtgttccgacgaaccttcgccatccgatcttacgaggtgggccccgaccgatctacctctatcgtggccgtcatgaaccacctccaagaggccgctctgaaccacgccaagtctgtgggcatcctcggcgacggcttcggcaccactctcgagatgtctaagcgagatctgatttgggtcgtgaagcgaacccacgtcgccgtcgagcgataccccgcctggggcgacaccgtcgaggtcgagtgctgggtgggcgcctctggcaacaacggccgacgacacgactttctggtgcgagactgcaagaccggcgagattctgacccgatgtacctctctgtctgtgatgatgaacacccgaactcgacgactgtctaagatccccgaggaagtgcgaggcgagatcggacccgccttcatcgacaacgtggccgtgaaggacgaggaaatcaagaagccccagaagctgaacgactctaccgccgactacatccaaggcggactgacccctcgatggaacgacctggacatcaaccagcacgtgaacaacatcaagtacgtggactggatcctcgagactgtgcccgactctatcttcgagtctcaccacatctcttcgttcaccatcgagtaccgacgagagtgcaccatggactctgtgctgcagtctctgaccaccgtgtctggcggctcctctgaggccggactggtgtgcgagcacctcctgcagctcgaaggcggctctgaggtcctgcgagccaagaccgagtggcgacccaagctgactgactctttccgaggcatctctgtgatccccgccgagtcctctgtgtaa

SEQ ID NO: 35 - protein, 382 aa, Cinnamomum camphora
MATTSLASAFCSMKAVMLARDGRGMKPRSSDLQLRAGNAQTSLKMINGTKFSYTESLKKLPDWSMLFAVITTIFSAAEKQWTNLEWKPKPNPPQLLDDHFGPHGLVFRRTFAIRSYEVGPDRSTSIVAVMNHLQEAALNHAKSVGILGDGFGTTLEMSKRDLIWVVKRTHVAVERYPAWGDTVEVECWVGASGNNGRRHDFLVRDCKTGEILTRCTSLSVMMNTRTRRLSKIPEEVRGEIGPAFIDNVAVKDEEIKKPQKLNDSTADYIQGGLTPRWNDLDINQHVNNIKYVDWILETVPDSIFESHHISSFTIEYRRECTMDSVLQSLTTVSGGSSEAGLVCEHLLQLEGGSEVLRAKTEWRPKLTDSFRGISVIPAESSV

SEQ ID NO: 36 - DNA, 1578 bp, Artificial sequence
  codon-optimized nucleotide sequence
  misc_feature (1)..(1578): Y.lipolytica codon-optimized nucleotide sequence of alcohol acetyltransferase from S. cerevisiae ATF1
atgaacgagatcgacgagaagaaccaggctcctgtgcagcaagagtgcctgaaggaaatgatccagaacggacacgcccgacgaatgggctctgtcgaggacctgtacgtggccctgaaccgacagaacctgtaccgaaacttctgcacctacggcgagctgtctgactactgcacccgagatcagctgaccctggctctgcgagagatctgcctgaagaaccctactctgctgcatatcgtgctgcccactcgatggcccaaccacgagaactactaccgatcttctgagtactactctcgaccccatcctgtgcacgactacatctccgtgctgcaagagctgaagctgtctggcgtggtgctgaacgagcagcccgagtactctgccgtgatgaagcagatcctggaagagttcaagaactctaagggctcttacaccgccaagatcttcaagctgactactaccctgaccattccttacttcggccccactggaccctcttggcgactgatctgtctgcccgaggaacacaccgagaagtggaagaagttcatcttcgtttctaaccactgcatgtctgacggacgatcctctatccacttctttcacgacctgcgagatgagctgaacaacatcaagacccctccaaagaagctggactacattttcaagtacgaagaggactaccagctgctgcgaaagctgcccgagcctatcgagaaggtgatcgacttccgacctccttacctgttcatccccaagtctctgctgtctggattcatctacaaccacctccgattctcttcgaagggcgtgtgcatgcgaatggacgacgtggaaaagaccgacgacgttgtgaccgagatcatcaacatctctcccaccgagttccaggccatcaaggccaacattaagtctaacatccagggcaagtgtaccatcactccctttctgcacgtgtgctggttcgtgtctctgcacaagtggggcaagttctttaagcccctgaacttcgagtggctgaccgacatcttcatccccgccgactgccgatctcagctgcctgacgacgacgagatgcgacagatgtaccgatacggcgccaacgtgggcttcatcgacttcaccccttggatctctgagttcgacatgaacgacaacaaggaaaacttctggcccctgatcgagcactaccacgaggtgatttctgaggccctgcgaaacaagaagcacctccacggcctgggcttcaacattcagggcttcgtccagaagtacgtcaacattgacaaggtgatgtgcgaccgagccatcggcaagcgacgaggcggcaccctgctgtctaacgtgggcctgttcaaccagctcgaggaacccgacgccaagtactctatctgcgacctggccttcggccagttccaaggctcttggcaccaggctttctccctgggcgtgtgttctaccaacgtgaagggcatgaacatcgtggtggcctctaccaagaacgtggtgggctctcaagagtctctggaagaactgtgctctatctacaaggccctgctgctgggcccctaa

SEQ ID NO: 37 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 38 - DNA, 2088 bp, Artificial sequence
  codon-optimized nucleotide sequence
  misc_feature (1)..(2088): DNA Codon-optimised sequence of peroxisomal oxidase
  misc_feature (1)..(2088): DNA Codon-optimised sequence of peroxisomal oxidase, Agrotis segetum
atgcccattttcatctgtatcatcacctcgcaagccatcatccgatcgaacgtggaacgagtggcagtcattcttaacatcaacatgggtaaggtgaacgaggacctggttcgagagcgtgcaaagtgtaccttcaacattgaggagctcacctactttcttgacggaggaaaagataaaaccttggaaagaaaggagaccgaacgagctatgttgaccaagcgagaagagctctttggaggcgttcccgacgaatacctgtcacacaaggagaagtacgagaactccatgcgaaaggctgttattctcttcggaatccttagaaagatccaaaaggataacaacaccgacctcaccaactaccggaatctgctgagcggagtgctgtccgtgtccatctcccaggatggctctcctttcggcctccactacatcatgttcatgcctgtgctgctgtctcaggctgatgaaaaacaacaagagaagtggctgaagcgagccatgaactgtgaaatcatcggctcttacgcccagaccgagctgggccacggcactttcatccgaggccttgagactactgccacttacgatcccgccacccaggagttcgtgctgcactctcctgctctttcgtcctacaagtggtggcctggaggcctcggaaacactgtcaattactgcatcgttattgcccagctgtactctaagggcgtctgtcacggcattcattccttcatcgtgcaggtgcgagacgaggacactcacatgccgttgcccggcatcaaggtcggagagatcggtgtgaagatgggtctgaactctgtcaacaacggattcctgggattcgagaacgtccgaatcccccgagtgaacatgcttatgaagcacgccaagatcctggaggatggaacttacgttaagtcgaagaacaacaagctcatctacggagcaatggtgttcgtccgagtggtgatcgtgttcgactctgtgaactacctggccaaggccatcaccattggcgcacgatactccctggtgcggcgacagtcgcaattaaaagctggagaacctgagcgacagatcctcgactatgtcacccagcagcacaagattctgcccgctatcgctggatgctacgccatgaagatgaacgcttggaggttgtgggacaccttcaacctgatcaacggacaactgcatcagggcaacatggaacggctgggcgagctgcatgccctcgcgtgctgcctcaaggctatctctaccaccgacgcggctatgttcacctctctgtgtcgactcggatgcggaggtcacggttacatgacttcttccaacctccctcccacatacgctttgacttctgcctcgtgcacttacgagggagacaacaccgttctgctgctgcagaccgctcgatttcttctcaagacctggcgacagattgacacccaccctctgactagaaccgtggcctacctgaagaccgtgtctgcccctggattctctgacagatgggagtcttccgtggagggcatcattcgaggcttccagaccgtcgctatgaaaaagatttcttcctgtctggacatcatgacttctaaggtgatgtctggaatgtcccaggaggatgcatggaacgctatttctatccagctggtttcggccgctgaatcccattctcgaggcaccgtgatctctacgttttacgaagacatgtctaaggccatgcgatccatgactgctcccttggcgaaggtgatgggtcagctggttgagctgtacgcggtttattggactctagagcgactgggagacatgttgcagtacaccagtatttctcacaccgacgttgttgacctccgatcctggtacgaagagctcctccgaaagatccgacctaacactatcggactggtggacgcgtttgatattattgatgaactgctccagtccaccctgggtgcttatgacggtcgtgtttacgaacgactgatggaagaagctttgaagtctcccctgaacgctgagcccgtgaaccagtccttccacaagtacctcaagccctttatgcagtctaagctgtaa

SEQ ID NO: 39 - protein, 695 aa, Agrotis segetum
MPIFICIITSQAIIRSNVERVAVILNINMGKVNEDLVRERAKCTFNIEELTYFLDGGKDKTLERKETERAMLTKREELFGGVPDEYLSHKEKYENSMRKAVILFGILRKIQKDNNTDLTNYRNLLSGVLSVSISQDGSPFGLHYIMFMPVLLSQADEKQQEKWLKRAMNCEIIGSYAQTELGHGTFIRGLETTATYDPATQEFVLHSPALSSYKWWPGGLGNTVNYCIVIAQLYSKGVCHGIHSFIVQVRDEDTHMPLPGIKVGEIGVKMGLNSVNNGFLGFENVRIPRVNMLMKHAKILEDGTYVKSKNNKLIYGAMVFVRVVIVFDSVNYLAKAITIGARYSLVRRQSQLKAGEPERQILDYVTQQHKILPAIAGCYAMKMNAWRLWDTFNLINGQLHQGNMERLGELHALACCLKAISTTDAAMFTSLCRLGCGGHGYMTSSNLPPTYALTSASCTYEGDNTVLLLQTARFLLKTWRQIDTHPLTRTVAYLKTVSAPGFSDRWESSVEGIIRGFQTVAMKKISSCLDIMTSKVMSGMSQEDAWNAISIQLVSAAESHSRGTVISTFYEDMSKAMRSMTAPLAKVMGQLVELYAVYWTLERLGDMLQYTSISHTDVVDLRSWYEELLRKIRPNTIGLVDAFDIIDELLQSTLGAYDGRVYERLMEEALKSPLNAEPVNQSFHKYLKPFMQSKL

SEQ ID NO: 40 - DNA, 2031 bp, Artificial sequence
  codon-optimized nucleotide sequence
  misc_feature (1)..(2031): DNA Codon-optimised sequence of peroxisomal oxidase 1
  misc_feature (1)..(2031): DNA Codon-optimised sequence of peroxisomal oxidase 1, Arabidopsis thaliana
atggaaggcatcgaccacctggccgacgagcgaaacaaggccgagttcgacgttgaggacatgaagatcgtgtgggccggctctcgacacgccttcgaggtgtctgaccgaatcgcccgactggtggcttctgaccccgtgttcgagaagtctaaccgagccagactgtctcgaaaggaactgttcaagtctaccctgcgaaagtgcgcccacgccttcaagcgaatcatcgagctgcgactgaacgaggaagaggccggacgactgcgacacttcattgaccagcctgcctacgtggacctgcactggggcatgttcgtgcccgccatcaagggccagggcaccgaggaacagcagaagaagtggctgtctctggccaacaagatgcagatcatcggctgctacgcccagaccgagcttggccacggctctaacgtgcagggcctcgagactaccgccactttcgaccccaagaccgacgagttcgtgattcacacccctactcagaccgcctctaagtggtggcccggtggcctcggcaaggtgtctacccacgccgtggtgtacgctcgactgatcaccaacggcaaggactacggcatccacggcttcatcgtgcagctgcgatctctcgaggaccactctcctctgcctaacatcaccgtgggcgacatcggcaccaagatgggcaacggcgcctacaactctatggacaacggcttcctgatgttcgaccacgtgcgaattccccgagatcagatgctgatgcgactgtctaaggtgacccgagagggcgagtacgtgccctctgacgtgcccaagcagctggtgtacggaaccatggtgtacgtgcgacagaccatcgtggccgacgcttctaacgccctgtctcgagccgtgtgtatcgctacccgatactctgccgtgcgacgacagttcggcgcccacaacggcggcatcgagactcaggtgatcgactacaagacccagcagaaccgactgttccctctgctggcctccgcctacgccttccgattcgtcggcgagtggctgaagtggctctacaccgacgtgaccgagcgactggccgcctctgacttcgccactctgcccgaggctcacgcctgcaccgccggactgaagtctctgaccaccaccgccaccgctgacggcatcgaagagtgccgaaagctgtgtggcggccacggatacctgtggtgctctggactgcccgagctgttcgccgtgtacgtccccgcctgtacctacgagggcgacaacgtggtgctgcagctccaggtggcccgattcctgatgaagaccgtcgctcagctcggctctggcaaggtgcccgtgggaaccaccgcctacatgggccgagccgctcacctcctgcagtgccgatctggcgtgcagaaggccgaggactggctgaaccccgacgtcgtgctcgaggctttcgaggcccgagcactgcgaatggccgtgacctgcgccaagaacctgtctaagttcgagaaccaggaacagggcttccaagagctgctggccgacctggtcgaggccgctatcgcccactgccagctgatcgtggtgtccaagttcattgctaagctcgagcaggacatcggcggcaagggcgtgaagaagcagctgaacaacctgtgctacatctacgccctgtacctgctgcacaagcacctgggcgactttctgtctaccaactgcattacccctaagcaggcctctctggctaacgaccagctccgatcgctgtacacccaggtgcgacccaacgctgtggccctggtggacgctttcaactacactgaccactacctgaactctgtgctgggccgatacgacggcaacgtgtaccccaagctgttcgaagaggccctgaaggaccctctgaacgactctgtggtgcccgacggctaccaagagtacctgcgacctgtcctgcagcagcagctccgaaccgctcgactcgaccagattacctctgtgggatcttcttcgaagctgtag

SEQ ID NO: 41 - protein, 676 aa, Arabidopsis thaliana
MEGIDHLADERNKAEFDVEDMKIVWAGSRHAFEVSDRIARLVASDPVFEKSNRARLSRKELFKSTLRKCAHAFKRIIELRLNEEEAGRLRHFIDQPAYVDLHWGMFVPAIKGQGTEEQQKKWLSLANKMQIIGCYAQTELGHGSNVQGLETTATFDPKTDEFVIHTPTQTASKWWPGGLGKVSTHAVVYARLITNGKDYGIHGFIVQLRSLEDHSPLPNITVGDIGTKMGNGAYNSMDNGFLMFDHVRIPRDQMLMRLSKVTREGEYVPSDVPKQLVYGTMVYVRQTIVADASNALSRAVCIATRYSAVRRQFGAHNGGIETQVIDYKTQQNRLFPLLASAYAFRFVGEWLKWLYTDVTERLAASDFATLPEAHACTAGLKSLTTTATADGIEECRKLCGGHGYLWCSGLPELFAVYVPACTYEGDNVVLQLQVARFLMKTVAQLGSGKVPVGTTAYMGRAAHLLQCRSGVQKAEDWLNPDVVLEAFEARALRMAVTCAKNLSKFENQEQGFQELLADLVEAAIAHCQLIVVSKFIAKLEQDIGGKGVKKQLNNLCYIYALYLLHKHLGDFLSTNCITPKQASLANDQLRSLYTQVRPNAVALVDAFNYTDHYLNSVLGRYDGNVYPKLFEEALKDPLNDSVVPDGYQEYLRPVLQQQLRTARLDQITSVGSSSKL

SEQ ID NO: 42 - DNA, 2115 bp, Artificial sequence
  codon-optimized nucleotide sequence
  misc_feature (1)..(2115): DNA Codon-optimised sequence of peroxisomal oxidase 2
  misc_feature (1)..(2115): DNA Codon-optimised sequence of peroxisomal oxidase 2, Arabidopsis thaliana
atggaatctcgacgagagaagaaccccatgaccgaggaagagtctgacggcctgatcgccgctcgacgaatccagcgactgtctctgcacctgtcgccttctctgaccccttctccttcgctgcccctggtgcagaccgagacttgctctgcccgatctaagaagctggacgtcaacggcgaggccctgtctctgtacatgcgaggcaagcacatcgacatccaagagaagattttcgacttcttcaactctcgacccgacctgcagacccctatcgagatctctaaggacgaccaccgagagctgtgcatgaaccagctgatcggcctggtgcgagaggccggcgtgcgacccttccgatacgtggctgacgaccctgagaagtactttgccatcatggaagccgtgggctctgtggacatgtctctgggcatcaagatgggcgtgcagtactctctgtggggcggctctgtgatcaacctgggcaccaagaagcaccgagacaagtacttcgacggcatcgacaacctggactacaccggctgcttcgctatgaccgagctgcaccacggctctaacgtgcagggactgcagaccaccgccactttcgaccctctgaaggacgagttcgtgatcgacacccctaacgacggcgccatcaagtggtggatcggcaacgccgccgtccacggcaagttcgccaccgtgttcgcccgactgattctgcccactcacgactctaagggcgtgtctgacatgggagtgcacgccttcatcgtgcccatccgagacatgaagacccaccagactctgcccggcgtcgagatccaggactgcggccacaaggtgggcctgaacggcgtggacaacggcgccctgcgattccgatctgtgcgaattccccgagacaacctgctgaaccgattcggcgacgtgtctcgagatggcacctacacctcttctctgcccaccatcaacaagcgattcggagctaccctgggcgagctggtcggcggacgagtcggcctggcctacgcctctgtgggcgtgctgaagatctccgccactatcgccatccgatactccctgctgcgacagcagttcggccctcctaagcagcccgaggtgtctattctggactaccagtctcagcagcacaagctgatgcccatgctggcctctacctacgcctaccacttcgctaccgtgtacctggtggaaaagtactctgagatgaagaagactcacgacgagcagctggtggccgacgtgcacgccctgtctgccggactgaagtcttacgtgacctcttacaccgccaaggctctgtctgtgtgccgagaggcctgtggcggccacggctacgccgctgtcaaccgatttggctctctgcgaaacgaccacgacatcttccagaccttcgagggcgacaacaccgtgctgctccagcaggtcgccgccgacctcctgaagcgatacaaggaaaagttccaaggcggcaccctgaccgtcacctggtcttacctgcgagagtctatgaacacctacctctcgcagcccaaccctgtgaccgctcgatgggaaggcgaggaccatctgcgagatcccaagtttcagctggacgctttccgataccgaacctctcgactgctgcagaacgtggctgcccgactgcagaagcactctaagaccctcggcggcttcggcgcctggaaccgatgcctgaaccatctgctgaccctggccgagtctcacatcgagactgtgatcctggccaagttcatcgaggccgtgaagaactgccccgatccttctgccaaggccgctctgaagctggcctgcgacctgtacgccctggaccgaatctggaaggacatcggcacctaccgaaacgtggactacgtggctcccaacaaggccaaggccatccacaagctcaccgagtacctgtctttccaggtgcgaaacgtcgccaaggaactggtggacgccttcgagctgcctgaccacgtgactcgagcccctattgccatgcagtctgacgcctactctcagtacacccaggtggtgggcttcgaccagattacctccgtgggatcttcttcgaagctgtag

SEQ ID NO: 43 - protein, 704 aa, Arabidopsis thaliana
MESRREKNPMTEEESDGLIAARRIQRLSLHLSPSLTPSPSLPLVQTETCSARSKKLDVNGEALSLYMRGKHIDIQEKIFDFFNSRPDLQTPIEISKDDHRELCMNQLIGLVREAGVRPFRYVADDPEKYFAIMEAVGSVDMSLGIKMGVQYSLWGGSVINLGTKKHRDKYFDGIDNLDYTGCFAMTELHHGSNVQGLQTTATFDPLKDEFVIDTPNDGAIKWWIGNAAVHGKFATVFARLILPTHDSKGVSDMGVHAFIVPIRDMKTHQTLPGVEIQDCGHKVGLNGVDNGALRFRSVRIPRDNLLNRFGDVSRDGTYTSSLPTINKRFGATLGELVGGRVGLAYASVGVLKISATIAIRYSLLRQQFGPPKQPEVSILDYQSQQHKLMPMLASTYAYHFATVYLVEKYSEMKKTHDEQLVADVHALSAGLKSYVTSYTAKALSVCREACGGHGYAAVNRFGSLRNDHDIFQTFEGDNTVLLQQVAADLLKRYKEKFQGGTLTVTWSYLRESMNTYLSQPNPVTARWEGEDHLRDPKFQLDAFRYRTSRLLQNVAARLQKHSKTLGGFGAWNRCLNHLLTLAESHIETVILAKFIEAVKNCPDPSAKAALKLACDLYALDRIWKDIGTYRNVDYVAPNKAKAIHKLTEYLSFQVRNVAKELVDAFELPDHVTRAPIAMQSDAYSQYTQVVGFDQITSVGSSSKL

SEQ ID NO: 44 - DNA, 2091 bp, Artificial sequence
  codon-optimized nucleotide sequence
  misc_feature (1)..(2091): DNA Codon-optimised sequence of peroxisomal oxidase
  misc_feature (1)..(2091): DNA Codon-optimised sequence of peroxisomal oxidase, Aspergillus nidulans
atgcccaaccctccgcctgcctgggtgcaagctctgaagcccgcttcgccccagggcaccgagctgctgacccaagagcgagcccagtctaacatcgacgtggacaccctgggcgacctgctgcacaccaaggaagccctgaagaagcaggacgagatcctgtctgtgctgaagtctgagaaggtgttcgacaagtctcgaaaccacgtgctgggccgaaccgagaagatccagctggccctggctcgaggcaagcgactgcagcagctgaagaaggcccacaactggtctgacgaggacgtccacgtggccaacgacctggtgtctgagcccactccttacggcctgcacgcctctatgtttctggtgaccctgcgagagcagggcacccctgagcagcacaagctgttctacgaacgagcccgaaactacgagatcatcggctgctacgcccagaccgagcttggccacggctctaacgtgcgaggactcgagactaccgccacttgggacccctctgaccagaccttcatcattcactctcccactctgaccgcctctaagtggtggatcggctctctgggacgaaccgccaaccacgccgtggtgatggcccagctgtacatcggcggcaagaactacggaccccatcctttcgtggtgcagatccgagacatggaaacccaccagcctctcgagaacgtgtacgtgggcgacatcggccccaagttcggctacaacaccatggacaacggctttctgctgttcaacaagctgaagattccccacgtgaacatgctggcccgattcgcccaggtggacaaggccaccaacaagtacattcgacccgcttctccctctctgatgtacggcaccatgacctgggtgcgatccaacatcgtgctgcaggctggcggcgtgctcgcccgaggcgtgaccattgccgtgcgatactgcgccgtgcgacgacagttccaggaccgagatgccaaggccaacgccgaagagaaccaggtgctgaactacaagatggtccagattcgactgctgcccctgctggccgccatgtacgccctgcacttcaccggccgaggcatgatgcgactgtacgaagaaaaccaagaacgaatgaaggctgccgctcaggccgaccaagagaagcgaggcgctggccccgagcagctgcgagccggatctgacctgctggctgacctgcacgctacctcttgcggcctgaaggccctggcctctaccaccgctggcgagggcctcgaggtgtgccgacgagcctgtggcggccacggatactctaactactctggcatcggaccctggtacgccgactacctgcctactctgacctgggagggcgacaactacatgctgactcagcaggttgcccgatacctgctcaagtctgcccgagccgtgctggccggcaagggcaccgccaacgacacctctcgaatcctgcaggcctacctcgctcgacgagacaagggcgcctctttcgacatcctgggcaacgacgccgacattgtggccgccttcgcctggcgaaccgctcacctgaccttcgagactctgaagtaccgagatgtcgagaagcgatcttggaactctctgctgatcaacttctggcgactgtctaccgctctgtctcagtacctggtggtgaagaacttctacgaggccgtgaactctcccgagatccgatcttctctggacaaggacactgcttctaccctgcgatctctgttccgactgcacgctctgcacaccctggaccgagaggcctccgagttcttctcttctgccgccgtgaccgtgcgacagatcggactgacccagacctctgaggtgcccaagctgctggacgagattcgaccccacgccgtccgactggtggactcttggaagatccccgactggcagctggactctgccctgggccgatctgacggcgacgtgtaccccgacctgttcaagcgagcctctatgcagaaccccgtgaacgacctcgtgttcgacccctatccttggaacgagaacgtcctgaagaacgccggtgagatcaagtctaagctgtag

SEQ ID NO: 45 - protein, 696 aa, Aspergillus nidulans
MPNPPPAWVQALKPASPQGTELLTQERAQSNIDVDTLGDLLHTKEALKKQDEILSVLKSEKVFDKSRNHVLGRTEKIQLALARGKRLQQLKKAHNWSDEDVHVANDLVSEPTPYGLHASMFLVTLREQGTPEQHKLFYERARNYEIIGCYAQTELGHGSNVRGLETTATWDPSDQTFIIHSPTLTASKWWIGSLGRTANHAVVMAQLYIGGKNYGPHPFVVQIRDMETHQPLENVYVGDIGPKFGYNTMDNGFLLFNKLKIPHVNMLARFAQVDKATNKYIRPASPSLMYGTMTWVRSNIVLQAGGVLARGVTIAVRYCAVRRQFQDRDAKANAEENQVLNYKMVQIRLLPLLAAMYALHFTGRGMMRLYEENQERMKAAAQADQEKRGAGPEQLRAGSDLLADLHATSCGLKALASTTAGEGLEVCRRACGGHGYSNYSGIGPWYADYLPTLTWEGDNYMLTQQVARYLLKSARAVLAGKGTANDTSRILQAYLARRDKGASFDILGNDADIVAAFAWRTAHLTFETLKYRDVEKRSWNSLLINFWRLSTALSQYLVVKNFYEAVNSPEIRSSLDKDTASTLRSLFRLHALHTLDREASEFFSSAAVTVRQIGLTQTSEVPKLLDEIRPHAVRLVDSWKIPDWQLDSALGRSDGDVYPDLFKRASMQNPVNDLVFDPYPWNENVLKNAGEIKSKL

SEQ ID NO: 46 - DNA, 1977 bp, Artificial sequence
  codon-optimized nucleotide sequence
  misc_feature (1)..(1977): DNA Codon-optimised sequence of peroxisomal oxidase, Cucurbita maxima
atggccgctggcaaggccaaggctaagatcgaggtggacatgggatctctgtctctgtacatgcgaggcaagcaccgagagatccaagagcgagtgttcgagtacttcaactctcgacccgagctgcagacccctgtgggcatctctatggccgaccaccgagagctgtgcatgaagcagctggtcggcctggtgcgagaggccggcattcgacccttccgattcgtgaacgaggaccccgccaagtacttcgccatcatggaagccgtgggctctgtggacgtgtctctggccatcaagatgggcgtgcagttctctctgtggggcggctctgtgatcaacctgggcaccaagaagcaccgggaccgattcttcgacggcatcgacaacgtggactaccccggctgcttcgccatgactgagctgcaccacggctctaacgtgcagggcctgcagaccaccgccactttcgaccccatcaccgacgagttcatcatcaacacccctaacgacggcgccatcaagtggtggatcggcaacgccgccgtccacggcaagttcgccaccgtgttcgccaagctggtgctgcccactcacgactctcgaaagaccgccgacatgggagtgcacgccttcatcgtgcccatccgagatctgaagtctcacaagaccctgcctggcatcgagatccacgactgcggccacaaggtgggcctgaacggcgtggacaacggcgccctgcgattccgatctgtgcgaattccccgagacaacctgctgaaccgattcggcgaggtgtctcgagatggcaagtacaagtcctctctgccctctatcaacaagcgattcgccgccactctgggcgagctggttggcggccgagtcggactggcctactcttctgcctctgtgctgaagatcgcctctactatcgccatccgatactccctgctgcgacagcagttcggccctcctaagcagcccgaggtgtccatcctggactaccagtctcagcagcacaagctgatgcccatgctggcctctacctacgccttccacttctctaccatgcagctcgtcgagaagtacgcccagatgaagaagacccacgacgaggaactggtgggcgacgtgcacgccctgtctgccggcctgaaggcctacgtgacctcttacaccgccaagtctctgtctacctgccgagaggcctgtggcggccacggatacgccgtggtcaaccgatttggcaccctgcgaaacgaccacgacatcttccagaccttcgagggcgacaacaccgtgctgctccagcaggtcgccgcctacctgctcaagcagtaccaagagaagttccaaggcggcaccctggccgtgacctggaactacctgcgagaatctatgaacacctacctctcgcagcccaaccctgtgaccgctcgatgggagtctgccgaccatctgcgagatcccaagtttcagctggacgctttccagtaccgaacctctcgactgctgcagtctgtggccgtgcgactgcgaaagcacaccaagaacctgggatctttcggcgcctggaaccgatgcctgaaccatctgctgaccctggctgagtctcacatcgagtctgtgattctggcccagttcatcgagtccgtgcagagatgtcccaacgctaacacccaggctaccctgaagctggtgtgcgacctgtacgctctggaccgaatctggaacgacatcggcacctaccgaaacgtcgactacgtggctcccaacaaggcaaaggccatccacaagctcaccgagtacctgtgcttccaggtgcgaaacattgcccaagagctggtggacgccttcgacctgcctgaccacgtgactcgagcccctattgccatgaagtctaacgcctactctcagtacacccagtacatcggcttcgaccagattacctctgtgggatcttcgtctaagctgtag

SEQ ID NO: 47 - protein, 658 aa, Cucurbita maxima
MAAGKAKAKIEVDMGSLSLYMRGKHREIQERVFEYFNSRPELQTPVGISMADHRELCMKQLVGLVREAGIRPFRFVNEDPAKYFAIMEAVGSVDVSLAIKMGVQFSLWGGSVINLGTKKHRDRFFDGIDNVDYPGCFAMTELHHGSNVQGLQTTATFDPITDEFIINTPNDGAIKWWIGNAAVHGKFATVFAKLVLPTHDSRKTADMGVHAFIVPIRDLKSHKTLPGIEIHDCGHKVGLNGVDNGALRFRSVRIPRDNLLNRFGEVSRDGKYKSSLPSINKRFAATLGELVGGRVGLAYSSASVLKIASTIAIRYSLLRQQFGPPKQPEVSILDYQSQQHKLMPMLASTYAFHFSTMQLVEKYAQMKKTHDEELVGDVHALSAGLKAYVTSYTAKSLSTCREACGGHGYAVVNRFGTLRNDHDIFQTFEGDNTVLLQQVAAYLLKQYQEKFQGGTLAVTWNYLRESMNTYLSQPNPVTARWESADHLRDPKFQLDAFQYRTSRLLQSVAVRLRKHTKNLGSFGAWNRCLNHLLTLAESHIESVILAQFIESVQRCPNANTQATLKLVCDLYALDRIWNDIGTYRNVDYVAPNKAKAIHKLTEYLCFQVRNIAQELVDAFDLPDHVTRAPIAMKSNAYSQYTQYIGFDQITSVGSSSKL

SEQ ID NO: 48 - DNA, 2019 bp, Artificial sequence
  codon-optimized nucleotide sequence
  misc_feature (1)..(2019): DNA Codon-optimised sequence of peroxisomal oxidase, Homo sapiens
atgaaccccgacctgcgacgagagcgagactctgcctctttcaaccccgagctgctgacccacatcctggacggctctcccgaaaagacccgacgacgacgagaaatcgagaacatgatcctgaacgaccccgacttccagcacgaggacctgaactttctgacccgatctcagcgatacgaggtggccgtgcgaaagtctgccatcatggtgaagaagatgcgagagttcggaatcgctgaccccgacgagatcatgtggttcaagaacttcgtgcaccgaggacgacccgagcctctggacctgcacctgggcatgtttctgcccactctgctgcaccaggccaccgccgagcagcaagagcgattcttcatgcccgcctggaacctcgagatcatcggcacctacgctcagaccgagatgggccacggcacccacctccgaggactcgagactaccgccacctacgatcccgagactcaagagttcatcctgaactctcccaccgtgacctctatcaagtggtggcccggtggactgggcaagacctctaaccacgccatcgtgctggcccagctgatcaccaagggcaagtgctacggcctgcacgccttcatcgtgcccatccgagagatcggaacccacaagcctctgcctggcatcaccgtgggcgacatcggccccaagttcggctacgacgagattgacaacggctacctgaagatggacaaccaccgaattcctcgagagaacatgctgatgaagtacgcccaggtgaagcccgacggaacctacgtgaagcccctgtctaacaagctgacctacggaaccatggtgttcgtgcgatctttcctggtcggcgaggccgctcgagccctgtccaaggcctgcaccattgccatccgatactctgccgtgcgacaccagtctgagatcaagcccggcgagcctgagcctcagatcctggactttcagacccagcagtacaagctgttccctctgctggccaccgcttacgccttccagttcgtgggcgcctacatgaaggaaacctaccatcgaatcaacgaaggcatcggccagggcgacctgtctgagctgcccgaactgcacgccctgaccgccggactgaaggctttcacctcttggaccgccaacaccggcatcgaggcctgccgaatggcctgtggcggccacggctactctcactgctctggactgcccaacatctacgtgaacttcaccccttcgtgtaccttcgagggcgagaacaccgtgatgatgctgcagaccgctcgattcctcatgaagtcttacgaccaggtgcactctggcaagctggtgtgcggcatggtgtcttacctgaacgatctgccctctcagcgaattcagcctcagcaggttgccgtgtggcccaccatggtcgacatcaactctcccgagtctctgaccgaggcctacaagctgcgagctgctcgactggtcgagatcgccgccaagaacctgcagaaggaagtcatccaccgaaagtctaaggaagtggcttggaacctgacctctgtggacctggtgcgagcttctgaggcccactgccactacgtggtggtgaagctgttctctgagaagctgctgaagatccaggacaaggccatccaggccgtgctgcgatctctgtgcctgctgtactctctgtacggcatctctcagaacgccggcgacttcctgcagggctctatcatgactgagccccagattacccaggtcaaccagcgagtgaaggaactgctcaccctgatccgatctgacgccgtggctctggtggacgccttcgactttcaggacgtgaccctgggctctgtgctgggccgatacgacggcaacgtgtacgagaacctgttcgagtgggccaagaactcgcccctgaacaaggccgaggtgcacgagtcttacaagcacctgaagtctctgcagtctaagctggaccagattacttctgtgggatcttcttcgaagctgtag

SEQ ID NO: 49 - protein, 672 aa, Homo sapiens
MNPDLRRERDSASFNPELLTHILDGSPEKTRRRREIENMILNDPDFQHEDLNFLTRSQRYEVAVRKSAIMVKKMREFGIADPDEIMWFKNFVHRGRPEPLDLHLGMFLPTLLHQATAEQQERFFMPAWNLEIIGTYAQTEMGHGTHLRGLETTATYDPETQEFILNSPTVTSIKWWPGGLGKTSNHAIVLAQLITKGKCYGLHAFIVPIREIGTHKPLPGITVGDIGPKFGYDEIDNGYLKMDNHRIPRENMLMKYAQVKPDGTYVKPLSNKLTYGTMVFVRSFLVGEAARALSKACTIAIRYSAVRHQSEIKPGEPEPQILDFQTQQYKLFPLLATAYAFQFVGAYMKETYHRINEGIGQGDLSELPELHALTAGLKAFTSWTANTGIEACRMACGGHGYSHCSGLPNIYVNFTPSCTFEGENTVMMLQTARFLMKSYDQVHSGKLVCGMVSYLNDLPSQRIQPQQVAVWPTMVDINSPESLTEAYKLRAARLVEIAAKNLQKEVIHRKSKEVAWNLTSVDLVRASEAHCHYVVVKLFSEKLLKIQDKAIQAVLRSLCLLYSLYGISQNAGDFLQGSIMTEPQITQVNQRVKELLTLIRSDAVALVDAFDFQDVTLGSVLGRYDGNVYENLFEWAKNSPLNKAEVHESYKHLKSLQSKLDQITSVGSSSKL

SEQ ID NO: 50 - DNA, 2148 bp, Artificial sequence
  codon-optimized nucleotide sequence
  misc_feature (1)..(2148): DNA Codon-optimised sequence of peroxisomal oxidase, Paenarthrobacter ureafaciens
atgaccgaggtggtggaccgagcctcttctcccgcctctcctggctctaccaccgccgctgccgacggcgccaaggtggccgtcgagcctcgagtggacgtggccgctctgggcgagcagctcctcggccgatgggccgacatccgactgcacgcccgagatctggccggacgagaggtggtgcagaaggtcgagggactgacccacaccgagcaccgatctcgagtgttcggccagctgaagtacctggtggacaacaacgccgtgcaccgagctttcccttctcgactcggcggatctgacgaccacggcggcaacattgccggcttcgaggaactggtgactgctgacccctcgctgcagatcaaggccggcgtccagtggggcctgttcggctctgccgtgatgcacctgggcacccgagagcaccacgacaagtggctgcccggcatcatgtctctcgagatccccggctgcttcgccatgaccgagactggccacggctctgacgtggcctctatcgccaccaccgccacctacgacgaagagactcaagagttcgtgatcgacacacccttccgagccgcctggaaggactacatcggcaacgccgccaacgacggcctggccgccgtcgtgttcgctcagctgatcacccgaaaggtgaaccacggcgtccacgccttctacgtggacctgcgagatcccgccaccggcgactttctgcccggaatcggcggcgaggacgacggcatcaagggcggcctgaacggcatcgacaacggacgactgcacttcaccaacgtgcgaattccccgaactaacctgctgaaccgatacggcgacgtggctgtggacggcacctactcttctaccatcgagtctcccggccgacgattcttcaccatgctgggaaccctggtgcagggccgagtgtctctggacggcgctgccgtggccgcctctaaggtggccctgcagtctgccatccactacgccgccgagcgacgacagttcaacgccacctctcctaccgaggaagaggtgctgctggactaccagcgacatcagcgacgactgtttacccgactggctactacctacgctgcctctttcgcccacgaacagctgctgcaaaagttcgacgacgtgttctctggcgcccacgacaccgacgccgaccgacaggacctcgagactctggctgccgctctgaagcccctgtctacctggcacgccctggacaccctgcaagagtgccgagaggcctgcggcggagccggcttcctgatcgagaaccgattcgcctctctgcgagctgacctggacgtgtacgtgaccttcgagggcgacaacaccgtgctgctgcagctggtggccaagcgactgctggccgactacgccaaggaattccgaggcgccaacttcggcgtgctggcccgatacgtcgtggaccaggccgctggcgtggctctgcaccgaaccggcctgcgacaggtggcccagttcgtggccgactccggctctgtgcagaagtctgccctggctctgcgagatgaggaaggccagcgaaccctgctgaccgaccgagtgcagtctatggtggccgaggtgggcgctgccctgaagggcgctggcaagctgccccagcaccaggctgctgccctgttcaaccagcatcagaacgagctgatcgaggccgctcaggcccacgccgagctgctccagtgggaagccttcaccgaggctctggccaaggtcgacgacgccggcaccaaggaagtgctgacccgactgcgggacctgttcggactgtctctgattgagaagcacctgtcttggtatctgatgaacggccgactgtctatgcagcggggacgaaccgtgggcacctacatcaaccgactgctcgtgaagattcgaccccacgctctggacctggtcgacgccttcggctacggcgctgagcatctgcgagccgccattgccaccggtgccgaggccactcgacaggacgaggcccgaacctacttccgacagcagcgagcctctggatctgcccctgccgacgaaaagaccctgctggccattaaggccggcaagtcccgagatcagattacctctgtgggatcttcttcgaagctgtag

SEQ ID NO: 51 - protein, 715 aa, Paenarthrobacter ureafaciens
MTEVVDRASSPASPGSTTAAADGAKVAVEPRVDVAALGEQLLGRWADIRLHARDLAGREVVQKVEGLTHTEHRSRVFGQLKYLVDNNAVHRAFPSRLGGSDDHGGNIAGFEELVTADPSLQIKAGVQWGLFGSAVMHLGTREHHDKWLPGIMSLEIPGCFAMTETGHGSDVASIATTATYDEETQEFVIDTPFRAAWKDYIGNAANDGLAAVVFAQLITRKVNHGVHAFYVDLRDPATGDFLPGIGGEDDGIKGGLNGIDNGRLHFTNVRIPRTNLLNRYGDVAVDGTYSSTIESPGRRFFTMLGTLVQGRVSLDGAAVAASKVALQSAIHYAAERRQFNATSPTEEEVLLDYQRHQRRLFTRLATTYAASFAHEQLLQKFDDVFSGAHDTDADRQDLETLAAALKPLSTWHALDTLQECREACGGAGFLIENRFASLRADLDVYVTFEGDNTVLLQLVAKRLLADYAKEFRGANFGVLARYVVDQAAGVALHRTGLRQVAQFVADSGSVQKSALALRDEEGQRTLLTDRVQSMVAEVGAALKGAGKLPQHQAAALFNQHQNELIEAAQAHAELLQWEAFTEALAKVDDAGTKEVLTRLRDLFGLSLIEKHLSWYLMNGRLSMQRGRTVGTYINRLLVKIRPHALDLVDAFGYGAEHLRAAIATGAEATRQDEARTYFRQQRASGSAPADEKTLLAIKAGKSRDQITSVGSSSKL

SEQ ID NO: 52 - DNA, 2022 bp, Artificial sequence
  codon-optimized nucleotide sequence
  misc_feature (1)..(2022): DNA Codon-optimised sequence of peroxisomal oxidase, Rattus norvegicus
atgaaccccgacctgcgaaaggaacgagcctctgccactttcaaccccgagctgatcacccacatcctggacggctctcccgagaacacccgacgacgacgagaaatcgagaacctgatcctgaacgaccccgacttccagcacgaggactacaactttctgacccgatctcagcgatacgaggtggccgtgaagaagtctgccaccatggtcaagaagatgcgagagtacggcatctctgaccccgaagagatcatgtggttcaagaactctgtgcaccgaggacaccctgagcctctggacctgcacctgggcatgtttctgcccactctgctgcaccaggctaccgccgagcagcaagagcgattcttcatgcccgcctggaacctcgagatcaccggcacctacgctcagaccgagatgggccacggcacccacctccgaggactcgagactaccgccacttacgaccccaagactcaagagttcatcctgaactctcccaccgtgacctctatcaagtggtggcccggtggcctgggcaagacctctaaccacgccatcgtgctggcccagctgattacccagggcgagtgctacggcctgcacgccttcgtggtgcccatccgagagatcggaacccacaagccactgcctggcatcaccgtgggcgacatcggccccaagttcggctacgaggaaatggacaacggctacctgaagatggacaactaccgaattcctcgagagaacatgctgatgaagtacgcccaggtgaagcccgacggaacctacgtgaagcccctgtctaacaagctgacctacggaaccatggtgttcgtgcgatctttcctggtgggcaacgccgctcagtctctgtctaaggcctgcaccattgccatccgatactctgccgtgcgacgacagtctgagatcaagcagtctgagcccgagcctcagatcctggactttcagacccagcagtacaagctgttccctctgctggccaccgcctacgccttccacttcgtgggccgatatatgaaggaaacctacctgcgaatcaacgagtctatcggccagggcgacctgtctgagctgcccgagctgcacgccctgaccgccggactgaaggctttcaccacctggaccgccaacgccggcatcgaggaatgccgaatggcctgtggcggccacggctactctcactcctctggcatccccaacatctacgtgaccttcactcccgcctgcaccttcgagggtgagaacaccgtgatgatgctgcagaccgctcgattcctgatgaagatctacgaccaggtgcgatctggcaagctggtcggcggcatggtgtcttacctgaacgatctgccctctcagcgaattcagcctcagcaggttgccgtgtggcccactatggtggacatcaactcgctcgagggcctgaccgaggcctacaagctgcgagccgctcgactggtcgagatcgccgccaagaacctgcagacccacgtgtctcaccgaaagtctaaggaagtggcttggaacctgacctctgtggacctggtgcgagcttctgaggcccactgccactacgtggtggtgaaggtgttctctgacaagctgcccaagatccaggacaaggctgtccaggccgtgctgcgaaacctgtgcctgctgtactctctgtacggaatctctcagaagggcggcgacttcctcgagggctctatcatcaccggcgctcagctgtctcaggtcaacgctcgaatcctcgagctgctgaccctgattcgacccaacgccgtggctctggtggacgctttcgacttcaaggacatgaccctgggctctgtgctgggacgatacgacggcaacgtgtacgagaacctcttcgagtgggccaagaagtctcccctgaacaagaccgaggtgcacgagtcttaccacaagcacctgaagcctctgcagtctaagctggaccagattacctccgtgggatcttcttcgaagctgtag

SEQ ID NO: 53 - protein, 673 aa, Rattus norvegicus
MNPDLRKERASATFNPELITHILDGSPENTRRRREIENLILNDPDFQHEDYNFLTRSQRYEVAVKKSATMVKKMREYGISDPEEIMWFKNSVHRGHPEPLDLHLGMFLPTLLHQATAEQQERFFMPAWNLEITGTYAQTEMGHGTHLRGLETTATYDPKTQEFILNSPTVTSIKWWPGGLGKTSNHAIVLAQLITQGECYGLHAFVVPIREIGTHKPLPGITVGDIGPKFGYEEMDNGYLKMDNYRIPRENMLMKYAQVKPDGTYVKPLSNKLTYGTMVFVRSFLVGNAAQSLSKACTIAIRYSAVRRQSEIKQSEPEPQILDFQTQQYKLFPLLATAYAFHFVGRYMKETYLRINESIGQGDLSELPELHALTAGLKAFTTWTANAGIEECRMACGGHGYSHSSGIPNIYVTFTPACTFEGENTVMMLQTARFLMKIYDQVRSGKLVGGMVSYLNDLPSQRIQPQQVAVWPTMVDINSLEGLTEAYKLRAARLVEIAAKNLQTHVSHRKSKEVAWNLTSVDLVRASEAHCHYVVVKVFSDKLPKIQDKAVQAVLRNLCLLYSLYGISQKGGDFLEGSIITGAQLSQVNARILELLTLIRPNAVALVDAFDFKDMTLGSVLGRYDGNVYENLFEWAKKSPLNKTEVHESYHKHLKPLQSKLDQITSVGSSSKL

SEQ ID NO: 54 - DNA, 1152 bp, Yarrowia lipolytica
atggaaccgttcaagctagctcccacgcccgtcactctgcccgagtactttgcttttaaagaattcactcctaagaaccccgaacagggtgtgagatacttccactccgtcatcaagccgtggcgccctttctcgtctcctggcacctttggaggattctgtctgagccagggagctctttgcgccgcctacacatgccccaagggctttgttgtgcacaaccagcactcgtacttccttcttccgggtcgttgggacgtgccgttcctatggagagtggaatctgtgcgagacggccgatcttactgcacccgagaagtcaaggcgtaccagcccgatctcggattcgaattccccgagtctccttaccaacagccctctgacttcgacttcacagaccccgccagcaaaaaatggctcgcctacacagcccattcctccatcaagctaccccacaaagacaccatgttccacgaaaagaagctacgacaggattttttcgccaaaaacgtgcctggaggagctgagggacacgatctggcacccgacattgacatccccatgtgggtggactggtccaaggaccctgctaacggctacaagctggaacctcatccgatagagatgcgcaaggtggacatggacaaggtgcttcctgcagtcaacaagggcaaggacgtggccgagagacgtcagctgtactttttccgagtgccctacaagctgcctgacgacatgaactaccatgtggcagccatgctctatctgtcagatcgaaactcgctcttcacctgtatgaacctgagagacaaggtgcccactttggcgcgcctggcatcgctggaccatcagtttactatgcatgacatgcactctcgtgtggacgagggctggatgcctatggagacctggactgactgggctggagactgtcgaggacagtaccagggccgactatttacagatgaaggcaagcttgtatgcacatttatgcaagacggtctgattcgaaccgttgaggaggaccacgatgacgacgacgacaagaaggaggatgacaaggctgataacaccacccgacaggttcctcgaagaagaaagaagaagactcctgctcagaatttgtttctcaagttcaaagctgtgctttaa

SEQ ID NO: 55 - DNA, 630 bp, Yarrowia lipolytica
atgtttcgtcacgcgatcagatcaattggcctcgtgactcctgtcagagtcattacccccactctacgtgcttctctggttctcggggcagttcgttctcagagttcgcacgccaaaccccaagcacccgattggatccagaatctgctcgatgagcacgagggcaaaggatacttgcttgccgacgctgggctgccgtctcagggcgtctcttggggagaaattgactccttccagcatgttaacaacaaggtgtatctggcttggttcgagaccgcccgtgttaacatgtttctcaagtggggcacggactttcagcggttcatgagtggccagtcggtggcaccggtcatgcggtcggtcaatctagcttggcgataccccatcaaattccccgaccaggtgacggtcgtccacaaaattgaccagattctcgatgaccggttcattctcaagggcgtcgtgataggccacaagtccaagaaggtgtgtgcacgaattgaagaggtgattgtcgctgtcgattacactaagggtgccaccaagtgttctattccggacgatatgagagagtttctggagcagaaaaagagagagcaaggcgcgggggagtatgatgcgcagatttag

SEQ ID NO: 56 - DNA, 1185 bp, Artificial Sequence
  codon-optimized nucleotide sequence
  misc_feature (1)..(1185): Y. lipolytica codon-optimised nucleotide sequence of thioesterase from Cuphea hookeriana ChFatB3
atggtggccgctgccgcctcttctgccttcttctcggtgcccactcctggcatctctcccaagcctggcaagttcggcaacggcggcttccaggtgaaggccaacgccaacgctcacccctcgctgaagtctggctctctcgagactgaggacgacacctcttcctcttctccaccacctcgaaccttcatcaaccagctgcctgactggtctatgctgctgtctgccatcaccaccatcttcggagccgccgagaagcagtggatgatgctggaccgaaagtctaagcgacccgacatgctgatggaacccttcggcgtggactctatcgtgcaggacggcgtgttcttccgacagtctttctctatccgatcttacgagattggcgccgaccgaaccacctctatcgagactctgatgaacatgtttcaagagacttctctgaaccactgcaagtctaacggcctgctgaacgacggcttcggacgaacccctgagatgtgcaagaagggcctgatctgggtcgtgaccaagatgcaggtcgaggtgaacagataccccatctggggcgactctattgaggtcaacacctgggtgtctgagtctggcaagaacggcatgggccgagactggctgatctctgactgctctaccggcgagatcctggtgcgagccacctctgtgtgggccatgatgaaccagaagacccgacgactgtctaagttcccattcgaggtgcgacaagagatcgctcccaacttcgtcgactctgtccccgtgatcgaggacgaccgaaagctgcacaagctggacgtcaagaccggcgactccatccacaacggactgacccctcgatggaacgacctggacgtgaaccagcacgtgaacaacgtgaagtacatcggctggatcctgaagtcggtgcccaccgacgtgttcgaggcccaagagctgtgcggcgtgaccctcgagtaccgacgagagtgcggacgagactccgtgatggaatctgtgaccgctatggacccctctaaagaaggcgaccgatctgtctaccagcacctcctgcgactcgaggacggcgccgacattgccatcggccgaaccgagtggcgacccaagaacgctggcgccaacggcgccatctctaccggaaagacctctaaccgaaactctgtgtcttaa

SEQ ID NO: 57 - protein, 394 aa, Cuphea hookeriana
MVAAAASSAFFSVPTPGISPKPGKFGNGGFQVKANANAHPSLKSGSLETEDDTSSSSPPPRTFINQLPDWSMLLSAITTIFGAAEKQWMMLDRKSKRPDMLMEPFGVDSIVQDGVFFRQSFSIRSYEIGADRTTSIETLMNMFQETSLNHCKSNGLLNDGFGRTPEMCKKGLIWVVTKMQVEVNRYPIWGDSIEVNTWVSESGKNGMGRDWLISDCSTGEILVRATSVWAMMNQKTRRLSKFPFEVRQEIAPNFVDSVPVIEDDRKLHKLDVKTGDSIHNGLTPRWNDLDVNQHVNNVKYIGWILKSVPTDVFEAQELCGVTLEYRRECGRDSVMESVTAMDPSKEGDRSVYQHLLRLEDGADIAIGRTEWRPKNAGANGAISTGKTSNRNSVS

SEQ ID NO: 58 - DNA, 5449 bp, Artificial sequence
  Artificial sequence
  misc_feature (1)..(5549): Fusion of a truncated version of FAS1 from Y. lipolytica and a truncated version of the thioesterase TesA from E. coli
gtgagtatcgaccgaagcaggatgatctctacatgagatatgcaacgcgtacgtgattcaattgatcctaacacagtaccctaccacaggtgtcaacaccccccagagcgccgcctcattaagaccactggtgctatcgcacggccaaactgagcactcgctgctggtgcccacctctctgtacatcaactgcaccacgctccgagaccagttctacgcctctctacctccagccactgaagacaaggccgacgatgatgagccctcctcctccacagagcttctagctgccttcctgggatttactgccaagaccgtcgaggaagagcccggaccatacgacgacgttctctctctcgtgcttaacgagtttgagacccggtacttgcgaggtaacgacatccacgctgtggcctcctccttgttacaagacgaggacgtgcctaccaccgttggtaagatcaagagggtgattcgagcctactacgccgcacgaattgcctgcaaccggcccatcaaggcccactcgtcggctctgttccgagccgcatctgaagactcggacaacgtctctctgtacgccatcttcggtggccagggaaacaccgaggactactttgaggaactgcgggagatttacgacatctaccaggggctggtcggcgacttcattcgggaatgtggagcccagcttctggcgctgtctcgagatcacattgctgctgagaaaatttataccaagggctttgatatcgtcaagtggctggaacaccccgagaccatccccgactttgagtacctaatttctgctcccatctctgtacccatcatcggtgttatccagctggcacactacgctgtcacctgtcgagttttgggtcttaatcctggccaggtccgagacaacctcaagggtgccactggccattctcagggtctgatcaccgcaattgccatctctgcctccgactcgtgggacgagttctataactctgcctctcgaattctcaagatcttcttcttcatcggtgtccgtgtccaacaggcttacccctccactttcctgcctccctccactctggaagacagtgtcaagcagggtgagggcaagcccactcccatgctgtccatccgagacctgtctctcaaccaggttcaggagttcgtcgatgccaccaacttgcatttgcccgaagataagcagatcgtcgtgtctctgatcaatggtcctcgaaacgttgtcgttactggccccccccagtctctgtatggtctgtgtcttgtgcttcgaaaacagaaggccgagaccggtctggaccaaagccgagtgccccacagtcagcgaaagctcaaattcacacatcgtttcctgcccatcacctctcctttccactcgtacctgctggagaagagcacggatctgatcatcaacgacctggagtcttccggtgtggagtttgtgtcctccgagctcaaggtgcctgtttacgacacctttgatggctccgtgctgtctcagctacccaagggtatcgtcagccgtctggtcaacctcatcactcatctgcccgtcaagtgggagaaggccactcagtttcaggcctcccacattgtggactttggtcccggtggcgcttctggtcttggtctgttgacccacaagaacaaggatggaactggagtgcgaactattcttgctggtgtcattgaccagcccctcgagttcggcttcaagcaggagctgtttgaccgacaggagtcgtccattgtttttgctcaaaactgggccaaggagttttctcccaagctcgtcaagatctcctccaccaacgaggtctatgtcgacaccaaattctctcgtctgactggccgagcccccatcatggtcgctggtatgacccctaccactgtcaaccccaaatttgtggctgccactatgaactccggctaccacatcgagcttggtggtggaggctactttgcccccggtatgatgaccaaggcccttgaacacattgagaagaacactcctcccggatccggtatcaccatcaacctgatctacgtcaaccctcgactgattcaatggggtattcctctgattcaggagcttcgacagaagggtttccccattgaaggtctcaccattggtgccggtgtgccctctctggaggttgctaacgagtggattcaggatctgggcgtcaagcacatcgccttcaagcctggatccatcgaggccatctcctcggtgattcgaatcgccaaggccaacccagactttcctatcatccttcagtggaccggaggtcgaggaggaggacatcattcgtttgaggacttccacgctcccattctgcagatgtactccaagatccgacgatgcagcaacattgtgctgattgccggatctggtttcggtgcttctaccgactcctacccatacctcaccggttcatggtcccgagactttgactaccctcccatgccctttgacggtatcctggttggttctcgagtcatggttgccaaggaggctttcacttctctgggagccaagcagctcattgttgactctccgggtgttgaggattctgagtgggagaaaacctacgacaagcccactggtggcgtcatcaccgttctctccgagatgggtgagcctatccacaagctcgccactcgaggtgtgctcttctggcacgagatggacaagaccgtgttctccctgcccaagaagaagcgtctggaagtgctcaagtccaagcgagcctacatcatcaagcgtctcaacgacgacttccagaagacttggtttgccaagaacgcccagggacaggtgtgtgatctcgaagacctcacctacgcggaggtcatccagcgacttgttgacctcatgtacgtgaagaaggaaagccgatggatcgatgtcactctccgaaatcttgccggcactttcattcgacgagttgaggagcgattctccaccgagacaggtgcctcttctgtgttgcagagcttttccgagctggattccgagcccgagaaggttgtcgagcgggtgtttgagctcttccctgcctctactacccagatcatcaacgctcaagacaaggaccacttcctcatgctgtgtctcaaccccatgcagaagcccgtgcccttcatccctgttctggatgacaactttgagttcttcttcaagaaggactctctgtggcagtgcgaggacctcgcagctgttgtggacgaagacgttggacgaatctgtattcttcagggtcccgttgctgtcaagcactccaagattgtcaacgagcccgtcaaggagattctcgactccatgcacgaaggtcacatcaagcagctgcttgaggatggcgagtacgctggcaacatggccaacatcccccaggtcgaatgctttggtggaaagcctgctcagaacttcggtgacgttgctctcgactctgtcatggttcttgatgacctcaacaagaccgtgttcaagattgagaccggcacctctgctctgccttctgctgcagattggttctctctgctggccggtgacaagaactcttggcgacaggtcttcctgtccactgacaccattgtgcagaccaccaagatgatctccaaccctctgcatcgacttctggagcccatcgcaggtttgcaggttgagattgagcaccctgatgagcccgagaacaccgtcatctctgctttcgagcccatcaacggcaaggtcaccaaggtgctggagctgcgaaagggtgccggagacgtcatttcgctgcagctgatcgaagcgcgtggcgttgaccgagtccccgttgctcttcctctggaattcaagtaccagccccagattggctacgctcccattgttgaggttatgaccgacaggaacacccgaatcaaggagttctactggaagctgtggtttggccaggactccaagtttgagattgacaccgacatcaccgaggaaatcattggcgatgacgttaccatctctggcaaggccattgccgactttgtccacgctgttggcaacaagggcgaggcctttgttggtcgatctacctctgctggtactgtcttcgctcccatggactttgccattgttttgggctggaaggccattatcaaggcaatctttccccgagcaattgatgctgacattctgcgtctggtacatctgtccaacggcttcaagatgatgcctggcgccgaccctctgcagatgggtgatgttgtttccgccactgccaagatcgacactgtcaagaactccgctaccggcaagactgttgctgttcgaggtcttctcacccgagacggcaagcctgtcatggaggttgtttccgaattcttctaccgaggcgaattctccgacttccagaacacttttgagcgacgagaggaggtacccatgcaactgaccctcaaggacgccaaggccgtggccattctctgctccaaggagtggtttgagtacaatggcgacgataccaaggacctcgagggcaagaccattgtgttccgaaactcgtcattcatcaagtacaagaatgagaccgtcttctcttctgtgcacaccaccggtaaggtattgatggagctgccctccaaggaggtcattgagattgccactgttaactaccaggctggcgagtctcatggcaatcccgtcattgattacctggagcgaaatggaaccaccattgagcagcctgttgagtttgagaagcccatccctctgtccaaggcagatgatcttctctccttcaaggctccttcttccaacgagccctacgctggtgtgtccggtgactacaatcccatccacgtgtctcgagcctttgcttcctatgcatcccttcctggagcagcggacacgttattgattctgggtgatagcctgagcgccgggtatcgaatgtctgccagcgcggcctggcctgccttgttgaatgataagtggcagagtaaaacgtcggtagttaatgccagcatcagcggcgacacctcgcaacaaggactggcgcgccttccggctctgctgaaacagcatcagccgcgttgggtgctggttgaactgggcggcaatgacggtttgcgtggttttcagccacagcaaaccgagcaaacgctgcgccagattttgcaggatgtcaaagccgccaacgctgaaccattgttaatgcaaatacgtctgcctgcaaactatggtcgccgttataatgaagcctttagcgccatttaccccaaactcgccaaagagtttgatgttccgctgctgcccttttttatggaagaggtctacctcaagccacaatggatgcaggatgacggtattcatcccaaccgcgacgcccagccgtttattgccgactggatggcgaagcagttgcagcctttagtaaatcatgactcataa

SEQ ID NO: 59 - protein, 2268 aa, Artificial sequence
  Artificial sequence
  MISC_FEATURE (1)..(2268): Fusion of a truncated version of FAS1 from Y. lipolytica and a truncated version of the thioesterase TesA from E. coli
YPTTGVNTPQSAASLRPLVLSHGQTEHSLLVPTSLYINCTTLRDQFYASLPPATEDKADDDEPSSSTELLAAFLGFTAKTVEEEPGPYDDVLSLVLNEFETRYLRGNDIHAVASSLLQDEDVPTTVGKIKRVIRAYYAARIACNRPIKAHSSALFRAASEDSDNVSLYAIFGGQGNTEDYFEELREIYDIYQGLVGDFIRECGAQLLALSRDHIAAEKIYTKGFDIVKWLEHPETIPDFEYLISAPISVPIIGVIQLAHYAVTCRVLGLNPGQVRDNLKGATGHSQGLITAIAISASDSWDEFYNSASRILKIFFFIGVRVQQAYPSTFLPPSTLEDSVKQGEGKPTPMLSIRDLSLNQVQEFVDATNLHLPEDKQIVVSLINGPRNVVVTGPPQSLYGLCLVLRKQKAETGLDQSRVPHSQRKLKFTHRFLPITSPFHSYLLEKSTDLIINDLESSGVEFVSSELKVPVYDTFDGSVLSQLPKGIVSRLVNLITHLPVKWEKATQFQASHIVDFGPGGASGLGLLTHKNKDGTGVRTILAGVIDQPLEFGFKQELFDRQESSIVFAQNWAKEFSPKLVKISSTNEVYVDTKFSRLTGRAPIMVAGMTPTTVNPKFVAATMNSGYHIELGGGGYFAPGMMTKALEHIEKNTPPGSGITINLIYVNPRLIQWGIPLIQELRQKGFPIEGLTIGAGVPSLEVANEWIQDLGVKHIAFKPGSIEAISSVIRIAKANPDFPIILQWTGGRGGGHHSFEDFHAPILQMYSKIRRCSNIVLIAGSGFGASTDSYPYLTGSWSRDFDYPPMPFDGILVGSRVMVAKEAFTSLGAKQLIVDSPGVEDSEWEKTYDKPTGGVITVLSEMGEPIHKLATRGVLFWHEMDKTVFSLPKKKRLEVLKSKRAYIIKRLNDDFQKTWFAKNAQGQVCDLEDLTYAEVIQRLVDLMYVKKESRWIDVTLRNLAGTFIRRVEERFSTETGASSVLQSFSELDSEPEKVVERVFELFPASTTQIINAQDKDHFLMLCLNPMQKPVPFIPVLDDNFEFFFKKDSLWQCEDLAAVVDEDVGRICILQGPVAVKHSKIVNEPVKEILDSMHEGHIKQLLEDGEYAGNMANIPQVECFGGKPAQNFGDVALDSVMVLDDLNKTVFKIETGTSALPSAADWFSLLAGDKNSWRQVFLSTDTIVQTTKMISNPLHRLLEPIAGLQVEIEHPDEPENTVISAFEPINGKVTKVLELRKGAGDVISLQLIEARGVDRVPVALPLEFKYQPQIGYAPIVEVMTDRNTRIKEFYWKLWFGQDSKFEIDTDITEEIIGDDVTISGKAIADFVHAVGNKGEAFVGRSTSAGTVFAPMDFAIVLGWKAIIKAIFPRAIDADILRLVHLSNGFKMMPGADPLQMGDVVSATAKIDTVKNSATGKTVAVRGLLTRDGKPVMEVVSEFFYRGEFSDFQNTFERREEVPMQLTLKDAKAVAILCSKEWFEYNGDDTKDLEGKTIVFRNSSFIKYKNETVFSSVHTTGKVLMELPSKEVIEIATVNYQAGESHGNPVIDYLERNGTTIEQPVEFEKPIPLSKADDLLSFKAPSSNEPYAGVSGDYNPIHVSRAFASYASLPGTITHGMYSSAAVRSLIEVWAAENNVSRVRAFSCQFQGMVLPNDEIVTRLEHVGMINGRKIIKVTSTNRETEAVVLSGEAEVEQPISTFVFTGQGSQEQGMGMDLYASSEVAKKVWDKADEHFLQNYGFSIIKIVVENPKELDIHFGGPKGKKIRDNYISMMFETIDEKTGNLISEKIFKEIDETTDSFTFKSPTGLLSATQFTQPALTLMEKASFEDMKAKGLVPVDATFAGHSLGEYSALASLGDVMPIESLVDVVFYRGMTMQVAVPRDAQGRSNYGMCAVNPSRISTTFNDAALRFVVDHISEQTKWLLEIVNYNVENSQYVTAGDLRALDTLTNVLNVLKLEKINIDKLLESLPLEKVKEHLSEIVTEVAKKSVAKPQPIELERGFAVIPLKGISVPFHSSYLRNGVKPFQNFLVKKVPKNAVKPANLIGKYIPNLTAKPFEITKEYFEEVYKLTGSEKVKSIINNWESYESKQAADTLLILGDSLSAGYRMSASAAWPALLNDKWQSKTSVVNASISGDTSQQGLARLPALLKQHQPRWVLVELGGNDGLRGFQPQQTEQTLRQILQDVKAANAEPLLMQIRLPANYGRRYNEAFSAIYPKLAKEFDVPLLPFFMEEVYLKPQWMQDDGIHPNRDAQPFIADWMAKQLQPLVNHDS

SEQ ID NO: 60 - DNA, 1402 bp, Agrotis ipsilon
aataaatagccacaatggccgtgatcatctcccgagaggaagagaagctgtctgtccccgagttctacgccggcaagtctatcttcattaccggcggcaccggattcctcggcaaggtgttcatcgagaagctgctctactcttgccccgacatcgacaagatctacatgctgatccgagagaagaagaacctctctatcgacgagcgaatgaccatgttcctggacgaccctctgttctctcgactgaaggaaaagcgacccggcgacgtcgagaagatcgtgctgatccccggcgacatctcttctcccaacctgggcctgtctgccgagaacgaacgaatcctgatcgagaacgtgtctgtgatcatccactctgccgccaccatcaagttcaacgagcccctgcctatcgcctggaagatcaacgtcgagggcacccgaatgctgatggacctgtctcgacgaatgaagcgaatcaaggtgtttatccacatctctaccgcctactctaacgccaactctgagcgagccgccgtggaagagattctgtaccccgctcctgccgacatggaccaggtgtaccagctcgtgaaggacggcgtgaccgaggaagaaaccgagatcctgctgaacggactgcccaacacctacaccttcaccaaggctctggccgagcacctggccgctgagcaccaggtgcacgtgcccaccgtgattattcgaccctctatcgtgggctctatcaaggacgagcccatccgaggctggctgtgcaactggttcggcgccaccggcatctctgtgttcaccgccaagggcctgaaccgagtgctgctcggaaaggcctctaacatcgtggacgtgatccccgtggactacgtggccaacctggtgatcgtggctggcgccaagaacggcggcgagaagtctgaggaactgaagatctataactgctgttcttctgactgcaaccccgtgaccgtgaagaagatcctgaaggaattcatcgacgacaccattaagaacaagtctcacatcatgcctctgcctggctggttcgtgttcaccaagtacaagtggctgatgaccctgctgaccatcatcttccagatgatccccatgtacctggccgacgtgtaccgagtcctgatgggcaagaaccctcggtacatgaagctgcaccacctggtcattcagacccgactggtgatcaacttcttcaccttccactcttgggtgatgaagaccgatcgagcccgagagctgttcggctctctgtctcccgttgagaagcacatgttcccttgggacccctctggcatcgactggaccgagtacctgcagtcttactgctacggcgtgcgacacttcctcgagaagcgaaagtagaatataaattt

SEQ ID NO: 61 - protein, 458 aa, Agrotis ipsilon
MAVIISREEEKLSVPEFYAGKSIFITGGTGFLGKVFIEKLLYSCPDIDKIYMLIREKKNLSIDERMTMFLDDPLFSRLKEKRPGDVEKIVLIPGDISSPNLGLSAENERILIENVSVIIHSAATIKFNEPLPIAWKINVEGTRMLMDLSRRMKRIKVFIHISTAYSNANSERAAVEEILYPAPADMDQVYQLVKDGVTEEETEILLNGLPNTYTFTKALAEHLAAEHQVHVPTVIIRPSIVGSIKDEPIRGWLCNWFGATGISVFTAKGLNRVLLGKASNIVDVIPVDYVANLVIVAGAKNGGEKSEELKIYNCCSSDCNPVTVKKILKEFIDDTIKNKSHIMPLPGWFVFTKYKWLMTLLTIIFQMIPMYLADVYRVLMGKNPRYMKLHHLVIQTRLVINFFTFHSWVMKTDRARELFGSLSPVEKHMFPWDPSGIDWTEYLQSYCYGVRHFLEKRK

SEQ ID NO: 62 - DNA, 1008 bp, Choristoneura rosaceana
atggctcccaacgtcgaggacatggaatctgacctgcctgagtctgaggaaaagctcgagaagctggtggctccccaggctgctccccgaaagtaccagatcatctacaccaacctgctgaccttcggctactggcacattgccggcctgtacggactgtacctgtgcttcacctctgccaagtggcagaccatcattctggccctgatcctgaacgagatggccattctgggcatcaccgctggcgcccaccgactgtgggctcaccgatcttacaaggccaccgtgcctctgcagatcatcctgatcatcttcaactccctgtctttccagaactctgccatccactggatccgagatcaccgaatgcaccacaagtactctgacaccgacggcgaccctcacaacgcctctcgaggcttcttctactctcacgtcggctggctgctggtgaagaagcaccccgaggtcaagaagcgagccaagaccatcgacatgtctgacatctactctaaccccatcctgcgattccagaagaagtacgctatccccttcatcggcatgatctgcttcgtgctgcccactattatccctatgtacttctggggcgagactctgtctaacgcctggcacatcaccatgctgcgatacgtgttctctctgaactctatcttcctggtgaactccgccgctcacctgtacggctaccgaccttacgacaagaacattctgcccgccgagaacaagatgaccttcattgcctgcctgggcgagaacttccacaactaccaccacgtgttcccttgggactaccgagcctctgagctgggcaacatcggaatgaactggaccgccaagttcatcgactttttcgcctggatcggctgggcctacgacctcaagaccgcctctgacgagaacatcaagtctcgaatgaagcgaaccggcgacggcaccgacgtgtctggacagaagtactcttgcgagtcctctgaggtgctgcagtaa

SEQ ID NO: 63 - protein, 335 aa, Choristoneura rosaceana
MAPNVEDMESDLPESEEKLEKLVAPQAAPRKYQIIYTNLLTFGYWHIAGLYGLYLCFTSAKWQTIILALILNEMAILGITAGAHRLWAHRSYKATVPLQIILIIFNSLSFQNSAIHWIRDHRMHHKYSDTDGDPHNASRGFFYSHVGWLLVKKHPEVKKRAKTIDMSDIYSNPILRFQKKYAIPFIGMICFVLPTIIPMYFWGETLSNAWHITMLRYVFSLNSIFLVNSAAHLYGYRPYDKNILPAENKMTFIACLGENFHNYHHVFPWDYRASELGNIGMNWTAKFIDFFAWIGWAYDLKTASDENIKSRMKRTGDGTDVSGQKYSCESSEVLQ

SEQ ID NO: 64 - DNA, 1005 bp, Choristoneura parallela
atggctcccaacgtcgaggacatggaatctgacatgcccgagtctgagaagtgggagaagctggtggctccccaggctgctccccgaaagtacgagatcatctacaccaacctgctgaccttcggctacggccacattgccggcctgtacggactgtacctgtgcttcacctctgccaagtggcagaccgtgatcctggccatcatcctgaacgagatggccattctgggcatcaccgctggcgcccaccgactgtggtcccaccgatcttacaaggccgctgtgcccctgcagatcattctgatgatcttcaactctctggccttccagaactctgccatcaactgggtgcgagatcaccgaatgcaccacaagtactctgacaccgacggcgaccctcacaacgcctctcgaggcttcttctactctcacgtcggctggctgctggtgaagaagcaccccgaggtcaaaaagcgaggcaagatgatcgacatgagcgacatctactctaaccccgtgctgcgattccagaagaagtacgctatccccttcatcggcatgatctgcttcgtgctgcccactattatccctatgtacttctggggcgagactctgtctaacgcctggcacatcaccatgctgcgatacgtgttctctctgaactctatcttcctggtgaactccgccgctcacctgtacggctaccgaccttacgacaagaacattctgcccgccgagaacaagatcgccctgatcgcctgcctgggcgactctttccacaactaccaccacgtgttcccttgggactaccgagcctctgagctgggcaacatcggaatgaactggaccgctcagttcatcgactttttcgcctggatcggctgggcctacgacctcaagaccgcctctgacgagaacatcaactctcgaatgaagcgaaccggcgacggcaccgacatctctggacagaagtactcttgcgagtcctctgaggtgctgcagtaa

SEQ ID NO: 65 - protein, 334 aa, Choristoneura parallela
MAPNVEDMESDMPESEKWEKLVAPQAAPRKYEIIYTNLLTFGYGHIAGLYGLYLCFTSAKWQTVILAIILNEMAILGITAGAHRLWSHRSYKAAVPLQIILMIFNSLAFQNSAINWVRDHRMHHKYSDTDGDPHNASRGFFYSHVGWLLVKKHPEVKKRGKMIDMSDIYSNPVLRFQKKYAIPFIGMICFVLPTIIPMYFWGETLSNAWHITMLRYVFSLNSIFLVNSAAHLYGYRPYDKNILPAENKIALIACLGDSFHNYHHVFPWDYRASELGNIGMNWTAQFIDFFAWIGWAYDLKTASDENINSRMKRTGDGTDISGQKYSCESSEVLQ

SEQ ID NO: 66 - DNA, 1050 bp, Artificial sequence
  Artificial sequence
  misc_feature (1)..(1050): Cpo_NPVE, codon-optimised for Y. lipolytica
atgccccctcagggacagcctcccgcctgggtcctggacgagtccgatgcagtgaccgaggacaaggacgtggccacccccgctcccgaagccgagaagcgaaagctgcagatcgtttggagaaacgtgaccctgttcgtgtttctgcacatcggagctctgtacggaggatacctgttctttaccaaggccatgtggactacccgaattttcactgtgctgctgtacattatgtctgggctgggtatcaccgccggcgcccatcgactctgggctcacaagtcttacaaggcccgactgcccctgcgactgctgctgaccctcttcaacaccatcgcctttcaggactccgttctggattgggcccgagatcaccgaatgcaccataagtactctgaaaccgacgcagatccccacaatgctacccgaggcttcttcttctctcacgtgggctggctgctggtgcgaaagcacccccagatcaaggccaagggacatactatcgacatgtctgacctgctggccgatcccgtgctgcgattccagaagaagtactacctgacactgatgcccttgtgctgcttcatcctgccctcttacattcccaccctctggggagagtctctgtggaacgcttactttgtgtgcgccatcttccgatactgttacgttctgaacgtgacttggctggtgaactccgctgcccacaaatggggtgaccgaccttacgacaagaacatcaaccctgtggagactaagcctgtgtctctggtggttttcggagagggattccacaactaccaccacaccttcccctgggattacaagaccgccgagctgggcggatactctctgaacctttccaaactgttcatcgatactatgtccaagattggatgggcctacgacctgaagtccgtttcccctgacatcgtggagaagcgagtgaagcgcaccggcgacggatctcaccacgtgtggggatgggacgatgctccctctgagcaaaaggtggctgccaccatcgtgaaccccgataagaccgagtaa

SEQ ID NO: 67 - protein, 349 aa, Cydia pomonella
MPPQGQPPAWVLDESDAVTEDKDVATPAPEAEKRKLQIVWRNVTLFVFLHIGALYGGYLFFTKAMWTTRIFTVLLYIMSGLGITAGAHRLWAHKSYKARLPLRLLLTLFNTIAFQDSVLDWARDHRMHHKYSETDADPHNATRGFFFSHVGWLLVRKHPQIKAKGHTIDMSDLLADPVLRFQKKYYLTLMPLCCFILPSYIPTLWGESLWNAYFVCAIFRYCYVLNVTWLVNSAAHKWGDRPYDKNINPVETKPVSLVVFGEGFHNYHHTFPWDYKTAELGGYSLNLSKLFIDTMSKIGWAYDLKSVSPDIVEKRVKRTGDGSHHVWGWDDAPSEQKVAATIVNPDKTE

SEQ ID NO: 68 - DNA, 1005 bp, Artificial sequence
  Artificial sequence
  misc_feature (1)..(1005): Cpo_SPTQ, codon-optimised for Y. lipolytica
atggccccctactctgaggagtacgagatcctgaaggagaatactaagcccgtatctccccaggccgcccccagagagtacaccgttgtgtactctgtggtgcttatctttgtttactggcacatcggagccctgtacggactgtacctgggcttcacctccgccaagtgggccaccatcatctttaactacctgatctacgtgtctggcggcttcgccattactgctggatcccatcgactgtggtctcaccgagccttcaaggctaagctccccctgcagatcctgctcatgcttctgcagaccatgtcttgtcagaagtctgtgctgaactgggtgcgagatcaccgactgcaccacatgtactgtgataccgatgccgacccttacaactctactcgaggaatcttctactctcacatcggctggctgatggtgaagaagcatcctgaggtgatccgaaagggccgaaccatcgacatgtccgatctggagaacaaccctgtgctgaagttccagaagaagttctaccccatcctcgtgaccctgatggcctttatcctgcctgccctgatccccgttattttctggcaggagtctctgaacatcgctcaccacgtttctcttgtgcacctggtcgtgggctcccacatgacctttgccattaactctattgcccacgccttcggatctaagccttgcgacaagaccatctctcccactcagtccatttccctgtctctggtgaccttcggcgaaggctaccataactaccaccacgtgttcccctttgattaccgagtggccgagctgggcaacaactacctgaacctgaccaccaacttcatcgacttcttcgcctggattggctgggcctacgacctgaagtacgcctctcccgatatggttgctaagcgagccaagcgaaccggcgacggaactgacctgtggggacgagctattgagcacgccgatattcaggctaagcgggtgcacccctcttaa

SEQ ID NO: 69 - protein, 334 aa, Cydia pomonella
MAPYSEEYEILKENTKPVSPQAAPREYTVVYSVVLIFVYWHIGALYGLYLGFTSAKWATIIFNYLIYVSGGFAITAGSHRLWSHRAFKAKLPLQILLMLLQTMSCQKSVLNWVRDHRLHHMYCDTDADPYNSTRGIFYSHIGWLMVKKHPEVIRKGRTIDMSDLENNPVLKFQKKFYPILVTLMAFILPALIPVIFWQESLNIAHHVSLVHLVVGSHMTFAINSIAHAFGSKPCDKTISPTQSISLSLVTFGEGYHNYHHVFPFDYRVAELGNNYLNLTTNFIDFFAWIGWAYDLKYASPDMVAKRAKRTGDGTDLWGRAIEHADIQAKRVHPS

SEQ ID NO: 70 - DNA, 1362 bp, Artificial sequence
  codon-optimized nucleotide sequence
  misc_feature (1)..(1362): S. cerevisiae-codon-optimized nucleotide sequence of H. subflexa fatty acyl reductase; mRNA-coding sequence.
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcaccaaccattcttgggttatgaaggctgatagagtcagagaattattcgcttctttgtctccagcagataagtacttgtttccatgtgatccagtcaacatcaattggagacaatatatccaagattactgctggggtgttagacatttcttggaaaaaaagacttaa

SEQ ID NO: 71 - protein, 453 aa, Heliothis subflexa
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTNHSWVMKADRVRELFASLSPADKYLFPCDPVNINWRQYIQDYCWGVRHFLEKKT

SEQ ID NO: 72 - DNA, 1371 bp, Artificial sequence
  codon-optimized nucleotide sequence
  misc_feature (1)..(1371): S. cerevisiae-codon-optimized nucleotide sequence of H. assulta fatty acyl reductase; mRNA-coding sequence.
atggttgtcttgacctccaaagaaactaagccatctgttgctgaattttacgctggtaagtctgttttcattactggtggtactggtttcttgggtaagatcttcattgaaaagttgttgtactcctgcccagatatcggtaatatctacatgttgatcagagaaaagaagggtttgtccgtttccgaaagaatcaagcaatttttggatgaccctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcattcctcttacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtatgtttttacaaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacacttcttggaaaaaaagactaccaacaagtaa

SEQ ID NO: 73 - protein, 456 aa, Helicoverpa assulta
MVVLTSKETKPSVAEFYAGKSVFITGGTGFLGKIFIEKLLYSCPDIGNIYMLIREKKGLSVSERIKQFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSYIVDLIPVDYVANLVIAAGAKSSKSTELKVYNCCSSACNPITIGKLMSMFAEDAIKQKSYAMPLPGWYVFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKTTNK

SEQ ID NO: 74 - DNA, 1362 bp, Helicoverpa virescens
atggttgtcttgacctccaaagaaactaagccatctgttgctgaattttacgctggtaagtctgttttcattactggtggtactggtttcttgggtaaggttttcattgaaaagttgttgtactcctgcccagatatcgttaacatctacatgttgatcagagaaaagaagggtttgtccgtttccgaaagaatcaagcaatttttggatgaccctttgttcaccagattgaaggacaaaagaccagctgatttggaaaagatcgttttgattccaggtgatattaccgctccagatttgggtattactgctgctaacgaaaagatgttgatcgaaaaggtttccgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagtttaccaatacgtcaaagaaggtatctccgaagaagataccgaaaaaatcttgaacggtagaccaaacacttacactttcactaaggctttgaccgaacatttggttgctgaaaatcaagcttacgttccaaccattatcgttagaccatcagttgttgctgccattaaggatgaaccattgaaaggttggttgggtaattggtttggtgctacaggtttgactgtttttactgctaagggtttgaacagagttatctacggtcattccaactacatcgttgatttgatcccagttgattacgttgccaacttggttattgctgctggtgctaaatctaacacctcttctgaattgaaggtctacaactgttgttcctcatcatgtaacccagttaagatcggtactttgatgtctatgtttgctgatgatgccatcaagcaaaagtcttatgctatgccattgccaggttggtacatttttactaagtacaagtggttggtcttgttgttgaccttcttgttccaagttattccagcctacattaccgatttgtcaagacacttggttggtaagagtccaagatatatcaagttgcaatccttggtcaatcaaaccagatcctccattgatttcttcaccaatcattcttgggttatgaaggccgatagagtcagagaattatacgcttctttgtctccagcagataagtacttgtttccatgtgatccagttaacatcaactggacccaatacttgcaagattactgttggggtgttagaaacttcttggaaaaaaagacttaa

SEQ ID NO: 75 - protein, 453 aa, Helicoverpa virescens
MVVLTSKETKPSVAEFYAGKSVFITGGTGFLGKVFIEKLLYSCPDIVNIYMLIREKKGLSVSERIKQFLDDPLFTRLKDKRPADLEKIVLIPGDITAPDLGITAANEKMLIEKVSVIIHSAATVKFNEPLPTAWKINVEGTRMMLALSRRMKRIEVFIHISTAYTNTNREVVDEILYPAPADIDQVYQYVKEGISEEDTEKILNGRPNTYTFTKALTEHLVAENQAYVPTIIVRPSVVAAIKDEPLKGWLGNWFGATGLTVFTAKGLNRVIYGHSNYIVDLIPVDYVANLVIAAGAKSNTSSELKVYNCCSSSCNPVKIGTLMSMFADDAIKQKSYAMPLPGWYIFTKYKWLVLLLTFLFQVIPAYITDLSRHLVGKSPRYIKLQSLVNQTRSSIDFFTNHSWVMKADRVRELYASLSPADKYLFPCDPVNINWTQYLQDYCWGVRNFLEKKT

SEQ ID NO: 76 - protein, 507 aa, Cydia pomonella
MDMIDEAEARGESQIQKFLSGSTILLTGGTGFLGKLLVEKLLRTCPDIKKIYLLARPKKNKEIQKRLQEQFEDPLYERLRKQVPDFMSKIGVVEGDVGKLGLGISESDRQTVVDEVDVIFHGAATLRFNEPLRDAVFINVRGTREMMLLARACTKLKAMVHISTAYSNCTLSEIDEVFYESPIPGDKLIDLAESLDEKTINSITPGLIGDFPNTYAYTKGVAEDVLQKYSQGLPVAVVRPSIVIGTAKDPVAGWIDNVYGPTGVIVGAELGLLHVLHAAPNASASLVPGDAVAAACVAAAWSVSRAENHQAPARDAPPLYHCVCSEKAPITWSQFMSLAETHGLVVPPMQAMWYYMLTLTNSKAMYTLLALLMHWIPAYIIDGVCMVLGKKPQLRKAYTKIEQFAAVIEFFALREWRFHNNNMTRLYNELCDADKHIYDFDTSAIDWNEFFANYMKGIRVYLLKDPVSTIPESLKRHKRLKWLHYALLTVLSLLVLRLLWFFVSFLF

SEQ ID NO: 77 - protein, 346 aa, Grapholita molesta
MPPESKNVPIQQNFRKPLEFLPRKYDVVYENVFLHIAGHISAAYGLYLCFTVAKWQTIALAFVWYHLGKIGIICGAHRLWSHRCYKAKMPLHIILMICNCIGFENTAINWVRNHRMHHKHSDTDGDPHNSNRGAFFSHIGWLCVRKHPETRNCKVDMSDIYSNPVLVFQKRYKYPLVGFLCYGLPTFIPMYFWGETLVTAWHVNILRYFLSMNAVFLVNSLAHLYGNKPYDISICPRQSPFVSLLTIGEGFHNYHHTFPWDYRAAELGNNYLNVGKWVIDFFAMIGWAYDLKTVPDETIKRRMKRTGDGTNCWGWGDKDMTREDRDIAKIIYPESISKEERDIIAM

SEQ ID NO: 78 - DNA, 1040 bp, Grapholita molesta
atgcctccggagtccaaaaacgttcctatccagcaaaattttaggaaaccactagaatttctcccgaggaaatatgatgtggtgtacgagaatgtatttcttcacatcgctggacatatatctgcagcttacggcttatatctctgcttcactgtggctaaatggcagactatcgcccttgcattcgtctggtaccacctgggcaagattggtataatctgtggcgcccaccggctttggtctcatcgctgctacaaagccaagatgcctctgcatattattcttatgatatgtaattgtataggtttcgaaaacacagccattaattgggtaaggaatcatagaatgcaccacaagcacagcgacacggacggtgatccccacaactcgaatagaggagctttcttttcccacatcggttggctgtgtgtcaggaaacatccggagactagaaactgtaaagtcgacatgagtgatatatacagcaatcctgtattggtgtttcagaagagatataaatatcctttggtcggatttctctgttacggtctacctacgtttatacccatgtatttttggggagagactttggtaacagcttggcatgtgaatattctgcgttactttttaagtatgaatgccgtttttctggtcaacagcttggcgcatttgtacggaaataagccttatgacatatcaatttgtccgcgacaaagtccttttgtgtcacttttgaccataggcgagggattccacaattatcaccatacgtttccttgggactatagggcggcagaactaggcaataactatctgaatgttggaaaatgggtcatagacttcttcgctatgatcggctgggcgtatgacctcaaaacagttccagatgaaacgataaagagaagaatgaaaaggactggagatggcaccaactgctggggatggggggacaaggacatgactagggaggacagagatatcgctaaaatcatctatcctgagtcgatatcgaaagaagaaagagatataattgcgatgga

SEQ ID NO: 79 - DNA, 1041 bp, Grapholita molesta
atgcctccggagtccaaaaacgttcctatccagcaaaattttaggaaaccactagaatttctcccgaggaaatatgatgtggtgtacgagaatgtatttcttcacatcgctggacatatatctgcagcttacggcttatatctctgcttcactgtggctaaatggcagactatcgcccttgcattcgtctggtaccacctgggcaagattggtataatctgtggcgcccaccggctttggtctcatcgctgctacaaagccaagatgcctctgcatattattcttatgatatgtaattgtataggtttcgaaaacacagccattaattgggtaaggaatcatagaatgcaccacaagcacagcgacacggacggtgatccccacaactcgaatagaggagctttcttttcccacatcggttggctgtgtgtcaggaaacatccggagactagaaactgtaaagtcgacatgagtgatatatacagcaatcctgtattggtgtttcagaagagatataaatatcctttggtcggatttctctgttacggtctacctacgtttatacccatgtatttttggggagagactttggtaacagcttggcatgtgaatattctgcgttactttttaagtatgaatgccgtttttctggtcaacagcttggcgcatttgtacggaaataagccttatgacatatcaatttgtccgcgacaaagtccttttgtgtcacttttgaccataggcgagggattccacaattatcaccatacgtttccttgggactatagggcggcagaactaggcaataactatctgaatgttggaaaatgggtcatagacttcttcgctatgatcggctgggcgtatgacctcaaaacagttccagatgaaacgataaagagaagaatgaaaaggactggagatggcaccaactgctggggatggggggacaaggacatgactagggaggacagagatatcgctaaaatcatctatcctgagtcgatatcgaaagaagaaagagatataattgcgatgtga

SEQ ID NO: 80 - DNA, 1524 bp, Cydia pomonella
atggacatgatcgacgaggccgaggctcgaggcgagtctcagatccagaagttcctgtctggctctaccatcctgctgaccggcggaaccggcttcctgggcaagctgctggtcgagaagctgctgcgaacctgtcctgacatcaagaagatctacctgctggctcgacccaagaagaacaaggaaatccagaagcgactgcaagagcagttcgaggaccctctgtacgagcgactccgaaagcaggtccccgacttcatgtctaagatcggcgtggtcgagggcgacgtgggaaagctcggcctgggcatctctgagtctgaccgacagaccgtggtggacgaggtggacgtgatcttccacggcgctgctaccctgcgattcaacgagcccctgcgagatgccgtgttcatcaacgtgcgaggcacccgagagatgatgctgctggcccgagcctgcaccaagctgaaggccatggtgcacatctctaccgcctactctaactgcaccctgtctgagattgacgaggtgttctacgagtctcccattcctggcgacaagctgatcgacctggccgagtctctggacgaaaagaccatcaactctatcacccctggcctgatcggcgacttccccaacacctacgcctacaccaagggcgtcgccgaggacgtgctgcagaagtactctcagggactgcccgtggccgtggtgcgaccctctatcgtgatcggcaccgctaaggaccccgtcgccggctggatcgacaacgtgtacggtcccaccggtgtgattgtgggtgctgagctgggcctgctgcacgtgctccacgctgctcccaacgcctctgcctctctggtgcccggtgacgctgtggctgctgcttgcgtggctgctgcttggtctgtgtctcgagccgagaaccatcaggctcccgctcgagatgcccctcctctgtaccactgcgtgtgctctgagaaggctcccatcacctggtcgcagttcatgtctctggccgagactcacggcctggtggtgcctccaatgcaggccatgtggtactacatgctgaccctgaccaactctaaggccatgtacaccctgctcgccctgctgatgcactggatccccgcctacatcatcgacggcgtgtgcatggtgctgggcaagaagccccagctgcgaaaggcttacaccaagatcgagcagtttgccgccgtgatcgagttcttcgctctgcgagagtggcgattccacaacaacaacatgacccgactgtacaacgagctgtgcgacgccgacaagcacatctacgacttcgacacctctgccatcgactggaacgagttctttgccaactacatgaagggcatccgagtgtacctgctgaaggaccctgtgtctactatccctgagtctctgaagcgacacaagcgactgaagtggctgcactacgccctgctcaccgtgctgtctctgctggtgctgcgactgctgtggttcttcgtgtctttcctgttttag

(19) United States
(12) Patent Application Publication
(10) Pub. No.: US 2024/0327874 A1
(43) Pub. Date: Oct. 3, 2024

(54) Yeast cells and methods for production of E8,E10-dodecadienyl coenzyme A, codlemone and derivatives thereof

(71) Applicant: FMC Agricultural Solutions A/S, Ronland (DK)

(72) Inventors: Irina Borodina, Copenhagen (DK); Carina Holkenbrink, Copenhagen (DK); Christer Löfstedt, Lund (SE); Baojian Ding, Södra Sandby (SE); Leonie Wenning, Copenhagen (DK)

(21) Appl. No.: 17/783,955

(22) PCT Filed: Dec. 18, 2020
(86) PCT No.: PCT/EP2020/086975

§ 371 Date: Jun. 9, 2022

(30) Foreign Application Priority Data
Dec. 20, 2019 (EP) 19218703.7

(51) Int. Cl. C12P 7/04; C12N 9/02; C12N 9/10; C12N 9/16; C12P 7/64; C12P 7/6409

(52) U.S. Cl. CPC C12P 7/04; C12N 9/0008; C12N 9/004; C12N 9/1029; C12N 9/16; C12P 7/64; C12P 7/6409; C12Y 102/01084; C12Y 106/02002; C12Y 203/01086

(57) ABSTRACT

The present invention relates to yeast cells engineered for the production of E8,E10-dodecadienyl coenzyme A, codlemone (E8,E10-dodecadien-1-ol), and optionally its derivatives E8,E10-dodecadienyl acetate and/or E8,E10-dodecadienal. Methods for production of E8,E10-dodecadienyl coenzyme A, codlemone (E8,E10-do-decadien-1-ol), and optionally its derivatives E8,E10-dodecadienyl acetate and/or E8,E10-dodecadienal are also provided. Nucleic acid constructs useful for obtaining such yeast cells are also provided.

### CROSS REFERENCE TO RELATED APPLICATIONS

This application is a U.S. National Stage under 35 U.S.C. § 371 of PCT/EP2020/086975 filed Dec. 18, 2020 and which depends from and claims priority to European application number 19218703.7 filed Dec. 20, 2019, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created Dec. 21, 2022, is named “2022-12-21 Sequence listing_ST25_Corrected BIOP0003PA” and is 298,199 bytes in size.

### TECHNICAL FIELD

The present invention relates to yeast cells engineered for the production of E8,E10-dodecadienyl coenzyme A, codlemone (E8,E10-dodecadien-1-ol), and optionally its derivatives E8,E10-dodecadienyl acetate and/or E8,E10-dodecadienal. Methods for production of E8,E10-dodecadienyl coenzyme A, codlemone (E8,E10-dodecadien-1-ol), and optionally its derivatives E8,E10-dodecadienyl acetate and/or E8,E10-dodecadienal are also provided. Nucleic acid constructs useful for obtaining such yeast cells are also provided.

### BACKGROUND

Integrated Pest Management (IPM) is expected to play a major role for both increasing the crop yield and for minimizing environmental impact and enabling organic food production. IPM employs alternative pest control methods, such as mating disruption using pheromones, mass trapping using pheromones, beneficial insects, etc.

Pheromones constitute a group of diverse chemicals that insects (like other organisms) use to communicate with individuals of the same species in various contexts, including mate attraction, alarm, trail marking and aggregation. Insect pheromones associated with long-range mate finding are already used in agriculture and forestry applications for monitoring and control of pests, as a safe and environmentally friendly alternative to pesticides.

Pheromones represent a health- and environment-friendly alternative to pesticides. Dispensing sex pheromones in the fields or orchards disrupts insect communication and prevents mating; thus no fertile eggs will be laid and no larval damage will occur to the crops. This method is called “mating disruption”. Pheromones are attractive alternatives to insecticides, because they are biodegradable, species-specific compounds, which neither harm beneficial species nor humans.

Application of insect pheromones for pest control became possible only after industrial-scale synthesis of pheromones started several decades ago. Nevertheless, the prices for chemically synthesized pheromones remain high and present a major barrier for expanding their usage in agriculture and forestry. Another drawback with the chemical production of pheromones is the requirement for toxic chemicals to be used as precursors, catalyzers and solvents, and large amounts of organic waste generated during the purification. The current production methods based on complex chemical synthesis-based processes thus make the products prohibitively expensive for widespread use in many of the potential applications in agriculture and forestry.

There are several advantages to biological production methods as compared to chemical production methods. First, all the reactions are carried out by engineered cells at ambient temperatures in fermentation tanks instead of multiple chemical reaction steps requiring different precursors, catalyzers and conditions (often high temperatures and pressures). Moreover, the engineered cells use cheap renewable materials, such as sugars or plant oils, instead of using multiple expensive specialty chemicals as precursors. While chemical reactions often suffer from low specificity, and thus require purification of intermediate compounds and extensive purification of the final product, biological reactions carried out by enzymes are typically very specific and formation of by-products is limited, thereby reducing the usage of organic solvents and other toxic chemicals for purification. Moreover, specific stereo-chemistry, which is often important for pheromone activity, can be very difficult to achieve by chemical methods, while enzymatic methods can take advantage of enzymes specific for one of the cis- or trans-isomers.

A specific pheromone of interest is codlemone, a di-unsaturated fatty alcohol with the formula E8,E10-dodecadien-1-ol (E8,E10-C12:OH, CAS nr. 33956-49-9). Codlemone is a sex pheromone component of a number of species, and the main sex pheromone of Cydia pomonella (codling moth), which belongs to the order of Lepidoptera and is a major pest of apples, pears, plums, and other fruits.

Ding 2014 discloses plant cells in which desaturases were expressed, and tested to determine whether they could produce moth pheromones. By using degenerate PCR approach, three desaturase from C. pomonella were found (Ding et al. On the way of making plants smell like moths—a synthetic biology approach. Lund University, Faculty of Science, Department of Biology).

Hence, there is a need for biological processes for production of insect pheromones, in particular codlemone. In addition to lower cost benefits, fermentation processes are inherently less hazardous and more environmentally friendly than chemical synthesis.

### SUMMARY

The invention is as defined in the claims.

Herein is provided a yeast cell capable of producing E8,E10-dodecadienyl coenzyme A and optionally E8,E10-dodecadien-1-ol, said yeast cell expressing at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA).

Herein is provided a yeast cell capable of producing E8,E10-dodecadien-1-ol, said yeast cell expressing:
i) At least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
ii) At least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol.

Also provided is a method for producing E8,E10-dodecadienyl coenzyme A and optionally E8,E10-dodecadien-1-ol in a yeast cell, said method comprising the steps of providing a yeast cell and incubating said yeast cell in a medium, wherein the yeast cell expresses:
i) At least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
ii) Optionally at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol, thereby producing E8,E10-dodecadienyl coenzyme A and optionally E8,E10-dodecadien-1-ol.

Also provided is a nucleic acid construct for modifying a yeast cell, said construct comprising:
i) At least one first polynucleotide encoding at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
ii) Optionally a second polynucleotide encoding at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol.

Also provided is a method of monitoring the presence of pest or disrupting the mating of pest, said method comprising the steps of:
i) Producing E8,E10-dodecadien-1-ol and optionally E8,E10-dodecadienyl acetate and/or E8,E10-dodecadienal by the methods described herein;
ii) Formulating said E8,E10-dodecadien-1-ol and optionally said E8,E10-dodecadienyl acetate and/or said E8,E10-dodecadienal as a pheromone composition; and
iii) Employing said pheromone composition as an integrated pest management composition.

Also provided herein are E8,E10-dodecadienyl coenzyme A, E8,E10-dodecadien-1-ol, E8,E10-dodecadienyl acetate and/or E8,E10-dodecadienal obtainable by the methods described herein.

Also provided herein is a kit of parts comprising instructions for use and:
a) the yeast cell described herein; and/or
b) the nucleic acid construct described herein for modifying a yeast cell and optionally the yeast cell to be modified, wherein upon expression of the polynucleotides comprised within the nucleic acid construct, the modified yeast cell is capable of producing E8,E10-dodecadienyl coenzyme A and optionally E8,E10-dodecadien-1-ol.

### DESCRIPTION OF THE DRAWINGS

FIG. 1. Proposed biosynthesis pathway for codlemone production (E8,E10-C12:OH) in yeast. ACC: acetyl-CoA-carboxylase; FA: fatty acids; FAS; fatty acid synthase; TE: thioesterase; FAA: Fatty acyl-CoA synthetase; L: lipids; FAE: fatty acid esters; FAD: fatty acyl desaturase; FAR: fatty acyl reductase; Comp. β-ox.: complete β-oxidation.

FIG. 2. GC-MS analysis of FAME extracts from yeast transformed with (A) empty plasmid or (B) Cpo_CPRQ containing vector fed with 12:Me, (C) mass spectrum of E9-12:Me; GC-MS analysis of FAME extracts from yeast transformed with (D) empty plasmid or (E) Cpo_CPRQ fed with E9-12:Me, (F) mass spectrum of E8,E10-12:Me.

### DETAILED DESCRIPTION

### Definitions

Biopesticide: the term ‘biopesticide’ is a contraction of ‘biological pesticide’ and refers to several types of pest management intervention: through predatory, parasitic, or chemical relationships. In the EU, biopesticides have been defined as “a form of pesticide based on micro-organisms or natural products”. In the US, they are defined by the EPA as “including naturally occurring substances that control pests (biochemical pesticides), microorganisms that control pests (microbial pesticides), and pesticidal substances produced by plants containing added genetic material (plant-incorporated protectants) or PIPs”. The present disclosure relates more particularly to biopesticides comprising natural products or naturally occurring substances. They are typically created by growing and concentrating naturally occurring organisms and/or their metabolites including bacteria and other microbes, fungi, nematodes, proteins, etc. They are often considered to be important components of integrated pest management (IPM) programmes, and have received much practical attention as substitutes to synthetic chemical plant protection products (PPPs). The Manual of Biocontrol Agents (2009: formerly the Biopesticide Manual) gives a review of the available biological insecticide (and other biology-based control) products.

Cloud concentration: the term will herein be used to refer to the concentration of a surfactant, in particular non-ionic, or a glycol solution, in a solution above which, at a given temperature, a mixture of said surfactant and said solution starts to phase-separate, and two phases appear, thus becoming cloudy. For example, the cloud concentration of a surfactant in an aqueous solution at a given temperature is the minimal concentration of said surfactant which, when mixed with the aqueous solution, gives rise to two phases. The cloud concentration can be obtained from the manufacturer of the surfactant, or it may be determined experimentally, by making a dosage curve and determining the concentration at which the mixture phase separates.

Cloud point: The cloud point of a surfactant, in particular non-ionic, or a glycol solution, in a solution, for example an aqueous solution, is the temperature at which a mixture of said surfactant and said solution, for example said aqueous solution, starts to phase-separate, and two phases appear, thus becoming cloudy. This behavior is characteristic of non-ionic surfactants containing polyoxyethylene chains, which exhibit reverse solubility versus temperature behavior in water and therefore “cloud out” at some point as the temperature is raised. Glycols demonstrating this behavior are known as “cloud-point glycols”. The cloud point is affected by salinity, being generally lower in more saline fluids.

Codlemone: the term refers to a di-unsaturated alcohol with the formula E8,E10-dodecadien-1-ol (E8,E10-C12:OH). Codlemone is the main sex pheromone component of a number of species, among others Cydia pomonella (codling moth), which belongs to the order of Lepidoptera and is a major pest of apples, pears, plums, and other fruits. The terms “codlemone”, “E8,E10-dodecadien-1-ol” and “E8,E10-C12:OH” will herein be used interchangeably.

Desaturated: the term “desaturated” will be herein used interchangeably with the term “unsaturated” and refers to a compound containing one or more double or triple carbon-carbon bonds.

Ethoxylated and propoxylated C₁₆-C₁₈ alcohol-based antifoaming agent: the term refers to a group of polyethoxylated, non-ionic surfactants which comprise or mainly consist of ethoxylated and propoxylated alcohols in C₁₆-C₁₈, for example CAS number 68002-96-0, also termed C₁₆-C₁₈ alkyl alcohol ethoxylate propoxylate or C₁₆-C₁₈ alcohols ethoxylated propoxylated polymer.

Extractant: the term “extractant” as used herein refers to a non-ionic surfactant such as an antifoaming agent which facilitates recovery of hydrophobic compounds produced in a fermentation, in particular a polyethoxylated surfactant selected from: a polyethylene polypropylene glycol, a mixture of polyether dispersions, an antifoaming agent comprising polyethylene glycol monostearate such as simethicone and ethoxylated and propoxylated C₁₆-C₁₈ alcohol-based antifoaming agents and combinations thereof.

Fatty acid: the term “fatty acid” refers to a carboxylic acid having a long aliphatic chain, i.e. an aliphatic chain between 4 and 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Most naturally occurring fatty acids are unbranched. They can be saturated, or desaturated.

Fatty alcohol acetate: the term will herein be used interchangeably with “fatty acetate” and refers to an acetate having a fatty carbon chain, i.e. an aliphatic chain between 4 and 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Fatty alcohol acetates can be saturated or desaturated.

Fatty acyl-CoA: the term will herein be used interchangeably with “fatty acyl-CoA ester”, and refers to compounds of general formula R-CO-SCoA, where R is a fatty carbon chain. The fatty carbon chain is joined to the —SH group of coenzyme A by a thioester bond. Fatty acyl-CoAs can be saturated or desaturated, depending on whether the fatty acid which it is derived from is saturated or desaturated.

Fatty alcohol: the term “fatty alcohol” refers herein to an alcohol derived from a fatty acyl-CoA, having a carbon chain length of 4 to 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Fatty alcohols can be saturated or desaturated.

Fatty aldehyde: the term refers herein to an aldehyde derived from a fatty acyl-CoA, having a carbon chain length of 4 to 28 carbon atoms, such as 4, 5, 6, 7, 8, 9, 10, 11, 12, 13, 14, 15, 16, 17, 18, 19, 20, 21, 22, 23, 24, 25, 26, 27 or 28 carbon atoms. Fatty aldehydes can be saturated or desaturated.

Heterologous: the term “heterologous” when referring to a polypeptide, such as a protein or an enzyme, or to a polynucleotide, shall herein be construed to refer to a polypeptide or a polynucleotide which is not naturally present in a wild type cell. For example, the term “heterologous Δ9 desaturase” when applied to Yarrowia lipolytica refers to a Δ9 desaturase which is not naturally present in a wild type Y. lipolytica cell, e.g. a Δ9 desaturase derived from Drosophila melanogaster.

Mixture of polyether dispersions: the term refers to a group of polyethoxylated non-ionic surfactants which comprise or mainly consist of a mixture of polyether dispersions, for example organic antifoam 204 from Sigma Aldrich (product number A6426 and A8311, MDL number MFCD00130523).

Native: the term “native” when referring to a polypeptide, such as a protein or an enzyme, or to a polynucleotide, shall herein be construed to refer to a polypeptide or a polynucleotide which is naturally present in a wild type cell. The term will be used interchangeably with the term “endogenous”.

Pest: as used herein, the term ‘pest’ shall refer to an organism, in particular an animal, detrimental to humans or human concerns, in particular in the context of agriculture or livestock production. A pest is any living organism which is invasive or prolific, detrimental, troublesome, noxious, destructive, a nuisance to either plants or animals, human or human concerns, livestock, human structures, wild ecosystems etc. The term often overlaps with the related terms vermin, weed, plant and animal parasites and pathogens. It is possible for an organism to be a pest in one setting but beneficial, domesticated or acceptable in another.

Pheromone: pheromones are naturally occurring compounds designated by an unbranched aliphatic chain (between 9 and 18 carbons) ending in an alcohol, aldehyde or acetate functional group and containing up to 3 double bonds in the aliphatic backbone. Pheromone compositions may be produced chemically or biochemically, for example as described herein. Pheromones may thus comprise desaturated fatty alcohols, fatty aldehydes or fatty alcohol acetates, such as can be obtained by the methods and cells described herein.

Polyethoxylated surfactant: the term herein refers to polyethoxylated surfactants, i.e. non-ionic surfactants.

Polyethylene polypropylene glycol: the term refers to a group of polyethoxylated non-ionic surfactants which comprise or mainly consist of PEG-PPG-PEG block copolymer antifoaming agents, for example Kollliphor® P407 (CAS number 9003-11-6), also termed poly(ethylene glycol)-block-poly(propylene glycol)-block-poly(ethylene glycol).

Reduced activity: the term “reduced activity” may herein refer to a total or a partial loss of activity of a given peptide, such as a protein or an enzyme. In some cases, peptides are encoded by essential genes, which cannot be deleted. In these cases, activity of the peptide can be reduced by methods known in the art, such as down-regulation of transcription or translation, or inhibition of the peptide. In other cases, the peptide is encoded by a non-essential gene, and the activity may be reduced or it may be completely lost, e.g. as a consequence of a deletion of the gene encoding the peptide. Reduced activity of an enzyme can also be achieved by repressing transcription of the gene encoding said enzyme as is known in the art, for example using a repressible promoter, by inhibiting the activity or by silencing at the translational level.

Saturated: the term “saturated” refers to a compound which is devoid of double or triple carbon-carbon bonds.

Simethicone: the term refers to a group of polyethoxylated non-ionic surfactants which comprise or mainly consist of simethicone, also termed simeticone (CAS number 8050-81-5), dimethyl polysiloxane, or activated Polymethylsiloxane. Simethicone is a silicone-based emulsion containing also 1.2-1.6% polyethylene glycol monostearate.

Surfactant: the term refers to compounds that lower the surface tension (or interfacial tension) between two liquids, between a gas and a liquid, or between a liquid and a solid. Surfactants may act as detergents, wetting agents, emulsifiers, antifoaming agents, and dispersants. Surfactants are usually organic compounds that are amphiphilic, meaning they contain both hydrophobic groups (their tails) and hydrophilic groups (their heads). Therefore, a surfactant typically contains both a water-insoluble (or oil-soluble) component and a water-soluble component. Most commonly, surfactants are classified according to polar head group. A non-ionic surfactant has no charged groups in its head.

Titer: the titer of a compound refers herein to the produced concentration of a compound. When the compound is produced by a cell, the term refers to the total concentration produced by the cell, i.e. the total amount of the compound divided by the volume of the culture medium. This means that, particularly for volatile compounds, the titer includes the portion of the compound which may have evaporated from the culture medium, and it is thus determined by collecting the produced compound from the fermentation broth and from potential off-gas from the fermenter.

### Codlemone (E8,E10-C12:OH)

The biosynthesis of codlemone is based on acetyl-coenzyme A (CoA), which is carboxylated to malonyl-CoA; the reaction is catalyzed by acetyl-CoA-carboxylase (ACC). Malonyl-CoA and acetyl-CoA are precursors used by fatty acid synthase (FAS) to synthesize fatty acyl-CoAs up to a chain length of C16/C18. It was hypothesised that C. pomonella peroxisomal oxidases (POXs) catalyze the chain shortening (−2 C) of C16:CoA via C14:CoA to C12:CoA (lauryl-CoA) (Ding, 2014). Evidence for a desaturase converting C12:CoA to E9-C12:CoA in C. pomonella has been found early on, but the gene coding for this desaturase has only been identified recently together with two more genes coding for additional desaturases (Cpo_SPTQ/Cpo_NPVE/Cpo_CPRQ). A first desaturation step results in conversion of C12:CoA to E/Z9-C12:CoA, which in a second desaturation step is converted to E8,E10-C12:CoA (E8,E10-dodecadienyl coenzyme A). A fatty acyl reductase (FAR) then presumably reduces the diene E8,E10-C12:CoA to finally form codlemone (E8,E10-C12:OH). The gene coding for the FAR in C. pomonella has so far not been identified (Ding 2014, Löfstedt et al., 1988).

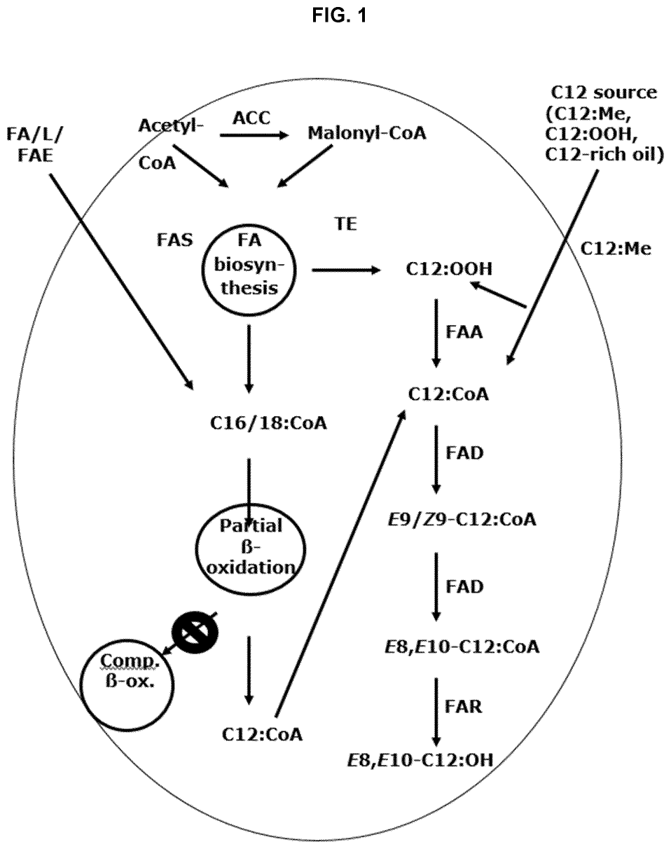

A proposed pathway for biosynthesis of codlemone is set out in FIG. 1.

### Production of Codlemone

The present disclosure relates to yeast cells capable of producing E8,E10-dodecadienyl coenzyme A and optionally codlemone (E8,E10-C12:OH or E8,E10-dodecadien-1-ol) and to methods for production of codlemone (E8,E10-C12:OH or E8,E10-dodecadien-1-ol) in a yeast cell.

The inventors have designed a heterologous pathway (outlined in FIG. 1 by way of example) for production of E8,E10-dodecadienyl coenzyme A and optionally E8,E10-dodecadien-1-ol in yeast.

Accordingly, herein is provided a method for production of E8,E10-dodecadienyl coenzyme A and optionally E8,E10-dodecadien-1-ol in a yeast cell, said method comprising the steps of providing a yeast cell and incubating said yeast cell in a medium, wherein the yeast cell expresses:
i) At least one heterologous desaturase capable of introducing one or more double bonds in a fatty acyl-CoA having a carbon chain length of 12, thereby converting at least part of said fatty acyl-CoA to E8,E10-dodecadienyl coenzyme A (E8,E10-C12:CoA); and
ii) Optionally at least one heterologous fatty acyl-CoA reductase (EC 1.2.1.84) capable of converting at least part of said E8,E10-dodecadienyl coenzyme A (E8,E10-C12:CoA) to E8,E10-dodecadien-1-ol, thereby producing E8,E10-dodecadienyl coenzyme A and optionally E8,E10-dodecadien-1-ol.

The present yeast cells and methods can thus be used to produce codlemone by producing E8,E10-dodecadienyl coenzyme A as described herein, which can then be converted to E8,E10-dodecadien-1-ol either in vivo by expressing a reductase in the yeast cell, or the E8,E10-dodecadienyl coenzyme A can be converted into a lipid such as a triacylglyceride or into a free fatty acid, which can then be recovered and converted to E8,E10-dodecadien-1-ol in vitro, as is known in the art, e.g. by contacting them with a reductase. In both cases, E8,E10-dodecadien-1-ol is produced.

### Yeast Cell

In a first step of the method, a yeast cell is provided, which can use acetyl-CoA and malonyl-CoA for the biosynthesis of longer acyl-CoAs. Any yeast cell capable of synthesising acyl-CoAs can be used for producing E8,E10-dodecadienyl coenzyme A and optionally E8,E10-dodecadien-1-ol as described herein. Alternatively, the yeast cell may be provided with suitable carbon sources as is known in the art. The yeast cell may be a non-naturally occurring yeast cell, for example a yeast cell which has been engineered to produce E8,E10-dodecadienyl coenzyme A and optionally E8,E10-dodecadien-1-ol, E8,E10-dodecadienyl acetate and/or E8,E10-dodecadienal as described herein.

Acetyl-CoA and malonyl-CoA can be converted to acyl-CoAs, in particular an acyl-CoA having a carbon chain length of 12. This can involve a step of converting dodecanoyl-CoA to dodecanoic acid (lauric acid), for example by the action of a native or heterologous acyl-CoA thioesterase (EC 3.1.2.20). The lauric acid can then be converted to dodecanoyl-CoA by the action of a native or heterologous fatty acyl-coenzyme A synthetase (FAA) (EC 6.2.1.3).

The yeast cell is thus also capable of converting acetyl-CoA and malonyl-CoA to fatty acyl-CoAs, in particular to a fatty acyl-CoA having a carbon chain length of 12. In some embodiments, the yeast cell thus expresses one or more fatty acyl-coenzyme synthetases (EC 6.2.1.3) and/or one or more acyl-CoA thioesterases (EC 3.1.2.20) capable of performing said reaction.

In some embodiments, the yeast cell is provided with lauric acid or methyl laureate or trilauroylglycerol or another fatty acid derivative in the culture medium. Where the yeast cell has been engineered to be able to shorten the carbon chain via β-oxidation as described in detail below, the cell can be provided with oil or fat or any fatty acid derivative that has a carbon chain length longer than 12.

In some embodiments, the cell has been modified at the genomic level, e.g. by gene editing in the genome. The cell may also be modified by insertion of at least one nucleic acid construct such as at least one vector. The vector may be designed as is known to the skilled person to either enable integration of nucleic acid sequences in the genome, or to enable expression of a polypeptide encoded by a nucleic acid sequence comprised in the vector without genome integration.

In some embodiments of the disclosure, yeast or fungi of genera including, but not limited to, Blakeslea, Candida, Cryptococcus, Cunninghamella, Lipomyces, Mortierella, Mucor, Phycomyces, Pythium, Rhodosporidium, Rhodotorula, Trichosporon, Saccharomyces and Yarrowia are employed. In certain particular embodiments, organisms of species that include, but are not limited to, Blakeslea trispora, Candida pulcherrima, C. revkaufi, C. tropicalis, Cryptococcus curvatus, Cunninghamella echinulata, C. elegans, C. japonica, Lipomyces starkeyi, L. lipoferus, Mortierella alpina, M. isabellina, M. ramanniana, M. vinacea, Mucor circinelloides, Phycomyces blakesleanus, Pythium irregulare, Rhodosporidium toruloides, Rhodotorula glutinis, R. gracilis, R. graminis, R. mucilaginosa, R. pinicola, Trichosporon pullans, T. cutaneum, Saccharomyces cerevisiae and Yarrowia lipolytica are used. In some embodiments, the yeast cell is a Yarrowia lipolytica cell or a Saccharomyces cerevisiae cell.

The yeast cell to be modified, which will also be referred to as the host cell, may express native enzymes which may have a negative impact on the titre of E8,E10-dodecadien-1-ol that can be obtained; the native enzymes may thus be inactivated by methods known in the art, such as gene editing. For example, the genes encoding the native enzymes having a negative impact on the titre may be deleted or mutated so as to lead to total or partial loss of activity of the native enzyme, as described herein below.

### Desaturase

The present methods rely on the yeast cell expressing the necessary enzymes for converting a fatty acyl-CoA having a carbon chain length of 12 to E8,E10-dodecadienyl coenzyme A and optionally E8,E10-dodecadien-1-ol. The first enzyme needed for this is a desaturase, which is capable of introducing one or more double bonds in said fatty acyl-CoA having a carbon chain length of 12, thereby converting said fatty acyl-CoA to a desaturated fatty acyl-CoA of carbon chain length 12 and having one or more double bonds. The desaturated fatty acyl-CoA of carbon chain length 12 may be a mixture of desaturated fatty acyl-CoAs of carbon chain length 12; said mixture comprises E8,E10-C12:CoA, but typically also comprises the monounsaturated fatty acyl-CoAs E9-C12:CoA and Z9-C12:CoA. Thus in some embodiments the yeast cell expresses a desaturase which is capable of introducing one or more double bonds in a fatty acyl-CoA having a carbon chain length of 12, thereby converting at least part of said fatty acyl-CoA to E8,E10-C12:CoA (E8,E10-dodecadienyl coenzyme A). Desaturases of the EC class EC 1.14.19. are capable of performing such reactions.

The production of codlemone relies on two desaturation steps. These may be performed by one desaturase, for example Cpo_CPRQ or Gmo_CPRQ, mutants and functional variants thereof, as described herein below, or by two different desaturases. In embodiments with two different desaturases, at least one of the desaturases is Cpo_CPRQ, a mutant thereof or a functional variant thereof as described herein below. In other embodiments with two different desaturases, at least one of the desaturases is Gmo_CPRQ, a mutant thereof or a functional variant thereof as described herein below. The other desaturase is capable of introducing at least one double bond in a fatty acyl-CoA of carbon chain length 12, or can introduce at least one double bond in a fatty acyl-CoA of carbon chain length 14, which can then be shortened to a desaturated fatty acyl-CoA of carbon chain length 12 as detailed below in the section “Chain shortening”. The fatty acyl-CoA of carbon chain length 12 or 14 having one double bond can then be further desaturated by e.g. Cpo_CPRQ, the mutant or functional variant thereof.

The desaturase is preferably a heterologous desaturase. In some embodiments, the desaturase is Cpo_CPRQ (SEQ ID NO: 2), which is a desaturase naturally found in C. pomonella. As demonstrated in example 16, Cpo_CPRQ expression alone is sufficient to produce E8,E10-C12:CoA. Expression of either Cpo_SPTQ or Cpo_NPVE alone did not result in production of E8,E10-C12:CoA. This finding is surprising in light of Ding 2014, in which functional assays of these three desaturases indicated that they work consecutively forming the conjugated double bonds in C. pomonella pheromone—this does not seem to be the case in yeast.

The heterologous desaturase may also be a functional variant of a heterologous desaturase such as Cpo_CPRQ, i.e. a variant which retains the ability to convert a fatty acyl-CoA having a carbon chain length of 12 to a desaturated fatty acyl-CoA of carbon chain length 12 such as E8,E10-C12:CoA. In some embodiments, the functional variant has at least 60% homology or identity, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Cpo_CPRQ (SEQ ID NO: 2).

The heterologous desaturase may also be a functional variant of a heterologous desaturase such as Cpo_CPRQ, i.e. a variant which retains the ability to convert a fatty acyl-CoA having a carbon chain length of 12 to a desaturated fatty acyl-CoA of carbon chain length 12 such as E8,E10-C12:CoA. In some embodiments, the functional variant has at least 60% homology or identity, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Cpo_CPRQ (SEQ ID NO: 2).

The desaturase is preferably a heterologous desaturase. In some embodiments, the desaturase is Gmo_CPRQ (SEQ ID NO: 77), which is a desaturase naturally found in Grapholita molesta, or a functional variant thereof which retains the ability to convert a fatty acyl-CoA having a carbon chain length of 12 to a desaturated fatty acyl-CoA of carbon chain length 12 such as E8,E10-C12:CoA. In some embodiments, the functional variant has at least 60% homology or identity, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Gmo_CPRQ (SEQ ID NO: 78).

In some embodiments, the desaturase is expressed by introducing a nucleic acid which encodes said desaturase, as is known in the art. Such nucleic acid may be codon-optimised as is known in the art. In particular embodiments, the nucleic acid encoding the desaturase is as set forth in SEQ ID NO: 1, or is a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 1. In other embodiments, the nucleic acid encoding the desaturase is as set forth in SEQ ID NO: 78, or is a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 78.

In some embodiments, the yeast cell expresses several desaturases capable of introducing one or more double bonds in a fatty acyl-CoA of carbon chain length 12. In such embodiments, preferably at least one of the several desaturases is Cpo_CPRQ, a mutant thereof or a functional variant thereof as detailed below. In other embodiments, preferably at least one of the several desaturases is Gmo_CPRQ, a mutant thereof or a functional variant thereof. The other desaturase may be for example Cpo_NPVE (accession number: AHW98355, SEQ ID NO: 67) or Cpo_SPTQ (accession number: AHW98356, SEQ ID NO: 69), or functional variants thereof having at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. Such desaturases may be expressed in the yeast cell after introduction of a nucleic acid, which may be codon-optimised for the yeast cell, for example a nucleic acid as set forth in SEQ ID NO: 66 or SEQ ID NO: 68, or a homologue thereof having at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

The yeast cell may be engineered to express several copies of the heterologous desaturase. This can be done as is known in the art. The desaturase or desaturases may also be expressed at a high level as is known in the art, for example by the use of a constitutive promoter leading to strong expression levels—such promoters are known in the art.

In some embodiments, the desaturase is a mutant Cpo_CPRQ such as a Cpo_CPRQ mutant having a mutation at position 85. In some embodiments, the mutation is an S85A mutation. The desaturase may also be a functional variant of said mutant, and has at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to a mutant Cpo_CPRQ having a mutation at position 85, such as an S85A mutant. In some embodiments, the mutant is an S85T mutant.

In some embodiments, the desaturase is a mutant Cpo_CPRQ such as a Cpo_CPRQ mutant having a mutation at position 82. In some embodiments, the mutation is an S82A mutation. The desaturase may also be a functional variant of said mutant, and has at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to a mutant Cpo_CPRQ having a mutation at position 82, such as an S82A mutant.

In some embodiments, the yeast cell expresses two or more heterologous desaturases. Said two or more desaturases may be identical or different. In a particular embodiment the yeast cell expresses Cpo_CPRQ as set forth in SEQ ID NO: 2 and a mutant Cpo_CPRQ such as having a mutation at position 85, such as an S85A mutant. In some embodiments, the yeast cell expresses a Cpo_CPRQ (SEQ ID NO: 2), a mutant Cpo_CPRQ or a functional variant thereof having at least 65% homology or identity thereto, and also expresses another desaturase capable of introducing at least one double bond in a fatty acyl-CoA of carbon chain length 12. In some embodiments, the yeast cell expresses Gmo_CPRQ as set forth in SEQ ID NO: 77 and Cpo_CPRQ as set forth in SEQ ID NO: 2 or a mutant or functional variant thereof as described herein.

In some embodiments, the other desaturase is Cpo_NPVE as set forth in SEQ ID NO: 67, a mutant thereof or a functional variant thereof having at least 65% homology or identity thereto, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. The yeast cell in some embodiments expresses Cpo_CPRQ, a mutant or functional variant thereof, and Cpo_NPVE, a mutant or functional variant thereof. In some embodiments, the yeast cell expresses Gmo_CPRQ, a mutant or functional variant thereof, and Cpo_NPVE or a mutant or functional variant thereof.

In other embodiments, the other desaturase is Cpo_SPTQ as set forth in SEQ ID NO: 69, a mutant thereof or a functional variant thereof having at least 65% homology or identity thereto, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto. The yeast cell in some embodiments expresses Cpo_CPRQ, a mutant or functional variant thereof, and Cpo_SPTQ, a mutant or functional variant thereof. In some embodiments, the yeast cell expresses Gmo_CPRQ, a mutant or functional variant thereof, and Cpo_SPTQ or a mutant or functional variant thereof.

In preferred embodiments, the at least one heterologous desaturase is Cpo_CPRQ or a mutant or functional variant thereof as described herein above.

Yeast cells which, beside the desaturase described herein, which can introduce one or two double bonds in a fatty acyl-CoA of carbon chain length 12, express a desaturase capable of introducing at least one double bond in a fatty acyl-CoA of carbon chain length >12, such as of carbon chain length 14 or more, must also express other enzymes capable of reducing the carbon chain length of the desaturated fatty aycl-CoA of carbon chain length >12. This is detailed below in the section “Chain shortening”.

The yeast cell may thus express a desaturase capable of introducing one or more double bonds in said fatty acyl-CoA having a carbon chain length of 12, thereby converting said fatty acyl-CoA to a desaturated fatty acyl-CoA of carbon chain length 12 and having one or more double bonds, such as any of the desaturases described herein above, or functional variants thereof which retain the capability to convert a fatty acyl-CoA to a desaturated fatty acyl-CoA of carbon chain length 12. The yeast cell may further express a desaturase capable of introducing at least one double bond in a fatty acyl-CoA of carbon chain length >12, such as of carbon chain length 14 or more, or functional variants thereof which retain the capability to introduce at least one double bond in a fatty acyl-CoA of carbon chain length >12, such as of carbon chain length 14 or more.

In order to test whether a desaturase or a functional variant thereof has the desired activity, methods known in the art can be employed. For example, the candidate enzyme to be tested can be introduced in the yeast cell, e.g. on a vector or in the genome of the yeast cell, incubating the yeast cell in an appropriate medium, extracting fatty alcohols and/or fatty acid methyl esters from the broth, and performing an analysis such as a GC-MS analysis to determine whether desaturated compounds are produced. It may be advantageous to test the activity in a yeast cell in which the native elongase gene(s) has/have been deleted. An example of such a procedure is described in example 4 or in Schneiter et al., 2000.

### Fatty Acyl-CoA Reductase (EC 1.2.1.84)

The terms “fatty acyl-CoA reductase”, “reductase” and “FAR” will be used herein interchangeably. FARs catalyse the two-step reaction:

acyl-CoA+2NADPH<=>CoA+alcohol+2NADP(+)

wherein in a first step, the fatty acyl-CoA is reduced to a fatty aldehyde, before the fatty aldehyde is further reduced into a fatty alcohol in a second step. The fatty acyl-CoA may be a desaturated fatty acyl-CoA, in particular E8,E10-C12:CoA, which is then converted into E8,E10-dodecadien-1-ol.

The FARs capable of catalyzing such reaction are alcohol-forming fatty acyl-CoA reductases with an EC number 1.2.1.84. The yeast cells used in the present method may thus express a heterologous FAR capable of catalyzing the above reaction. Alternatively, the E8,E10-C12:CoA can be converted into E8,E10-dodecadien-1-ol after recovery of the E8,E10-C12:CoA, and contacting said E8,E10-C12:CoA with a FAR in vitro.

The FAR is preferably be an insect FAR, such as a FAR native to an insect of the genus Agrotis, Heliothis, Helicoverpa or Cydia. For example, the FAR is native to Agrotis segetum, Agrotis ipsilon, Heliothis subflexa, Helicoverpa assulta, Helicoverpa virescens or Cydia pomonella.

In some embodiments the FAR is Ase_FAR (SEQ ID NO: 10), i.e. the FAR naturally occurring in Agrotis segetum. In some embodiments, the heterologous FAR is a functional variant of Ase_FAR, which retains the capability of converting E8,E10-C12:CoA to E8,E10-dodecadien-1-ol. For example, the functional variant has at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Ase_FAR (SEQ ID NO: 10).

In some embodiments the FAR is a mutant Ase_FAR, such as a mutant having a mutation at position 198 or 413. In some embodiments the Ase_FAR mutant is a T198A mutant. In other embodiments, the Ase_FAR mutant is an S413A mutant.

In some embodiments, Ase_FAR or a functional variant thereof is expressed by introducing a nucleic acid in the yeast cell encoding Ase_FAR or the functional variant thereof. For example, a nucleic acid as set forth in SEQ ID NO: 9 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 9.

In other embodiments, the FAR is Aip_FAR (SEQ ID NO: 61), i.e. the FAR naturally occurring in Agrotis ipsilon. In some embodiments, the heterologous FAR is a functional variant of Aip_FAR, which retains the capability of converting E8,E10-C12:CoA to E8,E10-dodecadien-1-ol. For example, the functional variant has at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Aip_FAR (SEQ ID NO: 61).

In some embodiments, Aip_FAR or a functional variant thereof is expressed by introducing a nucleic acid in the yeast cell encoding Aip_FAR or the functional variant thereof. For example, a nucleic acid as set forth in SEQ ID NO: 60 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 60.

In other embodiments, the FAR is Hs_FAR (SEQ ID NO: 71), i.e. the FAR naturally occurring in Heliothis subflexa. In some embodiments, the heterologous FAR is a functional variant of Hs_FAR, which retains the capability of converting E8,E10-C12:CoA to E8,E10-dodecadien-1-ol. For example, the functional variant has at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Hs_FAR (SEQ ID NO: 71).

In some embodiments, Hs_FAR or a functional variant thereof is expressed by introducing a nucleic acid in the yeast cell encoding Hs_FAR or the functional variant thereof. For example, a nucleic acid as set forth in SEQ ID NO: 70 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 70.

In other embodiments, the FAR is Has_FAR (SEQ ID NO: 73), i.e. the FAR naturally occurring in Helicoverpa assulta. In some embodiments, the heterologous FAR is a functional variant of Has_FAR, which retains the capability of converting E8,E10-C12:CoA to E8,E10-dodecadien-1-ol. For example, the functional variant has at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Has_FAR (SEQ ID NO: 73).

In some embodiments, Has_FAR or a functional variant thereof is expressed by introducing a nucleic acid in the yeast cell encoding Has_FAR or the functional variant thereof. For example, a nucleic acid as set forth in SEQ ID NO: 72 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 72.

In other embodiments, the FAR is Hv_FAR (SEQ ID NO: 75), i.e. the FAR naturally occurring in Helicoverpa virescens. In some embodiments, the heterologous FAR is a functional variant of Hv_FAR, which retains the capability of converting E8,E10-C12:CoA to E8,E10-dodecadien-1-ol. For example, the functional variant has at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Hv_FAR (SEQ ID NO: 75).

In some embodiments, Hv_FAR or a functional variant thereof is expressed by introducing a nucleic acid in the yeast cell encoding Hv_FAR or the functional variant thereof. For example, a nucleic acid as set forth in SEQ ID NO: 74 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 74.

In some embodiments, the yeast cell expresses a FAR from Cydia pomonella. In some embodiments, the FAR is Cpo_FAR (SEQ ID NO: 76), i.e. the FAR naturally occurring in Cydia pomonella. In some embodiments, the heterologous FAR is a functional variant of Cpo_FAR, which retains the capability of converting E8,E10-C12:CoA to E8,E10-dodecadien-1-ol. For example, the functional variant has at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Cpo_FAR (SEQ ID NO: 76).

In some embodiments, Cpo_FAR or a functional variant thereof is expressed by introducing a nucleic acid in the yeast cell encoding Cpo_FAR or the functional variant thereof. For example, a nucleic acid as set forth in SEQ ID NO: 76 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 76.

In some embodiments, the FAR is Har_FAR (SEQ ID NO: 12), i.e. the FAR naturally occurring in Helicoverpa armigera. In some embodiments, the heterologous FAR is a functional variant of Har_FAR, which retains the capability of converting E8,E10-C12:CoA to E8,E10-dodecadien-1-ol. For example, the functional variant has at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to Har_FAR (SEQ ID NO: 12).

In some embodiments, Har_FAR or a functional variant thereof is expressed by introducing a nucleic acid in the yeast cell encoding Har_FAR or the functional variant thereof. For example, a nucleic acid as set forth in SEQ ID NO: 11 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 11.

In some embodiments, the yeast cell expresses several copies of the FAR. For example, the FAR is expressed at high level as is known in the art.

In some embodiments, the yeast cell expresses a desaturase and a FAR as described herein. In specific embodiments, the yeast cell expresses Cpo_CPRQ (SEQ ID NO: 2) or a functional variant thereof having at least 65% homology or identity thereto, and Ase_FAR (SEQ ID NO: 10) or a functional variant thereof having at least 65% homology or identity thereto. In some embodiments, the yeast cell expresses Cpo_CPRQ (SEQ ID NO: 2) or a functional variant thereof having at least 65% homology or identity thereto, and the FAR is a mutant Ase_FAR such as a mutant having a mutation at position 198 or 413, for example a T198A mutant or an S413A mutant. In some embodiments, the desaturase is a mutant Cpo_CPRQ such as a mutant having a mutation at position 85, for example an S85A mutant, and the FAR is Ase_FAR or a functional variant thereof. In other embodiments, the desaturase is an S85A Cpo_CPRQ mutant and the FAR is a mutant Ase_FAR such as a mutant having a mutation at position 198 or 413, for example a T198A mutant or an S413A mutant. In some embodiments, the desaturase is two desaturases, such as two identical desaturases, for example Cpo_CPRQ or a mutant Cpo_CPRQ having a mutation at position 85, for example an S85A mutant, and the FAR is Ase_FAR or a functional variant thereof. In some embodiments, the desaturase is two desaturases, such as two identical desaturases, for example Cpo_CPRQ or a mutant Cpo_CPRQ having a mutation at position 85, for example an S85A mutant, and the FAR is a mutant Ase_FAR such as a mutant having a mutation at position 198 or 413, for example a T198A mutant or an S413A mutant. In other embodiments, the desaturase is two different desaturases, for example a Cpo_CPRQ desaturase and a mutant Cpo_CPRQ desaturase having a mutation at position 85, for example an S85A mutant, and the FAR is Ase_FAR or a functional variant thereof. In other embodiments, the desaturase is two different desaturases, for example a Cpo_CPRQ desaturase and a mutant Cpo_CPRQ desaturase having a mutation at position 85, for example an S85A mutant, and the FAR is a mutant Ase_FAR such as a mutant having a mutation at position 198 or 413, for example a T198A mutant or an S413A mutant. In other embodiments, the yeast cell expresses Gmo_CPRQ (SEQ ID NO: 77) and Ase_FAR or a mutant or functional variant thereof.

In specific embodiments, the yeast cell expresses Cpo_CPRQ (SEQ ID NO: 2) or a functional variant thereof having at least 65% homology or identity thereto, and Aip_FAR (SEQ ID NO: 61) or a functional variant thereof having at least 65% homology or identity thereto. In some embodiments, the desaturase is a mutant Cpo_CPRQ such as a mutant having a mutation at position 85, for example an S85A mutant, and the FAR is Aip_FAR or a functional variant thereof. In some embodiments, the desaturase is two desaturases, such as two identical desaturases, for example two Cpo_CPRQ desaturases or two mutant Cpo_CPRQ desaturases having a mutation at position 85, for example two S85A mutants, and the FAR is Aip_FAR or a functional variant thereof. In other embodiments, the desaturase is two different desaturases, for example a Cpo_CPRQ desaturase and a mutant Cpo_CPRQ desaturase having a mutation at position 85, for example an S85A mutant, and the FAR is Aip_FAR or a functional variant thereof. In other embodiments, the yeast cell expresses Gmo_CPRQ (SEQ ID NO: 77) and Aip_FAR or a mutant or functional variant thereof.

In some embodiments, the yeast cell expresses Cpo_CPRQ (SEQ ID NO: 2) or a functional variant thereof having at least 65% homology or identity thereto, and Hs_FAR (SEQ ID NO: 71) or a functional variant thereof having at least 65% homology or identity thereto. In some embodiments, the desaturase is a mutant Cpo_CPRQ such as a mutant having a mutation at position 85, for example an S85A mutant, and the FAR is Hs_FAR or a functional variant thereof. In some embodiments, the desaturase is two desaturases, such as two identical desaturases, for example two Cpo_CPRQ desaturases or two mutant Cpo_CPRQ desaturases having a mutation at position 85, for example two S85A mutants, and the FAR is Hs_FAR or a functional variant thereof. In other embodiments, the desaturase is two different desaturases, for example a Cpo_CPRQ desaturase and a mutant Cpo_CPRQ desaturase having a mutation at position 85, for example an S85A mutant, and the FAR is Hs_FAR or a functional variant thereof. In other embodiments, the yeast cell expresses Gmo_CPRQ (SEQ ID NO: 77) and Hs_FAR or a mutant or functional variant thereof.

In specific embodiments, the yeast cell expresses Cpo_CPRQ (SEQ ID NO: 2) or a functional variant thereof having at least 65% homology or identity thereto, and Has_FAR (SEQ ID NO: 73) or a functional variant thereof having at least 65% homology or identity thereto. In some embodiments, the desaturase is a mutant Cpo_CPRQ such as a mutant having a mutation at position 85, for example an S85A mutant, and the FAR is Hs_FAR or a functional variant thereof. In some embodiments, the desaturase is two desaturases, such as two identical desaturases, for example two Cpo_CPRQ desaturases or two mutant Cpo_CPRQ desaturases having a mutation at position 85, for example two S85A mutants, and the FAR is Hs_FAR or a functional variant thereof. In other embodiments, the desaturase is two different desaturases, for example a Cpo_CPRQ desaturase and a mutant Cpo_CPRQ desaturase having a mutation at position 85, for example an S85A mutant, and the FAR is Hs_FAR or a functional variant thereof. In other embodiments, the yeast cell expresses Gmo_CPRQ (SEQ ID NO: 77) and Has_FAR or a mutant or functional variant thereof.

In specific embodiments, the yeast cell expresses Cpo_CPRQ (SEQ ID NO: 2) or a functional variant thereof having at least 65% homology or identity thereto, and Hv_FAR (SEQ ID NO: 75) or a functional variant thereof having at least 65% homology or identity thereto. In some embodiments, the desaturase is a mutant Cpo_CPRQ such as a mutant having a mutation at position 85, for example an S85A mutant, and the FAR is Hv_FAR or a functional variant thereof. In some embodiments, the desaturase is two desaturases, such as two identical desaturases, for example two Cpo_CPRQ desaturases or two mutant Cpo_CPRQ desaturases having a mutation at position 85, for example two S85A mutants, and the FAR is Hv_FAR or a functional variant thereof. In other embodiments, the desaturase is two different desaturases, for example a Cpo_CPRQ desaturase and a mutant Cpo_CPRQ desaturase having a mutation at position 85, for example an S85A mutant, and the FAR is Hv_FAR or a functional variant thereof. In other embodiments, the yeast cell expresses Gmo_CPRQ (SEQ ID NO: 77) and Hv_FAR or a mutant or functional variant thereof.

In specific embodiments, the yeast cell expresses Cpo_CPRQ (SEQ ID NO: 2) or a functional variant thereof having at least 65% homology or identity thereto, and Cpo_FAR (SEQ ID NO: 76) or a functional variant thereof having at least 65% homology or identity thereto. In some embodiments, the desaturase is a mutant Cpo_CPRQ such as a mutant having a mutation at position 85, for example an S85A mutant, and the FAR is Cpo_FAR or a functional variant thereof. In some embodiments, the desaturase is two desaturases, such as two identical desaturases, for example two Cpo_CPRQ desaturases or two mutant Cpo_CPRQ desaturases having a mutation at position 85, for example two S85A mutants, and the FAR is Cpo_FAR or a functional variant thereof. In other embodiments, the desaturase is two different desaturases, for example a Cpo_CPRQ desaturase and a mutant Cpo_CPRQ desaturase having a mutation at position 85, for example an S85A mutant, and the FAR is Cpo_FAR or a functional variant thereof. In other embodiments, the yeast cell expresses Gmo_CPRQ (SEQ ID NO: 77) and Cpo_FAR or a mutant or functional variant thereof.

In some embodiments, the yeast cell expresses a desaturase as described above, such as Cpo_CPRQ or Gmo_CPRQ, a mutant or a functional variant thereof, a FAR as described herein above, in particular Ase_FAR, Aip_FAR, Hs_AR, Has_FAR or Hv_FAR, or a mutant or a functional variant thereof having at least 65% homology or identity thereto, and also expresses another desaturase capable of introducing at least one double bond in a fatty acyl-CoA of carbon chain length 12, such as Cpo_NPVE (SEQ ID NO: 67) or Cpo_SPTQ (SEQ ID NO: 69), a mutant or a functional variant thereof having at least 65% homology or identity to SEQ ID NO: 67 or SEQ ID NO: 69, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to SEQ ID NO: 67 or SEQ ID NO: 69.

In some embodiments, the FAR is not Har_FAR (FAR from Helicoverpa armigera, SEQ ID NO: 12). In some embodiments, the FAR is not Ta_FAR (FAR from Tyto alba, SEQ ID NO: 8).

The yeast cell may thus express a desaturase capable of introducing one or more double bonds in said fatty acyl-CoA having a carbon chain length of 12, thereby converting said fatty acyl-CoA to a desaturated fatty acyl-CoA of carbon chain length 12 and having one or more double bonds, such as any of the desaturases described herein above, or functional variants thereof which retain the capability to convert a fatty acyl-CoA to a desaturated fatty acyl-CoA of carbon chain length 12. The yeast cell may further express a desaturase capable of introducing at least one double bond in a fatty acyl-CoA of carbon chain length >12, such as of carbon chain length 14 or more, or functional variants thereof which retain the capability to introduce at least one double bond in a fatty acyl-CoA of carbon chain length >12, such as of carbon chain length 14 or more, as described above. Any of these yeast cells may further express a reductase as described herein above, or a functional variant thereof which retains reductase activity.

In order to test whether a reductase or a functional variant thereof has the desired activity, methods known in the art can be employed. For example, the candidate enzyme to be tested can be introduced in the yeast cell, e.g. on a vector or in the genome of the yeast cell, incubating the yeast cell in an appropriate medium, extracting fatty alcohols from the broth, and performing an analysis such as a GC-MS analysis to determine whether desaturated fatty alcohols are produced. It may be advantageous to test the activity in a yeast cell in which the native elongase gene(s) has/have been deleted. An example of such a procedure is described in example 4 or in Schneiter et al., 2000.

### Increased Availability of Precursors

In order to improve production of E8,E10-dodecadienyl coenzyme A and optionally E8,E10-dodecadien-1-ol and derivatives thereof, it may be advantageous to introduce additional modifications in the yeast cell in order to increase availability of the required precursors, in particular of E8,E10-C12:CoA. The yeast cell may thus be further modified with any of the modifications detailed below, in particular:
Expression of a heterologous cytochrome b5
Expression of a heterologous cytochrome b5 reductase
Expression of a hemoglobin
Inactivation of native elongase(s) resulting in total or partial loss of activity
Inactivation of native thioesterase(s) resulting in total or partial loss of activity
Inactivation or modification of activity of native fatty aldehyde dehydrogenase(s), fatty alcohol oxidase(s), peroxisome biogenesis factor and/or fatty acyl synthase(s)
Expression of a heterologous thioesterase gene
Expression of a fusion protein of a fatty acyl synthase and of a thioesterase

An enzyme, for example an elongase, a thioesterase, a fatty aldehyde dehydrogenase, a fatty alcohol oxidase, a peroxisome biogenesis factor or a fatty acyl synthase, can be inactivated for example by introducing one or more mutations, including total or partial deletions, insertions, substitutions or non-sense or missense mutations, in the gene, for example in the coding sequence, promoter, Kozak sequence, terminator or other regulatory element. For example the native promoter or the native terminator can be replaced by another, weaker promoter or by another terminator, respectively. Other inactivation methods resulting in partial or total loss of activity include repression of transcription as well as post-transcriptional inactivation, such as silencing, for example using an RNAi system or a CRISPR/Cas system resulting in the degradation of the relevant transcripts, thereby preventing or at least reducing translation, as well as post-translational inactivation, such as inhibition of the protein. Enzyme activities can be otherwise modified, e.g. to modify properties of the enzymes such as intracellular localisation, or to increase activity, using methods known in the art.

Elongase activity can be tested by analysing the fatty acid profile, for example as described in Schneiter et al., 2000.

Thioesterase activity can be tested by appropriate assays, such as the thioesterase activity assay described in Nancolas et al., 2017.

Fatty aldehyde dehydrogenase activity can be tested by appropriate assays, such as a fatty aldehyde degradation assay as described in Iwama et al., 2014.

Fatty alcohol oxidase activity can be tested by appropriate assays, such as a fatty alcohol degradation assay as described in Iwama et al., 2015.

Peroxisome biogenesis factor activity can be tested by appropriate assays, such as growth assays of yeast cells expressing candidate fatty alcohol oxidases in a medium comprising fatty acids as sole carbon source.

Fatty acyl synthase activity can be tested by testing cell growth, as fatty acyl synthases are essential genes.

Any of said modifications can be combined, i.e. the yeast cell may comprise several of said modifications.

### Expression of a Heterologous Cytochrome b5

One modification which the present inventors have found to be beneficial for production of codlemone and derivatives thereof is the expression of a heterologous cytochrome b5 in a yeast cell. This membrane bound hemoprotein functions as an electron carrier for several membrane bound oxygenases. As shown in the examples (example 6 in particular) expression of a heterologous cytochrome b5 was found to increase availability of fatty acid methyl esters, in particular E8,E10-C12:Me and E9/Z9-C12: Me. Such a modification is thus expected to increase production of E8,E10-dodecadienyl coenzyme A and optionally desaturated fatty alcohols having a carbon chain length of 12, such as codlemone.

In some embodiments, the cytochrome b5 is a cytochrome b5 which is native to a Lepidoptera species. In particular embodiments, the cytochrome b5 is a cytochrome b5 from a Helicoverpa species, preferably a cytochrome b5 from Helicoverpa armigera, such as set forth in SEQ ID NO: 4, or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 85%, such as at least 90%, such as at least 95%, such as at least 96%, such as at least 97%, such as at least 98%, such as at least 99% homology or identity thereto.

The cytochrome b5 may be expressed at high level.

The cytochrome b5 may be expressed by introducing a nucleic acid in the yeast cell encoding a cytochrome b5 or a homologue thereof. For example, a nucleic acid as set forth in SEQ ID NO: 3 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 3.

In order to test whether a functional variant of a cytochrome b5 retains the desired activity, methods known in the art can be employed; for example, a spectrophotometric assay as described in Lamb et al., 1999.

Thus in some embodiments, the yeast cell expresses a desaturase and a fatty acyl-CoA reductase as described above, and further expresses a heterologous cytochrome b5 as described herein. In particular, the yeast cell may express one or more desaturases selected from Cpo_CPRQ (SEQ ID NO: 2), a mutant Cpo_CPRQ such as an S82 mutant or an S85 mutant, preferably an S85 mutant such as an S85A mutant, and functional variants thereof, and one or more reductases selected from Ase_FAR (SEQ ID NO: 10), a mutant Ase_FAR such as a T198 mutant or an S413 mutant, preferably a T198A mutant or an S413A mutant, Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12) and functional variants thereof, and a cytochrome b5 as described herein above, such as the cytochrome b5 from Helicoverpa armigera (SEQ ID NO: 4) or a functional variant thereof. The yeast cell may, in addition to Cpo_CPRQ, a mutant or a functional variant thereof, also express another desaturase capable of introducing at least one double bond in a fatty acyl-CoA of carbon chain length 12, as described above, for example Cpo_NPVE, Cpo_SPTQ, a mutant or a functional variant thereof.

The yeast cell may be further modified with any of the modifications described herein, in particular by expression of a heterologous cytochrome b5 reductase, expression of a hemoglobin, mutation in native elongase gene(s) resulting in total or partial loss of activity, mutation in native thioesterase gene(s) resulting in total or partial loss of activity, mutations in native gene(s) encoding fatty aldehyde dehydrogenase(s), fatty alcohol oxidase(s), peroxisome biogenesis factor and/or fatty acyl synthase(s), expression of a heterologous thioesterase gene and/or expression of a fusion protein of a fatty acyl synthase and of a thioesterase.

### Expression of a Heterologous Cytochrome b5 Reductase (EC 1.6.2.2)

Another modification which may lead to increased production of E8,E10-dodecadienyl coenzyme A and optionally codlemone and derivatives thereof is the expression of a heterologous cytochrome b5 reductase (EC 1.6.2.2).

Cytochrome b5 reductase, also known as methemoglobin reductase, is an NADH-dependent enzyme converting methemoglobin to hemoglobin: NADH+H⁺+2 ferricytochrome b₅=NAD⁺+2 ferrocytochrome b₅ In some embodiments, the cytochrome b5 reductase is a cytochrome b5 reductase which is native to a Lepidoptera species. In particular embodiments, the cytochrome b5 reductase is a cytochrome b5 reductase from a Helicoverpa species, preferably a cytochrome b5 reductase from a Helicoverpa species such as Helicoverpa armigera, for example the cytochrome b5 reductase as set forth in SEQ ID NO: 24, or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 85%, such as at least 90%, such as at least 95%, such as at least 96%, such as at least 97%, such as at least 98%, such as at least 99% homology or identity thereto.

In order to test whether a functional variant of a cytochrome b5 reductase retains the desired activity, methods known in the art can be employed; for example, a spectrophotometric assay as described in Lamb et al., 1999.

The cytochrome b5 reductase may be expressed at high level.

The cytochrome b5 reductase may be expressed by introducing a nucleic acid in the yeast cell encoding said cytochrome b5 reductase or a homologue thereof. For example, a nucleic acid as set forth in SEQ ID NO: 23 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 23.

Thus in some embodiments, the yeast cell expresses a desaturase and a fatty acyl-CoA reductase as described above, and further expresses a heterologous cytochrome b5 reductase as described herein. The yeast cell may be further modified with any of the modifications described herein. In particular, the yeast cell may express one or more desaturases selected from Cpo_CPRQ (SEQ ID NO: 2), Gmo_CPRQ (SEQ ID NO: 77), a mutant Cpo_CPRQ such as an S82 mutant or an S85 mutant, preferably an S85 mutant such as an S85A mutant, and functional variants thereof, and one or more reductases selected from Ase_FAR (SEQ ID NO: 10), a mutant Ase_FAR such as a T198 mutant or an S413 mutant, preferably a T198A mutant or an S413A mutant, Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12) and functional variants thereof, and a cytochrome b5 reducatse as described herein above, such as the cytochrome b5 reductase from Helicoverpa armigera (SEQ ID NO: 25) or a functional variant thereof. The yeast cell may, in addition to Cpo_CPRQ or Gmo_CPRQ, a mutant or a functional variant thereof, also express another desaturase capable of introducing at least one double bond in a fatty acyl-CoA of carbon chain length 12, as described above, for example Cpo_NPVE, Cpo_SPTQ, a mutant or a functional variant thereof. The yeast cell may additionally express a cytochrome b5 as described herein above.

The yeast cell may be further modified with any of the modifications described herein, in particular by expression of a heterologous cytochrome b5, expression of a hemoglobin, mutation in native elongase gene(s) resulting in total or partial loss of activity, mutation in native thioesterase gene(s) resulting in total or partial loss of activity, mutations in native gene(s) encoding fatty aldehyde dehydrogenase(s), fatty alcohol oxidase(s), peroxisome biogenesis factor and/or fatty acyl synthase(s), expression of a heterologous thioesterase gene and/or expression of a fusion protein of a fatty acyl synthase and of a thioesterase.

### Expression of a Hemoglobin

Another modification which may be advantageous for production of E8,E10-dodecadienyl coenzyme A and optionally codlemone and derivatives thereof is the expression of a hemoglobin in the yeast cell, in particular a heterologous hemoglobin.

As shown in the examples, in particular example 6, expression of a hemoglobin in a yeast cell expressing a desaturase increased production of E8,E10-C12:Me and E9/Z9-C12:Me.

In some embodiments, the hemoglobin is a hemoglobin which is native to a Vitreoscilla species, such as Vitreoscilla stercoraria. In particular embodiments, the hemoglobin is as set forth in SEQ ID NO: 6, or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 85%, such as at least 90%, such as at least 95%, such as at least 96%, such as at least 97%, such as at least 98%, such as at least 99% homology or identity thereto.

In order to test whether a functional variant of a hemoglobin retains the desired activity appropriate assays as known in the art, such as colorimetric assays, can be performed.

The hemoglobin may be expressed at high level.

The hemoglobin may be expressed by introducing a nucleic acid in the yeast cell encoding said hemoglobin or a homologue thereof. For example, a nucleic acid as set forth in SEQ ID NO: 5 is introduced, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 5.

Thus in some embodiments, the yeast cell expresses a desaturase and a fatty acyl-CoA reductase as described above, and further expresses a hemoglobin as described herein. In particular, the yeast cell may express one or more desaturases selected from Cpo_CPRQ (SEQ ID NO: 2), Gmo_CPRQ (SEQ ID NO: 77), a mutant Cpo_CPRQ such as an S82 mutant or an S85 mutant, preferably an S85 mutant such as an S85A mutant, and functional variants thereof, and one or more reductases selected from Ase_FAR (SEQ ID NO: 10), a mutant Ase_FAR such as a T198 mutant or an S413 mutant, preferably a T198A mutant or an S413A mutant, Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12) and functional variants thereof, and a hemoglobin as described herein above, such as the hemoglobin from Vitreoscilla stercoraria (SEQ ID NO: 6) or a functional variant thereof. The yeast cell may, in addition to Cpo_CPRQ or Gmo_CPRQ, a mutant or a functional variant thereof, also express another desaturase capable of introducing at least one double bond in a fatty acyl-CoA of carbon chain length 12, as described above, for example Cpo_NPVE, Cpo_SPTQ, a mutant or a functional variant thereof.

The yeast cell may be further modified with any of the modifications described herein, in particular by expression of a heterologous cytochrome b5, expression of a cytochrome b5 reductase, mutation in native elongase gene(s) resulting in total or partial loss of activity, mutation in native thioesterase gene(s) resulting in total or partial loss of activity, mutations in native gene(s) encoding fatty aldehyde dehydrogenase(s), fatty alcohol oxidase(s), peroxisome biogenesis factor and/or fatty acyl synthase(s), expression of a heterologous thioesterase gene and/or expression of a fusion protein of a fatty acyl synthase and of a thioesterase.

### Mutation in Elongase Gene(s)

Another modification which may be advantageous for production of E8,E10-dodecadienyl coenzyme A and optionally codlemone and derivatives thereof is the mutation of certain genes in the yeast cell, in particular mutation of one or more elongase genes, where the mutation results in a partial or total loss of activity of the corresponding elongase. Elongases catalyse carbon chain extension of several molecules, including fatty acids. In some embodiments, the elongase is a medium chain acyl elongase. If a yeast cell is used which naturally comprises several genes encoding elongases, the yeast cell may be further engineered to comprise a mutation in one or more of said genes, resulting in partial or total loss of activity of the one or more elongases.

In some embodiments, the yeast cell is a Yarrowia lipolytica cell and the elongase is encoded by the ELO1 gene (SEQ ID NO: 13).

In some embodiments, the mutation is a deletion resulting in total loss of activity of the corresponding elongase. In other embodiments, the elongase is inactivated for example by introducing one or more mutations, including total or partial deletions, insertions, substitutions or non-sense or missense mutations, in the gene, for example in the coding sequence, promoter, Kozak sequence, terminator or other regulatory element. For example the native promoter or the native terminator can be replaced by another, weaker promoter or by another terminator, respectively. Other inactivation methods resulting in partial or total loss of activity include repression of transcription as well as post-transcriptional inactivation, such as silencing, for example using an RNAi system or a CRISPR/Cas system resulting in the degradation of the relevant transcripts, thereby preventing or at least reducing translation, as well as post-translational inactivation, such as inhibition of the protein. An example of how to test whether a protein retains elongase activity is described in example 4 or in Schneiter et al., 2000.

Thus in some embodiments, the yeast cell expresses a desaturase and a fatty acyl-CoA reductase as described above, and further comprises one or more mutations in one or more genes encoding an elongase, wherein said mutation results in partial or total loss of function, as described herein. In particular, the yeast cell may express one or more desaturases selected from Cpo_CPRQ (SEQ ID NO: 2), a mutant Cpo_CPRQ such as an S82 mutant or an S85 mutant, preferably an S85 mutant such as an S85A mutant, and functional variants thereof, and one or more reductases selected from Ase_FAR (SEQ ID NO: 10), a mutant Ase_FAR such as a T198 mutant or an S413 mutant, preferably a T198A mutant or an S413A mutant, Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12) and functional variants thereof, and may further comprise a mutation resulting in partial or total loss of activity of an elongase as described herein above. The yeast cell may, in addition to Cpo_CPRQ or Gmo_CPRQ, a mutant or a functional variant thereof, also express another desaturase capable of introducing at least one double bond in a fatty acyl-CoA of carbon chain length 12, as described above, for example Cpo_NPVE, Cpo_SPTQ, a mutant or a functional variant thereof.

The yeast cell may be further modified with any of the modifications described herein, in particular by expression of a heterologous cytochrome, expression of a heterologous cytochrome b5 reductase, expression of a hemoglobin, mutation in native thioesterase gene(s) resulting in total or partial loss of activity, mutations in native gene(s) encoding fatty aldehyde dehydrogenase(s), fatty alcohol oxidase(s), peroxisome biogenesis factor and/or fatty acyl synthase(s), expression of a heterologous thioesterase gene and/or expression of a fusion protein of a fatty acyl synthase and of a thioesterase.

### Mutation in Thioesterase Gene(s)

Another modification which may be advantageous for production of E8,E10-dodecadienyl coenzyme A and optionally codlemone and derivatives thereof is the mutation of certain genes in the yeast cell, in particular mutation of one or more thioesterase genes, where the mutation results in a partial or total loss of activity of the corresponding thioesterase. If a yeast cell is used which naturally comprises several genes encoding thioesterases, the yeast cell may be further engineered to comprise a mutation in one or more of said genes, resulting in partial or total loss of activity of the one or more thioesterases.

In some embodiments, the yeast cell is a Yarrowia lipolytica cell and the thioesterase is encoded by YAL10_F14729g gene (SEQ ID NO: 19), YALI0_E18876g (SEQ ID NO: 54) or YALI0_D03597g (SEQ ID NO: 55). Thus in some embodiments the Yarrowia lipolytica cell comprises a mutation, such as a deletion, of the YAL10_F14729g gene (SEQ ID NO: 19) resulting in partial or total loss of the corresponding thioesterase. In other embodiments the Yarrowia lipolytica cell comprises a mutation, such as a deletion, of the YALI0_E18876g gene (SEQ ID NO: 54) resulting in partial or total loss of the corresponding thioesterase. In other embodiments the Yarrowia lipolytica cell comprises a mutation, such as a deletion, of the YALI0_D03597g (SEQ ID NO: 55) resulting in partial or total loss of the corresponding thioesterase. In some embodiments, the Yarrowia lipolytica cell comprises a mutation in several thioesterase genes. For example, the cell may comprise a mutation, such as a deletion, of YAL10_F14729g (SEQ ID NO: 19) and of YALI0_E18876g (SEQ ID NO: 54); or a mutation, such as a deletion, of YAL10_F14729g (SEQ ID NO: 19) and of YALI0_D03597g (SEQ ID NO: 55); or a mutation, such as a deletion, of YALI0_E18876g (SEQ ID NO: 54) and of YALI0_D03597g (SEQ ID NO: 55). In some embodiments, the cell comprises a mutation, such as a deletion, of YAL10_F14729g (SEQ ID NO: 19), YALI0_E18876g (SEQ ID NO: 54) and YALI0_D03597g (SEQ ID NO: 55).

Thus in some embodiments, the yeast cell expresses a desaturase and a fatty acyl-CoA reductase as described above, and further comprises one or more mutations in one or more genes encoding a thioesterase, wherein said mutation results in partial or total loss of function, as described herein. In particular, the yeast cell may express one or more desaturases selected from Cpo_CPRQ (SEQ ID NO: 2), Gmo_CPRQ (SEQ ID NO: 77) a mutant Cpo_CPRQ such as an S82 mutant or an S85 mutant, preferably an S85 mutant such as an S85A mutant, and functional variants thereof, and one or more reductases selected from Ase_FAR (SEQ ID NO: 10), a mutant Ase_FAR such as a T198 mutant or an S413 mutant, preferably a T198A mutant or an S413A mutant, Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12) and functional variants thereof, and one or more mutations in one or more genes encoding a thioesterase, wherein said mutation results in partial or total loss of function as described herein above, such as a mutation in one or more of YAL10_F14729g (SEQ ID NO: 19), YALI0_E18876g (SEQ ID NO: 54) and YALI0_D03597g (SEQ ID NO: 55). The yeast cell may, in addition to Cpo_CPRQ or Gmo_CPRQ, a mutant or a functional variant thereof, also express another desaturase capable of introducing at least one double bond in a fatty acyl-CoA of carbon chain length 12, as described above, for example Cpo_NPVE. Cpo_SPTQ, a mutant or a functional variant thereof. The yeast cell may be further modified with any of the modifications described herein, in particular by expression of a heterologous cytochrome b5, expression of a heterologous cytochrome b5 reductase, expression of a hemoglobin, mutation in native elongase gene(s) resulting in total or partial loss of activity, mutations in native gene(s) encoding fatty aldehyde dehydrogenase(s), fatty alcohol oxidase(s), peroxisome biogenesis factor and/or fatty acyl synthase(s), expression of a heterologous thioesterase gene and/or expression of a fusion protein of a fatty acyl synthase and of a thioesterase.

### Additional Modifications

The yeast cell may further comprise other modifications, such as at least one mutation resulting in reduced activity of enzymes involved in fatty acid metabolism. In some embodiments, activity of the native fatty aldehyde dehydrogenase(s), fatty alcohol oxidase(s), peroxisome biogenesis factor and/or fatty acyl synthase(s) is modified, preferably the activity is reduced or abolished. For example, the yeast cell may further comprise one or more mutations in genes encoding a fatty aldehyde dehydrogenase, a fatty alcohol oxidase, and/or a peroxisome biogenesis factor. Any of these enzymes may be inactivated for example by introducing one or more mutations, including total or partial deletions, insertions, substitutions or non-sense or missense mutations, in the gene, for example in the coding sequence, promoter, Kozak sequence, terminator or other regulatory element. For example the native promoter or the native terminator can be replaced by another, weaker promoter or by another terminator, respectively. Other inactivation methods resulting in partial or total loss of activity include repression of transcription as well as post-transcriptional inactivation, such as silencing, for example using an RNAi system or a CRISPR/Cas system resulting in the degradation of the relevant transcripts, thereby preventing or at least reducing translation, as well as post-translational inactivation, such as inhibition of the protein. Enzyme activities can be otherwise modified, e.g. to modify properties of the enzymes such as intracellular localisation, or to increase activity, using methods known in the art.

In some embodiments, the yeast cell is a Yarrowia lipolytica cell as described herein above, further comprising a modification such as a mutation in at least one of HFD1, HFD2, HFD3, HFD4, FAO1, GPAT and PEX10, or a modification such as a mutation resulting in reduced activity of at least one protein having at least 60% homology or identity thereto, such as at least 65% homology or identity, such as at least 70% homology or identity, such as at least 75% homology or identity, such as at least 80% homology or identity, such as at least 81% homology or identity, such as at least 82% homology or identity, such as at least 83% homology or identity, such as at least 84% homology or identity, such as at least 85% homology or identity, such as at least 86% homology or identity, such as at least 87% homology or identity, such as at least 88% homology or identity, such as at least 89% homology or identity, such as at least 90% homology or identity, such as at least 91% homology or identity, such as at least 92% homology or identity, such as at least 93% homology or identity, such as at least 94% homology or identity, such as at least 95% homology or identity, such as at least 96% homology or identity, such as at least 97% homology or identity, such as at least 98% homology or identity, such as at least 99% homology or identity thereto.

In Yarrowia lipolytica, the fatty aldehyde dehydrogenase Hfd1 is encoded by HFD1 (YALI0_F23793g). It catalyses the oxidation of fatty aldehydes to fatty acids. As described in detail in application WO 2018/109163, reduced activity of Hfd1 results in increased titer of desaturated fatty alcohols in yeast cells. A Yarrowia lipolytica cell according to the present disclosure may thus further comprise a mutation, such as a deletion, of HFD1, resulting in partial or total loss of activity of Hfd1. Reduction of activity of Hfd1 can be achieved by other methods as described herein.

The fatty aldehyde dehydrogenase Hfd2 is encoded by HFD2 (YALI_0E15400g). It catalyses the oxidation of fatty aldehydes to fatty acids. A Yarrowia lipolytica cell according to the present disclosure may thus further comprise a mutation, such as a deletion, of HFD2, resulting in partial or total loss of activity of Hfd2. Reduction of activity of Hfd2 can be achieved by other methods as described herein.

The fatty aldehyde dehydrogenase Hfd3 is encoded by HFD3 (YALI0_A17875g). It catalyses the oxidation of fatty aldehydes to fatty acids. A Yarrowia lipolytica cell according to the present disclosure may thus further comprise a mutation, such as a deletion, of HFD3, resulting in partial or total loss of activity of Hfd3. Reduction of activity of Hfd3 can be achieved by other methods as described herein.

In Yarrowia lipolytica, the fatty aldehyde dehydrogenase Hfd4 is encoded by HFD4 (YALI0_B01298g). It catalyses the oxidation of fatty aldehydes to fatty acids. As described in detail in application WO 2018/109163, reduced activity of Hfd4 results in increased titer of desaturated fatty alcohols in yeast cells. A Yarrowia lipolytica cell according to the present disclosure may thus further comprise a mutation, such as a deletion, of HFD4, resulting in partial or total loss of activity of Hfd4. Reduction of activity of Hfd4 can be achieved by other methods as described herein.

In some embodiments, the yeast cell further comprises a modification, for example a mutation, such as a deletion, resulting in partial or total loss of activity of a fatty aldehyde dehydrogenase having at least 60% homology or identity to Hfd1, Hfd2, Hfd3 or Hfd4, such as at least 65% homology or identity, such as at least 70% homology or identity, such as at least 75% homology or identity, such as at least 80% homology or identity, such as at least 81% homology or identity, such as at least 82% homology or identity, such as at least 83% homology or identity, such as at least 84% homology or identity, such as at least 85% homology or identity, such as at least 86% homology or identity, such as at least 87% homology or identity, such as at least 88% homology or identity, such as at least 89% homology or identity, such as at least 90% homology or identity, such as at least 91% homology or identity, such as at least 92% homology or identity, such as at least 93% homology or identity, such as at least 94% homology or identity, such as at least 95% homology or identity, such as at least 96% homology or identity, such as at least 97% homology or identity, such as at least 98% homology or identity, such as at least 99% homology or identity to Hfd1, Hfd2, Hfd3 or Hfd4.

In Yarrowia lipolytica, the fatty alcohol oxidase Fao1 is encoded by FAO1 (YALI0B14014g). Its deletion results in increased accumulation of ω-hydroxy fatty acids. As described in detail in application WO 2018/109163, reduced activity of Fao1 results in increased titer of desaturated fatty alcohols in yeast cells. A Yarrowia lipolytica cell according to the present disclosure may thus further comprise a mutation, such as a deletion, of FAO1, resulting in partial or total loss of activity of Fao1. Reduction of activity of Fao1 can be achieved by other methods as described herein.

In some embodiments, the yeast cell further comprises a mutation, such as a deletion, resulting in partial or total loss of activity of a fatty alcohol oxidase having at least 60% homology or identity to Fao1, such as at least 65% homology or identity, such as at least 70% homology or identity, such as at least 75% homology or identity, such as at least 80% homology or identity, such as at least 81% homology or identity, such as at least 82% homology or identity, such as at least 83% homology or identity, such as at least 84% homology or identity, such as at least 85% homology or identity, such as at least 86% homology or identity, such as at least 87% homology or identity, such as at least 88% homology or identity, such as at least 89% homology or identity, such as at least 90% homology or identity, such as at least 91% homology or identity, such as at least 92% homology or identity, such as at least 93% homology or identity, such as at least 94% homology or identity, such as at least 95% homology or identity, such as at least 96% homology or identity, such as at least 97% homology or identity, such as at least 98% homology or identity, such as at least 99% homology or identity to Fao1.

In Yarrowia lipolytica, the peroxisome biogenesis factor 10 Pex10 is encoded by PEX10 (YALI0C01023g). As described in detail in application WO 2018/109163, reduced activity of Pex10 results in increased titer of desaturated fatty alcohols in yeast cells. A Yarrowia lipolytica cell according to the present disclosure may thus further comprise a mutation, such as a deletion, of PEX10, resulting in partial or total loss of activity of Pex10. Reduction of activity of Pex10 can be achieved by other methods as described herein.

In some embodiments, the yeast cell further comprises a mutation, such as a deletion, resulting in partial or total loss of activity of a peroxisome biogenesis factor having at least 60% homology or identity to Pex10, such as at least 65% homology or identity, such as at least 70% homology or identity, such as at least 75% homology or identity, such as at least 80% homology or identity, such as at least 81% homology or identity, such as at least 82% homology or identity, such as at least 83% homology or identity, such as at least 84% homology or identity, such as at least 85% homology or identity, such as at least 86% homology or identity, such as at least 87% homology or identity, such as at least 88% homology or identity, such as at least 89% homology or identity, such as at least 90% homology or identity, such as at least 91% homology or identity, such as at least 92% homology or identity, such as at least 93% homology or identity, such as at least 94% homology or identity, such as at least 95% homology or identity, such as at least 96% homology or identity, such as at least 97% homology or identity, such as at least 98% homology or identity, such as at least 99% homology or identity to Pex10.

In Yarrowia lipolytica, the glycerol-3-phosphate acyltransferase is encoded by GPAT (YALI0_C00209g). GPAT catalyzes the first reaction towards glycerolipids biosynthesis. The gene is essential in Yarrowia lipolytica. As described in detail in application WO 2018/109163, reduced activity of GPAT results in increased titer of desaturated fatty alcohols in yeast cells. A Yarrowia lipolytica cell according to the present disclosure may thus further comprise a mutation of GPAT, resulting in partial or total loss of activity of GPAT. Reduction of activity of GPAT can be achieved by other methods as described herein.

In some embodiments, the yeast cell further comprises a mutation resulting in partial or total loss of activity of a glycerol-3-phosphate acyltransferase having at least 60% homology or identity to GPAT, such as at least 65% homology or identity, such as at least 70% homology or identity, such as at least 75% homology or identity, such as at least 80% homology or identity, such as at least 81% homology or identity, such as at least 82% homology or identity, such as at least 83% homology or identity, such as at least 84% homology or identity, such as at least 85% homology or identity, such as at least 86% homology or identity, such as at least 87% homology or identity, such as at least 88% homology or identity, such as at least 89% homology or identity, such as at least 90% homology or identity, such as at least 91% homology or identity, such as at least 92% homology or identity, such as at least 93% homology or identity, such as at least 94% homology or identity, such as at least 95% homology or identity, such as at least 96% homology or identity, such as at least 97% homology or identity, such as at least 98% homology or identity, such as at least 99% homology or identity to GPAT.

Partial or total loss of activity of any of the above enzymes can also be achieved for example by introducing one or more mutations, including total or partial deletions, insertions, substitutions or non-sense or missense mutations, in the gene, for example in the coding sequence, promoter, Kozak sequence, terminator or other regulatory element. For example the native promoter or the native terminator can be replaced by another, weaker promoter or by another terminator, respectively. Other inactivation methods resulting in partial or total loss of activity include repression of transcription as well as post-transcriptional inactivation, such as silencing, for example using an RNAi system or a CRISPR/Cas system resulting in the degradation of the relevant transcripts, thereby preventing or at least reducing translation, as well as post-translational inactivation, such as inhibition of the protein. In order to determine whether a modification, such as a mutation or any modification described herein above, results in total or partial loss of activity, methods known in the art can be employed, such as detailed herein above. For example, in the case of a deletion or a modification leading to reduced transcription, amplification methods such as PCR may be employed to confirm absence of the relevant sequence. Protein expression may be investigated using appropriate assays, such as a Western blot or measuring expression levels using a marker such as a fluorescent marker.

It may also be advantageous for the yeast cell to express one or more modified fatty acyl synthases. This may help direct the metabolic flux towards production of desaturated products such as E8,E10-dodecadienyl coenzyme A and desaturated fatty alcohols and derivatives thereof, such as codlemone and derivatives thereof. Thus in some embodiments the yeast cell is further modified to express a fatty acyl synthase having a modified ketone synthase domain. In some embodiments, the yeast cell is a Yarrowia lipolytica cell as described herein, wherein the cell further expresses a modified fatty acid synthase complex. In one embodiment, the fatty acid synthase complex is modified by mutating the gene encoding the alpha subunit of the complex. In some embodiments, the mutation is in the gene encoding FAS2 (SEQ ID NO: 18).

In other embodiments, the mutation is in the gene encoding FAS1 (SEQ ID NO: 16). The mutation may result in modification of one or more of residue 123 (L123) of SEQ ID NO: 16. The mutation may result in modification of one or more of residue 1220 (11220), residue 1217 (M1217) or residue 1226 (M1226) of SEQ ID NO: 18, resulting in a variant FAS2. The skilled person will know how to design such mutations.

Preferably, a mutation in FAS2 results in an I1220F variant, an I1220W variant, an 11220Y variant or an 11220H variant of Fas2. In a specific embodiment, the mutation results in an I1220F variant. In some embodiments, the mutation results in an M1217F variant, an M1217W variant, an M1217Y variant or an M1217H variant. In other embodiments, the mutation results in an M1226F variant, an M1226W variant, an M1226Y variant or an M1226H variant.

Preferably, a mutation in FAS1 results in an L123V variant.

Yeast cells with more than one of the above mutations are also contemplated, such as two mutations or three mutations at residues 11220, M1217 or M1226 of FAS2, and/or one mutation at residue 123 of FAS1.

Thus in some embodiments, the yeast cell expresses a desaturase and a fatty acyl-CoA reductase as described above, and further comprises one or more modifications as described within the present section. In particular, the yeast cell may express one or more desaturases selected from Cpo_CPRQ (SEQ ID NO: 2), Gmo_CPRQ (SEQ ID NO: 77), a mutant Cpo_CPRQ such as an S82 mutant or an S85 mutant, preferably an S85 mutant such as an S85A mutant, and functional variants thereof, and one or more reductases selected from Ase_FAR (SEQ ID NO: 10), a mutant Ase_FAR such as a T198 mutant or an S413 mutant, preferably a T198A mutant or an S413A mutant, Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12) and functional variants thereof, and one or more modifications such as mutations resulting in partial or total loss of function of one or more of Hfd1, Hfd2, Hfd3, Hfd4, Fao1 and Pex10 as described above, and/or further expresses one or more modified fatty acyl synthases as described above. The yeast cell may, in addition to Cpo_CPRQ or Gmo_CPRQ, a mutant or a functional variant thereof, also express another desaturase capable of introducing at least one double bond in a fatty acyl-CoA of carbon chain length 12, as described above, for example Cpo_NPVE, Cpo_SPTQ, a mutant or a functional variant thereof.

The yeast cell may be further modified with any of the modifications described herein, in particular by expression of a heterologous cytochrome b5, expression of a heterologous cytochrome b5 reductase, expression of a hemoglobin, inactivation of native elongase(s) resulting in total or partial loss of activity, inactivation of native thioesterase(s) resulting in total or partial loss of activity, expression of a heterologous thioesterase gene and/or expression of a fusion protein of a fatty acyl synthase and of a thioesterase.

In particular, the yeast cell may express one or more desaturases selected from Cpo_CPRQ (SEQ ID NO: 2), Gmo_CPRQ (SEQ ID NO: 77) a mutant Cpo_CPRQ such as an S82 mutant or an S85 mutant, preferably an S85 mutant such as an S85A mutant, and functional variants thereof, and one or more reductases selected from Ase_FAR (SEQ ID NO: 10), a mutant Ase_FAR such as a T198 mutant or an S413 mutant, preferably a T198A mutant or an S413A mutant, Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12) and functional variants thereof and may further comprise mutations in: HFD1 and HFD2; HFD1 and HFD3; HFD1 and HFD4; HFD1 and FAO1; HFD1 and PEX10; HFD2 and HFD3; HFD2 and HFD4; HFD2 and FAO1; HFD2 and PEX10; HFD3 and HFD4; HFD3 and FAO1; HFD3 and PEX10; HFD4 and FAO1; HFD4 and PEX10; FAO1 and PEX10; HFD1, HFD2 and HFD3; HFD1, HFD2 and HFD4; HFD1, HFD2 and FAO1; HFD1, HFD2 and PEX10; HFD1, HFD3 and HFD4; HFD1, HFD3 and FAO1; HFD1, HFD3 and PEX10; HFD1, HFD4 and FAO1; HFD1, HFD4 and PEX10; HFD1, FAO1 and PEX10; HFD2, HFD3 and HFD4; HFD2, HFD3 and FAO1; HFD2, HFD3 and PEX10; HFD2, HFD4 and FAO1; HFD2, HFD4 and PEX10; HFD2, FAO1 and PEX10; HFD3, HFD4 and FAO1; HFD3, HFD4 and PEX10; HFD3, FAO1 and PEX10; HFD4, FAO1 and PEX10; HFD1, HFD2, HFD3 and HFD4; HFD1, HFD2, HFD3 and FAO1; HFD1, HFD2, HFD3 and PEX10; HFD1, HFD2, HFD4 and FAO1; HFD1, HFD2, HFD4 and PEX10; HFD1, HFD2, FAO1 and PEX10; HFD1, HFD3, HFD4 and FAO1; HFD1, HFD3, HFD4 and PEX10; HFD1, HFD3, FAO1 and PEX10; HFD1, HFD4, FAO1 and PEX10; HFD2, HFD3, HFD4 and FAO1; HFD2, HFD3, HFD4 and PEX10; HFD2, HFD3, FAO1 and PEX10; HFD2, HFD4, FAO1 and PEX10; HFD3, HFD4, FAO1 and PEX10; HFD1, HFD2, HFD3, HFD4 and FAO1; HFD1, HFD2, HFD3, HFD4 and PEX10; HFD1, HFD3, HFD4, FAO1 and PEX10; HFD2, HFD3, HFD4, FAO1 and PEX10; HFD1, HFD2, HFD3, HFD4, FAO1 and PEX10, or the afore-mentioned combinations of corresponding variants having at least 60% homology or identity thereto. In addition, the yeast cell may further express a modified fatty acyl synthase as described above, in particular a mutant Fas1 and/or a mutant Fas2. The yeast cell may, in addition to Cpo_CPRQ or Gmo_CPRQ, a mutant or a functional variant thereof, also express another desaturase capable of introducing at least one double bond in a fatty acyl-CoA of carbon chain length 12, as described above, for example Cpo_NPVE, Cpo_SPTQ, a mutant or a functional variant thereof.

### Expression of a Heterologous Thioesterase

It may be advantageous to further engineer the yeast cell by introducing a thioesterase, in particular a heterologous thioesterase. Thus in some embodiments, a nucleic acid encoding a thioesterase is introduced in the yeast cell, e.g. on a vector or by genomic integration. The thioesterase gene may be under the control of an inducible promoter, or under the control of a constitutive promoter. The nucleic acid encoding a thioesterase may be codon-optimised for the yeast cell, as is known in the art. In particular, the nucleic acid may be codon-optimised for a Yarrowia cell, such as a Yarrowia lipolytica cell. The thioesterase may be expressed at high level as is known in the art.

In some embodiments, the thioesterase is derived from an organism selected from Cuphea palustris, Cuphea hookeriana, Cinnamomum camphora, or from Escherichia coli. In preferred embodiments, the thioesterase is derived from Escherichia coli or Cinnamomum camphora. In some embodiments, the thioesterase has at least 60% homology or identity to a thioesterase selected from the thioesterase derived from Cuphea palustris as set forth in SEQ ID NO: 33, the thioesterase derived from Cuphea hookeriana as set forth in SEQ ID NO: 57, the thioesterase derived from Cinnamomum camphora as set forth in SEQ ID NO: 35, and the thioesterase derived from Escherichia coli as set forth in SEQ ID NO: 26. Preferably, the thioesterase has at least 60% homology or identity to the thioesterase derived from Cinnamomum camphora as set forth in SEQ ID NO: 35 or from Escherichia coli as set forth in SEQ ID NO: 26. In one embodiment, the thioesterase has at least 60% homology or identity to the thioesterase derived from Cinnamomum camphora as set forth in SEQ ID NO: 35. In another embodiment the thioesterase has at least 60% homology or identity to the thioesterase derived from Escherichia coli as set forth in SEQ ID NO: 26.

In another embodiment, the thioesterase has at least 60% homology or identity to the thioesterase derived from Cinnamomum camphora as set forth in SEQ ID NO: 35, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology or identity to the thioesterase derived from Cinnamomum camphora as set forth in SEQ ID NO: 35.

In another embodiment, the thioesterase has at least 60% homology or identity to the thioesterase derived from Escherichia coli as set forth in SEQ ID NO: 26, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology or identity to the thioesterase derived from Escherichia coli as set forth in SEQ ID NO: 26.

The nucleic acid encoding a thioesterase may be codon-optimised as is known in the art. In one embodiment, the yeast cell is a Yarrowia cell, preferably a Yarrowia lipolytica cell, and the nucleic acid is codon-optimised accordingly.

In one embodiment, the at least one thioesterase is encoded by a nucleic acid having at least 60% homology or identity to the nucleic acid encoding the thioesterase derived from Cinnamomum camphora as set forth in SEQ ID NO: 34, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology or identity to the nucleic acid encoding the thioesterase from Cinnamomum camphora as set forth in SEQ ID NO: 34.

In one embodiment, the at least one thioesterase is encoded by a nucleic acid having at least 60% homology or identity to the nucleic acid encoding the thioesterase derived from Escherichia coli as set forth in SEQ ID NO: 25, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology or identity to the nucleic acid encoding the thioesterase from Escherichia coli as set forth in SEQ ID NO: 25.

Thus in some embodiments, the yeast cell expresses a desaturase and a fatty acyl-CoA reductase as described above, and further expresses one or more thioesterases such as one or more heterologous thioesterases, as described herein. In particular, the yeast cell may express one or more desaturases selected from Cpo_CPRQ (SEQ ID NO: 2), Gmo_CPRQ (SEQ ID NO: 77), a mutant Cpo_CPRQ such as an S82 mutant or an S85 mutant, preferably an S85 mutant such as an S85A mutant, and functional variants thereof, and one or more reductases selected from Ase_FAR (SEQ ID NO: 10), a mutant Ase_FAR such as a T198 mutant or an S413 mutant, preferably a T198A mutant or an S413A mutant, Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO. 4) and functional variants thereof, and one or more heterologous thioesterases such as the thioesterases set forth in SEQ ID NO: 33, SEQ ID NO: 57, SEQ ID NO: 35 and/or SEQ ID NO: 26, or functional variants thereof. The yeast cell may, in addition to Cpo_CPRQ or Gmo_CPRQ, a mutant or a functional variant thereof, also express another desaturase capable of introducing at least one double bond in a fatty acyl-CoA of carbon chain length 12, as described above, for example Cpo_NPVE, Cpo_SPTQ, a mutant or a functional variant thereof.

The yeast cell may be further modified with any of the modifications described herein, in particular by expression of a heterologous cytochrome b5, a heterologous cytochrome b5 reductase, expression of a hemoglobin, mutation in native elongase gene(s) resulting in total or partial loss of activity, mutation in native thioesterase gene(s) resulting in total or partial loss of activity, mutations in native gene(s) encoding fatty aldehyde dehydrogenase(s), fatty alcohol oxidase(s), peroxisome biogenesis factor and/or fatty acyl synthase(s), and/or expression of a fusion protein of a fatty acyl synthase and of a thioesterase, as described herein above.

### Expression of a Fusion Protein of a Fatty Acyl Synthase and of a Thioesterase

In some embodiments, the yeast cell further expresses a fusion protein of a truncated fatty acyl synthase and of a truncated thioesterase, such as the fusion protein as set forth in SEQ ID NO: 59 or a homologue thereof having at least 60% homology or identity thereto. This fusion protein is a fusion of a truncated version of Fas1 from Y. lipolytica and of a truncated version of the thioesterase TesA from E. coli. It can be expressed by introduction of a nucleic acid such as set forth in SEQ ID NO: 58. The fusion protein may be expressed at high level.

Thus in some embodiments, the yeast cell further expresses a fusion protein as set forth in SEQ ID NO: 59 or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 59.

In some embodiments, the yeast cell comprises a nucleic acid encoding said fusion protein such as the nucleic acid as set forth in SEQ ID NO: 58 or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 58.

Thus in some embodiments, the yeast cell expresses a desaturase and a fatty acyl-CoA reductase as described above, and further expresses a fusion protein of a truncated fatty acyl synthase and of a truncated thioesterase, such as the fusion protein as set forth in SEQ ID NO: 59. In particular, the yeast cell may express one or more desaturases selected from Cpo_CPRQ (SEQ ID NO: 2), a mutant Cpo_CPRQ such as an S82 mutant or an S85 mutant, preferably an S85 mutant such as an S85A mutant, and functional variants thereof, and one or more reductases selected from Ase_FAR (SEQ ID NO: 10), a mutant Ase_FAR such as a T198 mutant or an S413 mutant, preferably a T198A mutant or an S413A mutant, Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12) and functional variants thereof, and a fusion protein of a truncated fatty acyl synthase and of a truncated thioesterase, such as the fusion protein as set forth in SEQ ID NO: 59 or a functional variant thereof. The yeast cell may, in addition to Cpo_CPRQ or Gmo_CPRQ, a mutant or a functional variant thereof, also express another desaturase capable of introducing at least one double bond in a fatty acyl-CoA of carbon chain length 12, as described above, for example Cpo_NPVE, Cpo_SPTQ, a mutant or a functional variant thereof. The yeast cell may be further modified with any of the modifications described herein, in particular by expression of a heterologous cytochrome b5, expression of a heterologous cytochrome b5 reductase, expression of a hemoglobin, mutation in native elongase gene(s) resulting in total or partial loss of activity, mutation in native thioesterase gene(s) resulting in total or partial loss of activity, mutations in native gene(s) encoding fatty aldehyde dehydrogenase(s), fatty alcohol oxidase(s), peroxisome biogenesis factor and/or fatty acyl synthase(s) and/or expression of a heterologous thioesterase gene, as described herein above.

### Titer

The yeast cells disclosed herein are capable of producing E8,E10-dodecadien-1-ol with a titre of at least 0.2 mg/L. In some embodiments, the titre of E8,E10-dodecadien-1-ol is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

Methods for determining the titer are known in the art.

Production of E8, E10-dodecadienyl acetate

Codlemone can be further converted to E8,E10-dodecadienyl acetate; this can be done ex vivo, as is known in the art, e.g. by chemical conversion, or it can be done in vivo by the action of an acetyltransferase (EC 2.3.1.84) capable of converting at least part of the E8,E10-dodecadien-1-ol produced by the cell into E8,E10-dodecadienyl acetate.

In some embodiments, the yeast cell is thus engineered so that it overexpresses a native acetyltransferase and/or so that it expresses a heterologous acetyltransferase, which is optionally expressed at high level. The yeast cell is in such embodiments capable of producing E8,E10-dodecadien-1-ol and E8,E10-dodecadienyl acetate.

In some embodiments, the yeast cell expresses an acetyltransferase capable of converting at least part of the E8,E10-dodecadien-1-ol produced by the cell into E8,E10-dodecadienyl acetate, such as the Sc_Atf1 acetyltransferase (SEQ ID NO: 37) or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 37.

Expression of the acetyltransferase may be achieved by introducing a nucleic acid, which may be codon-optimised for expression in the yeast cell, such as the nucleic acid as set forth in SEQ ID NO: 36, or a homologue thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to SEQ ID NO: 36.

Thus in some embodiments, the yeast cell expresses a heterologous desaturase as described herein above, a heterologous fatty acyl reductase as described herein above, and optionally any of the above described additional modifications, and also expresses an acetyltransferase capable of converting at least part of the produced E8,E10-dodecadien-1-ol into E8,E10-dodecadienyl acetate.

The yeast cells disclosed herein may thus be capable of producing E8,E10-dodecadienyl acetate with a titre of at least 0.2 mg/L. In some embodiments, the titre of E8,E10-dodecadienyl acetate is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

Methods for determining the titer are known in the art.

Production of E8,E10-dodecadienal

It may also be of interest to further convert at least part of the E8,E10-dodecadien-1-ol produced by the cell into E8,E10-dodecadienal. This can be done by chemical conversion or by further engineering the yeast cell.

In some embodiments, the yeast cell may be further engineered so that it is capable of converting at least part of the E8,E10-dodecadien-1-ol to E8,E10-dodecadienal. This can be done by engineering the yeast cell so that it further expresses an aldehyde-forming fatty acyl-CoA reductase (EC 1.2.1.50), an alcohol dehydrogenase (EC 1.1.1.2) and/or a fatty alcohol oxidase (EC 1.1.3.20) capable of converting at least part of the E8,E10-dodecadien-1-ol into E8,E10-dodecadienal. The yeast cell is in such embodiments capable of producing E8,E10-dodecadien-1-ol and E8,E10-dodecadienal.

A nucleic acid encoding an aldehyde-forming fatty acyl-CoA reductase (EC 1.2.1.50), an alcohol dehydrogenase (EC 1.1.1.2) and/or a fatty alcohol oxidase (EC 1.1.3.20) capable of converting at least part of the E8,E10-dodecadien-1-ol into E8,E10-dodecadienal, can thus be introduced in the yeast cell. The nucleic acid may be codon-optimised and may be expressed at high level. Thus in some embodiments, the yeast cell expresses a heterologous desaturase as described herein above, a heterologous fatty acyl reductase as described herein above, and optionally any of the above described additional modifications, and also expresses an aldehyde-forming fatty acyl-CoA reductase (EC 1.2.1.50), an alcohol dehydrogenase (EC 1.1.1.2) and/or a fatty alcohol oxidase (EC 1.1.3.20) capable of converting at least part of the E8,E10-dodecadien-1-ol into E8,E10-dodecadienal.

The yeast cells disclosed herein may thus be capable of producing E8,E10-dodecadienal with a titre of at least 0.2 mg/L. In some embodiments, the titre of E8,E10-dodecadienal is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

Methods for determining the titer are known in the art.

### Chain Shortening

In some embodiments, the yeast cell is further modified in order to increase availability of fatty acyl-CoAs of a given chain length by chain shortening. Without being bound by theory, such modifications are expected to increase availability of substrates having a desired carbon chain length, in particular having a carbon chain length of 12, whereby production of E8,E10-dodecadienyl coenzyme A and optionally E8,E10-dodecadien-1-ol, and optionally of E8,E10-dodecadienyl acetate and of E8,E10-dodecadienal, can be increased. This can achieved by reducing the activity of native acyl-CoA oxidases in a microbial production cell and by expressing specific acyl-CoA oxidases, desaturases, reductases, and acetyltransferases. Such modifications are described in detail in EP 19157910.1 (filed 19 Feb. 2019 by same applicant).

Thus in some embodiments, the yeast cell is any of the yeast cell described herein above, and further:
i) has one or more mutations resulting in reduced activity of one or more native acyl-CoA oxidases; and
ii) expresses at least one group of enzymes comprising at least one acyl-CoA oxidase capable of oxidising a fatty acyl-CoA, wherein the group of enzymes is capable of shortening a fatty acyl-CoA of a first carbon chain length X to a shortened fatty acyl-CoA having a second carbon chain length X′, wherein X′≤X-2.

The activity of the acyl-CoA oxidases normally present in the yeast cell, i.e. the native enzyme(s), is in such embodiments reduced or abolished by mutating the genes encoding said enzyme(s) in the cell. In order to direct carbon chain shortening to obtain fatty alcohols and derivatives thereof of a desired carbon chain length, one or more acyl-CoA oxidase is expressed in the yeast cell. These acyl-coA oxidases may be native to the yeast cell, or they may be derived from another organism. Genes encoding other enzymes required for oxidising a fatty acyl-CoA of a given chain length may also be introduced in the cell, if the cell does not express them already, or if increased activity or substrate specificity is desired. The acyl-CoA oxidase(s) thus expressed allow a fatty acyl-CoA to be oxidised and shortened to a fatty acyl-CoA having a shorter carbon chain length than the substrate. Thus in some embodiments, the reduced activity of the one or more native acyl-CoA oxidases is a reduced activity on acyl-CoAs having a carbon chain length smaller than X, such as smaller than X′.

The term acyl-CoA oxidase in the present disclosure refers to an enzyme such as an enzyme of EC number 1.3.3.6, capable of catalysing the following reaction:

acyl-CoA+O₂ [symbol] trans-2,3-dehydroacyl-CoA+H₂O₂

This enzyme belongs to the family of oxidoreductases, specifically those acting on the CH—CH group of donor with oxygen as acceptor. The systematic name of this enzyme class is acyl-CoA:oxygen 2-oxidoreductase. Other names use include fatty acyl-CoA oxidase, acyl coenzyme A oxidase, and fatty acyl-coenzyme A oxidase.

The yeast cell of the present disclosure may be engineered starting from a yeast cell which has one or more native acyl-CoA oxidases. The modified yeast cell disclosed herein preferably has reduced activity of said one or more native acyl-CoA oxidases; this can be achieved by using a yeast cell which has one or more mutations resulting in reduced activity of at least one of its native acyl-CoA oxidases. The native acyl-CoA oxidases may be peroxisomal, mitochondrial or cytosolic. In some embodiments, the one or more mutations results in reduced activity of all the native acyl-CoA oxidases. By reduced activity it is to be understood that the yeast cell due to said mutations has reduced ability to catalyse the above reaction, in particular to convert an acyl-CoA to the corresponding trans-2,3-dehydroacyl-CoA. In some embodiments, “reduced capability” means that the ability to catalyse said reaction is abolished completely or partially. In some embodiments, “reduced capability” means that the ability to catalyse the reaction is limited to a subgroup of the substrates which can be used for the reaction under normal circumstances, i.e. by using enzymes having normal capability.

The yeast cell of the present disclosure may express at least one group of enzymes comprising at least one acyl-CoA oxidase capable of oxidising a fatty acyl-CoA. The group of enzymes comprises, besides the at least one acyl-CoA oxidase, the other enzymes required for converting a fatty acyl-CoA of a given carbon chain length to a fatty acyl-CoA of a shorter carbon chain length. These other enzymes may preferably be native to the yeast cell; in such embodiments, only the introduction of a gene encoding an acyl-CoA oxidase is required for the yeast cell to express the group of enzymes.

In embodiments where the acyl-CoA oxidase is native to the yeast cell, said acyl-CoA oxidase may be modified as is known in the art, e.g. by the introduction of a promoter such as a constitutive or inducible promoter, or a promoter enabling overexpression of the acyl-CoA oxidase. The native acyl-CoA oxidase reintroduced in the first group of enzymes may be a mutated version with modified activity, such as modified substrate specificity and/or modified activity such as increased reaction efficiency.

In other embodiments, the acyl-CoA oxidase is derived from another organism. The acyl-CoA comprised in the first group of enzymes may be an acyl-CoA oxidase derived from a yeast, a fungus, an insect, a mammalian, a bird or a plant, such as the at least one acyl-CoA oxidase of the first group of enzymes is derived from a yeast, a fungus, an insect, a mammalian, a bird or a plant. For example, the acyl-CoA oxidase is derived from an organism of a genus selected from Yarrowia, Saccharomyces, Agrotis, Arabidopsis, Aspergillus, Cucurbita, Homo, Paenarthrobacter and Rattus, such as the at least one acyl-CoA oxidase of the first group of enzymes is derived from an organism of a genus selected from Yarrowia, Saccharomyces, Agrotis, Arabidopsis, Aspergillus, Cucurbita, Homo, Paenarthrobacter, and Rattus. In some embodiments, the at least one first group of enzymes comprises an acyl-CoA oxidase derived from Yarrowia lipolytica, Saccharomyces cerevisiae, Agrotis segetum, Arabidopsis thaliana, Aspergillus nidulans, Cucurbita maxima, Homo sapiens, Paenarthrobacter ureafaciens or Rattus norvegicus.

The acyl-CoA oxidase thus introduced in the yeast cell may be an acyl-CoA oxidase native to Yarrowia lipolytica, Agrotis segetum, Arabidopsis thaliana, Aspergillus nidulans, Cucurbita maxima, Homo sapiens, Paenarthrobacter ureafaciens or Rattus norvegicus. The yeast cell may be as described herein above.

The yeast cell of the present disclosure may thus express at least one group of enzymes comprising at least one acyl-CoA oxidase capable of oxidising a fatty acyl-CoA, wherein said at least one acyl-CoA oxidase is selected from the group Yli_POX1 (XP_504703), Yli_POX2 (XP_505264), Yli_POX3 (XP_503244), Yli_POX4 (XP_504475), Yli_POX5 (XP_502199), Yli_POX6 (XP_503632), Ase_POX (SEQ ID NO: 39), Ath_POX1 (SEQ ID NO: 41), Ath_POX2 (SEQ ID NO: 43), Ani_POX (SEQ ID NO: 45), Cma_POX (SEQ ID NO: 47), Hsa_POX1-2 (SEQ ID NO: 49), Pur_POX (SEQ ID NO: 51), Sc_POX1 (SEQ ID NO: 31) and Rno_POX2 (SEQ ID NO: 53), or a functional variant thereof having at least 60% homology or identity thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, expression of the at least one acyl-CoA oxidase is achieved by introducing a nucleic acid encoding said at least one acyl-CoA oxidase. For example, the yeast expresses YALI0_E32835g coding for Yli_POX1, YALI0_F10857g encoding Yli_POX2, YALI0_D24750g encoding Yli_POX3, YALI0_E27654g encoding Yli_POX4, YALI0_C23859g encoding Yli_POX5, YALI0_E06567g encoding Yli_POX6, SEQ ID NO: 38 encoding Ase_POX, SEQ ID NO: 40 encoding Ath_POX1, SEQ ID NO: 42 encoding Ath_POX2, SEQ ID NO: 44 encoding Ani_POX, SEQ ID NO: 46 encoding Cma_POX, SEQ ID NO: 48 encoding Hsa_POX, SEQ ID NO: 50 encoding Pur_POX, SEQ ID NO: 30 encoding Sc_POX1 or SEQ ID NO: 52 encoding Rno_POX2, or a homologue thereof having at least 60% homology or identity thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.

In some embodiments, X′=12.

Suitable acyl-CoA oxidases are described in detail in WO 2020/169389 (filed 10 Feb. 2020 by same applicant), in particular in the section “Acyl-CoA oxidase”.

In order to obtain E8,E10-dodecadienyl coenzyme A and optionally codlemone, in embodiments where chain shortening is taken advantage of, the yeast cell may thus, in addition to the at least one group of enzymes, also express an additional heterologous desaturase capable of introducing at least one double bond in E/Z conformations in a fatty acyl-CoA having a carbon chain length X or X′. X or X′ may be 8, 9, 10, 11, 12, 13, 14, 15, 16, 17, 18, 19, 20, 21 or 22 carbon atoms. In some embodiments, the desaturase is capable of introducing at least one double in E/Z conformations in a fatty acyl-CoA having a chain length of X′, wherein X′ is as defined above. Suitable desaturases are described in detail in WO 2020/169389 (filed 10 Feb. 2020 by same applicant), in particular in the section “Desaturase (FAD)”. In particular, desaturases capable of converting C14:CoA into Z11-C14:CoA and/or E11-C14:CoA are of interest. For example, the desaturases CroZ11 from Choristoneura rosaceana (SEQ ID NO: 63) or CpaE11 from Choristoneura parallela (SEQ ID NO: 65) or functional variants thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto, may be employed. The resulting Z11-C14:CoA and/or E11-C14:CoA may then be further chain shortened to give Z9-C12:CoA and/or E9-C12:CoA, which are then further desaturated by Cpo_CPRQ to give E8,E10-C12:CoA.

In such embodiments, the yeast cell thus expresses at least one desaturase as described herein above in the section “Desaturase”, for example Cpo_CPRQ or Gmo_CPRQ, preferably Cpo_CPRQ, a mutant or functional variant thereof, and further expresses an additional heterologous desaturase capable of introducing at least one double bond in E/Z conformations in a fatty acyl-CoA having a carbon chain length X or X′, where X and X′ are as described above. In particular, the desaturase may be capable of introducing at least one double bond in E/Z conformations in a fatty acyl-CoA having a carbon chain length of 14, which can then be shortened as described herein above to a fatty acyl-CoA of carbon chain length 12—this can then be further desaturated to E8,E10-C12:CoA, which can then be converted to E8,E10-dodecadien-1-ol by the action of the FAR as detailed herein above.

Methods for Production of E8, E10-dodecadienyl coenzyme A, E8, E10-dodecadien-1-ol, E8, E10-dodecadienyl acetate and/or E8, E10-dodecadienyl acetate

The yeast cells described herein above can be used in a method for producing E8,E10-dodecadienyl coenzyme A and optionally E8,E10-dodecadien-1-ol, which may be further converted into E8,E10-dodecadienyl acetate and/or E8,E10-dodecadienyl acetate.

Method for Production of E8,E10-dodecadien-1-ol

Herein is provided a method for producing E8,E10-dodecadienyl coenzyme A and optionally E8,E10-dodecadien-1-ol in a yeast cell, said method comprising the steps of providing a yeast cell and incubating said yeast cell in a medium, wherein the yeast cell expresses:
i) At least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
ii) Optionally at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol,
thereby producing E8,E10-dodecadienyl coenzyme A and optionally E8,E10-dodecadien-1-ol.

The yeast cell may be any of the yeast cells described herein above.

The present methods preferably allow production of E8,E10-dodecadienyl coenzyme A with a titre of at least 0.2 mg/L. In some embodiments, the titre of E8,E10-dodecadien-1-ol is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

The present methods allow production of E8,E10-dodecadien-1-ol with a titre of at least 0.2 mg/L. In some embodiments, the titre of E8,E10-dodecadien-1-ol is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

Methods for determining the titer are known in the art.

Method for Production of E8,E10-dodecadienyl acetate

In some embodiments, the method further comprises the step of converting at least part of the E8,E10-dodecadien-1-ol into E8,E10-dodecadienyl acetate by expression of an acetyltransferase or by chemical conversion. Accordingly, herein is disclosed a method for producing E8,E10-dodecadienyl acetate in a yeast cell, said method comprising the steps of:
a) providing a yeast cell and incubating said yeast cell in a medium, wherein the yeast cell expresses:
   i) At least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
   ii) at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol,
b) converting at least part of the E8,E10-dodecadien-1-ol into E8,E10-dodecadienyl acetate.

In some embodiments, the E8,E10-dodecadienyl acetate is obtained by engineering the yeast cell as described herein above in “Production of E8,E10-dodecadienyl acetate”.

In other embodiments, the conversion of E8,E10-dodecadien-1-ol produced by the cell into E8,E10-dodecadienyl acetate is performed chemically, as is known in the art. For example, the E8,E10-dodecadien-1-ol produced by the cell can be recovered, after which acetyl chloride is added to the E8,E10-dodecadien-1-ol, mixed and incubated, e.g. at room temperature, whereby at least part of the E8,E10-dodecadien-1-ol produced by the cell is converted into E8,E10-dodecadienyl acetate.

In other embodiments, the yeast cell produces E8,E10-dodecadienyl coenzyme A, which can be converted into a lipid such as a triacylglyceride or into a free fatty acid, recovering said lipid or free fatty acid, which in turn can be converted to E8,E10-dodecadien-1-ol. E8,E10-dodecadien-1-ol can then further be converted to E8,E10-dodecadien-1-ol in vitro, as described above. In such embodiments, conversion of E8,E10-dodecadien-1-ol produced by the cell into E8,E10-dodecadienyl acetate is performed chemically, as is known in the art.

The present methods may thus allow production of E8,E10-dodecadienyl acetate with a titre of at least 0.2 mg/L. In some embodiments, the titre of E8,E10-dodecadienyl acetate is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

Methods for determining the titer are known in the art.

Method for Production of E8,E10-dodecadienal

In some embodiments, the method further comprises the step of converting at least part of the E8,E10-dodecadien-1-ol into E8,E10-dodecadienal by further engineering the yeast cell or by chemical conversion. Accordingly, herein is disclosed a method for producing E8,E10-dodecadienal in a yeast cell, said method comprising the steps of:
a) providing a yeast cell and incubating said yeast cell in a medium, wherein the yeast cell expresses:
   i) At least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
   ii) At least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol,
b) converting at least part of the E8,E10-dodecadien-1-ol into E8,E10-dodecadienal.

In some embodiments, the E8,E10-dodecadienal is obtained by engineering the yeast cell as described herein above in “Production of E8,E10-dodecadienal”.

In other embodiments, the method comprises a step of converting at least part of the E8,E10-dodecadien-1-ol to E8,E10-dodecadienal by chemical conversion. The chemical conversion is based on the oxidation E8,E10-dodecadien-1-ol to E8,E10-dodecadienal. Methods for performing this conversion are known in the art. Preferred methods are environmentally friendly and minimize the amount of hazardous waste.

In other embodiments, the yeast cell produces E8,E10-dodecadienyl coenzyme A, which can be converted into a lipid such as a triacylglyceride or into a free fatty acid, which can then be recovered and converted to E8,E10-dodecadien-1-ol in vitro, as described above. In such embodiments, conversion of E8,E10-dodecadien-1-ol produced by the cell into E8,E10-dodecadienal is performed chemically, as is known in the art.

Thus in some embodiments, the chemical conversion may be metal free, avoiding toxic heavy metal based reagents such as manganese oxides, chromium oxides (Jones ox. PDC, PCC) or ruthenium compounds (TPAP, Ley-Griffith ox). In some embodiments, the conversion does not involve reactions involving activated dimethyl sulfoxide such as the Swern oxidation or the Pfitzner-Moffat type. Such reactions may involve the stereotypic formation of traces of intensively smelling organic sulfur compounds such as dimethyl sulfide which can be difficult to remove from the target product. In some embodiments, the method comprises a Dess-Martin reaction (Yadav et al., 2004, Meyer et al., 1994). In other embodiments, the chemical conversion comprises the oxidation with sodium hypochlorite under aqueous/organic two phase conditions (Okada et al., 2014; Tamura et al., 2012; Li et al., 2009). In some embodiments, the chemical oxidation can be performed with 1-chlorobenzotriazole in a medium of methylene chloride containing 25% pyridine (Ferrell and Yao, 1972).

Alternatively, the oxidation of E8,E10-dodecadien-1-ol to E8,E10-dodecadienal can be performed enzymatically by alcohol dehydrogenases. The skilled person will know how to carry out enzymatic oxidation. For example, enzymatic oxidation can be carried out by contacting purified enzymes, cell extracts or whole cells, with E8,E10-dodecadien-1-ol.

The methods disclosed herein thus in some embodiments allow production of E8,E10-dodecadienal with a titre of at least 0.2 mg/L. In some embodiments, the titre of E8,E10-dodecadienal is at least 0.25 mg/L, such as at least 0.3 mg/L, such as at least 0.4 mg/L, such as at least 0.5 mg/L, such as at least 0.75 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.

Methods for determining the titer are known in the art.

### Recovery

In some embodiments, the method further comprises a step of recovering the obtained products.

In some embodiments, the method is for production of E8,E10-dodecadien-1-ol and thus further comprises a step of recovering the produced E8,E10-dodecadien-1-ol. In other embodiments, the method is for production of E8,E10-dodecadienyl acetate and thus further comprises a step of recovering the produced E8,E10-dodecadienyl acetate. In other embodiments, the method is for production of E8,E10-dodecadienal and thus further comprises a step of recovering the produced E8,E10-dodecadienal.

Methods for recovering the products obtained by the present methods are known in the art and may comprise an extraction with a hydrophobic solvent such as decane, hexane or a vegetable oil.

Alternatively, the methods described in application PCT/EP2020/076351 (filed on 22 Sep. 2020 by same applicant) can also be used to recover the desired products. For example, said methods can be used to recover lipids such as triacylglycerides, or fatty acids, obtained from the conversion of E8,E10-dodecadienyl coenzyme A, or to recover the produced E8,E10-dodecadien-1-ol, the produced E8,E10-dodecadienyl acetate and/or the produced E8,E10-dodecadienal. Said methods take advantage of the addition of an extractant in the culture medium in an amount equal to or greater than its cloud concentration measured in an aqueous solution such as in the culture medium at the cultivation temperature, which greatly facilitates recovery of hydrophobic compounds such as fatty alcohols, fatty alcohol acetates and fatty aldehydes. Such methods can thus advantageously be used to facilitate recovery of the lipids such as triacylglycerides, or fatty acids, obtained from the conversion of E8,E10-dodecadienyl coenzyme A, of the E8,E10-dodecadien-1-ol, of the E8,E10-dodecadienyl acetate and of the E8,E10-dodecadienal produced by the present methods. In addition, the addition of an extractant in the culture medium was also found to generally increase titer of hydrophobic compounds produced by the cell, and to increase secretion of the produced hydrophobic compounds from the cell.

Thus in some embodiments, the medium used in the present methods comprises an extractant in an amount equal to or greater than its cloud concentration measured in an aqueous solution such as the culture medium at the cultivation temperature, wherein the extractant is a non-ionic surfactant, preferably a non-ionic ethoxylated surfactant such as an antifoaming agent, preferably a polyethoxylated surfactant selected from: a polyethylene polypropylene glycol, a mixture of polyether dispersions, an antifoaming agent comprising polyethylene glycol monostearate such as simethicone, fatty alcohol alkoxylates, polyethoxylated surfactants and ethoxylated and propoxylated C₁₆-C₁₈ alcohol-based antifoaming agents and combinations thereof.

The cloud concentration in an aqueous solution is determined at a given temperature, preferably at room temperature or at the temperature at which the fermentation is to be performed, for example 30° C., or at room temperature. The term “extractant” as used herein refers to a non-ionic surfactant, in particular an antifoaming agent, which facilitates recovery of hydrophobic compounds produced in a fermentation. For example, the non-ionic surfactant is a non-ionic ethoxylated surfactant, for example a polyethoxylated surfactant selected from: a polyethylene polypropylene glycol, a mixture of polyether dispersions, an antifoaming agent comprising polyethylene glycol monostearate such as simethicone, fatty alcohol alkoxylates, polyethoxylated surfactants and ethoxylated and propoxylated C₁₆-C₁₈ alcohol-based antifoaming agents and combinations thereof. Example 7 of PCT/EP2020/076351 describes how to determine the cloud concentration of a surfactant.

Non-ionic surfactants which are suitable extractants and suitable amounts of said non-ionic surfactants are described in detail in application PCT/EP2020/076351 (filed on 22 Sep. 2020 by same applicant), in particular in the section entitled “Non-ionic ethoxylated surfactant”.

In some embodiments, the culture medium used in the present methods thus comprises a non-ionic surfactant which is an ethoxylated and propoxylated C₁₆-C₁₈ alcohol-based antifoaming agent, such as C₁₆-C₁₈ alkyl alcohol ethoxylate propoxylate (CAS number 68002-96-0), and the culture medium comprises at least 1% vol/vol of C₁₆-C₁₈ alkyl alcohol ethoxylate propoxylate, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol C₁₆-C₁₈ alkyl alcohol ethoxylate propoxylate, or more.

In other embodiments, the culture medium used in the present methods comprises a non-ionic surfactant which is a polyethylene polypropylene glycol, for example Kollliphor® P407 (CAS number 9003-11-6), and the culture medium comprises at least 10% vol/vol of polyethylene polypropylene glycol such as Kolliphor® P407, such as at least 11% vol/vol, such as at least 12% vol/vol, such as at least 13% vol/vol, such as at least 14% vol/vol, such as at least 15% vol/vol, such as at least 16% vol/vol, such as at least 17% vol/vol, such as at least 18% vol/vol, such as at least 19% vol/vol, such as at least 20% vol/vol, such as at least 25% vol/vol, such as at least 30% vol/vol, such as at least 35% vol/vol of polyethylene polypropylene glycol such as Kolliphor® P407, or more.

In other embodiments, the culture medium used in the present methods comprises a non-ionic surfactant which is a mixture of polyether dispersions, such as antifoam 204, and the culture medium comprises at least 1% vol/vol of a mixture of polyether dispersions such as antifoam 204, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol of a mixture of polyether dispersions such as antifoam 204, or more.

In other embodiments, the culture medium used in the present methods comprises a non-ionic surfactant which comprises polyethylene glycol monostearate such as simethicone, and the culture medium comprises at least 1% vol/vol of polyethylene glycol monostearate or simethicone, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol polyethylene glycol monostearate or simethicone, or more.

In other embodiments, the culture medium used in the present methods comprises a non-ionic surfactant which is a fatty alcohol alkoxylate, and the culture medium comprises at least 1% vol/vol of fatty alcohol alkoxylate, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol fatty alcohol alkoxylate or more. Suitable fatty alcohol alkoxylates include Plurafac® LF300 (CAS number 196823-11-7), Plurafac® LF1300 (68002-96-0), Plurafac® SLF180 (CAS number 196823-11-7), Dehypon® 2574 (CAS number 68154-97-2), and Imbentin SG/251 (CAS number 68002-96-0), preferably Plurafac® LF300 or Dehypon® 2574.

In other embodiments, the culture medium used in the present methods comprises a non-ionic surfactant which is Agnique BP420 (CAS number 68002-96-0), and the culture medium comprises at least 1% vol/vol of Agnique BP420, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol Agnique BP420 or more.

In some embodiments, the culture medium comprises the extractant in an amount greater than its cloud concentration by at least 50%, such as at least 100%, such as at least 150%, such as at least 200%, such as at least 250%, such as at least 300%, such as at least 350%, such as at least 400%, such as at least 500%, such as at least 750%, such as at least 1000%, or more, and/or wherein the culture medium comprises the extractant in an amount at least 2-fold its cloud concentration, such as at least 3-fold its cloud concentration, such as at least 4-fold its cloud concentration, such as at least 5-fold its cloud concentration, such as at least 6-fold its cloud concentration, such as at least 7-fold its cloud concentration, such as at least 8-fold its cloud concentration, such as at least 9-fold its cloud concentration, such as at least 10-fold its cloud concentration, such as at least 12.5-fold its cloud concentration, such as at least 15-fold its cloud concentration, such as at least 17.5-fold its cloud concentration, such as at least 20-fold its cloud concentration, such as at least 25-fold its cloud concentration, such as at least 30-fold its cloud concentration.

The addition of an extractant, i.e. a non-ionic surfactant such as a polyethoxylated surfactant, for example any of the non-ionic surfactants, antifoaming agents or polyethoxylated surfactants described herein, results in the generation of an emulsion in the fermentation broth, where the hydrophobic compound produced by the microorganism, i.e. E8,E10-dodecadienyl coenzyme A (or the lipid or free fatty acid obtained by converting E8,E10-dodecadienyl coenzyme A), the E8,E10-dodecadien-1-ol, the E8,E10-dodecadienyl acetate and/or the E8,E10-dodecadienal, is present in the emulsion. In embodiments where the present methods are performed with a culture medium comprising an extractant, the methods thus may also comprise a step of breaking the emulsion to recover a product phase comprising the extractant and the hydrophobic compound. Once the emulsion is broken, the fermentation broth is separated in three phases: a water phase, comprising mainly water and aqueous compounds, a phase comprising cells and cellular debris, and a product phase mainly comprising the extractant and the E8,E10-dodecadienyl coenzyme A (or the lipid or free fatty acid obtained by converting E8,E10-dodecadienyl coenzyme A), the E8,E10-dodecadien-1-ol, the E8,E10-dodecadienyl acetate and/or the E8,E10-dodecadienal. Thus a composition is obtained consisting of three phases. This is described in detail in application PCT/EP2020/076351 (filed on 22 Sep. 2020 by same applicant), in particular in the section entitled “Product phase comprising the hydrophobic compound”.

In some embodiments, most of the E8,E10-dodecadienyl coenzyme A (or the lipid or free fatty acid obtained by converting E8,E10-dodecadienyl coenzyme A), and optionally most of the E8,E10-dodecadien-1-ol, the E8,E10-dodecadienyl acetate and/or the E8,E10-dodecadienal is present in the product phase. For example, at least 50% of the E8,E10-dodecadienyl coenzyme A (or the lipid or free fatty acid obtained by converting E8,E10-dodecadienyl coenzyme A), and optionally of the E8,E10-dodecadien-1-ol, the E8,E10-dodecadienyl acetate and/or the E8,E10-dodecadienal is present in the product phase, such as at least 55%, such as at least 60%, such as at least 65%, such as at least 70%, such as at least 75%, such as at least 80%, such as at least 85%, such as at least 90%, such as at least 95%, such as at least 96%, such as at least 97%, such as at least 98%, such as at least 99%, such as 100% of the E8,E10-dodecadienyl coenzyme A (or the lipid or free fatty acid obtained by converting E8,E10-dodecadienyl coenzyme A), and optionally of the E8,E10-dodecadien-1-ol, the E8,E10-dodecadienyl acetate and/or the E8,E10-dodecadienal is present in the product phase. In some embodiments, the product phase comprises at least 50% of the E8,E10-dodecadienyl coenzyme A (or the lipid or free fatty acid obtained by converting E8,E10-dodecadienyl coenzyme A), and optionally the E8,E10-dodecadien-1-ol, the E8,E10-dodecadienyl acetate and/or the E8,E10-dodecadienal initially present in the fermentation broth, such as at least 55%, such as at least 60%, such as at least 65%, such as at least 70%, such as at least 75%, such as at least 80%, such as at least 85%, such as at least 90%, such as at least 95%, such as at least 96%, such as at least 97%, such as at least 98%, such as at least 99%, such as 100% of the E8,E10-dodecadienyl coenzyme A (or the lipid or free fatty acid obtained by converting E8,E10-dodecadienyl coenzyme A), and optionally the E8,E10-dodecadien-1-ol, the E8,E10-dodecadienyl acetate and/or the E8,E10-dodecadienal initially present in the fermentation broth.

The step of breaking the emulsion may be performed as is known in the art, for example by submitting the emulsion to a step of phase separation, for example by centrifugation. Following the step of breaking the emulsion, the product phase comprising the extractant and the lipid or free fatty acid obtained by converting E8,E10-dodecadienyl coenzyme A, and optionally the E8,E10-dodecadien-1-ol, the E8,E10-dodecadienyl acetate and/or the E8,E10-dodecadienal may be recovered from the composition. The method may in such embodiments further comprise the step of separating the lipid or free fatty acid obtained by converting E8,E10-dodecadienyl coenzyme A, and optionally the E8,E10-dodecadien-1-ol, the E8,E10-dodecadienyl acetate and/or the E8,E10-dodecadienal from the extractant. This can be performed by methods known in the art, such as by distillation, for example distillation under reduced pressure, or by column purification, or any other suitable method. The extractant may be recirculated to the fermentor or bioreactor.

### Nucleic Acid Constructs

Also provided is a nucleic acid construct for modifying a yeast cell, said construct comprising:
i) At least one first polynucleotide encoding at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
ii) Optionally a second polynucleotide encoding at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol.

The nucleic acid constructs can be used to obtain a yeast cell as described herein, i.e. a yeast cell capable of producing E8,E10-dodecadienyl coenzyme A and optionally E8,E10-dodecadien-1-01. The term “nucleic acid construct” may refer here to a single physical entity, i.e. a single molecule, for example a vector or a plasmid in which the first polynucleotide and optionally the second polynucleotide are comprised, or it may refer to a plurality of nucleic acid molecules, e.g. the first polynucleotide is comprised within one plasmid or vector and the second polynucleotide is comprised within another plasmid or vector.

The nucleic acid construct may further comprise one or more of:
iii) a polynucleotide encoding a heterologous cytochrome b5, such as the polynucleotide as set forth in SEQ ID NO: 3 or a homologue thereof having at least 60% homology or identity thereto;
iv) a polynucleotide encoding a heterologous cytochrome b5 reductase, such as the polynucleotide as set forth in SEQ ID NO: 23 or a homologue thereof having at least 60% homology or identity thereto;
v) a polynucleotide encoding a hemoglobin, such as the polynucleotide as set forth in SEQ ID NO: 5 or a homologue thereof having at least 60% homology or identity thereto; and/or
vi) a polynucleotide encoding a thioesterase, such as the polynucleotide as set forth in SEQ ID NO: 25 or SEQ ID NO: 34 or a homologue thereof having at least 60% homology or identity thereto.

The polynucleotides may comprise several copies of any of the above genes, and may be codon-optimised for proper expression in the yeast cell in which they are to be introduced.

In some embodiments, the at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA) is Cpo_CPRQ (SEQ ID NO: 2) or a functional variant thereof having at least 60% homology or identity thereto, as described above. In such embodiments, the first polynucleotide comprises SEQ ID NO: 1 or a homologue thereof having at least 60% homology or identity thereto, as described herein above.

In some embodiments, the at least one heterologous desaturase is a mutant Cpo_CPRQ such as a Cpo_CPRQ mutant having a mutation at position 85, or a functional variant thereof having at least 60% homology or identity thereto. In some embodiments, the mutation is an S85A mutation. In some embodiments, the desaturase is a mutant Cpo_CPRQ such as a Cpo_CPRQ mutant having a mutation at position 82. In some embodiments, the mutation is an S82A mutation, or a functional variant thereof having at least 60% homology or identity thereto.

In other embodiments, the at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA) is Gmo_CPRQ (SEQ ID NO: 77) or a functional variant thereof having at least 60% homology or identity thereto, as described above. In such embodiments, the first polynucleotide comprises SEQ ID NO: 78 or a homologue thereof having at least 60% homology or identity thereto, as described herein above.

In some embodiments, the yeast cell expresses several desaturases capable of introducing one or more double bonds in a fatty acyl-CoA of carbon chain length 12. In such embodiments, preferably at least one of the several desaturases is Cpo_CPRQ, Gmo_CPRQ, a mutant thereof or a functional variant thereof as detailed herein, and the first polynucleotide comprises or consists of SEQ ID NO: 1 or a homologue thereof having at least 60% homology or identity thereto. The nucleic acid construct may in such embodiments comprise further first polynucleotides, each encoding a desaturase as described herein above. For example, the nucleic acid construct comprises a first polynucleotide encoding Cpo_CPRQ, Gmo_CPRQ or homologues thereof, and further comprises a further first polynucleotide encoding a further desaturase, preferably Cpo_NPVE (SEQ ID NO: 67) or Cpo_SPTQ (SEQ ID NO: 69), or a functional variant thereof having at least 60% homology or identity thereto. Thus in some embodiments, the further first polynucleotide comprises SEQ ID NO: 66 or SEQ ID NO: 68, or homologues thereof having at least 60% homology or identity thereto.

The nucleic acid construct may also comprise a second polynucleotide encoding a FAR. The FAR is preferably be an insect FAR, such as a FAR native to an insect of the genus Agrotis, Heliothis, Helicoverpa or Cydia. For example, the FAR is native to Agrotis segetum, Agrotis ipsilon, Heliothis subflexa, Helicoverpa assulta, Helicoverpa virescens or Cydia pomonella.

In some embodiments, the FAR is Ase_FAR (SEQ ID NO: 10), i.e. the FAR naturally occurring in Agrotis segetum. In some embodiments, the heterologous FAR is a functional variant of Ase_FAR, which retains the capability of converting E8,E10-C12:CoA to E8,E10-dodecadien-1-ol. For example, the functional variant has at least 65% homology or identity thereto. In some embodiments the FAR is a mutant Ase_FAR, such as a mutant having a mutation at position 198 or 413. In some embodiments the Ase_FAR mutant is a T198A mutant. In other embodiments, the Ase_FAR mutant is an S413A mutant. In such embodiments, the second polynucleotide comprises or consists of SEQ ID NO: 9 or a homologue thereof having at least 60% homology or identity thereto.

In other embodiments, the FAR is Aip_FAR (SEQ ID NO: 61), i.e. the FAR naturally occurring in Agrotis ipsilon. In some embodiments, the heterologous FAR is a functional variant of Aip_FAR, which retains the capability of converting E8,E10-C12:CoA to E8,E10-dodecadien-1-ol. In such embodiments, the second polynucleotide comprises or consists of SEQ ID NO: 60 or a homologue thereof having at least 60% homology or identity thereto.

In other embodiments, the FAR is Hs_FAR (SEQ ID NO: 71), i.e. the FAR naturally occurring in Heliothis subflexa. In some embodiments, the heterologous FAR is a functional variant of Hs_FAR, which retains the capability of converting E8,E10-C12:CoA to E8,E10-dodecadien-1-ol. In such embodiments, the second polynucleotide comprises or consists of SEQ ID NO: 70 or a homologue thereof having at least 60% homology or identity thereto.

In other embodiments, the FAR is Has_FAR (SEQ ID NO: 73), i.e. the FAR naturally occurring in Helicoverpa assulta. In some embodiments, the heterologous FAR is a functional variant of Has_FAR, which retains the capability of converting E8,E10-C12:CoA to E8,E10-dodecadien-1-ol. In such embodiments, the second polynucleotide comprises or consists of SEQ ID NO: 72 or a homologue thereof having at least 60% homology or identity thereto.

In other embodiments, the FAR is Hv_FAR (SEQ ID NO: 75), i.e. the FAR naturally occurring in Helicoverpa virescens. In some embodiments, the heterologous FAR is a functional variant of Hv_FAR, which retains the capability of converting E8,E10-C12:CoA to E8,E10-dodecadien-1-ol. In such embodiments, the second polynucleotide comprises or consists of SEQ ID NO: 74 or a homologue thereof having at least 60% homology or identity thereto.

In other embodiments, the FAR is Har_FAR (SEQ ID NO: 12), i.e. the FAR naturally occurring in Helicoverpa armigera. In some embodiments, the heterologous FAR is a functional variant of Har_FAR, which retains the capability of converting E8,E10-C12:CoA to E8,E10-dodecadien-1-ol. In such embodiments, the second polynucleotide comprises or consists of SEQ ID NO: 13 or a homologue thereof having at least 60% homology or identity thereto.

In other embodiments, the FAR is a Cydia pomonella FAR, for example Cpo_FAR or a functional variant thereof, which retains the capability of converting E8,E10-C12:CoA to E8,E10-dodecadien-1-ol. In such embodiments, the second polynucleotide comprises or consists of SEQ ID NO: 75 or a homologue thereof having at least 60% homology or identity thereto.

In embodiments where it is desirable to express several FARs, the second polynucleotide may be a plurality of second polynucleotides each encoding one FAR.

In some embodiments, the nucleic acid construct comprises at least one further polynucleotide, which may be a different nucleic acid molecule than the first and/or second polynucleotides, or which may be part of the same nucleic acid molecule as the first and/or second polynucleotides.

The further polynucleotide in some embodiments encodes a heterologous cytochrome b5, such as the cytochrome b5 as set forth in SEQ ID NO: 3 or a homologue thereof having at least 60% homology or identity thereto. In some embodiments, the cytochrome b5 is a cytochrome b5 which is native to a Lepidoptera species. In particular embodiments, the cytochrome b5 is a cytochrome b5 from a Helicoverpa species, preferably a cytochrome b5 from Helicoverpa armigera, such as set forth in SEQ ID NO: 4, or a functional variant thereof having at least 60% homology or identity thereto. In such embodiments, the further polynucleotide comprises or consists of SEQ ID NO: 3 or a homologue thereof having at least 60% homology thereto.

The further polynucleotide in some embodiments encodes a heterologous cytochrome b5 reductase, such as the cytochrome b5 reductase as set forth in SEQ ID NO: 24 or a homologue thereof having at least 60% homology or identity thereto. In some embodiments, the cytochrome b5 reductase is a cytochrome b5 reductase which is native to a Helicoverpa species. In particular embodiments, the cytochrome b5 reductase is a cytochrome b5 reductase from a Helicoverpa species, preferably a cytochrome b5 reductase from Helicoverpa armigera, such as set forth in SEQ ID NO: 24, or a functional variant thereof having at least 60% homology or identity thereto. In such embodiments, the further polynucleotide comprises or consists of SEQ ID NO: 23 or a homologue thereof having at least 60% homology thereto.

The further polynucleotide in some embodiments encodes a heterologous hemoglobin, such as the hemoglobin as set forth in SEQ ID NO: 6 or a homologue thereof having at least 60% homology or identity thereto. In some embodiments, the hemoglobin is an hemoglobin which is native to a Vitreoscilla species. In particular embodiments, the hemoglobin is a hemoglobin from Vitreoscilla stercoraria, such as set forth in SEQ ID NO: 6, or a functional variant thereof having at least 60% homology or identity thereto. In such embodiments, the further polynucleotide comprises or consists of SEQ ID NO: 5 or a homologue thereof having at least 60% homology thereto.

The further polynucleotide in some embodiments encodes a thioesterase, such as the thioesterase as set forth in SEQ ID NO: 6 or a homologue thereof having at least 60% homology or identity thereto. In some embodiments, the thioesterase is native to a Cuphea species, to a Cinnamomum species or to an Escherichia species. In particular embodiments, the thioesterase is a hemoglobin from Cuphea palustris, Cuphea hookeriana, Cinnamomum camphora, or Escherichia coli, such as set forth in SEQ ID NO: 33, SEQ ID NO: 57, SEQ ID NO: 35 or SEQ ID NO: 26, respectively, or a functional variant thereof having at least 60% homology or identity thereto. In such embodiments, the further polynucleotide comprises or consists of SEQ ID NO: 34, SEQ ID NO: 56, SEQ ID NO: 34 or SEQ ID NO: 25 a homologue thereof having at least 60% homology thereto.

In some embodiments, the nucleic acid construct comprises a first polynucleotide as described above, and optionally a second polynucleotide as described above, and further expresses one or more further polynucleotide as described above. In some embodiments, the nucleic acid construct thus comprises the first polynucleotide and optionally the second polynucleotide, and further comprises one of:
at least one further polynucleotide encoding a heterologous cytochrome b5; or
at least one further polynucleotide encoding a heterologous cytochrome b5 reductase; or
at least one further polynucleotide encoding a hemoglobin; or
at least one further polynucleotide encoding a thioesterase.

In other embodiments, the nucleic acid construct comprises the first polynucleotide and optionally the second polynucleotide, and further comprises:
at least one further polynucleotide encoding a heterologous cytochrome b5; and
at least one further polynucleotide encoding a heterologous cytochrome b5 reductase; or
at least one further polynucleotide encoding a heterologous cytochrome b5; and
at least one further polynucleotide encoding a hemoglobin;

or
at least one further polynucleotide encoding a heterologous cytochrome b5; and
at least one further polynucleotide encoding a thioesterase;

or:
at least one further polynucleotide encoding a heterologous cytochrome b5 reductase; and
at least one further polynucleotide encoding a hemoglobin;

or:
at least one further polynucleotide encoding a heterologous cytochrome b5 reductase; and
at least one further polynucleotide encoding a thioesterase:

or:
at least one further polynucleotide encoding a hemoglobin; and
at least one further polynucleotide encoding a thioesterase.

In other embodiments, the nucleic acid construct comprises the first polynucleotide and optionally the second polynucleotide, and further comprises:
at least one further polynucleotide encoding a heterologous cytochrome b5; and
at least one further polynucleotide encoding a heterologous cytochrome b5 reductase; and
at least one further polynucleotide encoding a hemoglobin; or
at least one further polynucleotide encoding a heterologous cytochrome b5; and
at least one further polynucleotide encoding a heterologous cytochrome b5 reductase; and
at least one further polynucleotide encoding a thioesterase;

or
at least one further polynucleotide encoding a heterologous cytochrome b5 reductase; and
at least one further polynucleotide encoding a hemoglobin; and
at least one further polynucleotide encoding a thioesterase;

or
at least one further polynucleotide encoding a heterologous cytochrome b5; and
at least one further polynucleotide encoding a hemoglobin; and
at least one further polynucleotide encoding a thioesterase.

In some embodiments, the nucleic acid construct comprises the first polynucleotide and optionally the second polynucleotide, and further comprises all of:
at least one further polynucleotide encoding a heterologous cytochrome b5; and
at least one further polynucleotide encoding a heterologous cytochrome b5 reductase; and
at least one further polynucleotide encoding a hemoglobin; and
at least one further polynucleotide encoding a thioesterase.

The nucleic acid construct may further comprise additional polynucleotides for introducing in the yeast cell any of the additional modifications described herein above, in particular polynucleotides which upon introduction in the yeast cell result in modified activity of the native fatty aldehyde dehydrogenase(s), fatty alcohol oxidase(s), peroxisome biogenesis factor and/or fatty acyl synthase(s) is modified; preferably the activity is reduced or abolished.

The nucleic acid constructs may comprise additional elements required for or facilitating expression of the polynucleotides comprised therein, such as promoters, for example inducible, repressible or constitutive promoters, located upstream of the coding sequences comprised in the polynucleotides, as is known in the art.

### Formulation as a Pheromone Composition

In some embodiments, the present methods further comprise a step of formulating the E8,E10-dodecadien-1-ol, E8,E10-dodecadienyl acetate and/or E8,E10-dodecadienal produced by the yeast cell as a pheromone composition, as is known in the art.

E8, E10-dodecadienyl coenzyme A, E8, E10-dodecadien-1-ol, E8, E10-dodecadienyl acetate and/or E8, E10-dodecadienal Obtainable by the Present Methods

The present disclosure also provides E8,E10-dodecadienyl coenzyme A (or the lipid or free fatty acid obtained by converting E8,E10-dodecadienyl coenzyme A), E8,E10-dodecadien-1-ol, E8,E10-dodecadienyl acetate and/or E8,E10-dodecadienal obtainable by the present methods. When expressing insect desaturases and/or reductases in a yeast cell, the resulting mix of products, e.g. E8,E10-dodecadienyl coenzyme A and/or fatty alcohols comprising E8,E10-dodecadien-1-ol and produced by the cell will typically have a similar composition as the one produced in the pheromone glands of the insects. This allows for the production of pheromone mixes suitable for various insects instead of producing individual pheromone components in separate processes that then need to be mixed in appropriate proportions. Nevertheless, the resulting mixture of products, e.g. E8,E10-dodecadienyl coenzyme A and/or fatty alcohols may contain by-products characteristic of biological production.

Thus in some embodiments where production of E8,E10-dodecadienyl coenzyme A is performed, the produced fatty acyl-CoAs comprise at least 1%, such as at least 2%, such as at least 3%, such as at least 4%, such as at least 5%, such as at least 10%, such as at least 15%, such as at least 20% of a desaturated fatty acyl-CoA having a desaturation at another position than the desired fatty acyl-CoA and/or at least 1%, such as at least 2%, such as at least 3%, such as at least 4%, such as at least 5%, such as at least 10%, such as at least 15%, such as at least 20% of the corresponding saturated fatty acyl-CoA.

In embodiments where production of E8,E10-dodecadien-1-ol is performed, the produced fatty alcohols comprise at least 1%, such as at least 2%, such as at least 3%, such as at least 4%, such as at least 5%, such as at least 10%, such as at least 15%, such as at least 20% of a desaturated fatty alcohol having a desaturation at another position than the desired fatty alcohol and/or at least 1%, such as at least 2%, such as at least 3%, such as at least 4%, such as at least 5%, such as at least 10%, such as at least 15%, such as at least 20% of the corresponding saturated fatty alcohol. If the mix of fatty alcohols recovered from the fermentation broth is chemically oxidized into aldehydes or acetylated into acetates, then corresponding mixes of aldehydes and acetates are produced.

In some embodiments, the present methods are for production of E8,E10-dodecadienal. In some embodiments, the present yeast cells and methods result in production of a mixture of fatty aldehydes which comprises E8,E10-dodecadienal, but also comprises odd-chain fatty aldehydes. The term “odd-chain” fatty aldehydes refers to fatty aldehydes having a carbon chain length which is an odd number of carbon atoms, such as 1, 3, 5, 7, 9, 11, 13, 15, 17, 19, 21, or 23 carbon atoms. The term “even-chain” fatty aldehydes refers to fatty aldehydes having a carbon chain length which is an even number of carbon atoms, such as 8, 10, 12, 14, 16, 18, 20 or 22 carbon atoms.

In some embodiments, the present methods are for production of E8,E10-dodecadienyl acetate. In some embodiments, the present yeast cells and methods result in production of a mixture of fatty alcohol acetates which comprises E8,E10-dodecadienyl acetate, but also comprises odd-chain fatty alcohol acetates. The term “odd-chain” fatty alcohol acetates refers to fatty alcohol acetates having a carbon chain length which is an odd number of carbon atoms, such as 1, 3, 5, 7, 9, 11, 13, 15, 17, 19, 21, or 23 carbon atoms. The term “even-chain” fatty alcohol acetates refers to fatty alcohol acetates having a carbon chain length which is an even number of carbon atoms, such as 8, 10, 12, 14, 16, 18, 20 or 22 carbon atoms.

Pheromone composition The E8,E10-dodecadien-1-ol, E8,E10-dodecadienyl acetate and/or E8,E10-dodecadienal produced by the yeast cell may be formulated as a pheromone composition, as is known in the art. Such pheromone compositions may be used as integrated pest management products, which can be used in a method of monitoring the presence of pest or in a method of disrupting the mating of pest.

Pheromone compositions as disclosed herein may be used as biopesticides. Such compositions can be sprayed or dispensed on a culture, in a field or in an orchard. They can also, as is known in the art, be soaked e.g. onto a rubber septa, or mixed with other components. This can result in mating disruption, thereby preventing pest reproduction, or it can be used in combination with a trapping device to entrap the pests. Non-limiting examples of pests against which the present pheromone compositions can be used are: cotton bollworm (Helicoverpa armigera), striped stemborer (Chilo suppressalis), diamond back moth (Plutella xylostella), cabbage moth (Mamestra brassicae), large cabbage-heart caterpillar (Crocidolomia binotalis), European corn stalk borer (Sesamia nonagrioides), currant clearwing (Synanthedon tipuliformis) and artichoke plume moth (Platyptilia carduidactylal). Accordingly, use of the present compositions on a culture can lead to increased crop yield, with substantially no environmental impact.

The relative amounts of the different compounds in the present pheromone compositions may vary depending on the nature of the crop and/or of the pest to be controlled; geographical variations may also exist. Determining the optimal relative amounts may thus require routine optimisation.

In some embodiments of the present disclosure, the pheromone composition may further comprise one or more additional compounds such as a liquid or solid carrier or substrate. For example, suitable carriers or substrate include vegetable oils, refined mineral oils or fractions thereof, rubbers, plastics, silica, diatomaceous earth, wax matrix and cellulose powder.

The pheromone composition may be formulated as is known in the art. For example, it may be under the form of a solution, a gel, a powder. The pheromone composition may be formulated so that it can be easily dispensed, as is known in the art.

### Kit

Provided herein is a kit of parts for performing the present methods. The kit of parts may comprise a yeast cell “ready to use” as described herein. In one embodiment, the yeast cell is a Yarrowia cell, such as a Yarrowia lipolytica cell, or a Saccharomyces cell such as a Saccharomyces cerevisiae cell.

Alternatively, the kit of parts may also comprise nucleic acid constructs encoding the activities of interest to be introduced in the yeast cell. The nucleic acid construct may be provided as a plurality of nucleic acid constructs, such as a plurality of vectors, wherein each vector encodes one or several of the desired activities. Useful nucleic acid constructs have been described above.

The kit of parts may also comprise nucleic acid constructs useful for introducing mutations resulting in partial or total loss of function such as any of the mutations described herein above.

The kit of parts may optionally comprise the yeast cell to be modified.

In some embodiments, the kit of parts comprises all of the above.

### Method for Monitoring the Presence of Pest or Disrupting the Mating of Pest

The E8,E10-dodecadien-1-ol and optionally E8,E10-dodecadienyl acetate and/or E8,E10-dodecadienal produced by the yeast cells and methods disclosed herein can be used in a method for monitoring the presence of pest or disrupting the mating of pest.

Accordingly, herein is also provided a method of monitoring the presence of pest or disrupting the mating of pest, said method comprising the steps of:
i) Producing E8,E10-dodecadien-1-ol and optionally E8,E10-dodecadienyl acetate and/or E8,E10-dodecadienal by the methods described herein;
ii) Formulating said E8,E10-dodecadien-1-ol and optionally said E8,E10-dodecadienyl acetate and/or said E8,E10-dodecadienal as a pheromone composition; and
iii) Employing said pheromone composition as an integrated pest management composition.

Any of the yeast cells and methods described herein above can be used in such methods.

### EXAMPLES

### Example 1: Construction of Biobricks

All heterologous genes were synthesized by GeneArt (Life Technologies) in codon-optimized versions for Yarrowia lipolytica. All the genes were amplified by PCR using Phusion U Hot Start DNA Polymerase (ThermoFisher) to obtain the fragments for cloning into yeast expression vectors. The primers are listed in Table 1 and the resulting DNA fragments (BioBricks) are listed in Table 2. The PCR products were separated on a 1%-agarose gel containing Midori Green Advance (Nippon Genetics Europe GmbH). PCR products of the correct size were excised from the gel and purified using the Nucleospin Gel and PCR Clean-up kit (Macherey-Nagel)

TABLE 1
primers

| | | NCBI accession | Hybridises |
|---|---|---|---|
| Primer name | Template | number | at positions |
| PR141 | pCfB4778 | — | 1806 . . . 1827 |
| PR142 | pCfB4778 | — | 3986 . . . 4010 |
| PR1852 | CEN.PK102.5B | NC_001139 | 920060 . . . 920080 |
| PR1853 | CEN.PK102.5B | NC_001139 | 920060 . . . 920080 |
| PR8330 | SEQ ID NO: 55 | — | 1 . . . 22 |
| PR8331 | SEQ ID NO: 55 | — | 1361 . . . 1377 |
| PR8348 | SEQ ID NO: 54 | — | 1 . . . 21 |
| PR8349 | SEQ ID NO: 54 | — | 1031 . . . 1047 |
| PR10595 | Yali0C | NC_006069 | 1244252 . . . 1244265 |
| PR11110 | pCfB6681 | — | 2306 . . . 2336 |
| PR11111 | pCfB6681 | — | 5129 . . . 5152 |
| | | | 1 . . . 13 |
| PR11138 | pCfB4132 | — | 2894 . . . 2923 |
| PR13163 | pCfB3405 | — | 1680 . . . 1695 |
| PR13494 | pCfB3405 | — | 1133 . . . 1149 |
| PR13495 | pCfB3636 | — | 2621 . . . 2639 |
| PR13513 | pCfB4132 | — | 4160 . . . 4186 |
| PR13514 | pCfB4132 | — | 4168 . . . 4193 |
| PR14149 | Yali0A | NC_006067 | 2188554 . . . 2188574 |
| PR14279 | Yali0C | NC_006069 | 1244254 . . . 1244265 |
| PR15521 | Yali0C | NC_006069 | 1684182 . . . 1684200 |
| PR15522 | Yali0C | NC_006069 | 1685164 . . . 1685182 |
| PR15781 | Yali0A | NC_006067 | 2188556 . . . 2188574 |
| PR15930 | Yali0C | NC_006069 | 1243743 . . . 1243762 |
| PR16463 | Yali0B | NC_006068 | 1856737 . . . 1856756 |
| PR16464 | Yali0B | NC_006068 | 1857235 . . . 1857256 |
| PR16465 | Yali0B | NC_006068 | 1859087 . . . 1859109 |
| PR16466 | Yali0B | NC_006068 | 1859566 . . . 1859586 |
| PR16594 | SEQ ID NO: 11 | — | 1 . . . 18 |
| PR16595 | SEQ ID NO: 11 | — | 2735 . . . 2755 |
| PR17976 | Yali0E | NC_006071 | 3759199 . . . 3759218 |
| PR17977 | Yali0E | NC_006071 | 3759199 . . . 3759218 |
| PR18066 | SEQ ID NO: 11 | — | 1 . . . 21 |
| PR18214 | Yali0C | NC_006069 | 1243743 . . . 1243761 |
| PR18928 | Yali0C | NC_006069 | 1244252 . . . 1244265 |
| PR18233 | Yali0C | NC_006069 | 1842838 . . . 1842857 |
| PR18234 | Yali0C | NC_006069 | 1842838 . . . 1842857 |
| PR18239 | Yali0C | NC_006069 | 568873 . . . 568892 |
| PR18240 | Yali0C | NC_006069 | 568873 . . . 568892 |
| PR18241 | Yali0D | NC_006070 | 2193231 . . . 2193250 |
| PR18242 | Yali0D | NC_006070 | 2193231 . . . 2193250 |
| PR18253 | Yali0E | NC_006071 | 1845879 . . . 1845898 |
| PR18254 | Yali0E | NC_006071 | 1845879 . . . 1845898 |
| PR18255 | Yali0E | NC_006071 | 2882070 . . . 2882089 |
| PR18256 | Yali0E | NC_006071 | 2882070 . . . 2882089 |
| PR18930 | Yali0C | NC_006069 | 1244252 . . . 1244265 |
| PR20733 | Yali0B | NC_006068 | 2567154 . . . 2567173 |
| PR20734 | Yali0B | NC_006068 | 2567154 . . . 2567173 |
| PR20762 | Yali0B | NC_006068 | 2566672 . . . 2566691 |
| PR20763 | Yali0B | NC_006068 | 2567146 . . . 2567162 |
| PR20764 | Yali0B | NC_006068 | 2567171 . . . 2567190 |
| PR20765 | Yali0B | NC_006068 | 2567645 . . . 2567662 |
| PR20766 | Yali0B | NC_006068 | 2567140 . . . 2567162 |
| PR21723 | SEQ ID NO: 73 | — | 4 . . . 22 |
| PR21724 | SEQ ID NO: 73 | — | 997 . . . 1014 |
| PR21925 | Yali0C | NC_006069 | 1664157 . . . 1664176 |
| PR21660 | Yali0E | NC_006071 | 3890734 . . . 3890752 |
| PR21661 | Yali0E | NC_006071 | 3891217 . . . 3891235 |
| PR21662 | Yali0E | NC_006071 | 3893283 . . . 3893298 |
| PR21663 | Yali0E | NC_006071 | 3893754 . . . 3893774 |
| PR21664 | Yali0F | NC_006072 | 1449003 . . . 1449023 |
| PR21665 | Yali0F | NC_006072 | 1449511 . . . 1449530 |
| PR21666 | Yali0F | NC_006072 | 1451632 . . . 1451653 |
| PR21667 | Yali0F | NC_006072 | 1452130 . . . 1452149 |
| PR21668 | Yali0D | NC_006070 | 3274947 . . . 3274966 |
| PR21669 | Yali0D | NC_006070 | 3275426 . . . 3275444 |
| PR21670 | Yali0D | NC_006070 | 3277548 . . . 3277567 |
| PR21671 | Yali0D | NC_006070 | 3277975 . . . 3277994 |
| PR21672 | Yali0E | NC_006071 | 3260813 . . . 3260833 |
| PR21673 | Yali0E | NC_006071 | 2773817 . . . 2773827 |
| PR21674 | Yali0E | NC_006071 | 3264041 . . . 3264062 |
| PR21675 | Yali0E | NC_006071 | 3263543 . . . 3263562 |
| PR21676 | Yali0C | NC_006069 | 3195071 . . . 3195092 |
| PR21677 | Yali0C | NC_006069 | 3195550 . . . 3195570 |
| PR21678 | Yali0C | NC_006069 | 3197667 . . . 3197688 |
| PR21679 | Yali0C | NC_006069 | 3198143 . . . 3198161 |
| PR21717 | SEQ ID NO: 68 | — | 4 . . . 21 |
| PR21718 | SEQ ID NO: 68 | — | 987 . . . 1005 |
| PR21719 | SEQ ID NO: 66 | — | 4 . . . 23 |
| PR21720 | SEQ ID NO: 66 | — | 1032 . . . 1050 |
| PR21722 | SEQ ID NO: 1 | — | 1029 . . . 1047 |
| PR21755 | SEQ ID NO: 71 | — | 1 . . . 19 |
| PR21756 | SEQ ID NO: 71 | — | 1995 . . . 2013 |
| PR21767 | Yali0C | NC_006069 | 1663175 . . . 1663193 |
| PR21768 | Yali0C | NC_006069 | 1664158 . . . 1664176 |
| PR21771 | Yali0C | NC_006069 | 1663175 . . . 1663193 |
| PR21868 | Yali0D | NC_006070 | 2212116 . . . 2212130 |
| PR21869 | Yali0D | NC_006070 | 2210056 . . . 2210070 |
| PR22075 | Yali0C | NC_006069 | 1244246 . . . 1244265 |
| PR22134 | Yali0C | NC_006069 | 1244252 . . . 1244265 |
| PR22187 | Yali0E | NC_006069 | 1600091 . . . 1600110 |
| PR22188 | Yali0E | NC_006069 | 1601100 . . . 1601119 |
| PR22191 | Yali0E | NC_006069 | 1600091 . . . 1600110 |
| PR22239 | SEQ ID NO: 5 | — | 1 . . . 21 |
| PR22240 | SEQ ID NO: 5 | — | 420 . . . 441 |
| PR22241 | SEQ ID NO: 3 | — | 1 . . . 20 |
| PR22242 | SEQ ID NO: 3 | — | 363 . . . 384 |
| PR22243 | SEQ ID NO: 23 | — | 1 . . . 24 |
| PR22244 | SEQ ID NO: 23 | — | 945 . . . 969 |
| PR22295 | SEQ ID NO: 1 | — | 4 . . . 17 |
| PR22296 | Yali0C | NC_006069 | 1243743 . . . 1243765 |
| PR22342 | SEQ ID NO: 15 | — | 4 . . . 23 |
| PR22343 | SEQ ID NO: 15 | — | 4877 . . . 4887 |
| PR22344 | SEQ ID NO: 25 | — | 76 . . . 93 |
| PR22345 | SEQ ID NO: 25 | — | 603 . . . 627 |
| PR22847 | SEQ ID NO: 50 | — | 9 . . . 24 |
| PR22848 | SEQ ID NO: 50 | — | 2146 . . . 2162 |
| PR22915 | Yali0F | NC_006072 | 993533 . . . 993552 |
| PR22916 | Yali0F | NC_006072 | 993533 . . . 993552 |
| PR22917 | Yali0F | NC_006072 | 992669 . . . 992688 |
| PR22918 | Yali0F | NC_006072 | 993146 . . . 993168 |
| PR22919 | Yali0F | NC_006072 | 994094 . . . 994117 |
| PR22920 | Yali0F | NC_006072 | 994595 . . . 994614 |
| PR23176 | Yali0E | NC_006071 | 2795526 . . . 2795545 |
| PR23177 | Yali0E | NC_006071 | 2795526 . . . 2795545 |
| PR23285 | Yali0E | NC_006071 | 2882054 . . . 2882073 |
| PR23286 | Yali0E | NC_006071 | 2882054 . . . 2882073 |
| PR23412 | Yali0F | NC_006072 | 1974881 . . . 1974900 |
| PR23413 | Yali0F | NC_006072 | 1974881 . . . 1974900 |
| PR23414 | Yali0F | NC_006072 | 1974682 . . . 1974726 |
| | | | 1975796 . . . 1975840 |
| PR23415 | Yali0F | NC_006072 | 1974682 . . . 1974726 |
| | | | 1975796 . . . 1975840 |
| PR23423 | Yali0E | NC_006071 | 2249605 . . . 2249624 |
| PR23424 | Yali0E | NC_006071 | 2249605 . . . 2249624 |
| PR23425 | Yali0E | NC_006071 | 2249902 . . . 2249946 |
| | | | 2248697 . . . 2248741 |
| PR23426 | Yali0E | NC_006071 | 2249902 . . . 2249946 |
| | | | 2248697 . . . 2248741 |
| PR23429 | Yali0D | NC_006070 | 455135 . . . 455154 |
| PR23430 | Yali0D | NC_006070 | 455135 . . . 455154 |
| PR23431 | Yali0D | NC_006070 | 455450 . . . 455494 |
| | | | 454756 . . . 454800 |
| PR23432 | Yali0D | NC_006070 | 455450 . . . 455494 |
| | | | 454756 . . . 454800 |
| PR23468 | SEQ ID NO: 1 | — | 221 . . . 240 |
| PR23469 | SEQ ID NO: 1 | — | 221 . . . 240 |
| PR23472 | SEQ ID NO: 1 | — | 197 . . . 276 |
| PR23473 | SEQ ID NO: 1 | — | 197 . . . 276 |
| PR23613 | SEQ ID NO: 7 | — | 1 . . . 12 |
| PR23615 | SEQ ID NO: 7 | — | 1566 . . . 1582 |
| PR23632 | SEQ ID NO: 9 | — | 1 . . . 15 |
| PR23633 | SEQ ID NO: 9 | — | 2752 . . . 2770 |
| PR23685 | SEQ ID NO: 9 | — | 561 . . . 580 |
| PR23686 | SEQ ID NO: 9 | — | 561 . . . 580 |
| PR23687 | SEQ ID NO: 9 | — | 539 . . . 618 |
| PR23688 | SEQ ID NO: 9 | — | 539 . . . 618 |
| PR23689 | SEQ ID NO: 9 | — | 1293 . . . 1312 |
| PR23690 | SEQ ID NO: 9 | — | 1293 . . . 1312 |
| PR23691 | SEQ ID NO: 9 | — | 1250 . . . 1329 |
| PR23692 | SEQ ID NO: 9 | — | 1250 . . . 1329 |
| PR23693 | SEQ ID NO: 9 | — | 1373 . . . 1391 |
| PR23694 | SEQ ID NO: 1 | — | 1 . . . 15 |
| PR23695 | SEQ ID NO: 1 | — | 1029 . . . 1047 |
| PR23698 | Yali0B | NC_006068 | 2006545 . . . 2006564 |
| PR23699 | Yali0B | NC_006068 | 2006545 . . . 2006564 |
| PR23700 | Yali0B | NC_006068 | 2006501 . . . 2006590 |
| PR23701 | Yali0B | NC_006068 | 2006501 . . . 2006590 |
| PR23938 | SEQ ID NO: 75 | — | 18 . . . 32 |
| PR23939 | SEQ ID NO: 75 | — | 1373 . . . 1391 |
| PR23940 | SEQ ID NO: 77/79/81 | — | 4 . . . 18/4 . . . |
| | | | 18/4 . . . 18 |
| PR23941 | SEQ ID NO: 77/79 | — | 1344 . . . 1362/ |
| | | | 1344 . . . 1362 |
| PR23942 | SEQ ID NO: 80 | — | 1348 . . . 1371 |
| attB1_Cpo_CPRQ_F | SEQ ID NO: 1 | — | 1 . . . 30 |
| | CpoCPRQ | | |
| attB2_Cpo_CPRQ_R | SEQ ID NO: 1 | — | 1015 . . . 1047 |
| | CpoCPRQ | | |
| PR23127 | Yali0F | NC_006072 | 3823780 . . . 3823799 |
| PR23218 | Yali0C | NC_006069 | 763304 . . . 763285 |
| PR22776 | Yali0A | NC_006067 | 356505 . . . 356486 |
| PR22777 | Yali0A | NC_006067 | 356486 . . . 356505 |
| PR22213 | Yali0C | NC_006069 | 826768 . . . 826740 |
| PR23702 | SEQ ID NO: 1 | — | 1032 . . . 1047 |
| PR23703 | SEQ ID NO: 9 | — | 1 . . . 15 |
| PR23704 | SEQ ID NO: 9 | — | 1359 . . . 1377 |
| PR22536 | Yali0F | NC_006072 | 2011914 . . . 2011929 |
| PR22539 | Yali0F | NC_006072 | 2013213 . . . 2013198 |
| PR22534 | Yali0F | NC_006072 | 3823853 . . . 3823869 |
| PR22535 | Yali0F | NC_006072 | 3824352 . . . 3824332 |
| PR14148 | Yali0E | NC_006071 | 784691 . . . 784708 |
| PR15781 | Yali0A | NC_006067 | 2188565 . . . 2188547 |
| PR22744 | Yali0A | NC_006067 | 356480 . . . 356458 |
| PR22745 | Yali0A | NC_006067 | 356511 . . . 356532 |
| PR22684 | Yali0A | NC_006067 | 355981 . . . 356001 |
| PR22685 | Yali0A | NC_006067 | 357012 . . . 356690 |

TABLE 2
DNA fragments (BioBricks) obtained by PCR using the indicated template and primers

| Gene | | | | |
|---|---|---|---|---|
| fragment | | | | |
| name | Gene | Fw_primer | Rv_primer | Template DNA |
| BB0410 | Promoter Tdh3 from S. cerevisiae | PR1852 | PR1853 | CEN.PK102.5B |
| BB0684 | Fatty acyl reductase from A. | PR8330 | PR8331 | pCfB2341 |
| | segetum codon optimized for | | | |
| | S. cerevisiae | | | |
| BB0693 | Fatty acid desaturase Cpo_CPRQ | PR8348 | PR8349 | pCfB2339 |
| | from C. pomonella codon optimized | | | |
| | for S. cerevisiae | | | |
| BB1135 | Easy clone vector backbone | PR11110 | PR11111 | pCfB6681 |
| BB1338 | HphSynMX cassette with universal | PR141 | PR142 | BB1339 |
| | EasyClone overhangs for use in | | | BB1340 |
| | knockout cassettes for Y. lipolytica | | | |
| BB1339 | HphMX_start | PR13513 | PR13495 | pCfB4346 |
| BB1340 | HphMX_end | PR11138 | PR13514 | pCfB4346 |
| BB1341 | NatSynMX_start | PR13513 | PR13163 | pCfB4253 |
| BB1342 | NatSynMX_end | PR13494 | PR13514 | pCfB4253 |
| BB1346 | loxP-PrTefintron-Nat-SCopti-Tcyc- | PR141 | PR142 | BB1341 |
| | loxP | | | BB1342 |
| BB1558 | Promoter Exp from Y. lipolytica | PR15521 | PR15522 | ST6629 |
| BB1688 | Promoter Tefintron from Y. lipolytica | PR14279 | PR15930 | ST6629 |
| BB1725 | Long-chain alcohol oxidase from Y. | PR16463 | PR16464 | ST6629 |
| | lipolytica | | | |
| BB1726 | Long-chain alcohol oxidase from Y. | PR16465 | PR16466 | ST6629 |
| | lipolytica | | | |
| BB1740 | Fatty acyl reductase from H. | PR16594 | PR16595 | pCfB5547 |
| | armigera codon optimized for | | | |
| | Y. lipolytica | | | |
| BB2068 | Fatty acyl reductase from H. | PR18066 | PR16595 | pCfB5547 |
| | armigera codon optimized for | | | |
| | Y. lipolytica | | | |
| BB2093 | Promoter Tefintron from Y. | PR10595 | PR18214 | ST6629 |
| | lipolytica | | | |
| BB2311 | Fatty acid synthase 2 from Y. | PR20762 | PR20763 | ST6629 |
| | lipolytica | | | |
| BB2312 | Fatty acid synthase 2 from Y. | PR20764 | PR20765 | ST6629 |
| | lipolytica | | | |
| BB2313 | Fatty acid synthase 2 from Y. | PR20762 | PR20765 | BB2311 |
| | lipolytica | | | BB2312 |
| BB2314 | Fatty acid synthase 2 from Y. | PR20762 | PR20766 | ST6629 |
| | lipolytica | | | |
| BB2315 | Fatty acid synthase 2 from Y. | PR20762 | PR20765 | BB2312 |
| | lipolytica | | | BB2314 |
| BB2646 | Peroxisomal oxidase 1 from Y. | PR21660 | PR21661 | ST6629 |
| | lipolytica | | | |
| BB2647 | Peroxisomal oxidase 1 from Y. | PR21662 | PR21663 | ST6629 |
| | lipolytica | | | |
| BB2648 | Peroxisomal oxidase 1 from Y. | PR21660 | PR21663 | BB2646 |
| | lipolytica | | | BB2647 |
| BB2649 | Peroxisomal oxidase 2 from Y. | PR21664 | PR21665 | ST6629 |
| | lipolytica | | | |
| BB2650 | Peroxisomal oxidase 2 from Y. | PR21666 | PR21667 | ST6629 |
| | lipolytica | | | |
| BB2651 | Peroxisomal oxidase 2 from Y. | PR21664 | PR21667 | BB2649 |
| | lipolytica | | | BB2650 |
| BB2652 | Peroxisomal oxidase 3 from Y. | PR21668 | PR21669 | ST6629 |
| | lipolytica | | | |
| BB2653 | Peroxisomal oxidase 3 from Y. | PR21670 | PR21671 | ST6629 |
| | lipolytica | | | |
| BB2654 | Peroxisomal oxidase 3 from Y. | PR21668 | PR21671 | BB2652 |
| | lipolytica | | | BB2653 |
| BB2655 | Peroxisomal oxidase 4 from Y. | PR21672 | PR21673 | ST6629 |
| | lipolytica | | | |
| BB2656 | Peroxisomal oxidase 4 from Y. | PR21674 | PR21675 | ST6629 |
| | lipolytica | | | |
| BB2657 | Peroxisomal oxidase 4 from Y. | PR21672 | PR21675 | BB2655 |
| | lipolytica | | | BB2656 |
| BB2658 | Peroxisomal oxidase 5 from Y. | PR21676 | PR21677 | ST6629 |
| | lipolytica | | | |
| BB2659 | Peroxisomal oxidase 5 from Y. | PR21678 | PR21679 | ST6629 |
| | lipolytica | | | |
| BB2660 | Peroxisomal oxidase 5 from Y. | PR21676 | PR21679 | BB2658 |
| | lipolytica | | | BB2659 |
| BB2690 | Fatty acid desaturase Cpo_SPTQ | PR-21717 | PR-21718 | pBP7890 |
| | from C. pomonella codon optimized | | | |
| | for Y. lipolytica | | | |
| BB2691 | Fatty acid desaturase Cpo_NPVE | PR-21719 | PR-21720 | pBP7891 |
| | from C. pomonella codon optimized | | | |
| | for Y. lipolytica | | | |
| BB2719 | Promoter Tef1 from Y. lipolytica | PR18928 | PR18214 | ST6629 |
| BB2720 | Promoter Tefintron from Y. lipolytica | PR18930 | PR18214 | ST6629 |
| BB2721 | Promoter Exp from Y. lipolytica | PR21767 | PR21768 | ST6629 |
| BB2723 | Promoter Exp from Y. lipolytica | PR21771 | PR15522 | ST6629 |
| BB8012 | Fatty acyl-CoA synthase from Y. | PR21868 | PR21869 | ST6629 |
| | lipolytica | | | |
| BB8018 | Double promoter Exp/Tefintron from | PR18214 | PR21925 | BB2720 |
| | Y. lipolytica | | | BB2723 |
| BB8138 | Promoter Yef3 from Y. lipolytica | PR22187 | PR22188 | ST6629 |
| BB8141 | Promoter Yef3 from Y. lipolytica | PR22191 | PR22188 | ST6629 |
| BB8167 | Fatty acyl reductase from H. | PR22134 | PR14149 | pCfB5547 |
| | armigera codon optimized for | | | |
| | Y. lipolytica | | | |
| BB8168 | Fatty acyl reductase from H. | PR22075 | PR15781 | pCfB5547 |
| | armigera codon optimized for | | | |
| | Y. lipolytica | | | |
| BB8214 | Hemoglobin from Vitreoscilla | PR22239 | PR22240 | pBP8074 |
| | stercoraria codon optimized for | | | |
| | Y. lipolytica | | | |
| BB8215 | Cytochrome b5 from H. armigera | PR22241 | PR22242 | pBP8075 |
| | codon optimized for | | | |
| | Y. lipolytica | | | |
| BB8216 | Cytochrome b5 reductase from H. | PR22243 | PR22244 | pBP8076 |
| | armigera codon optimized for | | | |
| | Y. lipolytica | | | |
| BB8246 | Promoter Tefintron from Y. lipolytica | PR10595 | PR22296 | ST6629 |
| BB8247 | Fatty acid desaturase Cpo_CPRQ | PR22295 | PR21722 | pBP7892 |
| | from C. pomonella codon optimized | | | |
| | for Y. lipolytica | | | |
| BB8286 | Shortened fatty acid synthase 1 from | PR22342 | PR22343 | ST6629 |
| | Y. lipolytica | | | |
| BB8287 | Thioesterase from Escherichia coli | PR-22344 | PR-22345 | pCfB7680 |
| | codon optimized for Y. lipolytica | | | |
| BB8313 | Hemoglobin from V. stercoraria | PR22187 | PR22240 | pBP8119 |
| | codon optimized for Y. lipolytica | | | |
| BB8526 | Peroxisomal oxidase from | PR22847 | PR22848 | pBP8308 |
| | Paenarthrobacter ureafaciens | | | |
| BB8562 | Fatty acid elongase 1 from | PR22917 | PR22918 | ST6629 |
| | Y. lipolytica | | | |
| BB8563 | Fatty acid elongase 1 from | PR22919 | PR22920 | ST6629 |
| | Y. lipolytica | | | |
| BB8564 | Fatty acid elongase 1 from | PR22917 | PR22920 | BB8562 |
| | Y. lipolytica | | | BB8563 |
| BB8724 | Fatty acyl reductase from | PR23005 | PR15781 | BB8614 |
| | H. armigera codon optimized for | | | BB2068 |
| | Y. lipolytica | | | |
| BB8790 | Fatty acyl reductase from T. alba | PR23613 | PR23615 | pBP8751 |
| | codon optimized for Y. lipolytica | | | |
| BB8816 | Fatty acyl reductase from | PR23632 | PR23633 | pBP8777 |
| | A. segetum codon optimized for | | | |
| | Y. lipolytica | | | |
| BB8824 | Fatty acid desaturase Cpo_CPRQ | PR22295 | PR21722 | ST9072 |
| | from C. pomonella codon optimized | | | |
| | for Y. lipolytica | | | |
| BB8829 | Fatty acyl reductase from | PR23632 | PR23693 | pBP8782 |
| | A. segetum codon optimized for | | | |
| | Y. lipolytica | | | |
| BB8830 | Fatty acid desaturase Cpo_CPRQ | PR23694 | PR23695 | pBP7911 |
| | from C. pomonella codon optimized | | | |
| | for Y. lipolytica | | | |
| BB8923 | Fatty acyl reductase from | PR23938 | PR23939 | pBP8925 |
| | A. ipsilon codon optimized for | | | |
| | Y. lipolytica | | | |
| BB8924 | Fatty acyl reductase from Heliothis | PR23940 | PR23941 | pBP2344 |
| | subflexa codon optimized for Y. | | | |
| | lipolytica | | | |
| BB8925 | Fatty acyl reductase from Heliothis | PR23940 | PR23941 | pBP2345 |
| | virescens codon optimized for Y. | | | |
| | lipolytica | | | |
| BB8926 | Fatty acyl reductase from H. assulta | PR23940 | PR23942 | pBP2346 |
| | codon optimized for Y. lipolytica | | | |
| BB8618 | Promoter Tefintron/GPD from Y. | PR18214 | PR22213 | pCfB3465¹ |
| | lipolytica | | | |
| BB8832 | Fatty acid desaturase Cpo_CPRQ | PR22295 | PR23702 | pBP7911 |
| | from C. pomonella codon optimized | | | |
| | for Y. lipolytica | | | |
| BB8833 | Fatty acyl reductase from | PR23703 | PR23704 | pBP8782 |
| | A. segetum codon optimized for | | | |
| | Y. lipolytica | | | |
| BB8386 | Genomic region upstream of | PR22536 | PR22539 | ST6629 |
| | integration site | | | |
| BB1631 | Y. lipolytica terminator regions of | PR14148 | PR15781 | pCfB45862 |
| | pex20 and lip2 | | | |
| BB8387 | Genomic region downstream of | PR22534 | PR22535 | ST6629 |
| | integration site | | | |
| BB8546 | Vector backbone of pBP8375 | PR22744 | PR22745 | pBP8375 |
| BB8390 | gfp expression cassette | PR22540 | PR22541 | pCfB5124² |
| BB8488 | Genomic region upstream of | PR22684 | PR22744 | ST6629 |
| | integration site | | | |
| BB8489 | Genomic region downstream of | PR22745 | PR22685 | ST6629 |
| | integration site | | | |

¹Holkenbrink et al. 2020
²Holkenbrink et al. 2017

### Example 2: Construction of Plasmids

Integrative yeast vectors with USER cassette were linearized with FastDigest SfaAl (ThermoFisher) for 2 hours at 37° C. and then nicked with Nb.Bsml (New England Biolabs) for 1 hour at 65° C. The resulting vectors containing sticky ends were separated by gel electrophoresis, excised from the gel, and gel-purified using the Nucleospin Gel and PCR Clean-up kit (Macherey-Nagel). The DNA fragments were cloned into the so prepared vectors by USER-cloning as described in Holkenbrink et al., 2017. The reaction was transformed into chemically competent E. coli DHα cells and the cells were plated on Lysogeny Broth (LB) agar plates with 100 mg/L ampicillin. The plates were incubated overnight at 37° C. and the resulting colonies were screened by colony PCR. The plasmids were purified from overnight E. coli liquid cultures and the correct cloning was confirmed by sequencing. The constructed vectors are listed in Table 3.

TABLE 3
Integrative expression vectors

| Integrative expression | Parent | DNA fragments cloned |
|---|---|---|
| vector name | vector | into parent vector |
| pCfB2227 | — | — |
| pCfB2500 | pCfB2227 | BB0410 |
| | | BB0693 |
| pCfB2501 | pCfB2190 | BB0410 |
| | | BB0684 |
| pBP7909 | pCfB6684 | BB2719 |
| | | BB2690 |
| pBP7910 | pCfB6684 | BB2719 |
| | | BB2691 |
| pBP7911 | pCfB6684 | BB8246 |
| | | BB8247 |
| pBP7912 | pCfB6684 | BB2719 |
| | | BB2693 |
| pBP7980 | pCfB6371 | BB1688 |
| | | BB1740 |
| pBP8053 | pCfB6677 | BB8167 |
| | | BB8168 |
| pBP8100 | pCfB6685 | BB8214 |
| | | BB8141 |
| | | BB2723 |
| | | BB8215 |
| pBP8114 | pCfB6681 | BB8012 |
| | | BB8138 |
| pBP8137 | pCfB6679 | BB8246 |
| | | BB8247 |
| pBP8175 | pCfB6682 | BB2093 |
| | | BB8286 |
| | | BB8287 |
| pBP8193 | pCfB6682 | BB1558 |
| | | BB8215 |
| pBP8194 | pCfB6682 | BB8216 |
| | | BB8141 |
| | | BB2723 |
| | | BB8215 |
| pBP8212 | pCfB6681 | BB8313 |
| pBP8350 | pCfB6371 | BB2721 |
| | | BB8526 |
| pBP8400 | pCfB6371 | BB1558 |
| | | BB2709 |
| pBP8781 | pBP8660 | BB2093 |
| | | BB8790 |
| pBP8782 | pBP8660 | BB2093 |
| | | BB8816 |
| pBP8795 | pCfB6684 | BB2093 |
| | | BB8824 |
| pBP8802 | pBP8620 | BB8825 |
| | | BB8724 |
| pBP8829 | pBP8862 | BB8018 |
| | | BB8829 |
| | | BB8830 |
| pBP8926 | pBP8660 | BB2093 |
| | | BB8923 |
| pBP8931 | pBP8660 | BB2093 |
| | | BB8924 |
| pBP8932 | pBP8660 | BB2093 |
| | | BB8925 |
| pBP8933 | pBP8660 | BB2093 |
| | | BB8926 |
| pBP9361 | pCfB6682 | BB2719 |
| | | BB2690 |
| pBP8826 | pBP8394 | BB8018 |
| | | BB8829 |
| | | BB8830 |
| pBP8828 | pBP8263 | BB8618 |
| | | BB8832 |
| | | BB8833 |
| pBP8394 | — | BB8546 |
| | | BB1631 |
| pBP8263 | — | BB1135 |
| | | BB8386 |
| | | BB1631 |
| | | BB8387 |
| pBP8375 | — | BB1135 |
| | | BB8390 |
| | | BB8488 |
| | | BB8489 |

### Example 3: Construction of Strains

Yeast strains were constructed by transformation of DNA vectors as described in Holkenbrink et al., 2017. Integrative vectors were linearized with FastDigest Notl prior to transformation. When needed, helper vectors to promote the integration into specific genomic regions were co-transformed with the integrative plasmid or DNA repair fragments listed in Table 4. Strains were selected on yeast peptone dextrose (YPD) agar with appropriate antibiotics selection. Correct genotype was confirmed by colony PCR and when needed by sequencing. A Y. lipolytica wild-type strain was transformed with the plasmid pCfB6364 (EP19204554), leading to strain ST6029, then the genes HFD1 (YALI0_F23793g), HFD2 (YALI0_E15400g), HFD3 (YALI0_A17875g), HFD4 (YALI0_B01298g), FAO1 (YALI0_B14014g), and PEX10 (YAL11_C01416g) were deleted, leading to strain ST6629 (Borodina et al., 2018). Strains ST6029 and ST6629 were used as parental strains to construct all other strains. The resulting strains are listed in Table 5.

TABLE 4
Helper vectors

| Vector | Selection | Parent | DNA fragments cloned |
|---|---|---|---|
| name | marker | vector | into parent vector |
| pCfB5573 | Hygromycin | — | BB1135 |
| | | | BB1338 |
| | | | BB1725 |
| | | | BB1726 |
| pCfB5574 | Nourseothricin | — | BB1135 |
| | N-acetyl | | BB1346 |
| | transferase | | BB1725 |
| | | | BB1726 |
| pCfB6627 | Nourseothricin | pCfB3405 | BB1635 |
| | N-acetyl | | BB1636 |
| | transferase | | PR18233 |
| | | | PR18234 |
| pCfB6630 | Nourseothricin | pCfB3405 | BB1635 |
| | N-acetyl | | BB1636 |
| | transferase | | PR18239 |
| | | | PR18240 |
| pCfB6631 | Nourseothricin | pCfB3405 | BB1635 |
| | N-acetyl | | BB1636 |
| | transferase | | PR18241 |
| | | | PR18242 |
| pCfB6637 | Nourseothricin | pCfB3405 | BB1635 |
| | N-acetyl | | BB1636 |
| | transferase | | PR18253 |
| | | | PR18254 |
| pCfB6638 | Nourseothricin | pCfB3405 | BB1635 |
| | N-acetyl | | BB1636 |
| | transferase | | PR18255 |
| | | | PR18256 |
| pCfB7088 | Nourseothricin | pCfB3405 | BB1635 |
| | N-acetyl | | BB1636 |
| | transferase | | PR20733 |
| | | | PR20734 |
| pBP8032 | Hygromycin | pCfB3431 | BB1635 |
| | | | BB1636 |
| | | | PR18239 |
| | | | PR18240 |
| pBP8033 | Hygromycin | pCfB3431 | BB1635 |
| | | | BB1636 |
| | | | PR18241 |
| | | | PR18242 |
| pBP8034 | Hygromycin | pCfB3431 | BB1635 |
| | | | BB1636 |
| | | | PR18245 |
| | | | PR18246 |
| pBP8035 | Hygromycin | pCfB3431 | BB1635 |
| | | | BB1636 |
| | | | PR18253 |
| | | | PR18254 |
| pBP8161 | Hygromycin | pCfB3431 | BB1635 |
| | | | BB1636 |
| | | | PR18255 |
| | | | PR18256 |
| pBP8185 | Hygromycin | pCfB3431 | BB1635 |
| | | | BB1636 |
| | | | PR17976 |
| | | | PR17977 |
| pBP8406 | Hygromycin | pCfB3431 | BB1635 |
| | | | BB1636 |
| | | | PR22915 |
| | | | PR22916 |
| pBP8535 | Hygromycin | pCfB3431 | BB1635 |
| | | | BB1636 |
| | | | PR23123 |
| | | | PR23124 |
| pBP8575 | Nourseothricin | pCfB3405 | BB1635 |
| | N-acetyl | | BB1636 |
| | transferase | | PR23190 |
| | | | PR23191 |
| pBP8568 | Hygromycin | pCfB3431 | BB1635 |
| | | | BB1636 |
| | | | PR23176 |
| | | | PR23177 |
| pBP8623 | Nourseothricin | pCfB3405 | BB1635 |
| | N-acetyl | | BB1636 |
| | transferase | | PR23192 |
| | | | PR23193 |
| pBP8634 | Nourseothricin | pCfB3405 | BB1635 |
| | N-acetyl | | BB1636 |
| | transferase | | PR23285 |
| | | | PR23286 |
| pBP8650 | Nourseothricin | pCfB3405 | BB1635 |
| | N-acetyl | | BB1636 |
| | transferase | | PR23308 |
| | | | PR23309 |
| pBP8657 | Nourseothricin | pCfB3405 | BB1635 |
| | N-acetyl | | BB1636 |
| | transferase | | PR21648 |
| | | | PR21649 |
| pBP8674 | Nourseothricin | pCfB3405 | BB1635 |
| | N-acetyl | | BB1636 |
| | transferase | | PR23176 |
| | | | PR23177 |
| pBP8704 | Nourseothricin | pCfB3405 | BB1635 |
| | N-acetyl | | BB1636 |
| | transferase | | PR23385 |
| | | | PR23386 |
| pBP8713 | Hygromycin | pCfB3431 | BB1635 |
| | | | BB1636 |
| | | | PR23412 |
| | | | PR23413 |
| pBP8716 | Hygromycin | pCfB3431 | BB1635 |
| | | | BB1636 |
| | | | PR23423 |
| | | | PR23424 |
| pBP8717 | Hygromycin | pCfB3431 | BB1635 |
| | | | BB1636 |
| | | | PR23429 |
| | | | PR23430 |
| pBP8734 | Hygromycin | pCfB3431 | BB1635 |
| | | | BB1636 |
| | | | PR23468 |
| | | | PR23469 |
| pBP8813 | Hygromycin | pCfB3431 | BB1635 |
| | | | BB1636 |
| | | | PR23671 |
| | | | PR23672 |
| pBP8815 | Hygromycin | pCfB3431 | BB1635 |
| | | | BB1636 |
| | | | PR23685 |
| | | | PR23686 |
| pBP8817 | Hygromycin | pCfB3431 | BB1635 |
| | | | BB1636 |
| | | | PR23689 |
| | | | PR23690 |
| pBP8871 | Hygromycin | pCfB3431 | BB1635 |
| | | | BB1636 |
| | | | PR23698 |
| | | | PR23699 |
| pBP8645 | Hygromycin | pCfB3431 | BB1635 |
| | | | BB1636 |
| | | | PR23127 |
| | | | PR23128 |
| pBP8328 | Nourseothricin | pCfB3405 | BB1635 |
| | N-acetyl | | BB1636 |
| | transferase | | PR22776 |
| | | | PR22777 |

TABLE 5
Yeast strains

| | Modified intrinsic | Overexpressed | Parent strain and |
|---|---|---|---|
| Strain name | genes | gene(s) | (integrated DNA fragments) |
| ST6629 | hfd1Δ hfd2Δ hfd3Δ | | |
| | hfd4Δ fao1Δ | | |
| | pex10Δ | | |
| ST3332 | Reference: | Cpo_CPRQ | CEN.PK102-5B (pBP2500) |
| | CEN.PK102-5B | | |
| ST8310 | ΔHFD1 | VHb | ST6629 (pBP8212/pBP6637) |
| | (YALI0_F23793g) | | |
| | ΔHFD2 | | |
| | (YALI0_E15400g) | | |
| | ΔHFD3 | | |
| | (YALI0_A17875g) | | |
| | ΔHFD4 | | |
| | (YALI0_B01298g) | | |
| | ΔFAO1 | | |
| | (YALI0_B14014g) | | |
| | ΔPEX10 | | |
| | (YALI0_C01023g) | | |
| ST8311 | ΔHFD1 | HarCyb5 | ST6629 (pBP8193/pBP6627) |
| | (YALI0_F23793g) | | |
| | ΔHFD2 | | |
| | (YALI0_E15400g) | | |
| | ΔHFD3 | | |
| | (YALI0_A17875g) | | |
| | ΔHFD4 | | |
| | (YALI0_B01298g) | | |
| | ΔFAO1 | | |
| | (YALI0_B14014g) | | |
| | ΔPEX10 | | |
| | (YALI0_C01023g) | | |
| ST8406 | ΔHFD1 | Cpo_CPRQ | ST6629 (pBP8137/pBP6638) |
| | (YALI0_F23793g) | | |
| | ΔHFD2 | | |
| | (YALI0_E15400g) | | |
| | ΔHFD3 | | |
| | (YALI0_A17875g) | | |
| | ΔHFD4 | | |
| | (YALI0_B01298g) | | |
| | ΔFAO1 | | |
| | (YALI0_B14014g) | | |
| | ΔPEX10 | | |
| | (YALI0_C01023g) | | |
| ST8411 | Reference: ST8311 | HarCyb5 | ST8311 (pBP8137/pBP8161) |
| | | Cpo_CPRQ | |
| ST8416 | Reference: ST8310 | VHb | ST8310 (pBP8137/pBP8161) |
| | | Cpo_CPRQ | |
| ST8494 | Reference: ST8406 | Cpo_CPRQ | ST8406 (pBP8053/pBP8034) |
| | | 2x Har_FAR | |
| ST9060 | Reference: ST8406 | Cpo_CPRQ | ST8406 (BB8564/pBP8406) |
| | ΔELO1 | | |
| | (YALI0_F06754g) | | |
| ST9061 | Reference: ST8406 | Cpo_CPRQ | ST8406 |
| | ΔYALI0_F14729g | | (PR23414/PR23415/pBP8713) |
| ST9062 | Reference: ST8406 | Cpo_CPRQ | ST8406 |
| | ΔYALI0_E18876g | | (PR23425/PR23426/pBP8716) |
| ST9063 | Reference: ST8406 | Cpo_CPRQ | ST8406 |
| | ΔYALI0_D03597g | | (PR23431/PR23432/pBP8717) |
| ST9064 | Reference: ST8406 | Cpo_CPRQ | ST8406 |
| | | CPO_SPTQ | (pBP7909/pBP8033) |
| ST9065 | Reference: ST8406 | Cpo_CPRQ | ST8406 |
| | | Cpo_NPVE | (pBP7910/pBP8033) |
| ST9066 | Reference: ST8406 | 2x Cpo_CPRQ | ST8406 (pBP7911/pBP8033) |
| ST9072 | Reference: ST8406 | Cpo_CPRQ_S85A | ST8406 |
| | | | (PR23472/PR23473/pBP8734) |
| ST9115 | Reference: ST8411 | HarCyb5 | ST8411 (pBP8212/pBP6637) |
| | | Cpo_CPRQ | |
| | | VHb | |
| ST9163 | Reference: ST6029 | — | ST6029 (BB2660/pBP8657) |
| | ΔPOX5 | | |
| | (YALI0_C23859g) | | |
| ST9179 | Reference: ST9163 | — | ST9163 (BB2654/pBP8704) |
| | ΔPOX3 | | |
| | (YALI0_D24750g) | | |
| ST9215 | Reference: ST9179 | — | ST9179 (BB2648/pBP8535) |
| | ΔPOX1 | | |
| | (YALI0_E32835g) | | |
| ST9249 | Reference: ST9066 | 2x Cpo_CPRQ | ST9066 (pBP8781/pBP8674) |
| | | Ta_FAR_w/o_SKL | |
| ST9250 | Reference: ST9066 | 2x Cpo_CPRQ | ST9066 (pBP8782/pBP8674) |
| | | Ase_FAR | |
| ST9252 | Reference: ST9215 | — | ST9215 (BB2657/pBP8650) |
| | ΔPOX4 | | |
| | (YALI0_E27654g) | | |
| ST9278 | Reference: ST9060 | 2x Cpo_CPRQ | ST9060 (pBP7911/pBP6631) |
| ST9279 | Reference: ST9060 | Cpo_CPRQ | ST9060 (pBP8795/pBP6631) |
| | | Cpo_CPRQ_S85A | |
| ST9331 | ΔPOX5 | — | ST9252 (BB2651/pBP8813) |
| | (YALI0_C23859g) | | |
| | ΔPOX3 | | |
| | (YALI0_D24750g) | | |
| | ΔPOX1 | | |
| | (YALI0_E32835g) | | |
| | ΔPOX4 | | |
| | (YALI0_E27654g) | | |
| | ΔPOX2 | | |
| | (YALI0_F10857g) | | |
| ST9335 | Reference: ST9250 | 2x Cpo_CPRQ | ST9250 |
| | | Ase_FAR_T198A | (PR23687/PR23688/pBP8815) |
| ST9336 | Reference: ST9250 | 2x Cpo_CPRQ | ST9250 |
| | | Ase_FAR_S423A | (PR23691/PR23692/pBP8817) |
| ST9355 | Reference: ST9279 | Cpo_CPRQ | ST9279 (pBP8100/pBP8185) |
| | | Cpo_CPRQ_S85A | |
| | | VHb | |
| | | HarCyb5 | |
| ST9356 | Reference: ST9355 | Cpo_CPRQ | ST9355 (pBP8194/pBP6627) |
| | | Cpo_CPRQ_S85A | |
| | | VHb | |
| | | 2x HarCyb5 | |
| | | HarCyb5R | |
| ST9357 | Reference: ST9356 | Cpo_CPRQ | ST9356 |
| | ΔYALI0_F14729g | Cpo_CPRQ_S85A | (PR23414/PR23415/pBP8713) |
| | | VHb | |
| | | 2x HarCyb5 | |
| | | HarCyb5R | |
| ST9358 | Reference: ST9357 | Cpo_CPRQ | ST9357 (pBP8782/pBP8674) |
| | | Cpo_CPRQ_S85A | |
| | | VHb | |
| | | 2x HarCyb5 | |
| | | HarCyb5R | |
| | | Ase_FAR | |
| ST9372 | Reference: ST6029 | Har_FAR | ST6029 (pBP7980/pBP8032) |
| ST9382 | Reference: ST6029 | Ase_FAR | ST6029 (pBP8782/pBP8674) |
| ST9387 | Reference: ST9279 | Cpo_CPRQ | ST9279 (pBP8782/pBP8568) |
| | | Cpo_CPRQ_S85A | |
| | | Ase_FAR | |
| ST9388 | Reference: ST9387 | Cpo_CPRQ | ST9387 (BB2313/pBP7088) |
| | FAS2(I1220F) | Cpo_CPRQ_S85A | |
| | | Ase_FAR | |
| ST9395 | Reference: ST9382 | Ase_FAR | ST9382 (pBP7911/pBP8033) |
| | | Cpo_CPRQ | |
| ST9397 | Reference: ST9388 | Cpo_CPRQ | ST9388 (pBP8175/pBP6627) |
| | FAS2(I1220F) | Cpo_CPRQ_S85A | |
| | | Ase_FAR | |
| | | FAS1‘EcTesA’ | |
| ST9398 | Reference: ST9397 | Cpo_CPRQ | ST9397 (pBP8114/pBP8035) |
| | FAS2(I1220F) | Cpo_CPRQ_S85A | |
| | | Ase_FAR | |
| | | FAS1‘EcTesA’ | |
| | | FAA1 | |
| ST9420 | Reference: ST9387 | Cpo_CPRQ | ST9387 (BB2315/pBP7088) |
| | FAS2(I1220W) | Cpo_CPRQ_S85A | |
| | | Ase_FAR | |
| ST9421 | Reference: ST9420 | Cpo_CPRQ | ST9420 |
| | FAS2I1220W | Cpo_CPRQ_S85A | (PR23700/PR23701/pBP8871) |
| | FAS1L123V | Ase_FAR | |
| ST9492 | Reference: ST9372 | Har_FAR | ST9372 (pBP8137/pBP8634) |
| | | Cpo_CPRQ | |
| ST9517 | Reference: ST8406 | Cpo_CPRQ | ST8406 (pBP8782/pBP8568) |
| | | Ase_FAR | |
| ST9519 | Reference: ST9331 | Ase_FAR | ST9331 (pBP8829/pBP8575) |
| | | CPO_CPRQ | |
| ST9520 | Reference: ST9519 | Ase_FAR | ST9519 (pBP8350/pBP8032) |
| | | CPO_CPRQ | |
| | | Pur POX | |
| ST9614 | Reference: ST9519 | Ase_FAR | ST9519 (pBP5573) |
| | ΔFAO1 | CPO_CPRQ | |
| | (YALI0_B14014g) | | |
| ST9615 | Reference: ST9520 | Ase_FAR | ST9520 (pBP5574) |
| | ΔFAO1 | CPO_CPRQ | |
| | (YALI0_B14014g) | Pur_POX | |
| ST9623 | Reference: ST9357 | Cpo_CPRQ | ST9357 (pBP8926/pBP8674) |
| | | Cpo_CPRQ_S85A | |
| | | VHb | |
| | | 2x HarCyb5 | |
| | | HarCyb5R | |
| | | AipFAR | |
| ST9628 | Reference: ST9357 | Cpo_CPRQ | ST9357 (pBP8931/pBP8674) |
| | | Cpo_CPRQ_S85A | |
| | | VHb | |
| | | 2x HarCyb5 | |
| | | HarCyb5R | |
| | | HsFAR | |
| ST9629 | Reference: ST9357 | Cpo_CPRQ | ST9357 (pBP8932/pBP8674) |
| | | Cpo_CPRQ_S85A | |
| | | VHb | |
| | | 2x HarCyb5 | |
| | | HarCyb5R | |
| | | HvFAR | |
| ST9630 | Reference: ST9357 | Cpo_CPRQ | ST9357 (pBP8933/pBP8674) |
| | | Cpo_CPRQ_S85A | |
| | | VHb | |
| | | 2x HarCyb5 | |
| | | HarCyb5R | |
| | | HasFAR | |
| ST9631 | Reference: ST3332 | Cpo_CPRQ | ST3332 (pBP2501) |
| | | Ase_FAR | |
| ST10136 | Reference: ST6629 | Cpo_SPTQ | ST6629 |
| | | | (pBP9361/pBP6627) |
| ST10137 | Reference: ST6629 | Cpo_NPVE | ST6629 |
| | | | (pBP7910/pBP6631) |
| ST10138 | Reference: ST9065 | Cpo_CPRQ | ST9065 |
| | | Cpo_NPVE | (pBP9361/pBP6627) |
| | | Cpo_SPTQ | |
| elo1Δ ole1Δ | Schneiter, 2000 | | elo14 ole14 (pYEX-CHT- |
| (Saccharomyces | | | CpoCPRQ) |
| cerevisiae) | | | |
| elo1Δ ole1Δ | Reference: elo1Δ | CpoCPRQ | |
| CpoCPRQ | ole1Δ | | |
| ST9493 | Reference: ST9357 | 2xCpo_CPRQ | ST9357 (pBP8826/pBP8328) |
| | | Cpo_CPRQ_S85A | |
| | | VHb | |
| | | 2x HarCyb5 | |
| | | HarCyb5R | |
| | | AseFAR | |
| ST9494 | Reference: ST9493 | 3xCpo_CPRQ | ST9493 (pBP8828/pBP8645) |
| | | Cpo_CPRQ_S85A | |
| | | VHb | |
| | | 2x HarCyb5 | |
| | | HarCyb5R | |
| | | 2xAseFAR | |
| ST9495 | Reference: ST9494 | 4xCpo_CPRQ | ST9494 (pBP8829/pBP8575) |
| | | Cpo_CPRQ_S85A | |
| | | VHb | |
| | | 2x HarCyb5 | |
| | | HarCyb5R | |
| | | 3xAseFAR | |

### Example 4: Cultivation of Strains, Extraction and Analysis of Fatty Acid Methyl Esters and Fatty Alcohols

Strains were inoculated from a YPD agar plate (10 g/L yeast extract, 10 g/L peptone, 20 g/L glucose, 15 g/L agar agar) to an initial OD₆₀₀ of 0.1-0.2 into 2.5 mL YPG medium (10 g/L yeast extract, 10 g/L peptone, 40 g/L glycerol) in 24 well-plates (EnzyScreen). The plates were incubated at 28° C., shaken at 300 rpm. After 22 h, the plates were centrifuged for 5 min at 4° C. and 3,000 xg. The supernatant was discarded and the cells were resuspended in 1.25 mL production medium per well (Borodina et al., 2018). The medium was supplemented with 2.5 μL methyl dodecanoate. The plate was incubated for 28 hours at 28° C., shaken at 300 rpm.

For analysis of fatty alcohols, 200 μL of the broth was extracted with 990 μL of ethyl acetate:ethanol (84:15) and 10 μL of Z10-17:Me (2 mg/mL) as internal standard. The samples were vortexed for 20 sec and incubated for 1 h at room temperature, followed by 5 min of vortexing. 300 μL of H₂O was added to each sample. The samples were vortexed and centrifuged for 5 min at 21° C. and 3,000×g. The upper organic phase was analyzed via gas chromatography-mass spectrometry (GC-MS). GC-MS analyses were performed on a Hewlett Packard 6890 GC coupled to a mass selective detector HP 5973. The GC was equipped with an INNOWax column (30 m×0.25 mm×0.25 μm), and helium was used as carrier gas (average velocity: 33 cm/s). The MS was operated in electron impact mode (70 eV), scanning between m/z 30 and 400, and the injector was configured in splitless mode at 220° C. The oven temperature was set to 80° C. for 1 min, then increased at a rate of 10° C./min to 210° C., followed by a hold at 210° C. for 15 min, and then increased at a rate of 10° C./min to 230° C. followed by a hold at 230° C. for 20 min. Compounds were identified by comparison of retention times and mass spectra with those of reference compounds available in laboratory collection. Compounds were quantified by the Total Ion Current (TIC) recorded. Data were analyzed by the Agilent ChemStation software and iWork Numbers.

For analysis of the fatty acids, 1 mL of each vial was harvested by centrifugation for 5 min at 4° C. and 3,000 xg. Each pellet was extracted with 1000 μL 1M HCl in Methanol (anhydrous). The samples were vortexed for 20 sec and placed in the 80° C. water bath for 2 h. The samples were vortexed every 30 min for 10 sec. After cooling down of the samples to room temperature, 1000 μL of 1M NaOH in Methanol (anhydrous), 500 μL of NaCl saturated H₂O, 990 μL of hexane and 10 μL of Z10-17:Me (2 mg/mL) as internal standard were added. The samples were vortexed and centrifuged for 5 min at 21° C. and 3,000 xg. The upper organic phase was analyzed via GC-MS as described above.

### Example 5: Production of E8,E10-C12:OH in Y. lipolytica

Strain ST8494, derived from strain ST6629, expresses the Helicoverpa armigera fatty acyl reductase Har_FAR (in two copies) and the Cydia pomonella desaturase Cpo_CPRQ. Strain ST6629 is a Y. lipolytica strain engineered for decreased fatty alcohols degradation and storage lipid accumulation (Holkenbrink et al., 2020).

The strain was cultivated, extracted and analyzed as described in example 4, with the exception that for analysis of the formed fatty alcohols, six vials (a 1.25 mL) were combined and harvested by centrifugation for 5 min at 4° C. and 3,000 g. The concentrations of fatty alcohols were calculated based on the internal standard.

Strain ST8494, combining the expression of the desaturase CpoCPRQ and fatty alcohol reductase HarFAR, in a strain engineered for lower fatty alcohol degradation showed the production of 4.4 mg/L E8,E10-C12:OH (Table 6).

TABLE 6
Concentrations of fatty alcohols in strain ST8494.

| Strain | E9IZ9-C12:OH (mg/L) | E8,E10-C12:OH (mg/L) |
|---|---|---|
| ST8494 | 20.1 | 4.4 |

### Example 6: Increased Production of E8,E10-C12:Me and E8,E10-C12:OH in Y. lipolytica

Strain ST8406 is derived from strain ST6629 and additionally expresses the CpoCPRQ desaturase. Strain ST9066, derived from ST8406, expresses two copies of Cpo_CPRQ. The strains were cultivated, extracted and analysed as described in example 4. The concentrations of fatty acid methyl esters and fatty alcohols were calculated based on the internal standard (Tables 7-10).

The expression of an additional copy of the desaturase Cpo_CPRQ from C. pomonella (ST9066) led to a 2.8- and 1.5-fold increase in production of E8,E10-C12:Me and E9/Z9-C12:Me, respectively (Table 7). This shows that overexpression of the desaturase can lead to an increase in production of E8,E10-C12:Me and E9/Z9-C12:Me.

TABLE 7
Concentrations of fatty acid methyl
esters in strains ST8406 and ST9066

| Strain | E9/Z9-C12:Me (mg/L) | E8,E10-C12:Me (mg/L) |
|---|---|---|
| ST8406 | 3.81 ± 0.52 | 0.43 ± 0.00 |
| ST9066 | 5.75 ± 1.12 | 1.22 ± 0.52 |

Strains ST8411 and ST8416 combining the expression of the desaturase Cpo_CPRQ from C. pomonella with either expression of the cytochrome b5 from H. armigera (HarCyb5, SEQ ID NO: 4) or with expression of hemoglobin from V. stercoraria (VHb, SEQ ID NO: 6), produced 18% and 22% more E8,E10-C12:Me, respectively, than the reference strain ST8406, only expressing the desaturase from C. pomonella. These strains also showed increased production of E9/Z9-C12:Me (Table 8). These data show that expression of a desaturase with a cytochrome b5 or with a hemoglobin can produce more E8,E10-C12:Me than a strain expressing only the desaturase.

TABLE 8
Concentrations of fatty acid methyl esters
in strains ST8406, ST8411 and ST8416

| Strain | E9/Z9-C12:Me (mg/L) | E8,E10-C12:Me (mg/L) |
|---|---|---|
| ST8406 | 1.29 ± 0.20 | 0.22 ± 0.09 |
| ST8411 | 1.60 ± 0.16 | 0.26 ± 0.03 |
| ST8416 | 1.68 ± 0.10 | 0.27 ± 0.02 |

Expression of the hemoglobin from V. stercoraria (VHb) (ST9115) in addition to the cytochrome b5 from H. armigera (HarCyb5) (strain ST8411) resulted in an additional 21% and 41% improvement in E8,E10-C12:Me and E9/Z9-C12:Me titre, respectively (Table 9). These data show that co-expression of a desaturase with a cytochrome b5 and a hemoglobin can produce more E8,E10-C12:Me and E9/Z9-C12:Me than a strain expressing only one of the three.

TABLE 9
Concentrations of fatty acid methyl esters
in strains ST8406, ST8411 and ST9115

| Strain | E9/Z9-C12:Me (mg/L) | E8,E10-C12:Me (mg/L) |
|---|---|---|
| ST8406 | 2.99 ± 0.64 | 0.34 ± 0.04 |
| ST8411 | 3.40 ± 0.57 | 0.42 ± 0.00 |
| ST9115 | 4.83 ± 0.62 | 0.51 ± 0.05 |

Strain ST9250 expressing the fatty acyl reductase from A. segetum (Ase_FAR) showed production of C12:OH, E9/Z9-C12:OH and E8,E10-C12:OH, while strain ST9249 expressing the fatty acyl reductase from T. alba (Ta_FAR, SEQ ID NO: 8) only showed production of C12:OH (Table 10).

TABLE 10
Concentrations of fatty alcohols in
strains ST9066, ST9249 and ST9250.

| | C12:OH | E9/Z9-C12:OH | E8,E10-C12:OH |
|---|---|---|---|
| Strain | (mg/L) | (mg/L) | (mg/L) |
| ST9066 | ND | ND | ND |
| ST9249 | 12.91 ± 0.71 | ND | ND |
| ST9250 | 12.37 ± 1.22 | 1.54 ± 0.52 | 0.66 ± 0.07 |

ND: not detected.

### Example 7: Increased Production of E8,E10-C12:Me in a Δelo1 Y. lipolytica Strain

The intrinsic Y. lipolytica gene ELO1 (YALI0_F06754g, SEQ ID NO: 13) was deleted in strain ST8406, leading to strain ST9060. The strains were cultivated, extracted and analysed as described in example 4. The concentrations of fatty acid methyl esters were calculated based on the internal standard (Table 11). Strain ST9060 showed a 2.2- and 1.6-fold increase in the production of E8,E10-C12:Me and E9/Z9-C12:Me, respectively, compared to strain ST8406. These data show that deletion of an elongase gene can increase production of E8,E10-C12:Me and E9/Z9-C12:Me.

TABLE 11
Concentrations of fatty acid methyl
esters in strains ST8406 and ST9060

| Strain | E9/Z9-C12:Me (mg/L) | E8,E10-C12:Me (mg/L) |
|---|---|---|
| ST8406 | 4.77 ± 0.65 | 0.63 ± 0.10 |
| ST9060 | 7.40 ± 1.93 | 1.39 ± 0.33 |

### Example 8: Increased Production of E8,E10-C12:Me in a Y. lipolytica Strain Containing a Deletion of the Gene YALI0_F14729g, YALI0_E18876g or YALI0_D03597g

The intrinsic Y. lipolytica genes YALI0_F14729g (SEQ ID NO: 19), YALI0_E18876g (SEQ ID NO: 54) and YALI0_D03597g (SEQ ID NO: 55), all encoding putative thioesterases, were deleted in strain ST8406, leading to strains ST9061, ST9062 and ST9063, respectively. The strains were cultivated, extracted and analyzed as described in example 4. The concentrations of fatty acid methyl esters were calculated based on the internal standard (Table 12). Strain ST9061 showed an 1.6- and 1.7-fold increase in the production of E8,E10-C12:Me and E9/Z9-C12:Me, respectively, compared to strain ST8406. Strain ST9062 showed an 1.2- and 1.3-fold increase in the production of E8,E10-C12:Me and E9/Z9-C12:Me, respectively, compared to strain ST8406. Strain ST9063 showed a 1.1-fold increase in the production of E8,E10-C12:Me and E9/Z9-C12:Me compared to strain ST8406. These data show that deletion of an endogenous putative thioesterase can increase production of E8,E10-C12:Me and E9/Z9-C12:Me.

TABLE 12
Concentrations of fatty acid methyl esters in
strains ST8406, ST9061, ST9062 and ST9063

| Strain | E9/Z9-C12:Me (mg/L) | E8,E10-C12:Me (mg/L) |
|---|---|---|
| ST8406 | 1.97 ± 0.12 | 0.29 ± 0.01 |
| ST9061 | 3.35 ± 0.44 | 0.46 ± 0.06 |
| ST9062 | 2.49 ± 0.34 | 0.35 ± 0.03 |
| ST9063 | 2.24 ± 0.47 | 0.31 ± 0.04 |

### Example 9: Production of E8,E10-C12:Me in Y. lipolytica Strains Containing Amino Acid Modifications in the Desaturase Cpo_CPRQ

The amino acid at position 85 in the protein Cpo_CPRQ was modified from serine (S) to alanine (A) in strain ST8406, leading to strain ST9072. The strains were cultivated, extracted and analysed as described in example 4. The concentrations of fatty acid methyl esters were calculated based on the internal standard (Table 13).

Strain ST9072, expressing Cpo_CPRQ_S85A, showed a 213% increased production of E8,E10-C12:Me compared to strain ST8406. These data show that Cpo_CPRQ can be engineered to increase production of E8,E10-C12:Me and E9/Z9-C12:Me.

TABLE 13
Concentrations of fatty acid methyl
esters in strains ST8406 and ST9072

| Strain | E9/Z9-C12:Me (mg/L) | E8,E10-C12:Me (mg/L) |
|---|---|---|
| ST8406 | 4.74 ± 0.26 | 0.75 ± 0.02 |
| ST9072 | 9.37 ± 1.56 | 1.60 ± 0.16 |

### Example 10: Production of E8,E10-C12:Me and E8,E10-C12:OH in Y. lipolytica Strains Containing an Amino Acid Modification in the Desaturase Cpo_CPRQ (S85A) in Combination with Other Beneficial Modifications

Strain ST9278, derived from ST9060, contains two copies of Cpo_CPRQ as well as ELO1 deletion. Strain ST9279, derived from ST9060, contains one copy of Cpo_CPRQ, one copy of Cpo_CPRQ_S85A as well as ELO1 deletion.

The strains were cultivated, extracted and analysed as described in example 4. The concentrations of fatty acid methyl esters were calculated based on the internal standard (Table 14).

Strain ST9278, expressing two copies of Cpo_CPRQ and having a deletion in the ELO1 gene, showed a lower production of E9/Z9-C12:Me and E8,E10-C12:Me compared to strain ST9279, expressing one copy of Cpo_CPRQ, one copy of Cpo_CPRQ_S85A and having a deletion in the ELO1 gene.

TABLE 14
Concentrations of fatty acid methyl esters
in strains ST9060, ST9278, ST9279

| Strain | E9/Z9-C12:Me (mg/L) | E8,E10-C12:Me (mg/L) |
|---|---|---|
| ST9060 | 12.63 ± 2.07 | 2.07 ± 0.10 |
| ST9278 | 20.70 ± 2.46 | 5.47 ± 0.74 |
| ST9279 | 21.91 ± 4.24 | 6.15 ± 1.62 |

Strain ST9355, derived from ST9279, expresses VHb and HarCyb5 in addition to other modifications. Strain ST9356, derived from ST9355, expresses HarCyb5 and HarCyb5 reductase (SEQ ID NO: 24) in addition to other modifications. Strain ST9357, derived from ST9356, contains a deletion of the intrinsic Y. lipolytica gene YALI0_F14729g in addition to other modifications. Strain ST9358, derived from ST9357, expresses Ase_FAR in addition to other modifications. Strain ST9387, derived from ST9279, expresses Ase_FAR in addition to other modifications. The strains were cultivated, extracted and analysed as described in example 4. The concentrations of fatty acid methyl esters and fatty alcohols were calculated based on the internal standard (Tables 15 and 16).

TABLE 15
Concentrations of fatty acid methyl esters
in strains ST9279, ST9355, ST9356

| Strain | E9/Z9-C12:Me (mg/L) | E8,E10-C12:Me (mg/L) |
|---|---|---|
| ST9279 | 12.6 ± 1.9 | 5.4 ± 0.7 |
| ST9355 | 11.9 ± 0.7 | 6.4 ± 0.5 |
| ST9356 | 12.8 ± 1.5 | 7.3 ± 0.1 |
| ST9357 | 11.3 ± 0.1 | 7.2 ± 0.2 |
| ST9358 | 11.1 ± 1.5 | 6.0 ± 1.2 |
| ST9387 | 11.0 ± 3.2 | 4.0 ± 0.7 |

TABLE 16
Concentrations of fatty alcohols in strains ST9279,
ST9355, ST9356, ST9357, ST9358 and ST9387

| | C12:OH | E9/Z9-C12:OH | E8,E10-C12:OH |
|---|---|---|---|
| Strain | (mg/L) | (mg/L) | (mg/L) |
| ST9279 | 0 ± 0 | 0 ± 0 | 0 ± 0 |
| ST9355 | 0 ± 0 | 0 ± 0 | 0 ± 0 |
| ST9356 | 0 ± 0 | 0 ± 0 | 0 ± 0 |
| ST9357 | 0 ± 0 | 0 ± 0 | 0 ± 0 |
| ST9358 | 41.41 ± 0.34 | 4.73 ± 0.34 | 1.98 ± 0.01 |
| ST9387 | 50.00 ± 1.10 | 7.59 ± 0.52 | 1.62 ± 0.29 |

These data show that beneficial modifications can be combined to achieve higher titres of E8,E10-C12:Me and E9/Z9-C12:Me as well as E8,E10-C12:OH and E9/Z9-C12:OH.

### Example 11: Production of E8,E10-C12:OH in Strains Containing Amino Acid Modifications in the Reductase Ase_FAR

The amino acid at position 198 in the protein Ase_FAR is modified from threonine (T) to alanine (A) in strain ST9250, leading to strain ST9335. The amino acid at position 423 in the protein Ase_FAR is modified from serine (S) to alanine (A) in strain ST9250, leading to strain ST9336. The strains are cultivated, extracted and analyzed as described in example 4. The concentrations of fatty alcohols are calculated based on the internal standard.

### Example 12: Production of E8,E10-C12:OH in Y. lipolytica Strains Containing Amino Acid Modifications in the Fatty Acid Synthase 1 (FAS1) and Fatty Acid Synthase 2 (FA S2)

The amino acid at position 1220 in the FAS2 (SEQ ID NO: 18) of Y. lipolytica is modified from isoleucine (I) to phenylalanine (F) in strain ST9387, leading to strain ST9388. The amino acid at position 1220 in the FAS2 of Y. lipolytica is modified from isoleucine (I) to tryptophan (W) in strain ST9387, leading to strain ST9420. The amino acid at position 123 in the FAS1 (SEQ ID NO: 16) of Y. lipolytica is modified from leucine (L) to valine (V) in strain ST9420, leading to strain ST9421. The strains are cultivated, extracted and analyzed as described in example 4, except from that no methyl dodecanoate is added to the production medium. The concentrations of fatty alcohols are calculated based on the internal standard.

### Example 13: Production of E8,E10-C12:OH in Y. lipolytica Strains Containing an Amino Acid Modification in FAS2 of Y. lipolytica (FAS2(I1220F)) as Well as a Thioesterase from E. coli for C12 Fatty Acid Formation

Strain ST9397 expresses a fusion of a truncated version of FAS1 from Y. lipolytica and a truncated version of the thioesterase TesA from E. coli (Xu et al., 2016) (SEQ ID NO: 59). Strain ST9397 is transformed with a plasmid containing the fatty acyl-CoA synthase from Y. lipolytica, leading to strain ST9398. The strains are cultivated, extracted and analyzed as described in example 4, with the exception that glass tubes were used and that fatty alcohols were extracted from the total broth. The concentrations of fatty alcohols are calculated based on the internal standard (Table 17).

The expression of the fatty acyl-CoA synthase from Y. lipolytica did not significantly affect the production of E8,E10-12:OH.

TABLE 17
Concentrations of E9/Z9-12:OH and E8,E10-
12:OH in strain ST9397 and ST9398

| Strain | E9/Z9-12:OH (mg/L) | E8,E10-C12:OH (mg/L) |
|---|---|---|
| ST9397 | 0.2 ± 0 | 0.1 ± 0 |
| ST9398 | 0.2 ± 0 | 0.1 ± 0 |

### Example 14: Production of E8,E10-C12:OH Via Chain Shortening in Peroxisomes in Y. lipolytica

To increase the amount of C12:CoA precursor in strain ST9395, the five endogenous peroxisomal oxidases of Y. lipolytica: POX1, POX2, POX3, POX4 and POX5 (YAL10_E32835g, YALI0_F10857g, YALI0_E32835g, YALI0_E27654g, YALI0_E27654g, respectively) are deleted and instead a heterologous peroxisomal oxidase as for example Cma_POX from Cucurbita maxima (SEQ ID NO: 47) is expressed.

To increase the amount of Δ9-12:CoA precursor, the above mentioned strain expresses additionally a Δ11-14 desaturase as for example CroZ11 from Choristoneura rosaceana (SEQ ID NO: 63) or CpaE11 from Choristoneura parallela (SEQ ID NO: 65). By this, Z/E11-14:CoA is produced and is shortened to Z/E9-12:CoA, which is then further converted to E8,E10-C12:Me by desaturase Cpo_CPRQ (SEQ ID NO:1).

The strains are cultivated, extracted and analysed as described in example 4. The cultures of strains ST9600, ST9607 and ST9616 are supplemented with methyl myristate. The concentrations of fatty alcohols are calculated based on the internal standard.

### Example 15: Production of E8,E10-C12:Me and E8,E10-C12:OH in Saccharomyces cerevisiae

The desaturase gene Cpo_CPRQ was amplified from cDNA of Cydia pomonella pheromone gland tissue using primer attB1_Cpo_CPRQ_F and attB1_Cpo_CPRQ_R.

The PCR product was separated by agarose gel electrophoresis and purified using the Wizard SV Gel and PCR Clean up system (Promega Biotech AB, Sweden). The purified DNA was cloned into the pDONR221 vector by the Gateway Cloning technology (Life technologies). The resulting vector was confirmed by Sanger sequencing and the gene was subcloned into vector pYEX-CHT (Patel et al, 2003), which then was transformed into a Saccharomyces cerevisiae strain deficient of OLE1 and ELO1 (MATa elo1::HIS3 ole1::LEU2 ade2 his3 leu2 ura3) (Schneiter et al., 2000). For selection of positive transformants, the cells were cultivated on synthetic complete medium containing 0.7% YNB (with ammonium sulfate), drop-out medium lacking uracil and leucine (Formedium LTD, England), 2% glucose, 1% tergitol (type Nonidet NP-40, Sigma-Aldrich, Sweden), 0.01% adenine (Sigma-Aldrich, Sweden) and 0.5 mM oleic acid (Sigma-Aldrich, Sweden). After incubation of the plates for four days at 30° C., individual colonies were inoculated into 10 ml selective medium. The cultures were incubated at 30° C. for 48 h and used to inoculate 10 ml of selective medium containing 2 mM CuSO₄ with supplementation of 0.5 mM fatty acid methyl ester precursor to an OD600 of 0.4. After 48 h of incubation the cells were harvested by centrifugation at 3000 rpm. The media supernatant was discarded and total lipids were extracted using 3.75 ml of methanol/chloroform (2:1, v/v), in a glass tube. One ml of HAc (0.15 M) and 1.25 ml of water were added and the tubes were vortexed. The tubes were centrifuged at 2000 rpm for 2 min and the bottom chloroform phase was transferred to a fresh glass tube. To convert the lipids into fatty acid methyl esters (FAME), the solvent was evaporated under nitrogen flow. One ml of 2% sulfuric acid in methanol was added, the suspension was vortexed and incubated at 90° C. for 1 h. Afterwards 1 ml of water was added, mixed and 1 ml of hexane was used to extract the FAMEs. The samples were subjected to GC-MS analysis on a Hewlett Packard 6890 GC coupled to a mass selective detector HP5973. The GC was equipped with a HP-88 column (30 m×0.25 mm×0.25 μm) and helium was used as carrier gas (average velocity: 33 ms). The MS was operated in electron impact mode (70 eV), and the injector was configured in splitless mode at 220° C. The oven temperature was set to 80° C. for 1 min, then increased at a rate of 10° C./min up to 210° C., followed by a hold of 210° C. for 15 min, and then increased at a rate of 10° C./min up to 230° C. followed by a hold at 230° C. for 20 min. As a reference standard, E8,E10-12:OAc was purchased from Bedoukian, USA and converted to the corresponding alcohol by hydrolysis using a 0.5 M solution of KOH in methanol. Fatty alcohols were oxidized to the corresponding acid with pyridinium dichromate in dimethylformamide as described (Bjostad and Roelofs, 1984)

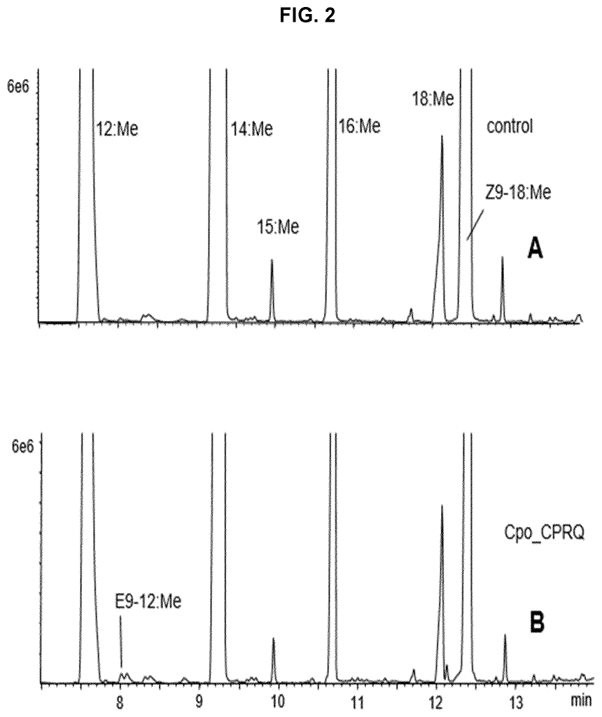

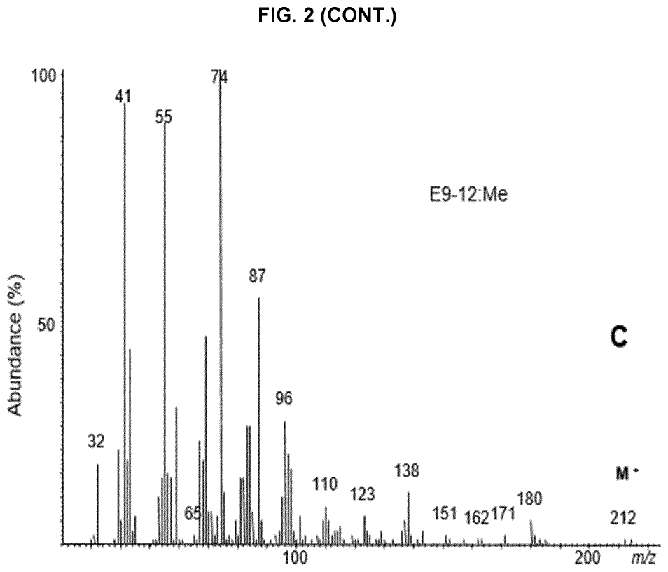

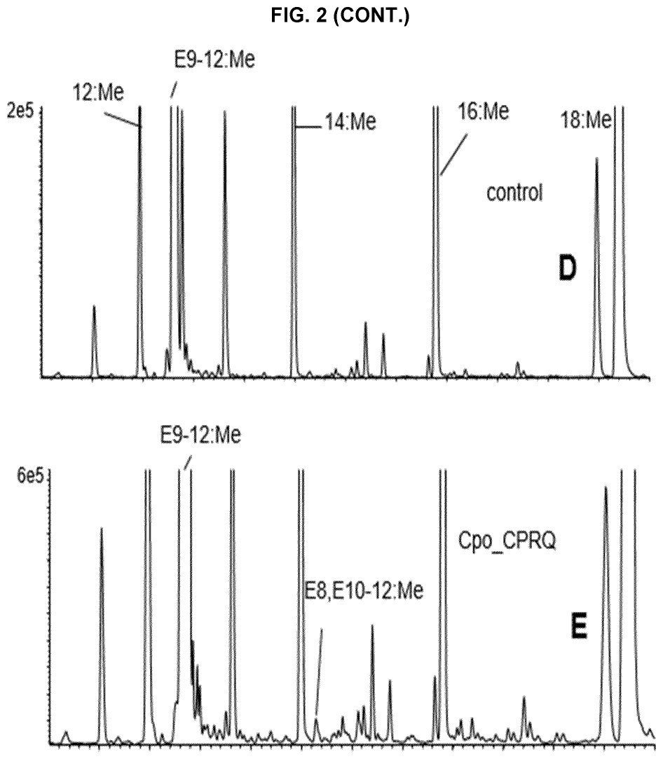

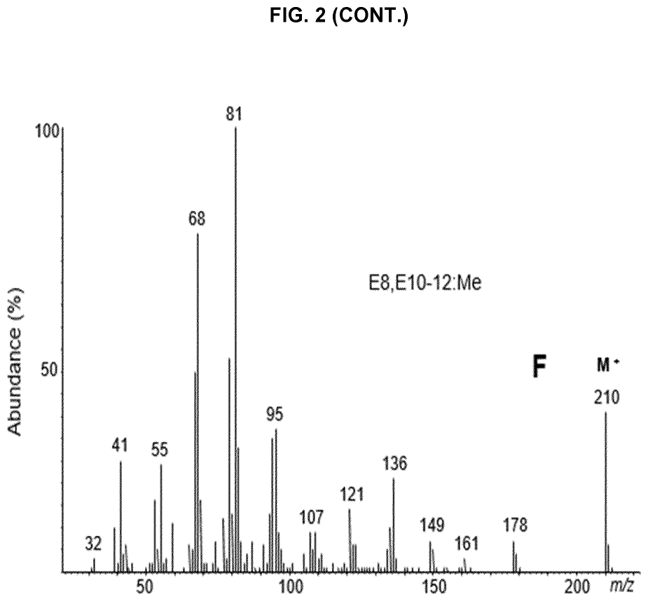

The chromatograms in FIG. 2 show that E9-12:Me and E8,E10-12:Me can be produced from 12:Me and E9-12:Me, respectively, in the S. cerevisiae strains expressing Cpo_CPRQ.

### Example 16: Production of E8,E10-C12:Me by Cpo_SPTQ, Cpo_NPVE and Cpo_CPRQ in Y. lipolytica

Strain ST10136, derived from ST6629, expresses one copy of Cpo_SPTQ. Strain ST10137, derived from ST6629, expresses one copy of Cpo_NPVE. Strain ST9064, derived from ST8406, expresses one copy of Cpo_CPRQ and one copy of Cpo_SPTQ. Strain ST9065, derived from ST8406, expresses one copy of Cpo_CPRQ and one copy of Cpo_NPVE. Strain ST9066, derived from ST8406, expresses two copies of Cpo_CPRQ. Strain ST10138, derived from ST9065, expresses one copy of Cpo_CPRQ, one copy of Cpo_NPVE and one copy of Cpo_SPTQ.

The strains were cultivated, extracted and analyzed as described in example 4. The concentrations of fatty acid methyl esters were calculated based on the internal standard (Table 18).

The expression of Cpo_SPTQ (ST10136) did not lead to the production of E9-C12:Me, Z9-C12:Me or E8,E10-C12:Me. The expression of Cpo_NPVE (ST10137) led to the production of E9-C12:Me and Z9-C12:Me, but not E8,E10-C12:Me. The additional expression of Cpo_SPTQ or Cpo_NPVE in ST8406 (ST9064 and ST9065, respectively) did not lead to an increase in E8,E10-C12:Me. The expression of an additional copy of Cpo_CPRQ in ST8406 (ST9066) led to a 2.8- and 2.1-fold increase in production of E8,E10-C12:Me and E9/Z9-C12:Me, respectively. The combined expression of Cpo_CPRQ, Cpo_SPTQ and Cpo_NPVE (ST10138) did not lead to an increase in E8,E10-C12:Me compared to ST8406. This shows that only the expression of Cpo_CPRQ leads to the production of E8,E10-C12:Me.

TABLE 18
Concentrations of fatty acid methyl esters in strains ST10136,
ST10137, ST8406, ST9064, ST9065, ST9066 and ST10138

| | E9-C12:Me | Z9-C12:Me | E8,E10-C12:Me |
|---|---|---|---|
| Strain | (mg/L) | (mg/L) | (mg/L) |
| ST10136 | 0.00 ± 0.00 | 0.00 ± 0.00 | 0.00 ± 0.00 |
| ST10137 | 2.06 ± 0.89 | 3.82 ± 1.80 | 0.00 ± 0.00 |
| ST8406 | 5.02 ± 0.88 | 0.33 ± 0.03 | 0.66 ± 0.15 |
| ST9064 | 4.42 ± 0.00 | 0.29 ± 0.00 | 0.63 ± 0.00 |
| ST9065 | 6.04 ± 0.22 | 2.29 ± 0.10 | 0.86 ± 0.10 |
| ST9066 | 10.55 ± 0.07 | 0.58 ± 0.04 | 1.87 ± 0.06 |
| ST10138 | 6.38 ± 0.99 | 2.66 ± 0.41 | 0.76 ± 0.06 |

### Example 17: Production of E8,E10-C12:OH by Expressing Multiple Copies of Biosynthetic Enzymes

Strain ST9358 is explained in Example 10. Strain ST9495 derives from strain ST9357 (described in Example 10) and expresses additional gene copies of the desaturase Cpo_CPRQ and the fatty acyl reductase Ase_FAR. The strains are cultivated, extracted and analyzed as described in example 4, with the exception that glass tubes were used and that fatty alcohols were extracted from the total broth. The concentrations of fatty alcohols are calculated based on the internal standard.

Table 19 shows that additional gene copies of Cpo_CPRQ and Ase_FAR can increase the production of E8,E10-12:OH to 7.1 mg/L.

TABLE 19
Concentrations of fatty alcohols in strains ST9358 and ST9495

| Strain | E9-C12:OH (mg/L) | E8,E10-C12:OH (mg/L) |
|---|---|---|
| ST9358 | 0.3 ± 0.0 | 0.1 ± 0.0 |
| ST9495 | 22.6 ± 4.5 | 7.1 ± 1.5 |

### Example 18: Codlemone Production by Expression of Various Fatty Acyl Reductases

Strains ST9358 and ST9623 are derived from strain ST9357. They additionally express the fatty acyl reductase from Agrotis segetum and Agrotis ipsilon, respectively. The strains were cultivated, extracted and analysed as described in example 4.

The results in table 20 show that both fatty acyl reductases are able to produce E9-C12:OH and E8,E10-C12:OH.

TABLE 20
Concentrations of fatty alcohols in
strains ST9357, ST9358 and ST9623

| Strain | E9-C12:OH (mg/L) | E8,E10-C12:OH (mg/L) |
|---|---|---|
| ST9357 | 0 ± 0 | 0 ± 0 |
| ST9358 | 1.1 ± 1.2 | 0.5 ± 0.5 |
| ST9623 | 1.9 ± 0.3 | 0.6 ± 0.1 |

### Sequences

| SEQ ID NO | Description | Organism |
|---|---|---|
| SEQ ID NO: 1 | DNA | Cydia pomonella |
| | CPO_CPRQ desaturase (AHW98354) codon | |
| | optimized for Y. lipolytica; mRNA-coding sequence | |
| SEQ ID NO: 2 | PRT | Cydia pomonella |
| | CPO_CPRQ desaturase (AHW98354) | |
| SEQ ID NO: 3 | DNA | Helicoverpa armigera |
| | cytochrome b5 (AAC33731) codon optimized for | |
| | Y. lipolytica; mRNA-coding sequence | |
| SEQ ID NO: 4 | PRT | Helicoverpa armigera |
| | cytochrome b5 (AAC33731) | |
| SEQ ID NO: 5 | DNA | Vitreoscilla stercoraria |
| | hemoglobin (AAT01097) codon optimized for | |
| | Y. lipolytica; mRNA-coding sequence | |
| SEQ ID NO: 6 | PRT | Vitreoscilla stercoraria |
| | hemoglobin (AAT01097) | |
| SEQ ID NO: 7 | DNA | Tyto alba |
| | fatty acyl reductase (NP_001289627) codon | |
| | optimized for Y. lipolytica; mRNA-coding sequence | |
| SEQ ID NO: 8 | PRT | Tyto alba |
| | fatty acyl reductase (NP_001289627) | |
| SEQ ID NO: 9 | DNA | Agrotis segetum |
| | fatty acyl reductase (AGP26039) codon optimized | |
| | for Y. lipolytica; mRNA-coding sequence | |
| SEQ ID NO: 10 | PRT | Agrotis segetum |
| | fatty acyl reductase (AGP26039) | |
| SEQ ID NO: 11 | DNA | Helicoverpa armigera |
| | fatty acyl reductase (ATJ44471) codon optimized | |
| | for Y. lipolytica; mRNA-coding sequence | |
| SEQ ID NO: 12 | PRT | Helicoverpa armigera |
| | fatty acyl reductase (ATJ44471) | |
| SEQ ID NO: 13 | DNA | Yarrowia lipolytica |
| | fatty acid elongase 1 (ELO1, YALI0_F06754g) | |
| SEQ ID NO: 14 | PRT | Yarrowia lipolytica |
| | fatty acid elongase 1 (ELO1, XP_505094) | |
| SEQ ID NO: 15 | DNA | Yarrowia lipolytica |
| | fatty acid synthase 1 (FAS1, YALI0_B15059g) | |
| SEQ ID NO: 16 | PRT | Yarrowia lipolytica |
| | fatty acid synthase 1 (Fas1, XP_500912) | |
| SEQ ID NO: 17 | DNA | Yarrowia lipolytica |
| | fatty acid synthase 2 (FAS2, YALI0_B19382g) | |
| SEQ ID NO: 18 | PRT | Yarrowia lipolytica |
| | fatty acid synthase 2 (Fas2, XP_501096) | |
| SEQ ID NO: 19 | DNA | Yarrowia lipolytica |
| | YALI0_F14729g | |
| SEQ ID NO: 20 | PRT | Yarrowia lipolytica |
| | XP_505426 | |
| SEQ ID NO: 21 | DNA | Yarrowia lipolytica |
| | fatty acyl-CoA synthase (YALI0_D17864g) | |
| SEQ ID NO: 22 | PRT | Yarrowia lipolytica |
| | fatty acyl-CoA synthase (XP_502959) | |
| SEQ ID NO: 23 | DNA | Helicoverpa armigera |
| | cytochrome b5 reductase (XP_021183830) codon | |
| | optimized for Y. lipolytica; mRNA-coding sequence | |
| SEQ ID NO: 24 | PRT | Helicoverpa armigera |
| | cytochrome b5 reductase (XP_021183830) | |
| SEQ ID NO: 25 | DNA | Escherichia coli |
| | thioesterase (AAB40248) codon optimized for | |
| | Y. lipolytica; mRNA-coding sequence | |
| SEQ ID NO: 26 | PRT | Escherichia coli |
| | thioesterase (AAB40248) | |
| SEQ ID NO: 27 | DNA | Yarrowia lipolytica |
| | TEFintron promoter | |
| SEQ ID NO: 28 | DNA | Yarrowia lipolytica |
| | EXP promoter | |
| SEQ ID NO: 29 | DNA | Yarrowia lipolytica |
| | YEF3 promoter | |
| SEQ ID NO: 30 | DNA | Saccharomyces |
| | Peroxisomal oxidase POX1 | cerevisiae |
| SEQ ID NO: 31 | PRT | Saccharomyces |
| | Peroxisomal oxidase POX1 | cerevisiae |
| SEQ ID NO: 32 | DNA | Cuphea palustris |
| | codon-optimized nucleotide sequence of | |
| | thioesterase CpFATB2 | |
| SEQ ID NO: 33 | PRT | Cuphea palustris |
| | thioesterase CpFATB2 | |
| SEQ ID NO: 34 | DNA | Cinnamomum |
| | codon-optimised nucleotide sequence of | camphora |
| | thioesterase CcFatB1 | |
| SEQ ID NO: 35 | PRT | Cinnamomum |
| | thioesterase CcFatB1 | camphora |
| SEQ ID NO: 36 | DNA | Saccharomyces |
| | Codon-optimised sequence of acetyltransferase | cerevisiae |
| | Atf1 | |
| SEQ ID NO: 37 | PRT | Saccharomyces |
| | Acetyltransferase Atf1 | cerevisiae |
| SEQ ID NO: 38 | DNA | Agrotis segetum |
| | Codon-optimised sequence of peroxisomal | |
| | oxidase | |
| SEQ ID NO: 39 | PRT | Agrotis segetum |
| | Peroxisomal oxidase | |
| SEQ ID NO: 40 | DNA | Arabidopsis thaliana |
| | Codon-optimised sequence of peroxisomal | |
| | oxidase 1 | |
| SEQ ID NO: 41 | PRT | Arabidopsis thaliana |
| | Peroxisomal oxidase 1 | |
| SEQ ID NO: 42 | DNA | Arabidopsis thaliana |
| | Codon-optimised sequence of peroxisomal | |
| | oxidase 2 | |
| SEQ ID NO: 43 | PRT | Arabidopsis thaliana |
| | Peroxisomal oxidase 2 | |
| SEQ ID NO: 44 | DNA | Aspergillus nidulans |
| | Codon-optimised sequence of peroxisomal | |
| | oxidase | |
| SEQ ID NO: 45 | PRT | Aspergillus nidulans |
| | Peroxisomal oxidase | |
| SEQ ID NO: 46 | DNA | Cucurbita maxima |
| | Codon-optimised sequence of peroxisomal | |
| | oxidase | |
| SEQ ID NO: 47 | PRT | Cucurbita maxima |
| | Peroxisomal oxidase | |
| SEQ ID NO: 48 | DNA | Homo sapiens |
| | Codon-optimised sequence of peroxisomal | |
| | oxidase | |
| SEQ ID NO: 49 | PRT | Homo sapiens |
| | Peroxisomal oxidase | |
| SEQ ID NO: 50 | DNA | Paenarthrobacter |
| | Codon-optimised sequence of peroxisomal | ureafaciens |
| | oxidase | |
| SEQ ID NO: 51 | PRT | Paenarthrobacter |
| | Peroxisomal oxidase | ureafaciens |
| SEQ ID NO: 52 | DNA | Rattus norvegicus |
| | Codon-optimised sequence of peroxisomal | |
| | oxidase | |
| SEQ ID NO: 53 | PRT | Rattus norvegicus |
| | Peroxisomal oxidase | |
| SEQ ID NO: 54 | DNA | Yarrowia lipolytica |
| | YALI0_E18876g | |
| SEQ ID NO: 55 | DNA | Yarrowia lipolytica |
| | YALI0_D03597g | |
| SEQ ID NO: 56 | DNA | Cuphea hookeriana |
| | Thioesterase | |
| SEQ ID NO: 57 | PRT | Cuphea hookeriana |
| | Thioesterase | |
| SEQ ID NO: 58 | DNA | |
| | Fusion of a truncated version of FAS1 from | |
| | Y. lipolytica and a truncated version of the | |
| | thioesterase TesA from E. coli | |
| SEQ ID NO: 59 | PRT | |
| | Fusion of a truncated version of FAS1 from | |
| | Y. lipolytica and a truncated version of the | |
| | thioesterase TesA from E. coli | |
| SEQ ID NO: 60 | DNA | Agrotis ipsilon |
| | Aip_FAR | |
| SEQ ID NO: 61 | PRT | Agrotis ipsilon |
| | Aip_FAR | |
| SEQ ID NO: 62 | DNA | Choristoneura |
| | CroZ11 desaturase | rosaceana |
| SEQ ID NO: 63 | PRT | Choristoneura |
| | CroZ11 desaturase | rosaceana |
| SEQ ID NO: 64 | DNA | Choristoneura parallela |
| | CpaE11 desaturase | |
| SEQ ID NO: 65 | PRT | Choristoneura parallela |
| | CpaE11 desaturase | |
| SEQ ID NO: 66 | DNA | Cydia pomonella |
| | Cpo_NPVE, codon-optimised for Y. lipolytica | |
| SEQ ID NO: 67 | PRT | Cydia pomonella |
| | Cpo_NPVE | |
| SEQ ID NO: 68 | DNA | Cydia pomonella |
| | Cpo_SPTQ, codon-optimised for Y. lipolytica | |
| SEQ ID NO: 69 | PRT | Cydia pomonella |
| | Cpo_SPTQ | |
| SEQ ID NO: 70 | DNA, codon-optimized for S. cerevisiae; mRNA- | Heliothis subflexa |
| | coding sequence | |
| | Hs_FAR | |
| SEQ ID NO: 71 | PRT | Heliothis subflexa |
| | Hs_FAR | |
| SEQ ID NO: 72 | DNA, codon-optimized for S. cerevisiae; mRNA- | Helicoverpa assulta |
| | coding sequence | |
| | Has_FAR | |
| SEQ ID NO: 73 | PRT | Helicoverpa assulta |
| | Has_FAR | |
| SEQ ID NO: 74 | DNA | Helicoverpa virescens |
| | Hv_FAR | |
| SEQ ID NO: 75 | PRT | Helicoverpa virescens |
| | Hv_FAR | |
| SEQ ID NO: 76 | PRT | Cydia pomonella |
| | Cpo_FAR | |
| SEQ ID NO: 77 | PRT | Grapholita molesta |
| | Gmo_CPRQ | |
| SEQ ID NO: 78 | DNA | Grapholita molesta |
| | Gmo_CPRQ | |
| SEQ ID NO: 79 | DNA | Grapholita molesta |
| | Gmo_CPRQ, modified | |
| SEQ ID NO: 80 | DNA | Cydia pomonella |
| | Cpo_FAR | |

### REFERENCES

Bjostadt B L, Roelofs L W, 1984. Sex pheromone biosynthetic precursors in Bombyx mori. Insect Biochem. 14, 275-278
Borodina I, Holkenbrink C, Dam M, Löfstedt C, Ding B, Wang H-L. Production of desaturated fatty alcohols and desaturated fatty acyl acetates in yeast. 2018
Ding B-J. On the way of making plants smell like moths: a synthetic approach. 2014
Ferrell, Yao, 1972. Reductive and oxidative synthesis of saturated and unsaturated fatty aldehydes, J Lipid Res. 13(1):23-6.
Holkenbrink C, Dam M I, Kildegaard K R, Beder J, Dahlin J, Doménech Belda D, et al. EasyCloneYALI: CRISPR/Cas9-based synthetic toolbox for engineering of the yeast Yarrowia lipolytica. Biotechnol J. 2017; 1700543:1-8
Holkenbrink C, Ding B J, Wang H L, Dam M I, Petkevicius K, Kildegaard K R, Wenning L, Sinkwitz C, Lorántfy B, Koutsoumpeli E, França L, Pires M, Bernardi C, Urrutia W, Mafra-Neto A, Ferreira B S, Raptopoulos D, Konstantopoulou M, Löfstedt C, Borodina I. Production of moth sex pheromones for pest control by yeast fermentation. Metab Eng. 2020 November; 62:312-321. doi: 10.1016/j.ymben.2020.10.001. Epub 2020 Oct. 9.lwama R, Kobayashi S, Ohta A, Horiuchi H, Fukuda R. Fatty aldehyde dehydrogenase multigene family involved in the assimilation of n-alkanes in Yarrowia lipolytica. J Biol Chem. 2014; 289(48):33275-33286. doi:10.1074/jbc.M114.596890
Iwama R, Kobayashi S, Ohta A, Horiuchi H, Fukuda R. Alcohol dehydrogenases and an alcohol oxidase involved in the assimilation of exogenous fatty alcohols in Yarrowia lipolytica. FEMS Yeast Res. 2015 May; 15(3):fov014. doi: 10.1093/femsyr/fov014. Epub 2015 Mar. 23. PMID: 25805841.
Lamb D C, Kelly D E, Manning N J, Kaderbhai M A, Kelly S L. Biodiversity of the P450 catalytic cycle: yeast cytochrome b5/NADH cytochrome b5 reductase complex efficiently drives the entire sterol 14-demethylation (CYP51) reaction. FEBS Lett. 1999 Dec. 3; 462(3):283-8. doi: 10.1016/s0014-5793(99)01548-3. PMID: 10622712.
Li, Zhang, 2009. An environmentally benign TEMPO-catalyzed efficient alcohol oxidation system with a recyclable hypervalent iodine(III) reagent andilts facile preparation. Synthesis, 1163-1169a.
Löfstedt C, Bengtsson M. Sex pheromone biosynthesis of (E,E)-8,10-dodecadienol in codling moth Cydia pomonella involves E9 desaturation. J Chem Ecol. 1988; 14:903-15 Meyer, Schreiber, 1994. Acceleration of the Dess-Martin oxidation by water J. Org. Chem., 59, 7549-7552
Nancolas B, Bull I D, Stenner R, Dufour V, Curnow P. Saccharomyces cerevisiae Atf1p is an alcohol acetyltransferase and a thioesterase in vitro. Yeast. 2017; 34(6):239-251. doi:10.1002/yea.3229
Okada, Asawa, Sugiyama, Kirihara, Iwai, Kimura, 2014. Sodium hypochlorite pentahydrate (NaOCl·5H2O) crystals as an extraordinary oxidant for primary and secondary alcohols. Synlett, 25, 596-598
Patel O, Fernley R, MacReadie I. 2003. Saccharomyces cerevisiae expression vectors with thrombin-cleavable N- and C-terminal 6×(His)tags. Biotechnol. Lett 25: 331-334.
Schneiter R, Tatzer V, Gogg G, Leitner E, Kohlwein S D, 2000. Elo1p-dependent carboxy-terminal elongation of C14:Δ9 to C16:Δ11 fatty acids in Saccharomyces cerevisiae. J. Bacteriol. 182: 3655-3660Schneiter R, Tatzer V, Gogg G, Leitner E, Kohlwein SD. Elo1p-dependent carboxy-terminal elongation of C14:1Delta(9) to C16:1Delta(11) fatty acids in Saccharomyces cerevisiae. J Bacteriol. 2000; 182(13):3655-3660. doi:10.1128/jb.182.13.3655-3660.2000 Tamura, Aoyama, Takido, Kodomari, 2012. Novel [4-Hydroxy-TEMPO+NaCl]/SiO2 as a reusable catalyst for aerobic oxidation of alcohols to carbonyls. Synlett, 23, 1397-1407.
Xu P, Qiao K, Ahn W S, Stephanopoulos G. Engineering Yarrowia lipolytica as a platform for synthesis of drop-in transportation fuels and oleochemicals. Proc Natl Acad Sci. 2016; 113:10848-53
Yadav, Reddy, Basak, Narsaiah, 2004. Recyclable 2nd generation ionic liquids as green solvents for the oxidation of alcohols with hypervalent iodine reagents, Tetrahedron, 60, 2131-2135

### ITEMS

1. A yeast cell capable of producing E8,E10-dodecadienyl coenzyme A and optionally E8,E10-dodecadien-1-ol, said yeast cell expressing at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA), optionally wherein the yeast cell belongs to a genus selected from Blakeslea, Candida, Cryptococcus, Cunninghamella, Lipomyces, Mortierella, Mucor, Phycomyces, Pythium, Rhodosporidium, Rhodotorula, Trichosporon, Saccharomyces and Yarrowia, optionally wherein the yeast cell belongs to a species selected from Blakeslea trispora, Candida pulcherrima, C. revkaufi, C. tropicalis, Cryptococcus curvatus, Cunninghamella echinulata, C. elegans, C. japonica, Lipomyces starkeyi, L. lipoferus, Mortierella alpina, M. isabellina, M. ramanniana, M. vinacea, Mucor circinelloides, Phycomyces blakesleanus, Pythium irregulare, Rhodosporidium toruloides, Rhodotorula glutinis, R. gracilis, R. graminis, R. mucilaginosa, R. pinicola, Trichosporon pullans, T. cutaneum, Saccharomyces cerevisiae and Yarrowia lipolytica, preferably the yeast cell is a Yarrowia lipolytica cell or a Saccharomyces cerevisiae cell.
2. A yeast cell capable of producing E8,E10-dodecadienyl coenzyme A and optionally E8,E10-dodecadien-1-ol, said yeast cell expressing at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA).
3. The yeast cell according to any one of the preceding items, wherein the yeast cell is capable of producing E8,E10-dodecadien-1-ol, said yeast cell further expressing at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol.
4. A yeast cell capable of producing E8,E10-dodecadien-1-ol, said yeast cell expressing:
   i) At least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
   ii) At least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol.
5. The yeast cell according to any one of the preceding items, wherein the yeast cell belongs to a genus selected from Blakeslea, Candida, Cryptococcus, Cunninghamella, Lipomyces, Mortierella, Mucor, Phycomyces, Pythium, Rhodosporidium, Rhodotorula, Trichosporon, Saccharomyces and Yarrowia.
6. The yeast cell according to any one of the preceding items, wherein the yeast cell belongs to a species selected from Blakeslea trispora, Candida pulcherrima, C. revkaufi, C. tropicalis, Cryptococcus curvatus, Cunninghamella echinulata, C. elegans, C. japonica, Lipomyces starkeyi, L. lipoferus, Mortierella alpina, M. isabellina, M. ramanniana, M. vinacea, Mucor circinelloides, Phycomyces blakesleanus, Pythium irregulare, Rhodosporidium toruloides, Rhodotorula glutinis, R. gracilis, R. graminis, R. mucilaginosa, R. pinicola, Trichosporon pullans, T. cutaneum, Saccharomyces cerevisiae and Yarrowia lipolytica.
7. The yeast cell according to any one of the preceding items, wherein the yeast cell is of the genus Yarrowia or Saccharomyces, preferably the yeast cell is a Yarrowia lipolytica cell or a Saccharomyces cerevisiae cell.
8. The yeast cell according to any one of the preceding items, wherein the at least one desaturase is Gmo_CPRQ (SEQ ID NO: 77) or Cpo_CPRQ (SEQ ID NO: 2), or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to SEQ ID NO: 77 or SEQ ID NO: 2, preferably the at least one desaturase is Cpo_CPRQ or a functional variant thereof; or wherein the at least one desaturase is at least two desaturases, wherein at least one of said two desaturases is Gmo_CPRQ (SEQ ID NO: 77) or Cpo_CPRQ (SEQ ID NO: 2), or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to SEQ ID NO: 77 or SEQ ID NO: 2, preferably the at least one desaturase is Cpo_CPRQ or a functional variant thereof, and the other desaturase is a desaturase capable of introducing at least one double bond in a fatty acyl-CoA having a carbon chain length of 12, such as a Z9-12 desaturase, preferably Cpo_NPVE (SEQ ID NO: 67) or Cpo_SPTQ (SEQ ID NO: 69) or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to SEQ ID NO: 67 or SEQ ID NO: 69.
9. The yeast cell according to any one of the preceding items, wherein the desaturase is a mutant of Cpo_CPRQ having a mutation at position 85, such as an S85A mutation.
10. The yeast cell according to any one of the preceding items, wherein the at least one heterologous desaturase is at least two different heterologous desaturases, such as Cpo_CPRQ as set forth in SEQ ID NO: 2 and a mutant of Cpo_CPRQ having a mutation at position 85 such as an S85A mutation.
11. The yeast cell according to any one of the preceding items, wherein the fatty acyl-CoA reductase is selected from the group consisting of Ase_FAR (SEQ ID NO: 10), Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12), Cpo_FAR (SEQ ID NO: 76) and functional variants thereof having at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.
12. The yeast cell according to any one of the preceding items, wherein the fatty acyl-CoA reductase is a mutant of Ase_FAR, such as having a mutation at position 198 or 413, preferably a T198A mutation or an S413A mutation.
13. The yeast cell according to any one of the preceding items, wherein the heterologous desaturase is expressed at high level.
14. The yeast cell according to any one of the preceding items, wherein the heterologous fatty acyl-CoA reductase is expressed at high level.
15. The yeast cell according to any one of the preceding items, wherein the yeast cell is further modified to increase availability of E8,E10-C12:CoA.
16. The yeast cell according to any one of the preceding items, further expressing a heterologous cytochrome b5, such as a cytochrome b5 from a Lepidotera species, such as a cytochrome b5 from Helicoverpa armigera, preferably the cytochrome b5 HarCyb5 as set forth in SEQ ID NO: 4 or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 85%, such as at least 90%, such as at least 95%, such as at least 96%, such as at least 97%, such as at least 98%, such as at least 99% homology or identity thereto.
17. The yeast cell according to any one of the preceding items, further expressing a heterologous cytochrome b5 reductase (EC 1.6.2.2), such as a cytochrome b5 reductase from a Lepidoptera species, such as Helicoverpa armigera, preferably the cytochrome b5 reductase is the cytochrome b5 reductase from Helicoverpa armigera as set forth in SEQ ID NO: 24 or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 85%, such as at least 90%, such as at least 95%, such as at least 96%, such as at least 97%, such as at least 98%, such as at least 99% homology or identity thereto.
18. The yeast cell according to any one of the preceding items, further expressing a hemoglobin, such as a hemoglobin from Vitreoscilla stercoraria, preferably the hemoglobin from Vitreoscilla stercoraria as set forth in SEQ ID NO: 6 or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 85%, such as at least 90%, such as at least 95%, such as at least 96%, such as at least 97%, such as at least 98%, such as at least 99% homology or identity thereto.
19. The yeast cell according to any one of the preceding items, further comprising a mutation of one or more genes encoding an elongase and resulting in a partial or total loss of elongase activity, such as a mutation of the ELO1 gene (SEQ ID NO: 13) resulting in a partial or total loss of Elo1 activity, preferably wherein said mutation is a deletion.
20. The yeast cell according to any one of the preceding items, further comprising a mutation of one or more genes encoding a thioesterase and resulting in a partial or total loss of thioesterase activity, such as a mutation of the YAL10_F14729g gene (SEQ ID NO: 19), a mutation of the YALI0_E18876g gene (SEQ ID NO: 54) or a mutation of YALI0_D03597g (SEQ ID NO: 55), preferably wherein said mutation is a deletion.
21. The yeast cell according to any one of the preceding items, further comprising at least one modification such as at least one mutation resulting in reduced activity of at least one of Hfd1, Hfd2, Hfd3, Hfd4, Fao1, GPAT and Pex10, or having at least one modification such as at least one mutation resulting in reduced activity of at least one protein having at least 60% homology or identity thereto, such as at least 65% homology or identity, such as at least 70% homology or identity, such as at least 75% homology or identity, such as at least 80% homology or identity, such as at least 81% homology or identity, such as at least 82% homology or identity, such as at least 83% homology or identity, such as at least 84% homology or identity, such as at least 85% homology or identity, such as at least 86% homology or identity, such as at least 87% homology or identity, such as at least 88% homology or identity, such as at least 89% homology or identity, such as at least 90% homology or identity, such as at least 91% homology or identity, such as at least 92% homology or identity, such as at least 93% homology or identity, such as at least 94% homology or identity, such as at least 95% homology or identity, such as at least 96% homology or identity, such as at least 97% homology or identity, such as at least 98% homology or identity, such as at least 99% homology or identity thereto.
22. The yeast cell according to any one of the preceding items, wherein the yeast cell further expresses a fatty acyl synthase variant having a modified ketone synthase domain, wherein said fatty acyl synthase variant is a variant of Fas1 (SEQ ID NO: 16) or Fas2 (SEQ ID NO: 18) such as a mutant Fas1 having a mutation at position 123, preferably an L123V mutation, or a mutant Fas2 having a mutation at position 1220, preferably an I1220F or an I1220W mutation.
23. The yeast cell according to any one of the preceding items, wherein the yeast cell further expresses a thioesterase such as a heterologous thioesterase, optionally wherein the thioesterase is expressed at high level.
24. The yeast cell according to item 23, wherein the thioesterase has at least 60% homology or identity to the thioesterase from Cuphea palustris as set forth in SEQ ID NO: 33, to the thioesterase from Cuphea hookeriana as set forth in SEQ ID NO: 57, to the thioesterase from Cinnamomum camphora as set forth in SEQ ID NO: 35, or to the thioesterase from Escherichia coli as set forth in SEQ ID NO: 26, preferably the thioesterase has at least 60% homology or identity to the thioesterase from Cinnamomum camphora as set forth in SEQ ID NO: 35, or to the thioesterase from Escherichia coli as set forth in SEQ ID NO: 26.
25. The yeast cell according to any one of the preceding items, wherein the yeast cell further expresses a fusion protein of a truncated fatty acyl synthase and of a truncated thioesterase, such as the fusion protein as set forth in SEQ ID NO: 59 or a homologue thereof having at least 60% homology or identity thereto.
26. The yeast cell according to any one of the preceding items, wherein the yeast cell comprises a nucleic acid encoding said heterologous desaturase and a nucleic acid encoding said heterologous fatty acyl-CoA reductase.
27. The yeast cell according to item 26, wherein the nucleic acid encoding said heterologous desaturase and/or the nucleic acid encoding said heterologous fatty acyl-CoA reductase are present in a high copy number.
28. The yeast cell according to any one of items 26 to 27, wherein the nucleic acid encoding said heterologous desaturase is as set forth in SEQ ID NO: 1 or a homologue thereof having at least 60% homology or identity thereto, or as set forth in SEQ ID NO: 78 or a homologue thereof having at least 60% homology or identity thereto.
29. The yeast cell according to any one of items 26 to 28, wherein the nucleic acid encoding said heterologous fatty acyl-CoA reductase is as set forth in SEQ ID NO: 9 or a homologue thereof having at least 60% homology or identity thereto.
30. The yeast cell according to any one of the preceding items, wherein the yeast cell comprises a nucleic acid encoding said heterologous cytochrome b5, a nucleic acid encoding said heterologous cytochrome b5 reductase, a nucleic acid encoding said hemoglobin, a nucleic acid encoding said fatty acid synthase variant, a nucleic acid encoding said thioesterase, and/or a nucleic acid encoding said fusion protein.
31. The yeast cell according to item 30, wherein the nucleic acid encoding said heterologous cytochrome b5, the nucleic acid encoding said heterologous cytochrome b5 reductase, the nucleic acid encoding said hemoglobin, the nucleic acid encoding said fatty acid synthase variant, and/or the nucleic acid encoding said thioesterase are present in high copy number.
32. The yeast cell according to any one of the preceding items, wherein the nucleic acid encoding said heterologous desaturase, the nucleic acid encoding said heterologous fatty acyl-CoA reductase, the nucleic acid encoding said heterologous cytochrome b5, the nucleic acid encoding said heterologous cytochrome b5 reductase, the nucleic acid encoding said hemoglobin, the nucleic acid encoding said fatty acid synthase variant, and/or the nucleic acid encoding said thioesterase are codon-optimised for expression in the yeast cell.
33. The yeast cell according to any one of items 30 to 32, wherein the nucleic acid encoding said heterologous cytochrome b5 is as set forth in SEQ ID NO: 3 or a homologue thereof having at least 60% homology or identity thereto, the nucleic acid encoding said heterologous cytochrome b5 reductase is as set forth in SEQ ID NO: 23 or a homologue thereof having at least 60% homology or identity thereto, the nucleic acid encoding said hemoglobin is as set forth in SEQ ID NO: 5 or a homologue thereof having at least 60% homology or identity thereto, and/or the nucleic acid encoding said thioesterase is as set forth in SEQ ID NO: 25 or SEQ ID NO: 34 or a homologue of SEQ ID NO: 25 or SEQ ID NO: 34 having at least 60% homology or identity thereto.
34. The yeast cell according to any one of the preceding items, wherein the yeast cell is capable of producing E8,E10-dodecadien-1-ol with a titer of at least 0.5 mg/L, such as at least 0.6 mg/L, such as at least 0.7 mg/L, such as at least 0.8 mg/L, such as at least 0.9 mg/L, such as at least 1 mg/L, such as at least 1.5 mg/L, such as at least 2.5 mg/L, such as at least 5.0 mg/L, such as at least 10 mg/L, such as at least 15 mg/L, such as at least 20 mg/L, such as 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L or more.
35. The yeast cell according to any one of the preceding items, wherein the yeast cell further expresses an acetyltransferase (EC 2.3.1.84) capable of converting at least part of the E8,E10-dodecadien-1-ol into E8,E10-dodecadienyl acetate, whereby the yeast cell is capable of producing E8,E10-dodecadienyl acetate.
36. The yeast cell according to item 35, wherein the acetyltransferase is a heterologous acetyltransferase (AcT) expressed from said yeast cell or a native acetyltransferase overexpressed from said yeast cell.
37. The yeast cell according to any one of items 35 or 36, wherein the acetyltransferase is Sc_Atf1 (SEQ ID NO: 37) or a variant thereof having at least 60% homology or identity thereto, such as at least 61% homology or identity, such as at least 62% homology or identity, such as at least 63% homology or identity, such as at least 64% homology or identity, such as at least 65% homology or identity, such as at least 66% homology or identity, such as at least 67% homology or identity, such as at least 68% homology or identity, such as at least 69% homology or identity, such as at least 70% homology or identity, such as at least 71% homology or identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to Sc_Atf1 (SEQ ID NO: 37).
38. The yeast cell according to any one of the preceding items, wherein the yeast cell further expresses an aldehyde-forming fatty acyl-CoA reductase (EC 1.2.1.50), an alcohol dehydrogenase (EC 1.1.1.2) and/or a fatty alcohol oxidase (EC 1.1.3.20) capable of converting at least part of the E8,E10-dodecadien-1-ol into E8,E10-dodecadienal.
39. The yeast cell according to any one of the preceding items, wherein the yeast cell further:
   i) has one or more mutations resulting in reduced activity of one or more native acyl-CoA oxidases; and
   ii) expresses at least one group of enzymes comprising at least one acyl-CoA oxidase capable of oxidising a fatty acyl-CoA, wherein the group of enzymes is capable of shortening a fatty acyl-CoA of a first carbon chain length X to a shortened fatty acyl-CoA having a second carbon chain length X′, wherein X′≤X-2.
40. The yeast cell according to item 39, wherein X′=12.
41. The yeast cell according to any one of items 39 to 40, wherein the yeast cell further expresses a desaturase capable of introducing at least one double bond in the fatty acyl-CoA of carbon chain length X, such as CroZ11 desaturase (SEQ ID NO: 63) or CpaE11 desaturase (SEQ ID NO: 65) or a functional variant thereof having at least 65% homology or identity, such as at least 70% homology or identity, such as at least 71% homology or identity, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to SEQ ID NO: 63, SEQ ID NO: 65.
42. The yeast cell according to any one of items 39 to 42, wherein the native acyl-CoA oxidase of i) and/or the acyl-CoA oxidase of ii) is a peroxisomal acyl-CoA oxidase.
43. The yeast cell according to any one of items 39 to 41, wherein the at least one acyl-CoA oxidase of ii) is a native acyl-CoA oxidase or a heterologous acyl-CoA oxidase, which is optionally overexpressed compared to a reference yeast strain not expressing said at least one group of enzymes, preferably the at least one acyl-CoA oxidase of the group of enzymes of ii) is a heterologous acyl-CoA oxidase.
44. The yeast cell according to any one of items 39 to 43, wherein the group of enzymes of ii) comprises an acyl-CoA oxidase derived from an organism of a genus selected from Yarrowia, Agrotis, Arabidopsis, Aspergillus, Cucurbita, Homo, Paenarthrobacter and Rattus preferably the at least one first group of enzymes comprises an acyl-CoA oxidase derived from Yarrowia lipolytica, Agrotis segetum, Arabidopsis thaliana, Aspergillus nidulans, Cucurbita maxima, Homo sapiens, Paenarthrobacter ureafaciens or Rattus norvegicus, preferably the at least one acyl-CoA oxidase of the first group of enzymes is an acyl-CoA oxidase selected from the group consisting of Yli_POX1 (XP_504703), Yli_POX2 (XP_505264), Yli_POX3 (XP_503244), Yli_POX4 (XP_504475), Yli_POX5 (XP_502199), Yli_POX6 (XP_503632), Ase_POX (SEQ ID NO: 39), Ath_POX1 (SEQ ID NO: 41), Ath_POX2 (SEQ ID NO: 43), Ani_POX (SEQ ID NO: 45), Cma_POX (SEQ ID NO: 47), Hsa_POX1-2 (SEQ ID NO: 49), Pur_POX (SEQ ID NO: 51), Sc_POX1 (SEQ ID NO: 31) and Rno_POX2 (SEQ ID NO: 53), or a functional variant thereof having at least 60% homology or identity thereto, such as at least 65%, such as at least 70%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.
45. A method for producing E8,E10-dodecadienyl coenzyme A and optionally E8,E10-dodecadien-1-ol in a yeast cell, said method comprising the steps of providing a yeast cell and incubating said yeast cell in a medium, wherein the yeast cell expresses:
   i) At least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
   ii) Optionally at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol, thereby producing E8,E10-dodecadienyl coenzyme A and optionally E8,E10-dodecadien-1-ol.
46. The method according to item 45, wherein the yeast cell is as defined in any one of items 1 to 44.
47. The method according to any one of items 45 to 46, further comprising the step of converting the E8,E10-dodecadienyl coenzyme A into a lipid such as a triacylglyceride or into a free fatty acid, recovering said lipid or free fatty acid and converting said lipid or free fatty acid to E8,E10-dodecadien-1-ol.
48. The method according to any one of items 45 to 47, further comprising the step of recovering said E8,E10-dodecadien-1-ol.
49. The method according to any one of items 45 to 48, further comprising the step of converting at least part of the E8,E10-dodecadien-1-ol into E8,E10-dodecadienyl acetate by expression of an acetyltransferase or by chemical conversion.
50. The method according to item 49, wherein the acetyltransferase is a heterologous acetyltransferase (EC 2.3.1.84) expressed from said yeast cell or a native acetyltransferase overexpressed from said yeast cell, wherein said acetyltransferase is capable of converting at least part of the E8,E10-dodecadien-1-ol into E8,E10-dodecadienyl acetate, thereby further producing E8,E10-dodecadienyl acetate.
51. The method according to item 50, wherein the acetyltransferase is Sc_Atf1 (SEQ ID NO: 37) or a variant thereof having at least 75% homology or identity, such as at least 80% homology or identity, such as at least 85% homology or identity, such as at least 90% homology or identity, such as at least 91% homology or identity, such as at least 92% homology or identity, such as at least 93% homology or identity, such as at least 94% homology or identity, such as at least 95% homology or identity, such as at least 96% homology or identity, such as at least 97% homology or identity, such as at least 98% homology or identity, such as at least 99% homology or identity, such as 100% homology or identity to Sc_Atf1 (SEQ ID NO: 37).
52. The method according to any one items 45 to 51, further comprising the step of recovering said E8,E10-dodecadienyl acetate.
53. The method according to any one items 45 to 52, further comprising the step of converting at least part of the E8,E10-dodecadien-1-ol into E8,E10-dodecadienal by expression of an aldehyde-forming fatty acyl-CoA reductase (EC 1.2.1.50), an alcohol dehydrogenase (EC 1.1.1.2) and/or a fatty alcohol oxidase (EC 1.1.3.20) capable of converting at least part of the E8,E10-dodecadien-1-ol into E8,E10-dodecadienal, or by chemical conversion, thereby further producing E8,E10-dodecadienal.
54. The method according to item 53, further comprising the step of recovering said E8,E10-dodecadienal.
55. The method according to any one of items 45 to 54, wherein the medium comprises an extractant in an amount equal to or greater than its cloud concentration in an aqueous solution, wherein the extractant a non-ionic ethoxylated surfactant such as an antifoaming agent, preferably a polyethoxylated surfactant selected from: a polyethylene polypropylene glycol, a mixture of polyether dispersions, an antifoaming agent comprising polyethylene glycol monostearate such as simethicone, fatty alcohol alkoxylates, polyethoxylated surfactants and ethoxylated and propoxylated C₁₆-C₁₈ alcohol-based antifoaming agents and combinations thereof.
56. The method according to item 55, wherein:
   the non-ionic ethoxylated surfactant is an ethoxylated and propoxylated C₁₆-C₁₈ alcohol-based antifoaming agent, such as C₁₆-C₁₈ alkyl alcohol ethoxylate propoxylate (CAS number 68002-96-0), and wherein the culture medium comprises at least 1% vol/vol of C₁₆-C₁₈ alkyl alcohol ethoxylate propoxylate, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol C₁₆-C₁₈ alkyl alcohol ethoxylate propoxylate, or more,
   the non-ionic ethoxylated surfactant is a polyethylene polypropylene glycol, for example Kollliphor® P407 (CAS number 9003-11-6), and wherein the culture medium comprises at least 10% vol/vol of polyethylene polypropylene glycol such as Kolliphor® P407, such as at least 11% vol/vol, such as at least 12% vol/vol, such as at least 13% vol/vol, such as at least 14% vol/vol, such as at least 15% vol/vol, such as at least 16% vol/vol, such as at least 17% vol/vol, such as at least 18% vol/vol, such as at least 19% vol/vol, such as at least 20% vol/vol, such as at least 25% vol/vol, such as at least 30% vol/vol, such as at least 35% vol/vol of polyethylene polypropylene glycol such as Kolliphor® P407, or more,
   the non-ionic ethoxylated surfactant is a mixture of polyether dispersions, such as antifoam 204, and wherein the culture medium comprises at least 1% vol/vol of a mixture of polyether dispersions such as antifoam 204, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol of a mixture of polyether dispersions such as antifoam 204, or more; and/or
   the non-ionic ethoxylated surfactant is a non-ionic ethoxylated surfactant comprising polyethylene glycol monostearate such as simethicone, and wherein the culture medium comprises at least 1% vol/vol of polyethylene glycol monostearate or simethicone, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol polyethylene glycol monostearate or simethicone, or more;
   the non-ionic ethoxylated surfactant is a fatty alcohol alkoxylate, preferably selected from Plurafac® LF300 (CAS number 196823-11-7), Plurafac® LF1300 (68002-96-0), Plurafac® SLF180 (CAS number 196823-11-7), Dehypon® 2574 (CAS number 68154-97-2), and Imbentin SG/251 (CAS number 68002-96-0), preferably Plurafac® LF300 or Dehypon® 2574, and wherein the culture medium comprises at least 1% vol/vol of fatty alcohol alkoxylate, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol fatty alcohol alkoxylate or more;
   the non-ionic ethoxylated surfactant is Agnique BP420 (CAS number 68002-96-0), and wherein the culture medium comprises at least 1% vol/vol of Agnique BP420, such as at least 1.5%, such as at least 2%, such as at least 2.5%, such as at least 3%, such as at least 3.5%, such as at least 4%, such as at least 5%, such as at least 6%, such as at least 7%, such as at least 8%, such as at least 9%, such as at least 10%, such as at least 12.5%, such as at least 15%, such as at least 17.5%, such as at least 20%, such as at least 22.5%, such as at least 25%, such as at least 27.5%, such as at least 30% vol/vol Agnique BP420 or more.
57. The method according to any one of items 45 to 56, wherein the culture medium comprises the extractant in an amount greater than its cloud concentration by at least 50%, such as at least 100%, such as at least 150%, such as at least 200%, such as at least 250%, such as at least 300%, such as at least 350%, such as at least 400%, such as at least 500%, such as at least 750%, such as at least 1000%, or more, and/or wherein the culture medium comprises the extractant in an amount at least 2-fold its cloud concentration, such as at least 3-fold its cloud concentration, such as at least 4-fold its cloud concentration, such as at least 5-fold its cloud concentration, such as at least 6-fold its cloud concentration, such as at least 7-fold its cloud concentration, such as at least 8-fold its cloud concentration, such as at least 9-fold its cloud concentration, such as at least 10-fold its cloud concentration, such as at least 12.5-fold its cloud concentration, such as at least 15-fold its cloud concentration, such as at least 17.5-fold its cloud concentration, such as at least 20-fold its cloud concentration, such as at least 25-fold its cloud concentration, such as at least 30-fold its cloud concentration.
58. The method according to any one of items 45 to 57, wherein the E8,E10-dodecadienyl coenzyme A is converted into a lipid or a free fatty acid, and wherein said lipid or free fatty acid, said E8,E10-dodecadien-1-ol, and optionally said E8,E10-dodecadienyl acetate and/or said E8,E10-dodecadienal produced by the yeast cell is present in an emulsion in the fermentation broth, the method further comprising a step of breaking said emulsion, thereby obtaining a composition comprising a product phase comprising the extractant and the lipid or free fatty acid, the E8,E10-dodecadien-1-ol, and optionally the E8,E10-dodecadienyl acetate and/or the E8,E10-dodecadienal, optionally wherein:
   the step of breaking the emulsion comprises or consists of a step of phase separation, such as a step of centrifugation, of the fermentation broth, thereby obtaining a composition consisting of three phases: a water phase, a phase comprising cells and cellular debris, and the product phase comprising the extractant and the lipid or free fatty acid, E8,E10-dodecadien-1-ol, and optionally the E8,E10-dodecadienyl acetate and/or the E8,E10-dodecadienal, and/or
   wherein the product phase comprises at least 50% of the lipid or free fatty acid, E8,E10-dodecadien-1-ol, and optionally of the E8,E10-dodecadienyl acetate and/or of the E8,E10-dodecadienal initially present in the fermentation broth, such as at least 55%, such as at least 60%, such as at least 65%, such as at least 70%, such as at least 75%, such as at least 80%, such as at least 85%, such as at least 90%, such as at least 95% or more.
59. The method according to any one of items 45 to 58, further comprising the steps of:
   recovering the lipid or free fatty acid, the E8,E10-dodecadien-1-ol, and optionally the E8,E10-dodecadienyl acetate and/or the E8,E10-dodecadienal preferably by a distillation step such as a distillation under reduced pressure, or by a column purification,
   chemically converting at least part of the E8,E10-dodecadien-1-ol to E8,E10-dodecadienal and/or to E8,E10-dodecadienyl acetate,
   optionally, recovering said E8,E10-dodecadienal and/or to E8,E10-dodecadienyl acetate.
60. The method according to any one of items 45 to 59, further comprising the step of formulating the recovered E8,E10-dodecadien-1-ol, the E8,E10-dodecadienyl acetate and/or the E8,E10-dodecadienal into a pheromone composition.
61. The method according to any one of items 45 to 60, wherein the pheromone composition further comprises one or more additional compounds such as a liquid or solid carrier or substrate.
62. A nucleic acid construct for modifying a yeast cell, said construct comprising:
   i) At least one first polynucleotide encoding at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); and
   ii) Optionally a second polynucleotide encoding at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol.
63. The nucleic acid construct according to item 62, wherein:
   a) the at least one desaturase is Gmo_CPRQ (SEQ ID NO: 77), Cpo_CPRQ (SEQ ID NO: 2), or a functional variant thereof having at least 80% identity thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to SEQ ID NO: 77 or SEQ ID NO: 2, preferably the at least one desaturase is Cpo_CPRQ or a functional variant thereof; or
   b) the at least one desaturase is at least two desaturases, wherein at least one of said two desaturases is Gmo_CPRQ (SEQ ID NO: 77), Cpo_CPRQ (SEQ ID NO: 2), or a functional variant thereof having at least 80% identity thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to SEQ ID NO: 2, preferably the at least one desaturase is Cpo_CPRQ or a functional variant thereof, and the other desaturase is a desaturase capable of introducing at least one double bond in a fatty acyl-CoA having a carbon chain length of 12, such as a Z9-12 desaturase.
64. The nucleic acid construct according to any one of items 62 to 63, wherein the at least one heterologous desaturase is at least two desaturases, and wherein the other desaturase is selected from Cpo_NPVE (SEQ ID NO: 67), Cpo_SPTQ (SEQ ID NO: 69) and functional variants thereof having at least 60% homology or identity thereto, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to SEQ ID NO: 67 or SEQ ID NO: 69.
65. The nucleic acid construct according to any one of items 62 to 64, wherein the first polynucleotide comprises SEQ ID NO: 1 or SEQ ID NO: 78, preferably SEQ ID NO: 1, or a homologue thereof having at least 60% homology or identity thereto, such as 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity to SEQ ID NO: 1 or SEQ ID NO: 78, preferably to SEQ ID NO: 1.
66. The nucleic acid construct according to any one of items 62 to 65, wherein the at least one heterologous desaturase is at least two heterologous desaturases, and wherein the first polynucleotide further comprises a nucleic acid as set forth in SEQ ID NO: 66 or SEQ ID NO: 68, or a homologue thereof having at least 60% homology or identity thereto, such as 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.
67. The nucleic acid construct according to any one of items 62 to 66, wherein the heterologous desaturase is as defined in any one of items 1 to 44.
68. The nucleic acid construct according to any one of items 62 to 67, wherein the at least one desaturase is a mutant of Cpo_CPRQ having a mutation at position 85, such as an S85A mutation.
69. The nucleic acid construct according to any one of items 62 to 68, wherein the heterologous fatty acyl-CoA reductase is as defined in any one of items 1 to 44.
70. The nucleic acid construct according to any one of items 62 to 69, wherein the second polynucleotide comprises or consists of SEQ ID NO: 9, SEQ ID NO: 60, SEQ ID NO: 70, SEQ ID NO: 72, SEQ ID NO: 74, SEQ ID NO: 11, SEQ ID NO: 76 and homologues thereof having at least 60% homology or identity thereto, such as at least 65%, such as at least 70%, such as at least 71%, such as at least 72%, such as at least 73%, such as at least 74%, such as at least 75%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% homology or identity thereto.
71. The nucleic acid construct according to any one of items 62 to 70, further comprising one or more of:
iii) a polynucleotide encoding a heterologous cytochrome b5, such as the polynucleotide as set forth in SEQ ID NO: 3 or a homologue thereof having at least 60% homology or identity thereto;
iv) a polynucleotide encoding a heterologous cytochrome b5 reductase, such as the polynucleotide as set forth in SEQ ID NO: 23 or a homologue thereof having at least 60% homology or identity thereto;
v) a polynucleotide encoding a hemoglobin, such as the polynucleotide as set forth in SEQ ID NO: 5 or a homologue thereof having at least 60% homology or identity thereto;
vi) a polynucleotide encoding a fatty acyl synthase variant having a modified ketone synthase domain; and/or
vii) a polynucleotide encoding a thioesterase, such as the polynucleotide as set forth in SEQ ID NO: 25 or SEQ ID NO: 34 or a homologue thereof having at least 60% homology or identity thereto.
72. The nucleic acid construct according to any one of items 62 to 71, wherein the heterologous cytochrome b5, the heterologous cytochrome b5 reductase, the hemoglobin, the fatty acyl synthase variant and/or the thioesterase are as defined in any one of items 1 to 44.
73. A method of monitoring the presence of pest or disrupting the mating of pest, said method comprising the steps of:
i) Producing E8,E10-dodecadien-1-ol and optionally E8,E10-dodecadienyl acetate and/or E8,E10-dodecadienal by the method according to any one of items 45 to 61;
ii) Formulating said E8,E10-dodecadien-1-ol and optionally said E8,E10-dodecadienyl acetate and/or said E8,E10-dodecadienal as a pheromone composition; and
iii) Employing said pheromone composition as an integrated pest management composition.
74. E8,E10-dodecadienyl coenzyme A, E8,E10-dodecadien-1-ol, E8,E10-dodecadienyl acetate and/or E8,E10-dodecadienal obtainable by the method according to any one of items 45 to 61.
75. A kit of parts comprising instructions for use and:
a) the yeast cell according to any one of items 1 to 44; and/or
b) the nucleic acid construct according to any one of items 62 to 72 for modifying a yeast cell and optionally a yeast cell to be modified, wherein upon expression of the polynucleotides comprised within the nucleic acid construct, the modified yeast cell is capable of producing E8,E10-dodecadienyl coenzyme A and optionally E8,E10-dodecadien-1-ol.

1. A yeast cell capable of producing E8,E10-dodecadienyl coenzyme A, said yeast cell expressing at least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA), wherein:
a) the at least one desaturase is Cpo_CPRQ (SEQ ID NO: 2), or a functional variant thereof having at least 80% identity thereto; or
b) the at least one desaturase is at least two desaturases, wherein at least one of said two desaturases is Cpo_CPRQ (SEQ ID NO: 2), or a functional variant thereof having at least 80% identity thereto, and the other desaturase is a desaturase capable of introducing at least one double bond in a fatty acyl-CoA having a carbon chain length of 12.

2. The yeast cell according to claim 1, wherein the at least one desaturase is at least two desaturases, and wherein the other desaturase is selected from Cpo_NPVE (SEQ ID NO: 67), Cpo_SPTQ (SEQ ID NO: 69) and functional variants thereof having at least 80% identity thereto.

3. The yeast cell according to claim 1, wherein the yeast cell belongs to a genus selected from the group consisting of Blakeslea, Candida, Cryptococcus, Cunninghamella, Lipomyces, Mortierella, Mucor, Phycomyces, Pythium, Rhodosporidium, Rhodotorula, Trichosporon, Saccharomyces and Yarrowia.

4. The yeast cell according to claim 1, wherein the yeast cell is further capable of producing E8,E10-dodecadien-1-ol, said yeast cell further expressing at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol.

5. The yeast cell according to claim 1, wherein the at least one desaturase is a mutant of Cpo_CPRQ having a mutation at position 85, and/or wherein the at least one heterologous desaturase is at least two different heterologous desaturases.

6. The yeast cell according to claim 4, wherein the fatty acyl-CoA reductase is selected from the group consisting of Ase_FAR (SEQ ID NO: 10), Aip_FAR (SEQ ID NO: 61), Hs_FAR (SEQ ID NO: 71), Has_FAR (SEQ ID NO: 73), Hv_FAR (SEQ ID NO: 75), Har_FAR (SEQ ID NO: 12), Cpo_FAR (SEQ ID NO: 76) and functional variants thereof having at least 80% identity thereto.

7. The yeast cell according to claim 1, further having one or more of the following:
expressing a heterologous cytochrome b5;
expressing a heterologous cytochrome b5 reductase (EC 1.6.2.2);
expressing a hemoglobin;
comprising a mutation of one or more genes encoding an elongase and resulting in a partial or total loss of elongase activity;
comprising a mutation of one or more genes encoding a thioesterase and resulting in a partial or total loss of thioesterase activity;
comprising at least one mutation resulting in reduced activity of at least one of Hfd1, Hfd2, Hfd3, Hfd4, Fao1, GPAT and Pex10, or having a mutation resulting in reduced activity of at least one protein having at least 80% identity thereto;
expressing a fatty acyl synthase variant having a modified ketone synthase domain, wherein said fatty acyl synthase variant is a variant of Fas1 (SEQ ID NO: 16) or Fas2 (SEQ ID NO: 18);
expressing a thioesterase; or
expressing a fusion protein of a truncated fatty acyl synthase and of a truncated thioesterase.

8. (canceled)

9. The yeast cell according to claim 4, wherein the yeast cell is capable of producing E8,E10-dodecadien-1-ol with a titer of at least 0.5 mg/L.

10. The yeast cell according to claim 4, wherein the yeast cell further expresses an acetyltransferase (EC 2.3.1.84) capable of converting at least part of the E8,E10-dodecadien-1-ol into E8,E10-dodecadienyl acetate, whereby the yeast cell is capable of producing E8,E10-dodecadienyl acetate.

11. The yeast cell according to claim 4, wherein the yeast cell further expresses an aldehyde-forming fatty acyl-CoA reductase (EC 1.2.1.50), an alcohol dehydrogenase (EC 1.1.1.2) and/or a fatty alcohol oxidase (EC 1.1.3.20) capable of converting at least part of the E8,E10-dodecadien-1-ol into E8,E10-dodecadienal.

12. The yeast cell according to claim 1, wherein the yeast cell further:
i) has one or more mutations resulting in reduced activity of one or more native acyl-CoA oxidases; and
ii) expresses at least one group of enzymes comprising at least one acyl-CoA oxidase capable of oxidising a fatty acyl-CoA, wherein the group of enzymes is capable of shortening a fatty acyl-CoA of a first carbon chain length X to a shortened fatty acyl-CoA having a second carbon chain length X′, wherein X′≤X-2.

13. The yeast cell according to claim 11, further expressing a desaturase capable of introducing at least one double bond in the fatty acyl-CoA of carbon chain length X.

14. A method for producing E8,E10-dodecadienyl coenzyme A, said method comprising the steps of providing a yeast cell and incubating said yeast cell in a medium, wherein the yeast cell expresses:
i) At least one heterologous desaturase capable of introducing one or more double bonds in a fatty acyl-CoA having a carbon chain length of 12, thereby converting said fatty acyl-CoA to a desaturated fatty acyl-CoA, wherein at least part of said desaturated fatty acyl-CoA is E8,E10-dodecadienyl coenzyme A (E8,E10-C12:CoA); wherein:
a) the at least one desaturase is Cpo_CPRQ (SEQ ID NO: 2), or a functional variant thereof having at least 80% identity thereto; or
b) the at least one desaturase is at least two desaturases, wherein at least one of said two desaturases is Cpo_CPRQ (SEQ ID NO: 2), or a functional variant thereof having at least 80% identity thereto, and the other desaturase is a desaturase capable of introducing at least one double bond in a fatty acyl-CoA having a carbon chain length of 12;
thereby producing E8,E10-dodecadienyl coenzyme A.

15. The method according to claim 13, wherein the method further comprises the steps of converting the E8,E10-dodecadienyl coenzyme A into a lipid or into a free fatty acid, recovering said lipid or free fatty acid and converting said lipid or free fatty acid to E8,E10-dodecadien-1-ol.

16. The method according to claim 26, wherein the method further comprises the step of recovering said E8,E10-dodecadien-1-ol

17. The method according to claim 26, further comprising the step of
converting at least part of the E8,E10-dodecadien-1-ol into E8,E10-dodecadienyl acetate by expression of an acetyltransferase or by chemical conversion, thereby further producing E8,E10-dodecadienyl acetate.

18. The method according to claim 26, further comprising the step of converting at least part of the E8,E10-dodecadien-1-ol into E8,E10-dodecadienal by expression of an aldehyde-forming fatty acyl-CoA reductase (EC 1.2.1.50), an alcohol dehydrogenase (EC 1.1.1.2) and/or a fatty alcohol oxidase (EC 1.1.3.20) capable of converting at least part of the E8,E10-dodecadien-1-ol into E8,E10-dodecadienal, or by chemical conversion, thereby further producing E8,E10-dodecadienal.

19. The method according to claim 26, wherein the medium comprises an extractant in an amount equal to or greater than its cloud concentration in an aqueous solution, wherein the extractant is a non-ionic ethoxylated surfactant.

20. The method according to claim 19, wherein the medium comprises the extractant in an amount greater than its cloud concentration by at least 50% and/or wherein the culture medium comprises the extractant in an amount at least 2-fold its cloud concentration, wherein the cloud concentration is measured in the medium.

21. The method according to claim 19, further comprising the step of converting the E8,E10-dodecadienyl coenzyme A into a lipid or a free fatty acid, and wherein said lipid or free fatty acid and/or said E8,E10-dodecadien-1-ol produced by the yeast cell is present in an emulsion in the fermentation broth, the method further comprising a step of breaking said emulsion, thereby obtaining a composition comprising a product phase comprising the extractant and the lipid or free fatty acid and/or the E8,E10-dodecadien-1-ol.

22. The method according to claim 21, wherein the step of breaking the emulsion comprises or consists of a step of phase separation of the fermentation broth, thereby obtaining a composition consisting of three phases: a water phase, a phase comprising cells and cellular debris, and the product phase comprising the extractant and the lipid or free fatty acid and/or E8,E10-dodecadien-1-ol.

23. The method according to claim 21, wherein the product phase comprises at least 50% of the lipid or free fatty acid and/or E8,E10-dodecadien-1-ol initially present in the fermentation broth.

24. The method according to claim 21, further comprising the steps of:
recovering the lipid or free fatty acid and/or the E8,E10-dodecadien-1-ol,
chemically converting at least part of the E8,E10-dodecadien-1-ol to E8,E10-dodecadienal and/or to E8,E10-dodecadienyl acetate

25. The method according to claim 24, further comprising the steps of
recovering said E8,E10-dodecadienal and/or E8,E10-dodecadienyl acetate, and
formulating the recovered E8,E10-dodecadien-1-ol, the E8,E10-dodecadienyl acetate and/or the E8,E10-dodecadienal into a pheromone composition.

26. The method according to claim 13, wherein the yeast cell expresses at least one heterologous fatty acyl-CoA reductase (EC 1.2.1.84) capable of converting at least part of said desaturated fatty acyl-CoA to a desaturated fatty alcohol, wherein the fatty acyl-CoA reductase is capable of converting at least part of said E8,E10-dodecadienyl coenzyme A (E8,E10-C12:CoA) to E8,E10-dodecadien-1-ol, thereby producing E8,E10-dodecadien-1-ol.
